(19) United States
(12) Patent Application Publication
(10) Pub. No.: US 2026/0317333 A1
(43) Pub. Date: Oct. 15, 2026

(54) Heteroaryl Compounds as Ligand Directed Degraders of IRAK4

(71) Applicant: Celgene Corporation, Princeton, NJ (US)

(72) Inventors: Geraint Davies, Arlington, MA (US); Paul E. Gormisky, Belmont, MA (US); Timothy Gordon Rasmusson, Sudbury, MA (US)

(73) Assignee: Celgene Corporation, Princeton, NJ (US)

(21) Appl. No.: 19/481,287

(22) PCT Filed: May 9, 2024
(86) PCT No.: PCT/US2024/028512

§ 371 Date: Nov. 4, 2025

Related U.S. Application Data
(60) Provisional application No. 63/465,602, filed on May 11, 2023.

(51) Int. Cl. C07D 487/04; A61K 31/519; C07D 519/00

(52) U.S. Cl. CPC C07D 487/04; A61K 31/519; C07D 519/00

(57) ABSTRACT

Provided herein are compounds and compositions thereof for modulating IRAK4. In some embodiments, the compounds and compositions are provided for treatment of inflammatory or autoimmune diseases.

### CROSS-REFERENCE TO RELATED APPLICATIONS

This application claims priority to U.S. Provisional Application No. 63/465,602, filed on May 11, 2023, which is incorporated herein by reference in its entirety for any purpose.

### FIELD

The present disclosure relates generally to compounds, compositions, and methods for their preparation and use of the compounds and compositions for treating inflammatory or autoimmune diseases.

### BACKGROUND

The recruitment of immune cells to sites of injury involves the concerted interactions of a large number of soluble mediators. Several cytokines appear to play key roles in these processes, including interleukin-1 (IL-1). IL-1 produces proinflammatory responses and contributes to the tissue degeneration observed in chronic inflammatory conditions. IL-1 has also been implicated in the process of bone resorption and adipose tissue regulation. Thus, IL-1 plays a key role in a large number of pathological conditions including rheumatoid arthritis, inflammatory bowel disease, multiple sclerosis, diabetes, obesity, cancer, and sepsis.

IL-1 treatment of cells induces the formation of a complex consisting of the two IL-1 receptor chains, IL-1R1 and IL-1RAcP, and the resulting heterodimer recruits an adaptor molecule designated as MyD88, which binds to IL-1 receptor associated kinase (IRAK) (Wesche et al., J. Biol. Chem. 1999, 274, 19403-19410; O'Neill et al., J. Leukoc. Biol. 1998, 63, 650-657; Auron, Cytokine Growth Factor Rev. 1998, 9:221-237; and O'Neill, Biochem. Soc. Trans. 2000, 28, 557-563). Four members of the IRAK family have been identified: IRAK1, IRAK2, IRAK3, and IRAK4. These proteins are characterized by a typical N-terminal death domain that mediates interaction with MyD88-family adaptor proteins and a centrally located kinase domain. Of the four members in the mammalian IRAK family, IRAK-4 is considered to be the “master IRAK.” IRAK-4 is a serine/threonine kinase that plays an essential role in signal transduction by Toll/IL-1 receptors (TIRs). Under overexpression conditions, all IRAKs can mediate the activation of nuclear factor-kappa B and stress-induced mitogen activated protein kinase (MAPK)-signaling cascades. Studies have shown that IRAK4 kinase activity is essential for cytokine production, activation of MAPKs, and induction of NF-kappa B regulated genes in response to TLR ligands (Koziczak-Holbro M. et al., J. Biol. Chem. 2007, 282, 13552-13560). Given the central role of IRAK4 in Toll-like/IL-1R signaling and immunological protection, compounds that modulate the function of IRAK4 may be useful in treating inflammatory, cell proliferative, and immune-related conditions and diseases associated with IRAK-mediated signal transduction such as rheumatoid arthritis, inflammatory bowel disease, multiple sclerosis, diabetes, obesity, allergic disease, psoriasis, asthma, graft rejection, cancer and sepsis.

Protein degradation is a highly regulated and essential process that maintains cellular homeostasis. Selective identification and removal of damaged, misfolded, or excess proteins is achieved through the ubiquitin-proteasome pathway (UPP). The UPP is central to the regulation of almost all cellular processes. Ubiquitination of the protein is accomplished by an E3 ubiquitin ligase that binds to a protein and adds ubiquitin molecules to the protein, thus marking the protein for proteasome degradation.

Harnessing the UPP for therapeutic use has received significant interest (Zhou et al., Mol. Cell 2000, 6, 751-756). One promising therapy uses proteolysis targeting chimeras, commonly referred to as PROTACs, to effect removal of unwanted proteins by protein degradation (Scheepstra et al., Comp. Struct. Biotech. J. 2019, 17, 160-176). PROTACS are ligand directed degraders that bring together an E3 ligase and a target protein that is to be degraded. These bivalent molecules usually consist of an E3 ligase ligand connected through a linker moiety to small molecule that binds to the target protein. A PROTAC positions the E3 ligase at the appropriate distance and orientation to the target protein, allowing the latter to be ubiquitinated. The ubiquitinated target protein is subsequently recognized by the proteasome, where it is degraded.

Accordingly, in one aspect, provided herein are compounds that target IRAK4 for degradation.

### SUMMARY

Described herein, in certain embodiments, are compounds and compositions thereof for degrading IRAK4. In various embodiments, the compounds and compositions thereof may be used in treatment of inflammatory or autoimmune diseases.

The present embodiments can be understood more fully by reference to the detailed description and examples, which are intended to exemplify non-limiting embodiments.

Embodiment 1 is a compound of Formula (I):

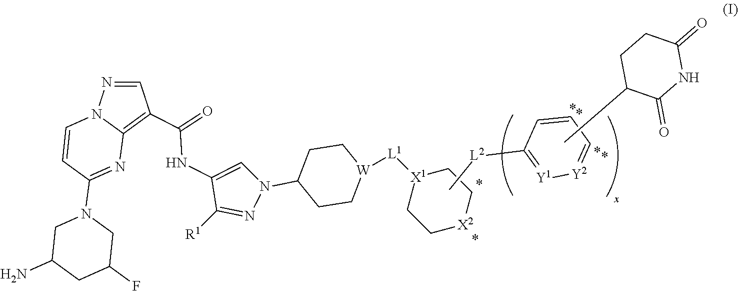

or a pharmaceutically acceptable salt thereof, wherein:
R¹ is C₁-C₆ haloalkyl;
W is CH or N;
X¹ is CH or N;
X² is CH₂, CH, C, N, or NH;
Y¹ and Y² are independently CH or N;
L¹ is —C(O)N(H)—, —C(O)—, or C₁-C₆ alkylene;
L² is a bond, —C(O)—, —NR²—, —CH₂N(R²)—, —CH₂—, or

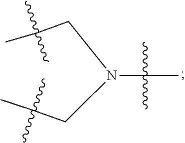 [SMILES: *CN(*)C*]

R² is H or C₁-C₆ alkyl;
x is 0 or 1;
*indicates an attachment point for L²; and
** indicates an attachment point for the piperidinyl-2,6-dione moiety.

Embodiment 2 is the compound of embodiment 1, or a pharmaceutically acceptable salt thereof, wherein the compound is of Formula (IIa), (IIb), or (IIc):

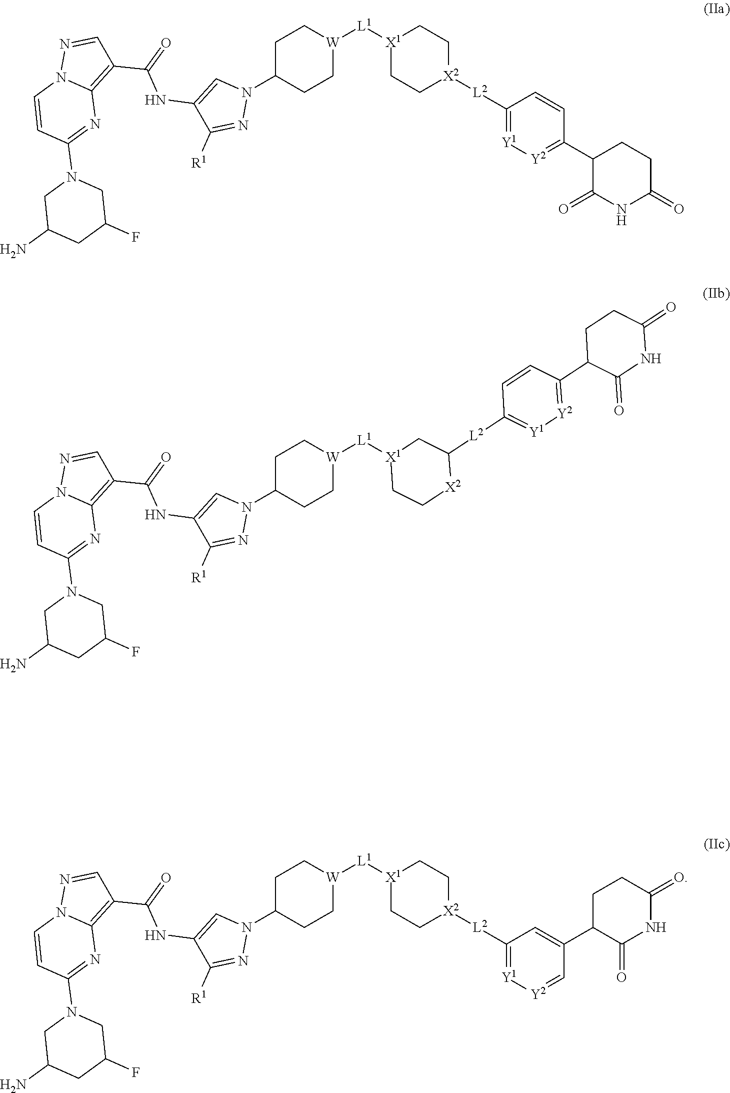

Embodiment 3 is the compound of embodiment 1 or 2, or a pharmaceutically acceptable salt thereof, wherein:
R¹ is C₁-C₃ haloalkyl.

Embodiment 4 is the compound of embodiment 3, or a pharmaceutically acceptable salt thereof, wherein:
R¹ is —CHF₂ or —CF₃.

Embodiment 5 is the compound of any one of embodiments 1-4, or a pharmaceutically acceptable salt thereof, wherein:
W is CH.

Embodiment 6 is the compound of any one of embodiments 1-5, or a pharmaceutically acceptable salt thereof, wherein:
X¹ is CH.

Embodiment 7 is the compound of any one of embodiments 1-5, or a pharmaceutically acceptable salt thereof, wherein:
X¹ is N.

Embodiment 8 is the compound of any one of embodiments 1-7, or a pharmaceutically acceptable salt thereof, wherein:
Y¹ is CH.

Embodiment 9 is the compound of any one of embodiments 1-8, or a pharmaceutically acceptable salt thereof, wherein:
Y² is CH.

Embodiment 10 is the compound of any one of embodiments 1-9, or a pharmaceutically acceptable salt thereof, wherein:
L¹ is —C(O)N(H)—, —C(O)—, or C₁-C₃ alkylene.

Embodiment 11 is the compound of embodiment 10, or a pharmaceutically acceptable salt thereof, wherein:
L¹ is —C(O)N(H)—, —C(O)—, —CH₂—, or —CH₂CH₂—.

Embodiment 12 is the compound of any one of embodiments 1-11, or a pharmaceutically acceptable salt thereof, wherein:
L² is a bond, —C(O)—, —NR²—, —CH₂N(R²)—, —CH₂—, or and

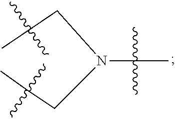 [SMILES: *CN(*)C*]

R² is H or C₁-C₃ alkyl.

Embodiment 13 is the compound of embodiment 12, or a pharmaceutically acceptable salt thereof, wherein:
R² is H or —CH₃.

Embodiment 14 is the compound of any one of embodiments 1-13, or a pharmaceutically acceptable salt thereof, wherein:

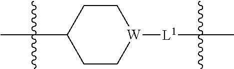

is

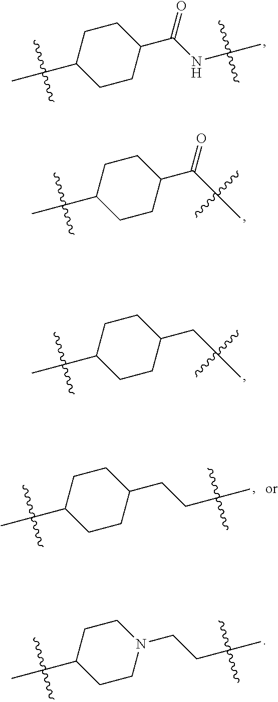 [SMILES: *NC(=O)C1CCC(*)CC1 ; *C(=O)C1CCC(*)CC1 ; *CC1CCC(*)CC1 ; *CCC1CCC(*)CC1 ; *CCN1CCC(*)CC1]

Embodiment 15 is the compound of any one of embodiments 1-14, or a pharmaceutically acceptable salt thereof, wherein:

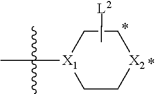

is

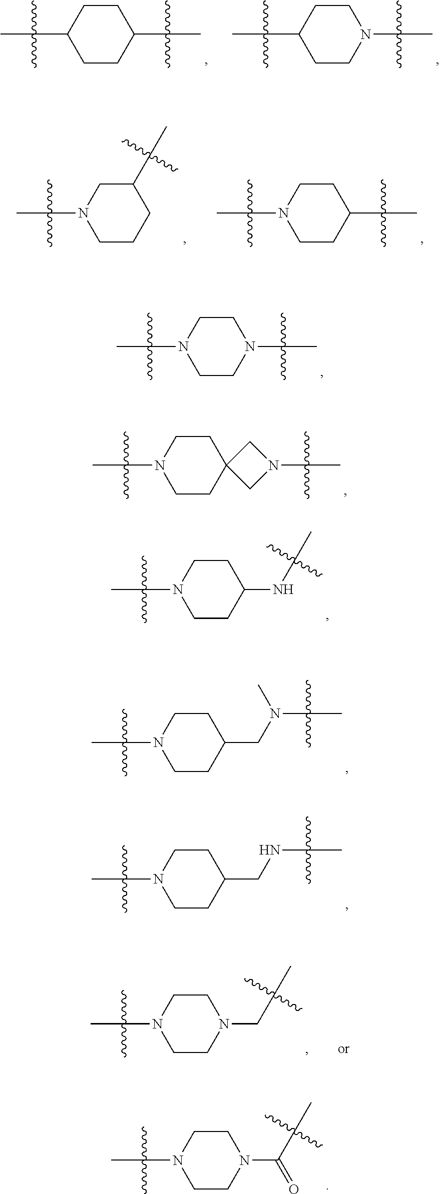

Embodiment 16 is the compound of any one of embodiments 1-15, or a pharmaceutically acceptable salt thereof, wherein:

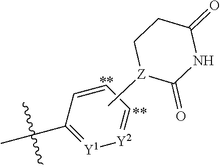

is

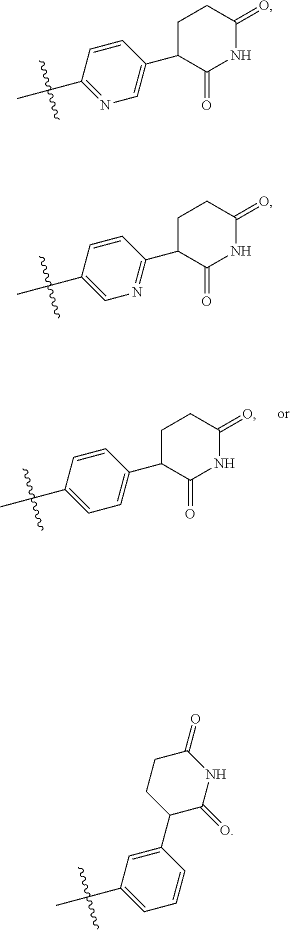 [SMILES: *c1ccc(C2CCC(=O)NC2=O)cn1 ; *c1ccc(C2CCC(=O)NC2=O)nc1 ; *c1ccc(C2CCC(=O)NC2=O)cc1 ; *c1cccc(C2CCC(=O)NC2=O)c1]

Embodiment 17 is a compound selected from the compounds of Table 1 and pharmaceutically acceptable salts thereof.

Embodiment 18 is a pharmaceutical composition comprising the compound of any one of embodiments 1-17, or a pharmaceutically acceptable salt thereof, and a pharmaceutically acceptable excipient.

Embodiment 19 is a method of modulating interleukin-1 (IL1) receptor-associated kinase 4 (IRAK4) comprising contacting IRAK4 with an effective amount of the compound of any one of embodiments 1-17, or a pharmaceutically acceptable salt thereof, or the pharmaceutical composition of embodiment 18.

Embodiment 20 is a method of treating an inflammatory or autoimmune disease in a subject in need thereof, comprising administering to the subject an effective amount of the compound of any one of embodiments 1-17, or a pharmaceutically acceptable salt thereof, or the pharmaceutical composition of embodiment 18, optionally wherein the inflammatory or autoimmune disease is atopic dermatitis, asthma, lupus, rheumatoid arthritis, familial mediterranean fever, psoriasis, generalized pustular psoriasis, cryoprin-associated periodic syndrome, hidradenitis suppurativa, Bechet's syndrome, or familial cold autoinflammatory syndrome.

### DETAILED DESCRIPTION

### Definitions

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments of the invention.

The term “consisting of” means that a subject-matter has at least 90%, 95%, 97%, 98% or 99% of the stated features or components of which it consists. In another embodiment the term “consisting of” excludes from the scope of any succeeding recitation any other features or components, excepting those that are not essential to the technical effect to be achieved.

As used herein, the term “or” is to be interpreted as an inclusive “or” meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size, or thickness, are to be understood to include any integer within the recited range, unless otherwise indicated. As used herein, the terms “about” and “approximately” mean±20%, +10%, +5%, or +1% of the indicated range, value, or structure, unless otherwise indicated.

An “alkyl” group is a saturated, partially saturated, or unsaturated straight chain or branched non-cyclic hydrocarbon having from 1 to 10 carbon atoms (C₁-C₁₀ alkyl), typically from 1 to 8 carbons (C₁-C₈ alkyl) or, in some embodiments, from 1 to 6 (C₁-C₆ alkyl), 1 to 3 (C₁-C₃ alkyl), or 2 to 6 (C₂-C₆ alkyl) carbon atoms. In some embodiments, the alkyl group is a saturated alkyl group. Representative saturated alkyl groups include -methyl, -ethyl, -n-propyl, -n-butyl, -n-pentyl and -n-hexyl; while saturated branched alkyls include -isopropyl, -sec-butyl, -isobutyl, tert-butyl, -isopentyl, -neopentyl, tertpentyl, -2-methylpentyl, -3-methylpentyl, -4-methylpentyl, -2,3-dimethylbutyl and the like. In some embodiments, an alkyl group is an unsaturated alkyl group, also termed an alkenyl or alkynyl group. An “alkenyl” group is an alkyl group that contains one or more carbon-carbon double bonds. An “alkynyl” group is an alkyl group that contains one or more carbon-carbon triple bonds. Examples of unsaturated alkyl groups include, but are not limited to, vinyl, allyl, CH═CH(CH₃), —CH═C(CH₃)₂, —C(CH₃)═CH₂, —C(CH₃)═CH(CH₃), —C(CH₂CH₃)═CH₂, —C═CH, —C≡C(CH₃), —C≡C(CH₂CH₃), —CH₂C≡CH, CH₂C═C(CH₃) and CH₂C═C(CH₂CH₃), among others. An alkyl group can be substituted or unsubstituted. When the alkyl groups described herein are said to be “substituted,” they may be substituted with any substituent or substituents as those found in the exemplary compounds and embodiments disclosed herein, as well as halogen; hydroxy; alkoxy; cycloalkyloxy, aryloxy, heterocyclyloxy, heteroaryloxy, heterocycloalkyloxy, cycloalkylalkyloxy, aralkyloxy, heterocyclylalkyloxy, heteroarylalkyloxy, heterocycloalkylalkyloxy; oxo (═O); amino, alkylamino, cycloalkylamino, arylamino, heterocyclylamino, heteroarylamino, heterocycloalkylamino, cycloalkylalkylamino, aralkylamino, heterocyclylalkylamino, heteroaralkylamino, heterocycloalkylalkylamino; imino; imido; amidino; guanidino; enamino; acylamino; sulfonylamino; urea, nitrourea; oxime; hydroxylamino; alkoxyamino; aralkoxyamino; hydrazino; hydrazido; hydrazono; azido; nitro; thio (—SH), alkylthio; ═S; sulfinyl; sulfonyl; aminosulfonyl; phosphonate; phosphinyl; acyl; formyl; carboxy; ester; carbamate; amido; cyano; isocyanato; isothiocyanato; cyanato; thiocyanato; or —B(OH)₂. In certain embodiments, when the alkyl groups described herein are said to be “substituted,” they may be substituted with any substituent or substituents as those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; B(OH)₂, or O(alkyl)aminocarbonyl.

A “cycloalkyl” group is a saturated, or partially saturated cyclic alkyl group of from 3 to 10 carbon atoms (C₃-C₁₀ cycloalkyl) having a single cyclic ring or multiple condensed or bridged rings that can be optionally substituted. In some embodiments, the cycloalkyl group has 3 to 8 ring carbon atoms (C₃-C₈ cycloalkyl), whereas in other embodiments the number of ring carbon atoms ranges from 3 to 5 (C₃-C₈ cycloalkyl), 3 to 6 (C₃-C₆ cycloalkyl), or 3 to 7 (C₃-C₇ cycloalkyl). In some embodiments, the cycloalkyl groups are saturated cycloalkyl groups. Such saturated cycloalkyl groups include, by way of example, single ring structures such as cyclopropyl, cyclobutyl, cyclopentyl, cyclohexyl, cycloheptyl, cyclooctyl, 1methylcyclopropyl, 2methylcyclopentyl, 2-methylcyclooctyl, and the like, or multiple or bridged ring structures such as 1-bicyclo[1.1.1]pentyl, bicyclo[2.1.1]hexyl, bicyclo[2.2.1]heptyl, bicyclo[2.2.2]octyl, adamantyl and the like. In other embodiments, the cycloalkyl groups are unsaturated cycloalkyl groups. Examples of unsaturared cycloalkyl groups include cyclohexenyl, cyclopentenyl, cyclohexadienyl, butadienyl, pentadienyl, hexadienyl, among others. A cycloalkyl group can be substituted or unsubstituted. Such substituted cycloalkyl groups include, by way of example, cyclohexanol and the like.

An “aryl” group is an aromatic carbocyclic group of from 6 to 14 carbon atoms (C₆-C₁₄ aryl) having a single ring (e.g., phenyl) or multiple condensed rings (e.g., naphthyl or anthryl). In some embodiments, aryl groups contain 6-14 carbons (C₆-C₁₄ aryl), and in others from 6 to 12 (C₆-C₁₂ aryl) or even 6 to 10 carbon atoms (C₆-C₁₀ aryl) in the ring portions of the groups. Particular aryls include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

A “halogen” or “halo” is fluorine, chlorine, bromine or iodine.

“Haloalkyl” refers to an alkyl radical, as defined above, that is substituted by one or more halo radicals, as defined above, e.g., trifluoromethyl, difluoromethyl, trichloromethyl, 2,2,2trifluoroethyl, 1,2difluoroethyl, 3bromo2fluoropropyl, 1,2dibromoethyl, and the like. In some embodiments, the haloalkyl group has one to six carbon atoms and is substituted by one or more halo radicals (C₁-C₆ haloalkyl), or the haloalkyl group has one to three carbon atoms and is substituted by one or more halo radicals (C₁-C₃ haloalkyl). The halo radicals may be all the same or the halo radicals may be different. Unless specifically stated otherwise, a haloalkyl group is optionally substituted.

A “heteroaryl” group is an aromatic ring system having one to four heteroatoms as ring atoms in a heteroaromatic ring system, wherein the remainder of the atoms are carbon atoms. In some embodiments, heteroaryl groups contain 3 to 6 ring atoms, and in others from 6 to 9 or even 6 to 10 atoms in the ring portions of the groups. Suitable heteroatoms include oxygen, sulfur and nitrogen. In certain embodiments, the heteroaryl ring system is monocyclic or bicyclic. Non-limiting examples include but are not limited to, groups such as pyrrolyl, pyrazolyl, imidazolyl, triazolyl, tetrazolyl, oxazolyl, isoxazolyl, benzisoxazolyl (e.g., benzo[d]isoxazolyl), thiazolyl, pyrolyl, pyridazinyl, pyrimidyl, pyrazinyl, thiophenyl, benzothiophenyl, furanyl, benzofuranyl, indolyl (e.g., indolyl-2-onyl or isoindolin-1-onyl), azaindolyl (pyrrolopyridyl or 1Hpyrrolo[2,3b]pyridyl), indazolyl, benzimidazolyl (e.g., 1Hbenzo[d]imidazolyl), imidazopyridyl (e.g., azabenzimidazolyl or 1Himidazo[4,5b]pyridyl), pyrazolopyridyl, triazolopyridyl, benzotriazolyl (e.g., 1Hbenzo[d][1,2,3]triazolyl), benzoxazolyl (e.g., benzo[d]oxazolyl), benzothiazolyl, benzothiadiazolyl, isoxazolopyridyl, thianaphthalenyl, purinyl, xanthinyl, adeninyl, guaninyl, quinolinyl, isoquinolinyl (e.g., 3,4dihydroisoquinolin-1 (2H)-onyl), tetrahydroquinolinyl, quinoxalinyl, and quinazolinyl groups. A heteroaryl group can be substituted or unsubstituted.

A “heterocyclyl” is a non-aromatic cycloalkyl in which one to four of the ring carbon atoms are independently replaced with a heteroatom selected from O, S and N. In some embodiments, heterocyclyl groups include 3 to 10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocycloalkyl group can be substituted or unsubstituted. Heterocyclyl groups encompass saturated and partially saturated ring systems. Further, the term heterocyclyl is intended to encompass any non-aromatic ring containing at least one heteroatom, which ring may be fused to an aryl or heteroaryl ring, regardless of the attachment to the remainder of the molecule. The phrase also includes bridged polycyclic ring systems containing a heteroatom. Representative examples of a heterocyclyl group include, but are not limited to, aziridinyl, azetidinyl, azepanyl, pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dithianyl, 1,4dioxaspiro[4.5]decanyl, homopiperazinyl, quinuclidyl, or tetrahydropyrimidin-2 (1H)-one. Representative substituted heterocyclyl groups may be monosubstituted or substituted more than once, such as, but not limited to, pyridyl or morpholinyl groups, which are 2-, 3-, 4-, 5-, or 6substituted, or disubstituted with various substituents such as those listed below.

When the groups described herein, with the exception of alkyl group, are said to be “substituted,” they may be substituted with any appropriate substituent or substituents. Illustrative examples of substituents are those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; oxygen (═O); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazinyl, pyridazinyl, pyrimidyl, benzimidazolyl, benzothiophenyl, or benzofuranyl) aryloxy; aralkyloxy; heterocyclyloxy; and heterocyclyl alkoxy.

Certain commonly used alternative chemical names may be used. For example, a divalent group such as a divalent “alkyl” group, a divalent “phenyl” group, a divalent “heteroaryl” group, a divalent “heterocyclyl” group etc., may also be referred to as an “alkylene” group, a “phenylene” group, a “heteroarylene” group, or a “heterocyclylene” group, respectively.

Embodiments of the disclosure are meant to encompass pharmaceutically acceptable salts, tautomers, isotopologues, and stereoisomers of the compounds provided herein, such as the compounds of Formula (I).

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts of the compounds of formula (I) include, but are not limited to metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (Nmethyl-glucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and ptoluenesulfonic acid. Specific non-toxic acids include hydrochloric, hydrobromic, maleic, phosphoric, sulfuric, and methanesulfonic acids. Examples of specific salts thus include hydrochloride, formic, and mesylate salts. Others are well-known in the art, see for example, Remington's Pharmaceutical Sciences, 18^{th} eds., Mack Publishing, Easton PA (1990) or Remington: The Science and Practice of Pharmacy, 19^{th} eds., Mack Publishing, Easton PA (1995).

As used herein and unless otherwise indicated, the term “stereoisomer” or “stereoisomerically pure” means one stereoisomer of a particular compound that is substantially free of other stereoisomers of that compound. For example, a stereoisomerically pure compound having one chiral center will be substantially free of the opposite enantiomer of the compound. A stereoisomerically pure compound having two chiral centers will be substantially free of other diastereomers of the compound. A typical stereoisomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds disclosed herein can have chiral centers and can occur as racemates, individual enantiomers or diastereomers, and mixtures thereof. All such isomeric forms are included within the embodiments disclosed herein, including mixtures thereof.

The use of stereoisomerically pure forms of the compounds disclosed herein, as well as the use of mixtures of those forms, are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (WileyInterscience, New York, 1981); Wilen, S. H., et al., Tetrahedron 33:2725 (1977); Eliel, E. L., Stereochemistry of Carbon Compounds (McGrawHill, NY, 1962); Wilen, S. H., Tables of Resolving Agents and Optical Resolutions p. 268 (E. L. Eliel, Ed., Univ. of Notre Dame Press, Notre Dame, IN, 1972); Todd, M., Separation Of Enantiomers: Synthetic Methods (Wiley-VCH Verlag GmbH & Co. KGaA, Weinheim, Germany, 2014); Toda, F., Enantiomer Separation: Fundamentals and Practical Methods (Springer Science & Business Media, 2007); Subramanian, G. Chiral Separation Techniques: A Practical Approach (John Wiley & Sons, 2008); Ahuja, S., Chiral Separation Methods for Pharmaceutical and Biotechnological Products (John Wiley & Sons, 2011).

It should also be noted the compounds disclosed herein can include E and Z isomers, or a mixture thereof, and cis and trans isomers or a mixture thereof. In certain embodiments, the compounds are isolated as either the E or Z isomer. In other embodiments, the compounds are a mixture of the E and Z isomers.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

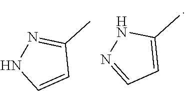

As readily understood by one skilled in the art, a wide variety of functional groups and other structures may exhibit tautomerism and all tautomers of compounds of Formula (I) are within the scope of the present disclosure.

It should also be noted the compounds disclosed herein can contain unnatural proportions of atomic isotopes at one or more of the atoms. For example, the compounds may be radiolabeled with radioactive isotopes, such as for example tritium (³H), iodine-125 (¹²⁵I), sulfur35 (³⁵S), or carbon-14 (¹⁴C), or may be isotopically enriched, such as with deuterium (²H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer therapeutic agents, research reagents, e.g., binding assay reagents, and diagnostic agents, e.g., in vivo imaging agents. All isotopic variations of the compounds as described herein, whether radioactive or not, are intended to be encompassed within the scope of the embodiments provided herein. In some embodiments, there are provided isotopologues of the compounds disclosed herein, for example, the isotopologues are deuterium, carbon-13, and/or nitrogen-15 enriched compounds. As used herein, “deuterated”, means a compound wherein at least one hydrogen (H) has been replaced by deuterium (indicated by D or ²H), that is, the compound is enriched in deuterium in at least one position.

It is understood that, independently of stereoisomerical or isotopic composition, each compound disclosed herein can be provided in the form of any of the pharmaceutically acceptable salts discussed herein. Equally, it is understood that the isotopic composition may vary independently from the stereoisomerical composition of each compound referred to herein. Further, the isotopic composition, while being restricted to those elements present in the respective compound or salt thereof disclosed herein, may otherwise vary independently from the selection of the pharmaceutically acceptable salt of the respective compound.

It should be noted that if there is a discrepancy between a depicted structure and a name for that structure, the depicted structure is to be accorded more weight.

“Treating” as used herein, means an alleviation, in whole or in part, of a disorder, disease or condition, or one or more of the symptoms associated with a disorder, disease, or condition, or slowing or halting of further progression or worsening of those symptoms, or alleviating or eradicating the cause(s) of the disorder, disease, or condition itself. In one embodiment, the disorder is a neurodegenerative disease, as described herein, or a symptom thereof.

“Preventing” as used herein, means a method of delaying and/or precluding the onset, recurrence or spread, in whole or in part, of a disorder, disease or condition; barring a subject from acquiring a disorder, disease, or condition; or reducing a subject's risk of acquiring a disorder, disease, or condition. In one embodiment, the disorder is a neurodegenerative disease, as described herein, or symptoms thereof.

The term “effective amount” in connection with a compound disclosed herein means an amount capable of treating or preventing a disorder, disease or condition, or symptoms thereof, disclosed herein.

The term “subject” or “patient” as used herein include an animal, including, but not limited to, an animal such a cow, monkey, horse, sheep, pig, chicken, turkey, quail, cat, dog, mouse, rat, rabbit or guinea pig, in one embodiment a mammal, in another embodiment a human. In one embodiment, a subject is a human having or at risk for having an SIP5 mediated disease, or a symptom thereof.

Although various features of the invention may be described in the context of a single embodiment, the features may also be provided separately or in any suitable combination. Conversely, although the invention may be described herein in the context of separate embodiments for clarity, the invention may also be implemented in a single embodiment.

### Compounds

In one aspect, provided herein is a compound of Formula (I):

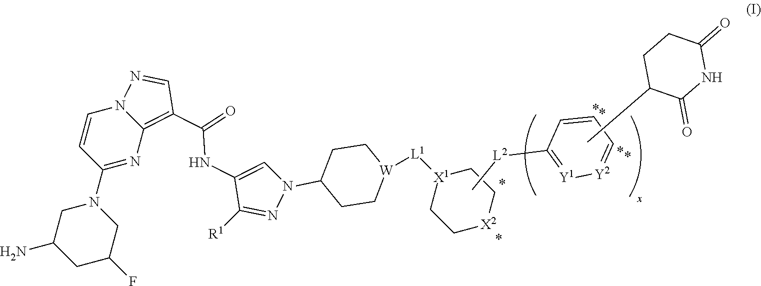

or a pharmaceutically acceptable salt thereof, wherein:
R¹ is C₁-C₆ haloalkyl;
W is CH or N;
X¹ is CH or N;
X² is CH₂, CH, C, N, or NH;
Y¹ and Y² are independently CH or N;
L¹ is —C(O)N(H)—, —C(O)—, or C₁-C₆ alkylene;
L² is a bond, —C(O)—, —NR²—, —CH₂N(R²)—, —CH₂—, or

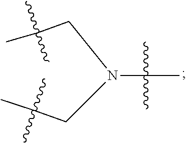 [SMILES: *CN(*)C*]

R² is H or C₁-C₆ alkyl;
x is 0 or 1;
* indicates an attachment point for L²; and
** indicates an attachment point for the piperidinyl-2,6-dione moiety.

In some embodiments, R¹ is C₁-C₆ haloalkyl. In some embodiments, R¹ is C₁-C₆ haloalkyl containing 1-13 halogen atoms. In some embodiments, R¹ is C₁-C₃ haloalkyl. In some embodiments, R¹ is C₁-C₃ haloalkyl containing 1-7 halogen atoms. In some embodiments, R¹ is —CF₃, —CHF₂, —CH₂F, —CCl₃, —CHC₂, —CH₂Cl, —CF₂Cl, —CFCl₂, —CH₂CF₃, —CH₂CHF₂, or —CH₂CCl₃. In some embodiments, R¹ is —CHF₂ or —CF₃. In some embodiments, R¹ is —CF₃. In some embodiments, R¹ is —CHF₂.

In some embodiments, W is CH or N. In some embodiments, W is CH. In some embodiments, W is N.

In some embodiments, X¹ is CH or N. In some embodiments, X¹ is CH. In some embodiments, X¹ is N.

In some embodiments, X² is CH₂, CH, C, N, or NH. In some embodiments, X² is CH₂, CH, or C. In some embodiments, X² is CH₂. In some embodiments, X² is CH. In some embodiments, X² is C. In some embodiments, X² is N or NH. In some embodiments, X² is N. In some embodiments, X² is NH.

In some embodiments, Y¹ and Y² are independently CH or N. In some embodiments, Y¹ is CH. In some embodiments, Y¹ is N. In some embodiments, Y² is CH. In some embodiments, Y² is N. In some embodiments, Y¹ is CH and Y² is N. In some embodiments, Y² is CH and Y¹ is N. In some embodiments, Y¹ and Y² are each CH. In some embodiments, Y¹ and Y² are each N.

In some embodiments, L¹ is —C(O)N(H)—, —C(O)—, or C₁-C₆ alkylene. In some embodiments, L¹ is —C(O)N(H)—, —C(O)—, or C₁-C₃ alkylene. In some embodiments, L¹ is —C(O)N(H)—, —C(O)—, —CH₂—, or —CH₂CH₂—.

In some embodiments, L¹ is —C(O)N(H)—. In some embodiments, L¹ is —C(O)—. In some embodiments, L¹ is C₁-C₃ alkylene. In some embodiments, L¹ is —CH₂—, —CH₂CH₂—, or —CH₂CH₂CH₂—. In some embodiments, L¹ is —CH₂—. In some embodiments, L¹ is —CH₂CH₂—. In some embodiments, L¹ is —CH₂CH₂CH₂—.

In some embodiments, L² is a bond, —C(O)—, —NR²—, —CH₂N(R²)—, —CH₂—, or

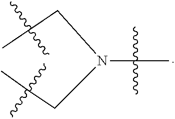 [SMILES: *CN(*)C*]

In some embodiments, L² is a bond, —C(O)—, —NR²—, —CH₂N(R²)—, —CH₂—, or

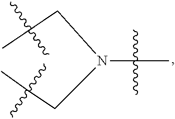

wherein R² is H or C₁-C₃ alkyl.

In some embodiments, L² is a bond. In some embodiments, L² is —C(O)—. In some embodiments, L² is —NR²—. In some embodiments, L² is —CH₂N(R²)—. In some embodiments, L² is —CH₂—. In some embodiments, L² is

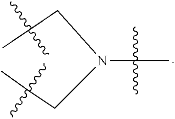 [SMILES: *CN(*)C*]

In some embodiments, R² is H or C₁-C₆ alkyl. In some embodiments, R² is H or C₁-C₃ alkyl. In some embodiments, R² is H or —CH₃.

In some embodiments, R² is H.

In some embodiments, R² is C₁-C₆ alkyl. In some embodiments, R² is C₁-C₃ alkyl. In some embodiments, R² is methyl, ethyl, n-propyl, or isopropyl. In some embodiments, R² is methyl. In some embodiments, R² is ethyl. In some embodiments, R² is n-propyl. In some embodiments, R² is isopropyl.

In some embodiments, is:

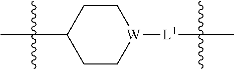

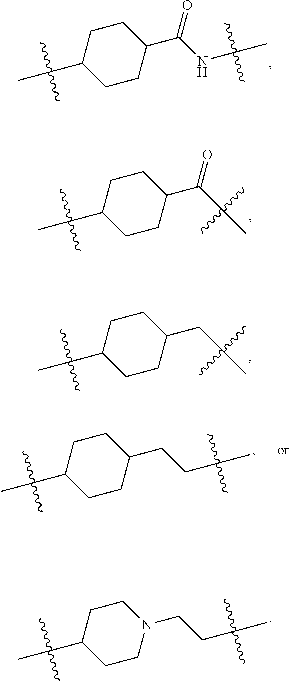 [SMILES: *NC(=O)C1CCC(*)CC1 ; *C(=O)C1CCC(*)CC1 ; *CC1CCC(*)CC1 ; *CCC1CCC(*)CC1 ; *CCN1CCC(*)CC1]

In some embodiments, is:

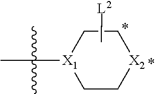

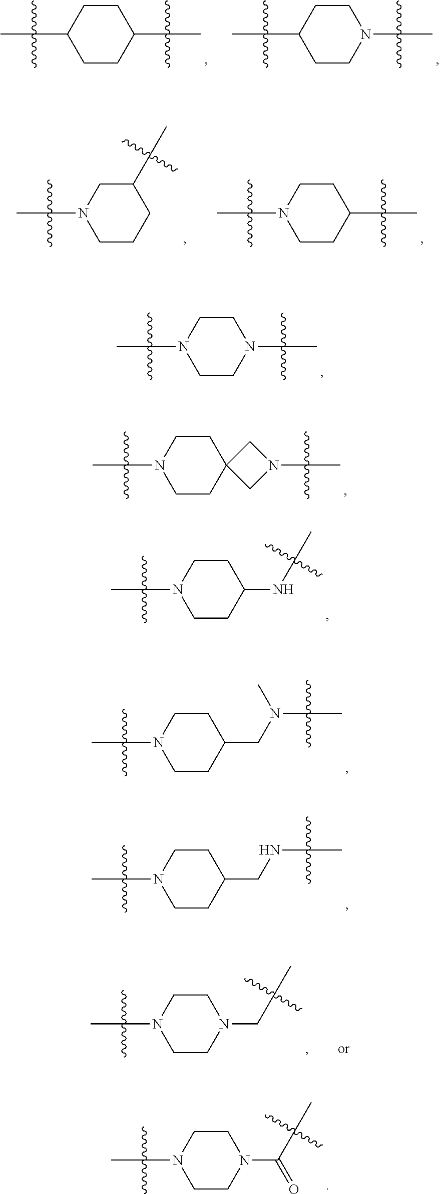

In some embodiments, x is 0 or 1. In some embodiments, x is 0. In some embodiments, x is 1.

In some embodiments,

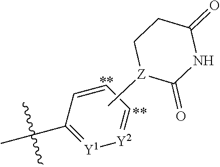

is:

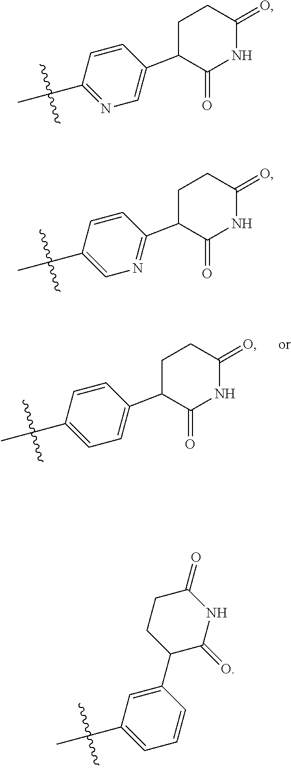 [SMILES: *c1ccc(C2CCC(=O)NC2=O)cn1 ; *c1ccc(C2CCC(=O)NC2=O)nc1 ; *c1ccc(C2CCC(=O)NC2=O)cc1 ; *c1cccc(C2CCC(=O)NC2=O)c1]

In some embodiments, the compound of Formula (I) is a compound of Formula (I-A):

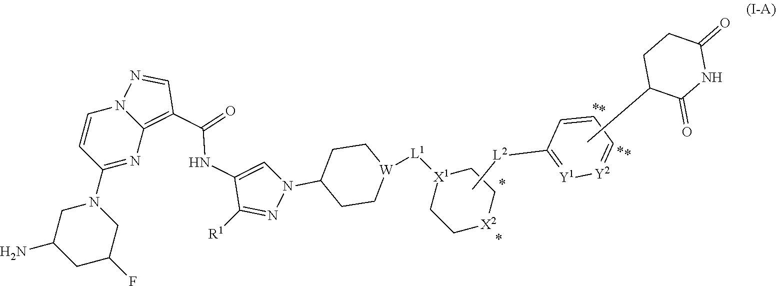

wherein R¹, L¹, L², W, X¹, X², Y¹, Y², *, and ** are as described for Formula (I).

In some embodiments, the compound of Formula (I) is a compound of Formula (I-B-i) or (I-B-ii):

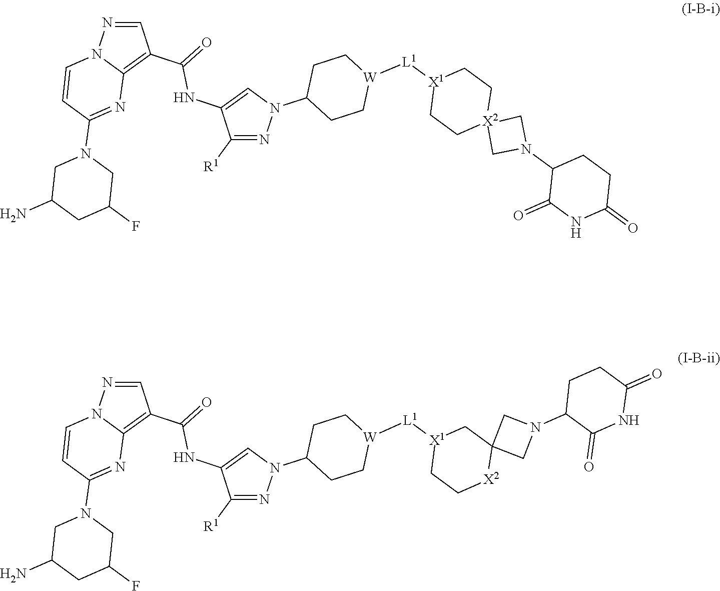

wherein R¹, L¹, W, X¹, and X² are as described for Formula (I).

In some embodiments, the compound of Formula (I) is a compound of Formula (IIa), (IIb), (IIc), or (IId):

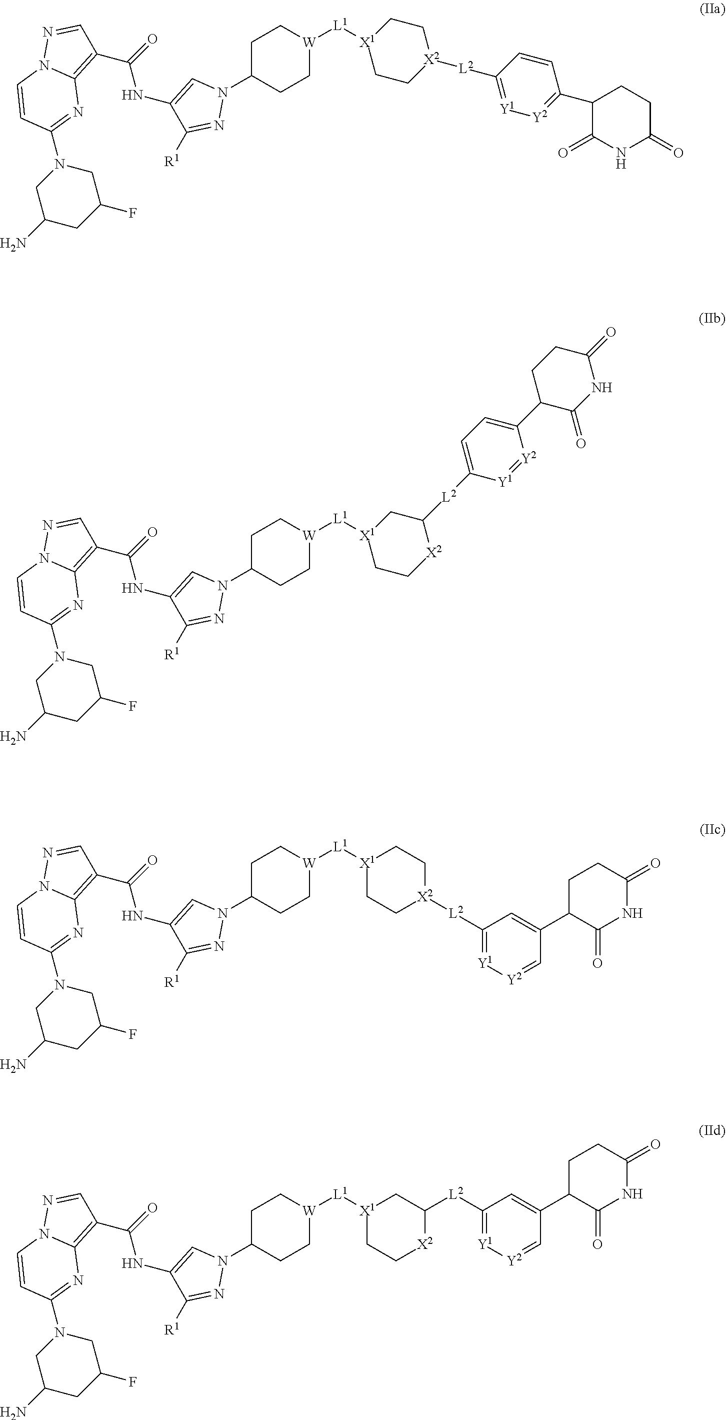

wherein R¹, L¹, L², W, X¹, X², Y¹, and Y² are as described for Formula (I).

In some embodiments, the compound of Formula (I) is a compound of Formula (IIIa), (IIIb), or (IIIc):

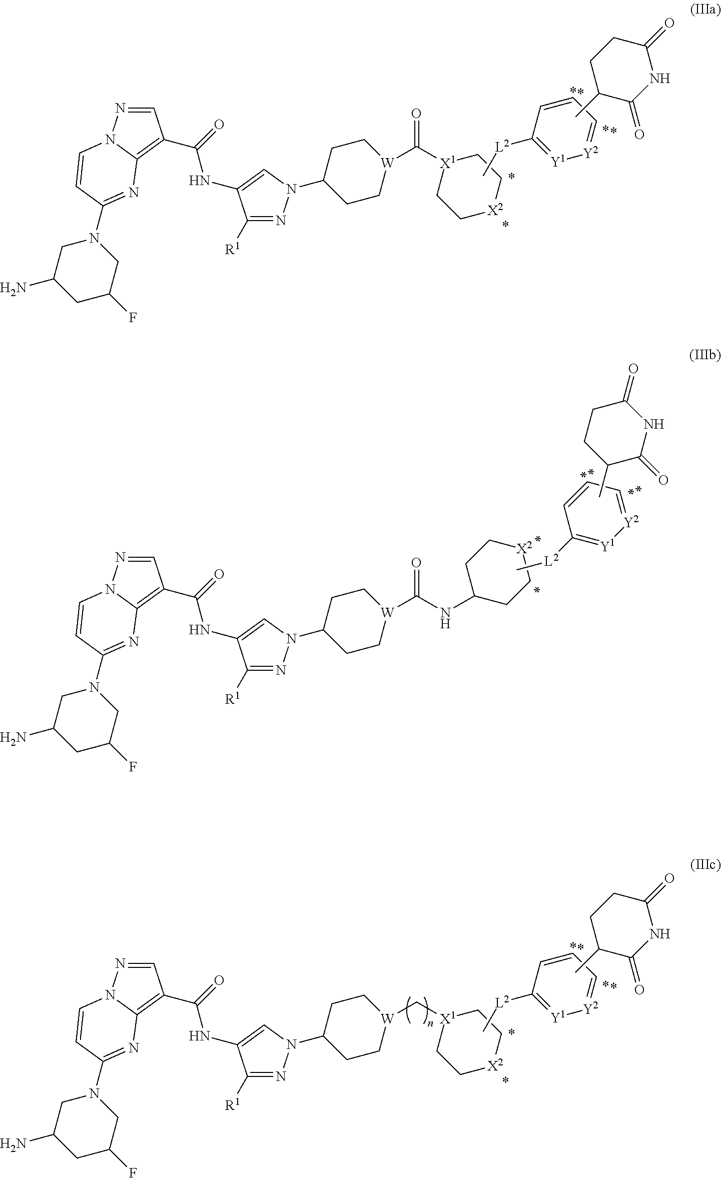

wherein R¹, L², W, X¹, X², Y¹, Y², *, and ** are as described for Formula (I); and wherein n is an integer 1-6.

In some embodiments, the compound of Formula (I) is a compound of Formula (IVa), (IVb), (IVc), (IVd), (IVe), or (IVf):

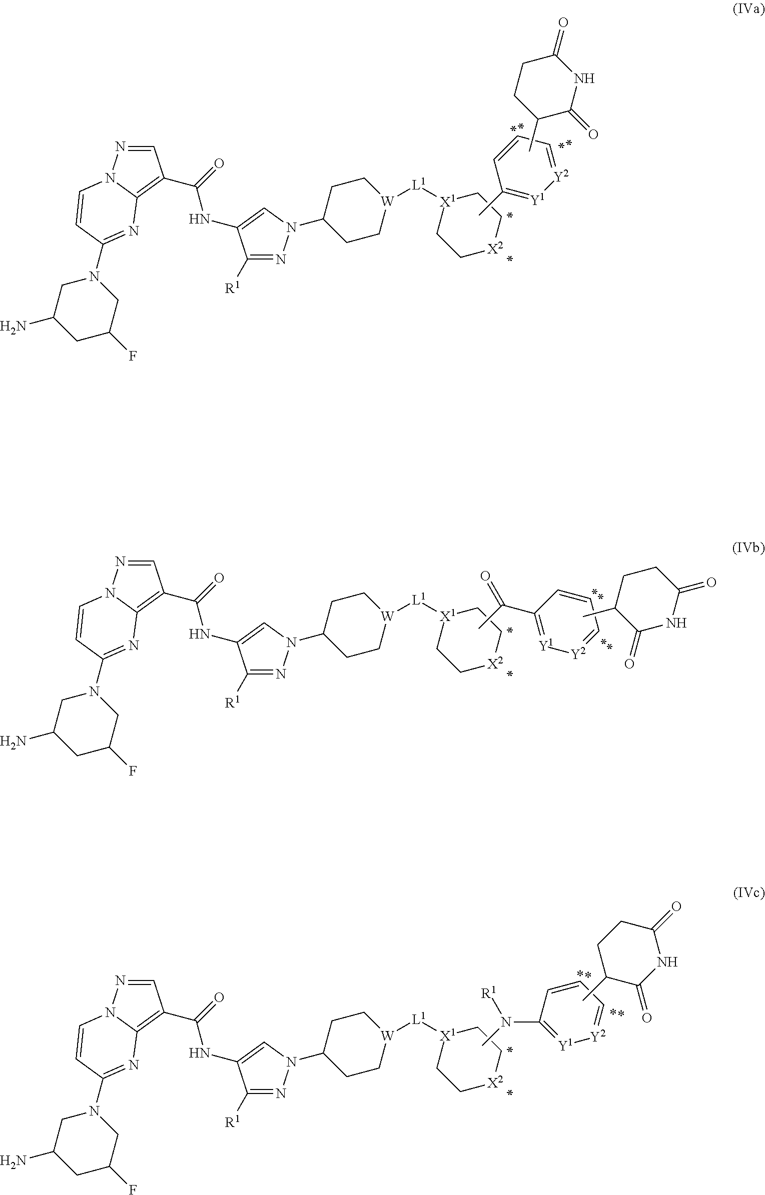

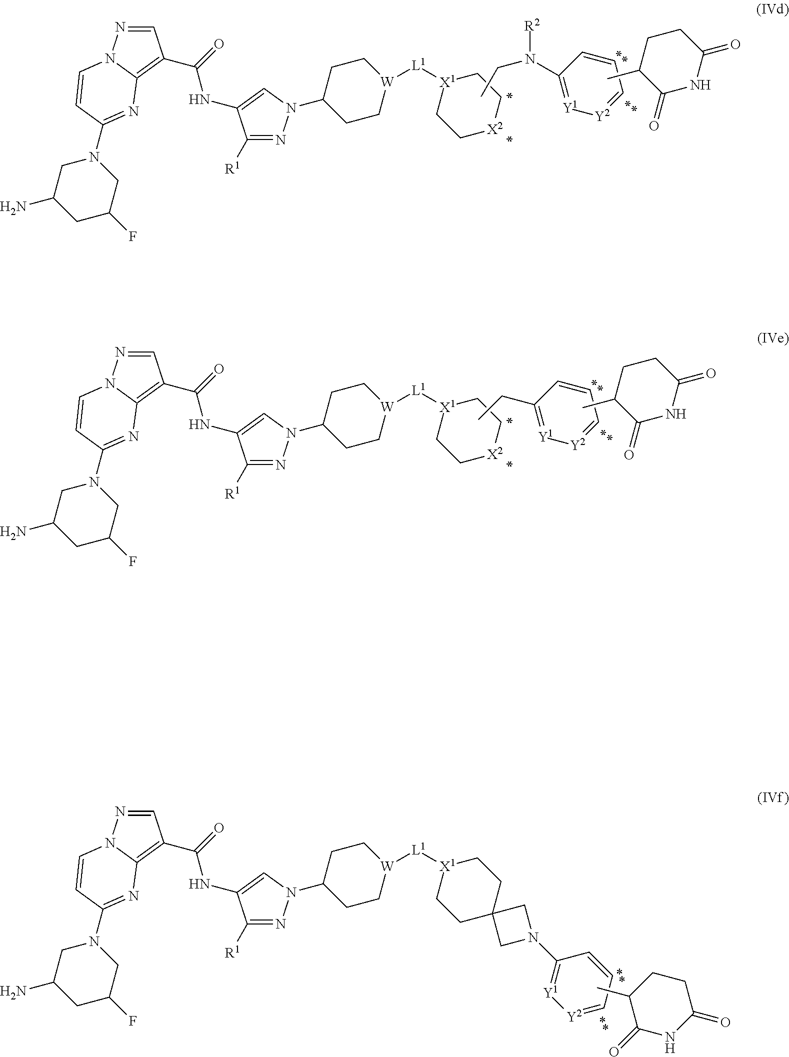

wherein R¹, R², L¹, W, X¹, X², Y¹, Y², *, and ** are as described for Formula (I).

In some embodiments, the compound of Formula (I) is a compound of Formula (Va), (Vb), (Vc), (Vd), or (Ve):

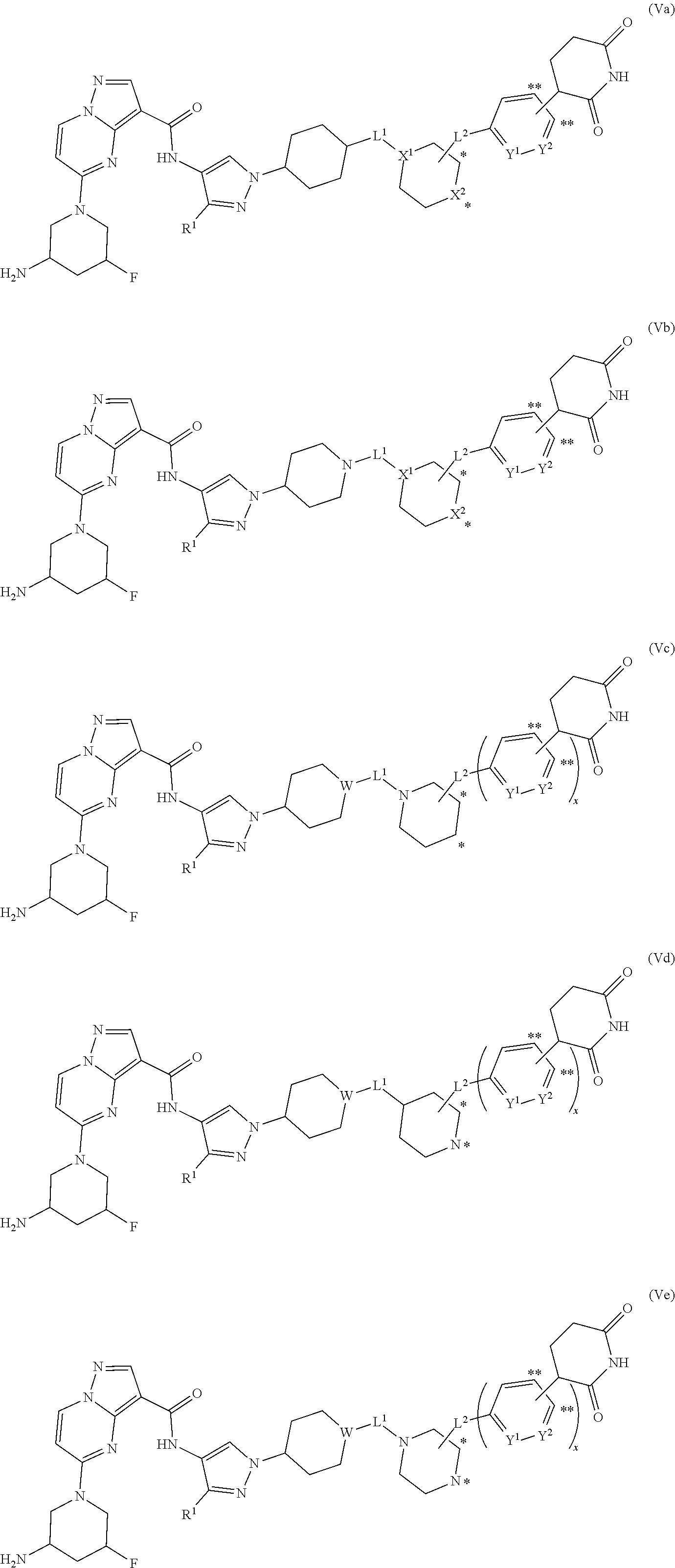

wherein R¹, L¹, L², W, X¹, X², Y¹, Y², *, and ** are as described for Formula (I).

In the descriptions herein, it is understood that every description, variation, embodiment, or aspect of a moiety may be combined with every description, variation, embodiment, or aspect of other moieties the same as if each and every combination of descriptions is specifically and individually listed. For example, every description, variation, embodiment, or aspect provided herein with respect to R¹ of Formula (I) may be combined with every description, variation, embodiment, or aspect of W, X¹, X², Y¹, Y², L¹, L², R² and x the same as if each and every combination were specifically and individually listed. It is also understood that all descriptions, variations, embodiments, or aspects of Formula (I), where applicable, apply equally to other formulae detailed herein, and are equally described, the same as if each and every description, variation, embodiment, or aspect were separately and individually listed for all formulae. For example, all descriptions, variations, embodiments, or aspects of Formula (I), where applicable, apply equally to any of the formulae as detailed herein, such as Formulae (I-A), (I-B-i), (I-B-ii), (IIa), (IIb), (IIc), (IId), (IIIa), (IIIb), (IIIc), (IVa), (IVb), (IVc), (IVd), (IVe), (IVf), (Va), (Vb), (Vc), (Vd), and (Ve) are equally described, the same as if each and every description, variation, embodiment, or aspect were separately and individually listed for all formulae.

In some embodiments, provided is a compound selected from the compounds in Table 1 or a pharmaceutically acceptable salt thereof. Although certain compounds described in the present disclosure, including in Table 1, are presented as specific stereoisomers and/or in a non-stereochemical form, it is understood that any or all stereochemical forms, including any enantiomeric or diastereomeric forms, and any tautomers or other forms of any of the compounds of the present disclosure, including in Table 1, are herein described.

TABLE 1

| Compound | |
|---|---|
| No. | Structure |
| P-1 | 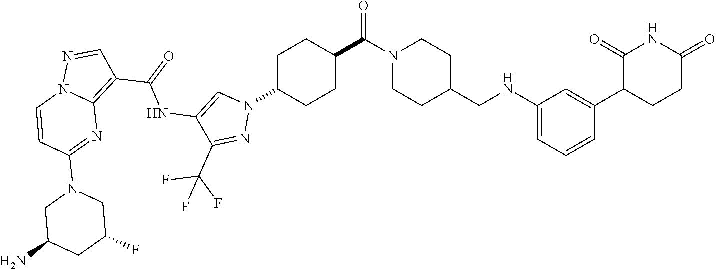 [SMILES: N[C@@H]1C[C@@H](F)CN(c2ccn3ncc(C(=O)Nc4cn([C@H]5CC[C@H](C(=O)N6CCC(CNc7cccc(C8CCC(=O)NC8=O)c7)CC6)CC5)nc4C(F)(F)F)c3n2)C1] |
| P-2 | 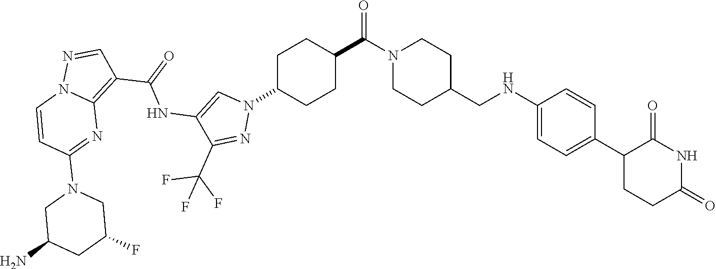 [SMILES: N[C@@H]1C[C@@H](F)CN(c2ccn3ncc(C(=O)Nc4cn([C@H]5CC[C@H](C(=O)N6CCC(CNc7ccc(C8CCC(=O)NC8=O)cc7)CC6)CC5)nc4C(F)(F)F)c3n2)C1] |
| P-3 | 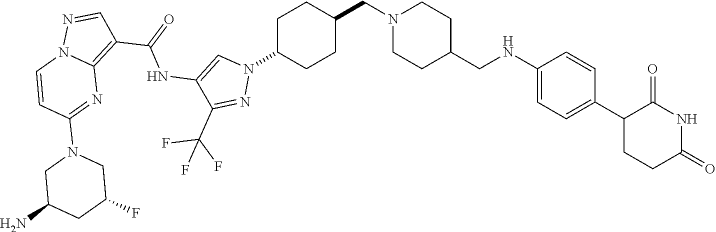 [SMILES: N[C@@H]1C[C@@H](F)CN(c2ccn3ncc(C(=O)Nc4cn([C@H]5CC[C@H](CN6CCC(CNc7ccc(C8CCC(=O)NC8=O)cc7)CC6)CC5)nc4C(F)(F)F)c3n2)C1] |
| P-4 | 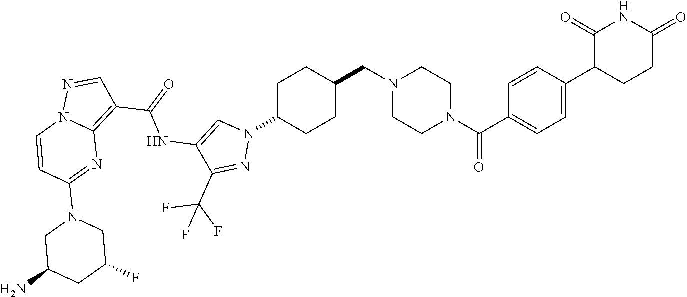 [SMILES: N[C@@H]1C[C@@H](F)CN(c2ccn3ncc(C(=O)Nc4cn([C@H]5CC[C@H](CN6CCN(C(=O)c7ccc(C8CCC(=O)NC8=O)cc7)CC6)CC5)nc4C(F)(F)F)c3n2)C1] |
| P-5 | 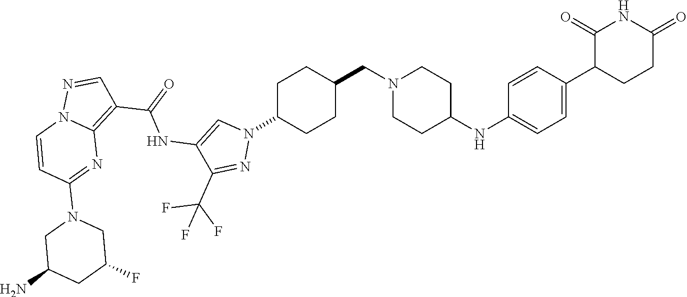 [SMILES: N[C@@H]1C[C@@H](F)CN(c2ccn3ncc(C(=O)Nc4cn([C@H]5CC[C@H](CN6CCC(Nc7ccc(C8CCC(=O)NC8=O)cc7)CC6)CC5)nc4C(F)(F)F)c3n2)C1] |
| P-6 | 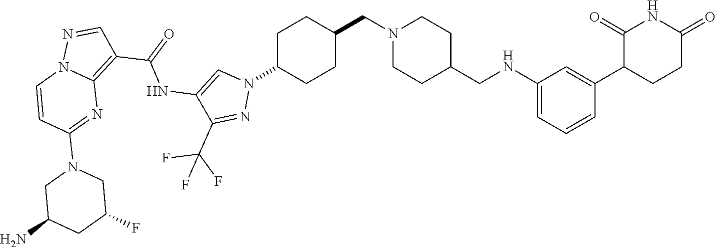 [SMILES: N[C@@H]1C[C@@H](F)CN(c2ccn3ncc(C(=O)Nc4cn([C@H]5CC[C@H](CN6CCC(CNc7cccc(C8CCC(=O)NC8=O)c7)CC6)CC5)nc4C(F)(F)F)c3n2)C1] |
| P-7 | 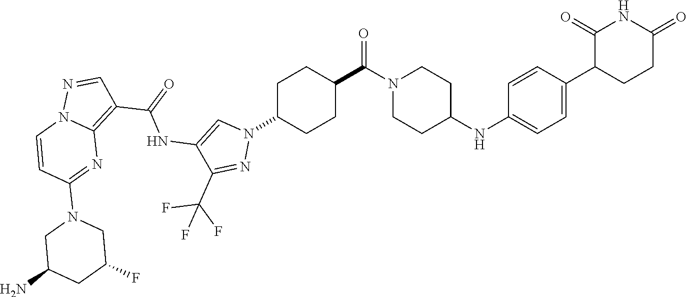 [SMILES: N[C@@H]1C[C@@H](F)CN(c2ccn3ncc(C(=O)Nc4cn([C@H]5CC[C@H](C(=O)N6CCC(Nc7ccc(C8CCC(=O)NC8=O)cc7)CC6)CC5)nc4C(F)(F)F)c3n2)C1] |
| P-8 | 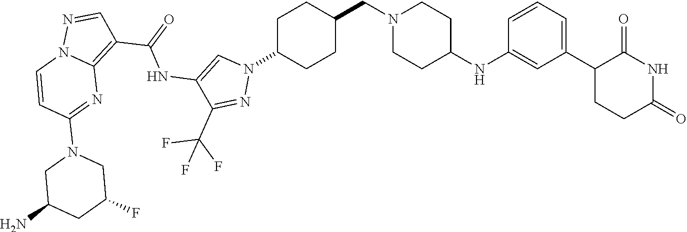 [SMILES: N[C@@H]1C[C@@H](F)CN(c2ccn3ncc(C(=O)Nc4cn([C@H]5CC[C@H](CN6CCC(Nc7cccc(C8CCC(=O)NC8=O)c7)CC6)CC5)nc4C(F)(F)F)c3n2)C1] |
| P-9 | 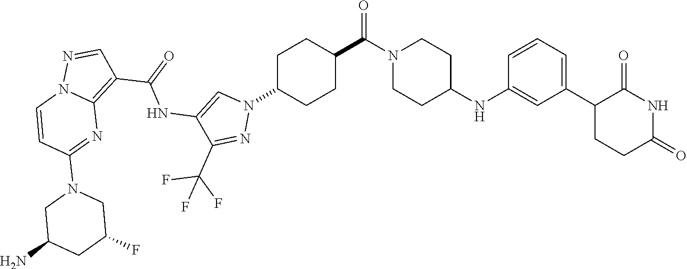 [SMILES: N[C@@H]1C[C@@H](F)CN(c2ccn3ncc(C(=O)Nc4cn([C@H]5CC[C@H](C(=O)N6CCC(Nc7cccc(C8CCC(=O)NC8=O)c7)CC6)CC5)nc4C(F)(F)F)c3n2)C1] |
| P-10 | 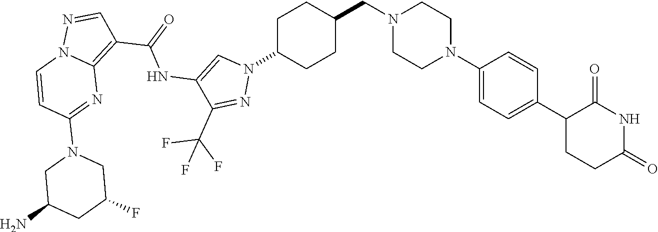 [SMILES: N[C@@H]1C[C@@H](F)CN(c2ccn3ncc(C(=O)Nc4cn([C@H]5CC[C@H](CN6CCN(c7ccc(C8CCC(=O)NC8=O)cc7)CC6)CC5)nc4C(F)(F)F)c3n2)C1] |
| P-11 | 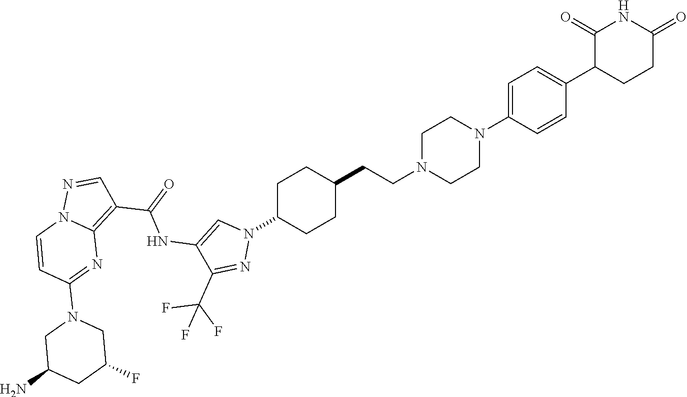 [SMILES: N[C@@H]1C[C@@H](F)CN(c2ccn3ncc(C(=O)Nc4cn([C@H]5CC[C@H](CCN6CCN(c7ccc(C8CCC(=O)NC8=O)cc7)CC6)CC5)nc4C(F)(F)F)c3n2)C1] |
| P-12 | 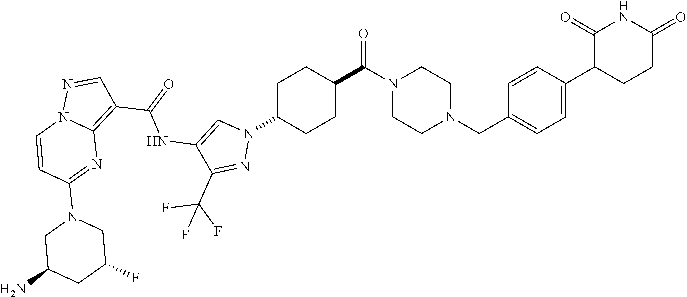 [SMILES: N[C@@H]1C[C@@H](F)CN(c2ccn3ncc(C(=O)Nc4cn([C@H]5CC[C@H](C(=O)N6CCN(Cc7ccc(C8CCC(=O)NC8=O)cc7)CC6)CC5)nc4C(F)(F)F)c3n2)C1] |
| P-13 | 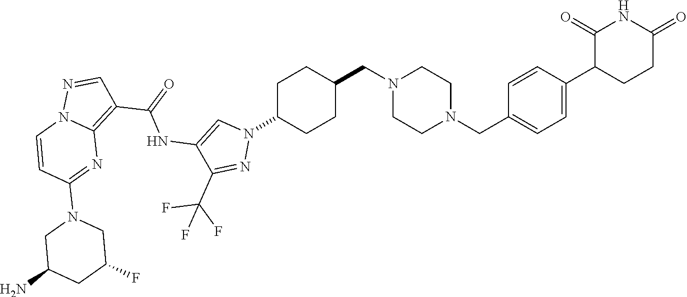 [SMILES: N[C@@H]1C[C@@H](F)CN(c2ccn3ncc(C(=O)Nc4cn([C@H]5CC[C@H](CN6CCN(Cc7ccc(C8CCC(=O)NC8=O)cc7)CC6)CC5)nc4C(F)(F)F)c3n2)C1] |
| P-14 | 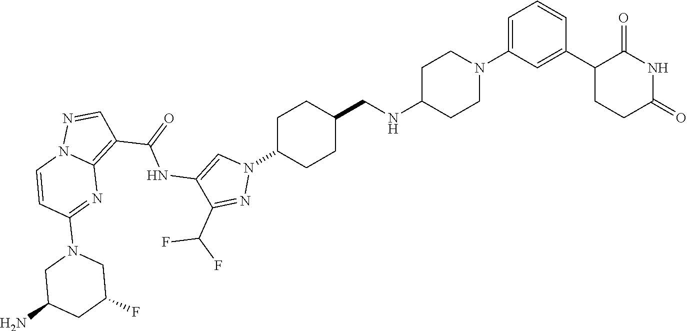 [SMILES: N[C@@H]1C[C@@H](F)CN(c2ccn3ncc(C(=O)Nc4cn([C@H]5CC[C@H](CNC6CCN(c7cccc(C8CCC(=O)NC8=O)c7)CC6)CC5)nc4C(F)F)c3n2)C1] |
| P-15 | 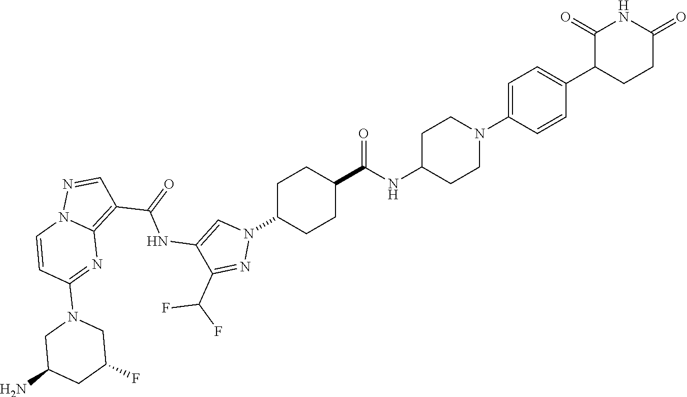 [SMILES: N[C@@H]1C[C@@H](F)CN(c2ccn3ncc(C(=O)Nc4cn([C@H]5CC[C@H](C(=O)NC6CCN(c7ccc(C8CCC(=O)NC8=O)cc7)CC6)CC5)nc4C(F)F)c3n2)C1] |
| P-16 | 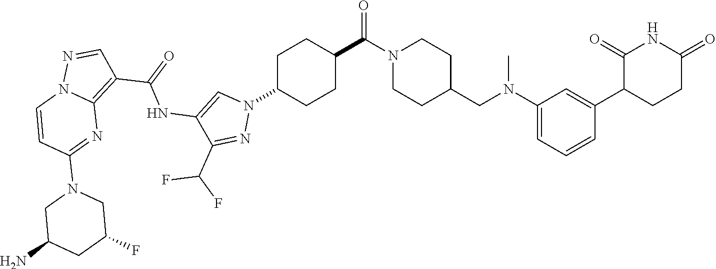 [SMILES: CN(CC1CCN(C(=O)[C@H]2CC[C@H](n3cc(NC(=O)c4cnn5ccc(N6C[C@H](N)C[C@@H](F)C6)nc45)c(C(F)F)n3)CC2)CC1)c1cccc(C2CCC(=O)NC2=O)c1] |
| P-17 | 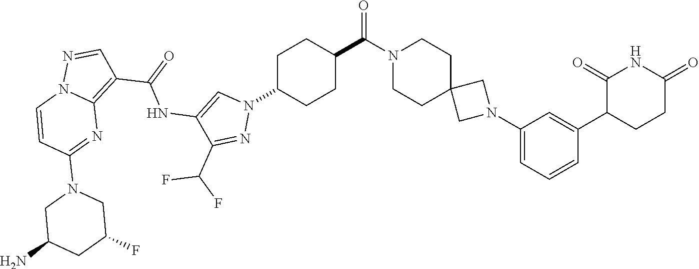 [SMILES: N[C@@H]1C[C@@H](F)CN(c2ccn3ncc(C(=O)Nc4cn([C@H]5CC[C@H](C(=O)N6CCC7(CC6)CN(c6cccc(C8CCC(=O)NC8=O)c6)C7)CC5)nc4C(F)F)c3n2)C1] |
| P-18 | 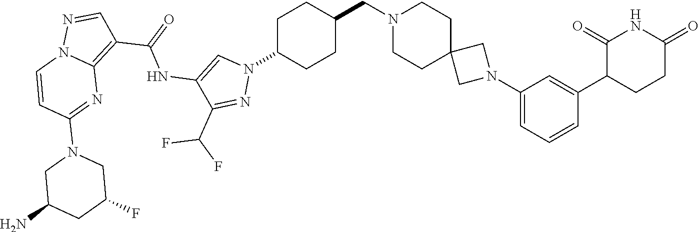 [SMILES: N[C@@H]1C[C@@H](F)CN(c2ccn3ncc(C(=O)Nc4cn([C@H]5CC[C@H](CN6CCC7(CC6)CN(c6cccc(C8CCC(=O)NC8=O)c6)C7)CC5)nc4C(F)F)c3n2)C1] |
| P-19 | 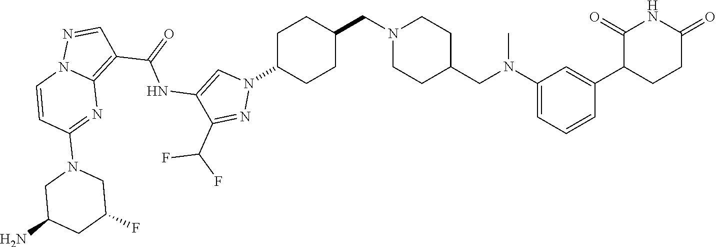 [SMILES: CN(CC1CCN(C[C@H]2CC[C@H](n3cc(NC(=O)c4cnn5ccc(N6C[C@H](N)C[C@@H](F)C6)nc45)c(C(F)F)n3)CC2)CC1)c1cccc(C2CCC(=O)NC2=O)c1] |
| P-20 | 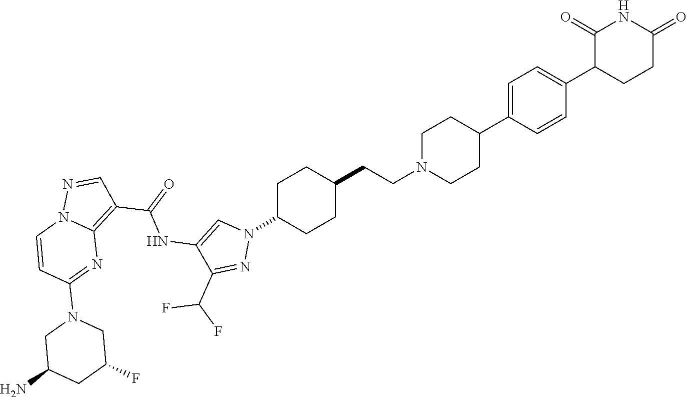 [SMILES: N[C@@H]1C[C@@H](F)CN(c2ccn3ncc(C(=O)Nc4cn([C@H]5CC[C@H](CCN6CCC(c7ccc(C8CCC(=O)NC8=O)cc7)CC6)CC5)nc4C(F)F)c3n2)C1] |
| P-21 | 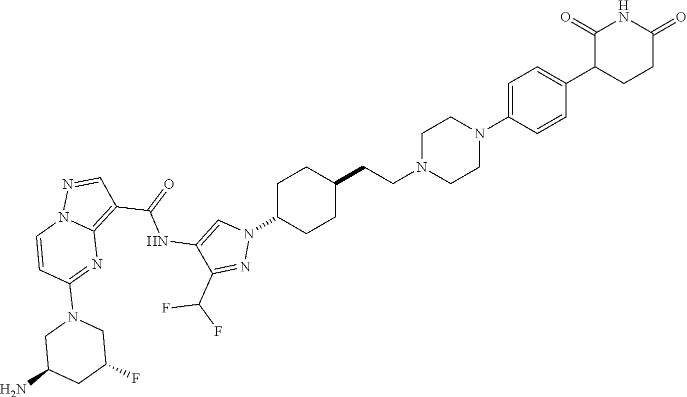 [SMILES: N[C@@H]1C[C@@H](F)CN(c2ccn3ncc(C(=O)Nc4cn([C@H]5CC[C@H](CCN6CCN(c7ccc(C8CCC(=O)NC8=O)cc7)CC6)CC5)nc4C(F)F)c3n2)C1] |
| P-22 | 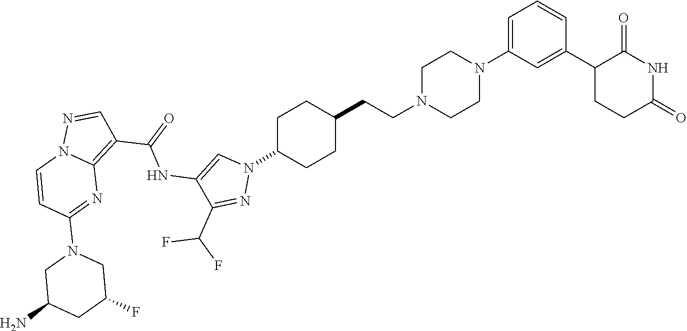 [SMILES: N[C@@H]1C[C@@H](F)CN(c2ccn3ncc(C(=O)Nc4cn([C@H]5CC[C@H](CCN6CCN(c7cccc(C8CCC(=O)NC8=O)c7)CC6)CC5)nc4C(F)F)c3n2)C1] |
| P-23 | 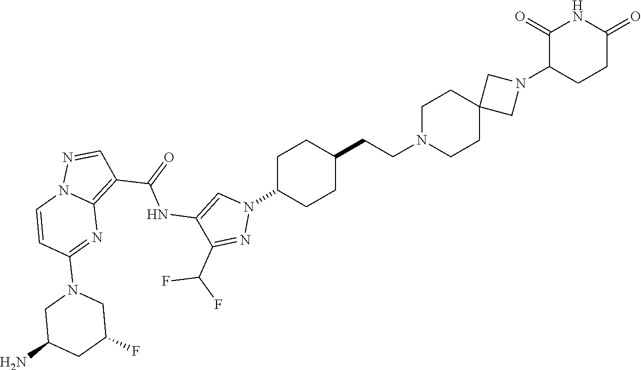 [SMILES: N[C@@H]1C[C@@H](F)CN(c2ccn3ncc(C(=O)Nc4cn([C@H]5CC[C@H](CCN6CCC7(CC6)CN(C6CCC(=O)NC6=O)C7)CC5)nc4C(F)F)c3n2)C1] |
| P-24 | 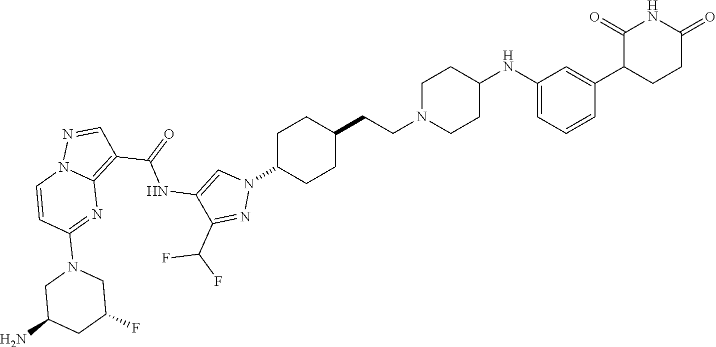 [SMILES: N[C@@H]1C[C@@H](F)CN(c2ccn3ncc(C(=O)Nc4cn([C@H]5CC[C@H](CCN6CCC(Nc7cccc(C8CCC(=O)NC8=O)c7)CC6)CC5)nc4C(F)F)c3n2)C1] |
| P-25 | 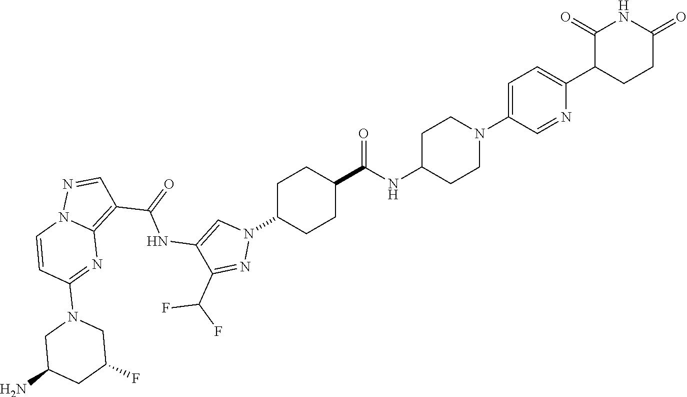 [SMILES: N[C@@H]1C[C@@H](F)CN(c2ccn3ncc(C(=O)Nc4cn([C@H]5CC[C@H](C(=O)NC6CCN(c7ccc(C8CCC(=O)NC8=O)nc7)CC6)CC5)nc4C(F)F)c3n2)C1] |
| P-26 | 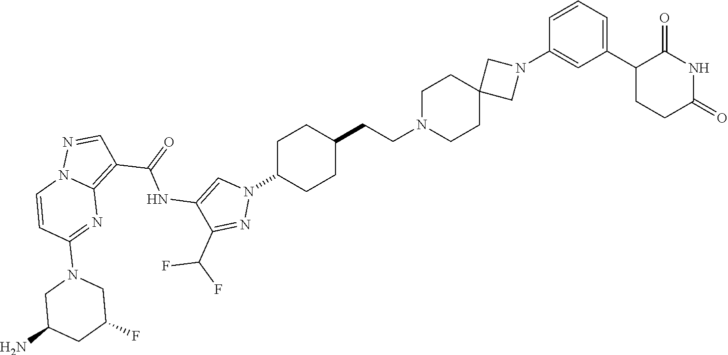 [SMILES: N[C@@H]1C[C@@H](F)CN(c2ccn3ncc(C(=O)Nc4cn([C@H]5CC[C@H](CCN6CCC7(CC6)CN(c6cccc(C8CCC(=O)NC8=O)c6)C7)CC5)nc4C(F)F)c3n2)C1] |
| P-27 | 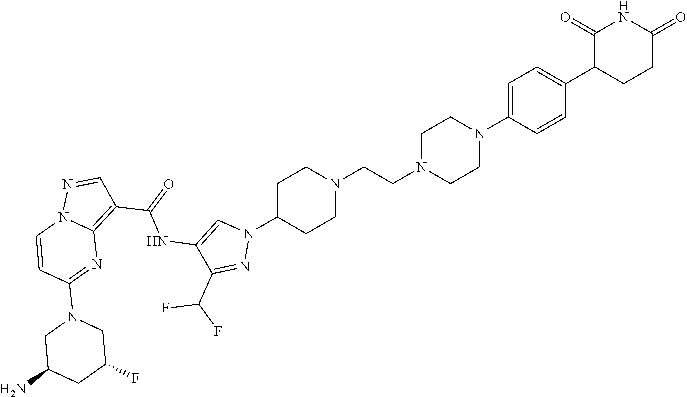 [SMILES: N[C@@H]1C[C@@H](F)CN(c2ccn3ncc(C(=O)Nc4cn(C5CCN(CCN6CCN(c7ccc(C8CCC(=O)NC8=O)cc7)CC6)CC5)nc4C(F)F)c3n2)C1] |
| P-28 | 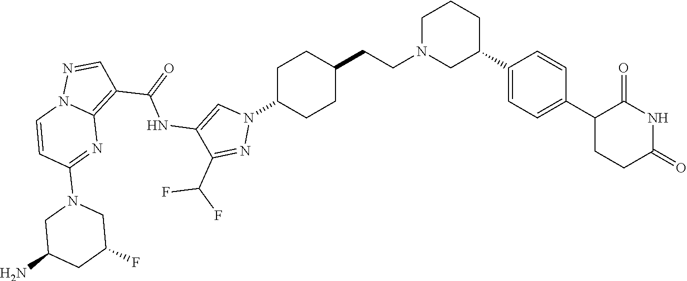 [SMILES: N[C@@H]1C[C@@H](F)CN(c2ccn3ncc(C(=O)Nc4cn([C@H]5CC[C@H](CCN6CCC[C@H](c7ccc(C8CCC(=O)NC8=O)cc7)C6)CC5)nc4C(F)F)c3n2)C1] |
| P-29 | 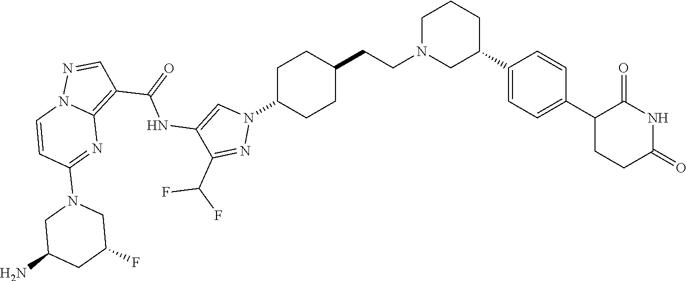 [SMILES: N[C@@H]1C[C@@H](F)CN(c2ccn3ncc(C(=O)Nc4cn([C@H]5CC[C@H](CCN6CCC[C@H](c7ccc(C8CCC(=O)NC8=O)cc7)C6)CC5)nc4C(F)F)c3n2)C1] |
| P-30 | 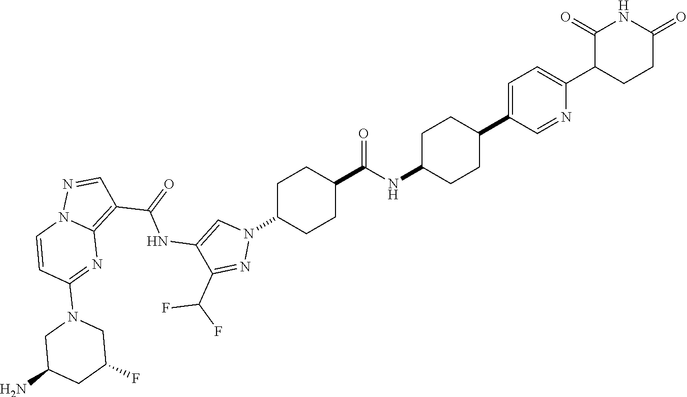 [SMILES: N[C@@H]1C[C@@H](F)CN(c2ccn3ncc(C(=O)Nc4cn([C@H]5CC[C@H](C(=O)N[C@H]6CC[C@@H](c7ccc(C8CCC(=O)NC8=O)nc7)CC6)CC5)nc4C(F)F)c3n2)C1] |
| P-31 | 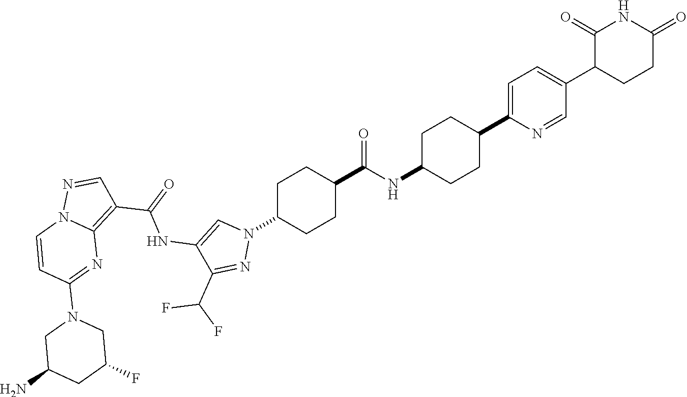 [SMILES: N[C@@H]1C[C@@H](F)CN(c2ccn3ncc(C(=O)Nc4cn([C@H]5CC[C@H](C(=O)N[C@H]6CC[C@@H](c7ccc(C8CCC(=O)NC8=O)cn7)CC6)CC5)nc4C(F)F)c3n2)C1] |
| P-32 | 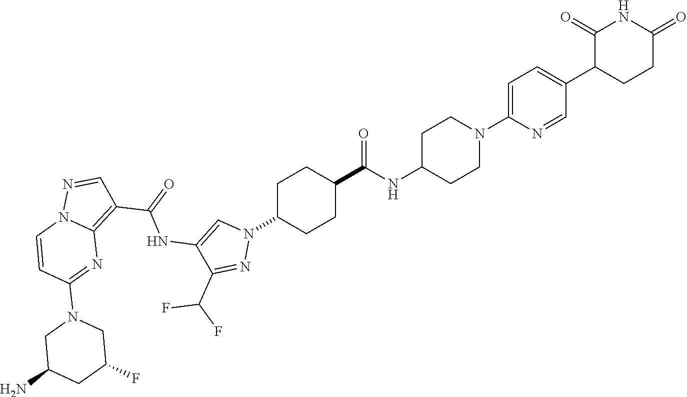 [SMILES: N[C@@H]1C[C@@H](F)CN(c2ccn3ncc(C(=O)Nc4cn([C@H]5CC[C@H](C(=O)NC6CCN(c7ccc(C8CCC(=O)NC8=O)cn7)CC6)CC5)nc4C(F)F)c3n2)C1] |
| P-33 | 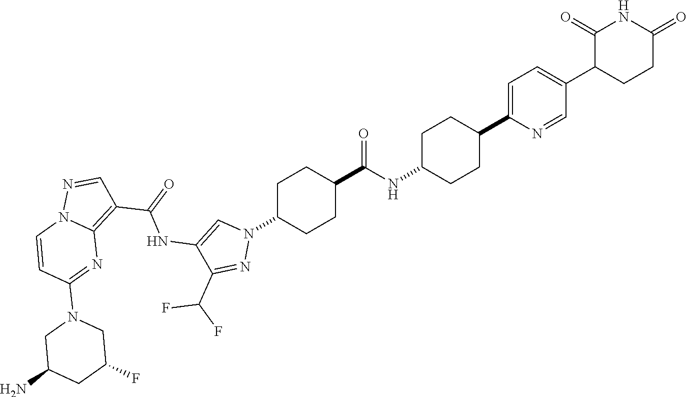 [SMILES: N[C@@H]1C[C@@H](F)CN(c2ccn3ncc(C(=O)Nc4cn([C@H]5CC[C@H](C(=O)N[C@H]6CC[C@H](c7ccc(C8CCC(=O)NC8=O)cn7)CC6)CC5)nc4C(F)F)c3n2)C1] |
| P-34 | 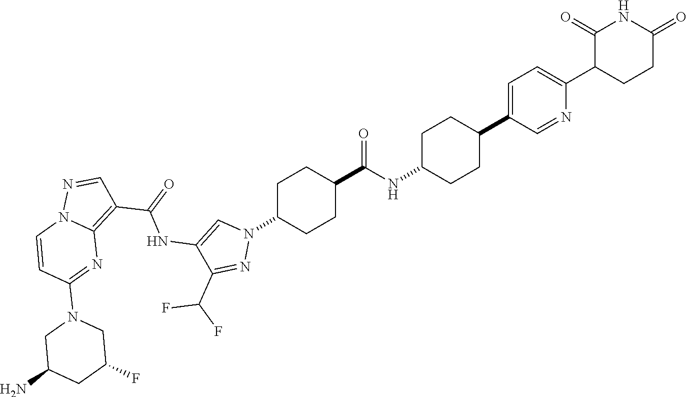 [SMILES: N[C@@H]1C[C@@H](F)CN(c2ccn3ncc(C(=O)Nc4cn([C@H]5CC[C@H](C(=O)N[C@H]6CC[C@H](c7ccc(C8CCC(=O)NC8=O)nc7)CC6)CC5)nc4C(F)F)c3n2)C1] |

or a pharmaceutically acceptable salt thereof.

It is understood that in the present description, combinations of substituents and/or variables of the depicted formulae are permissible only if such contributions result in stable compounds.

Furthermore, all compounds of Formula (I) that exist in free base or acid form can be converted to their pharmaceutically acceptable salts by treatment with the appropriate inorganic or organic base or acid by methods known to one skilled in the art. Salts of the compounds of Formula (I) can be converted to their free base or acid form by standard techniques.

### Methods of Synthesis

The compounds described herein can be made using conventional organic syntheses and commercially available starting materials, or the methods provided herein. By way of example and not limitation, compounds of Formula (I) can be prepared as outlined in Schemes 1-3, as well as in the examples set forth herein. It should be noted that one skilled in the art would know how to modify the procedures set forth in the illustrative schemes and examples to arrive at the desired products.

Compounds of Formula A can be prepared as outlined in Scheme 1. Coupling of intermediate a-1 with intermediate a-2 under basic conditions, such as in the presence of DIPEA, forms intermediate a-3, which is then deprotected under basic conditions to form intermediate a-4. Subsequent coupling of intermediate a-4 with intermediate a-5 using TCFH/NMI affords intermediate a-6, which is then deprotected under basic conditions to yield intermediate a-7, followed by coupling with intermediate a-8 (for example, using HATU) to generate intermediate a-9. Deprotection of a-9 under acidic conditions affords compounds of Formula A.

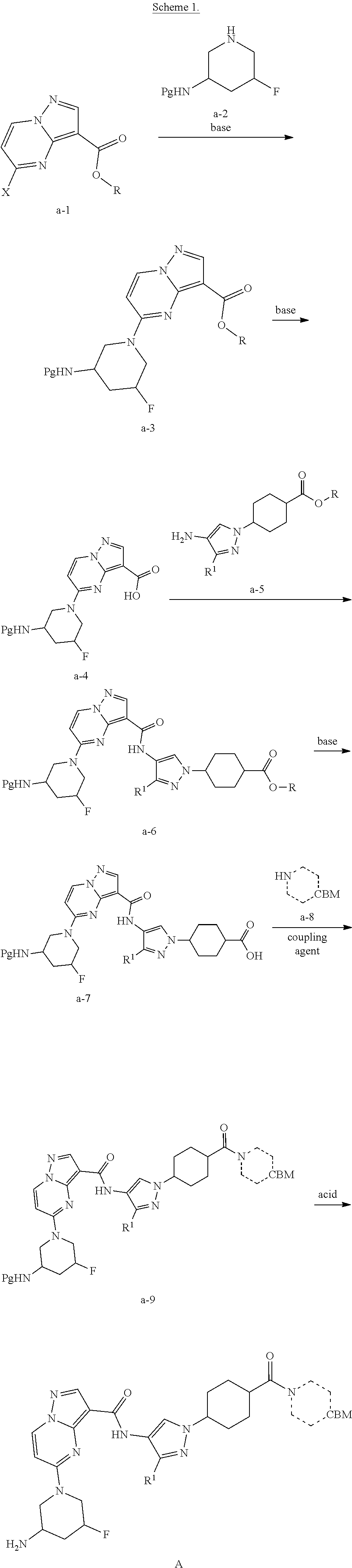

wherein X is halo such as chloro; Pg is a protecting group such as Boc; each R is independently alkyl such as methyl or ethyl; R¹ is as described for Formula (I);

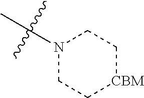

and represents the portion of Formula (I) corresponding to

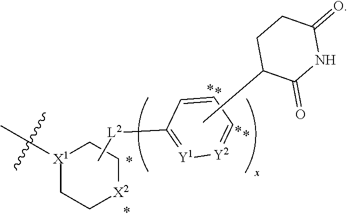

Compounds of Formula B can be prepared as outlined in Scheme 2. Reduction of intermediate a-5 (for example, using NaBH₄) affords intermediate b-1. Next, coupling of intermediate b-1 with intermediate a-4 using TCFH/NMI affords intermediate b-2, which is then oxidized, followed by reductive amination with intermediate a-8 (for example, using IBX followed by NaBH(OAc)₃) to generate intermediate b-3. Subsequent deprotection of b-3 under acidic conditions affords compounds of Formula B.

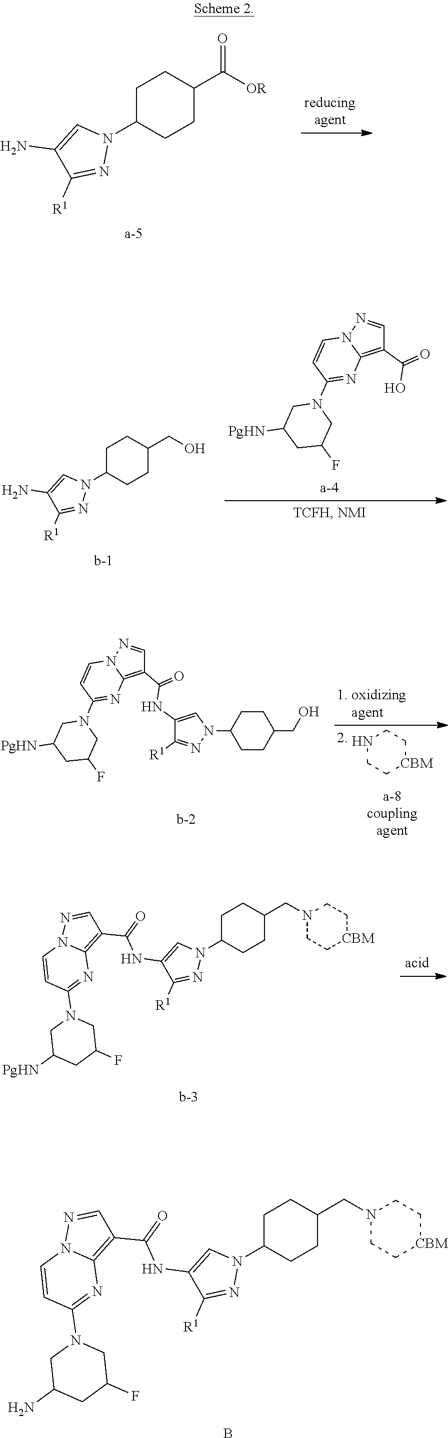

wherein Pg is a protecting group such as Boc; R is alkyl such as methyl or ethyl; R¹ is as described for Formula (I); and

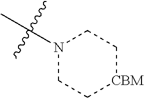

represents the portion of Formula (I) corresponding to

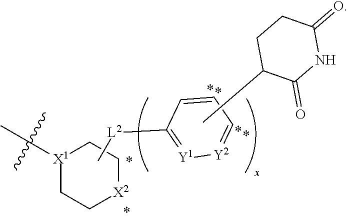

Compounds of Formula C can be prepared as outlined in Scheme 3. Reduction of intermediate c-1 (for example, using NaBH₄) affords intermediate c-2, followed by conversion to c-3. Next, cyanylation affords intermediate c-4, which is then converted to intermediate c-5 (for example, using DIBAL-H). Subsequent reduction of c-5 (for example, with NaBH₄) yields intermediate c-6, which is then hydrogenated to give intermediate c-7. Coupling of intermediate c-7 with intermediate a-4 using TCFH/NMI affords intermediate c-8, which is then oxidized, followed by reductive amination with intermediate a-8 (for example, using IBX followed by NaBH(OAc)₃) to generate intermediate c-9. Subsequent deprotection of c-9 under acidic conditions affords compounds of Formula C.

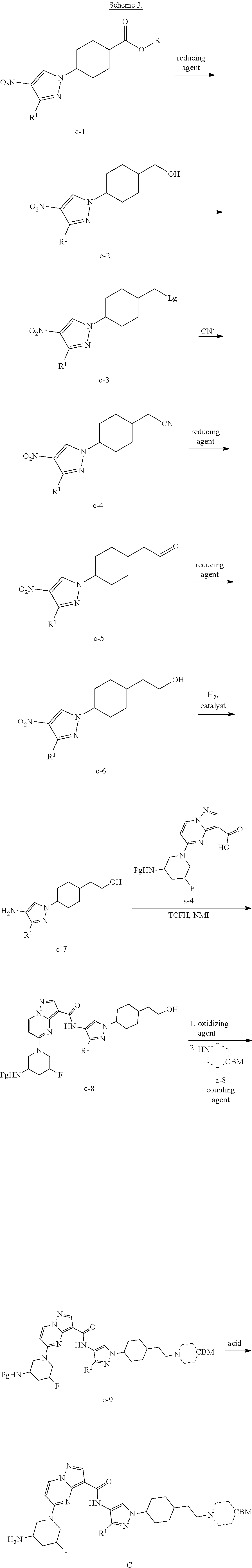

wherein Pg is a protecting group such as Boc; Lg is a leaving group, such as mesylate; R is alkyl such as methyl or ethyl; R¹ is as described for Formula (I); and

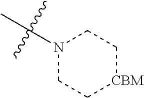

represents the portion of Formula (I) corresponding to

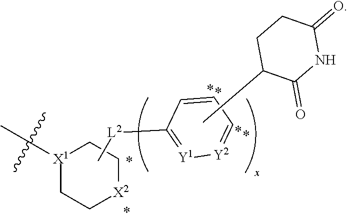

### Methods of Use

Embodiments of the present disclosure provide a method for modulating IRAK4 in a subject in need thereof, the method comprising administering to the subject an effective amount of a compound of Formula (I). Modulation (e.g., inhibition or activation) of IRAK 4 can be assessed and demonstrated by a wide variety of ways known in the art. Kits and commercially available assays can be utilized for determining whether and to what degree IRAK4 has been modulated (e.g., inhibited or activated).

In one aspect, provided herein is a method of modulating IRAK4 comprising contacting IRAK4 with an effective amount of a compound of Formula (I) or any embodiment or variation thereof. In some embodiments, the compound of Formula (I) inhibits IRAK4. In other embodiments, the compound of Formula (I) activates IRAK4. In some embodiments, the compound of Formula (I) is an agonist of IRAK4. In some embodiments, the compound of Formula (I) is an antagonist of IRAK4.

In some embodiments, provided herein is a method for targeting IRAK4 for degradation comprising contacting IRAK4 with an effective amount of a compound of Formula (I) or any embodiment or variation thereof.

In some embodiments, a compound of Formula (I) modulates the activity of IRAK4 by about 1%, 5%, 10%, 15%, 20%, 25%, 30%, 35%, 40%, 45%, 50%, 55%, 60%, 65%, 70%, 75%, 80%, 85%, 90%, 95%, or 100%. In some embodiments, a compound of Formula (I) modulates the activity of IRAK4 by about 1-100%, 5-100%, 10-100%, 15-100%, 20-100%, 25-100%, 30-100%, 35-100%, 40-100%, 45-100%, 50-100%, 55-100%, 60-100%, 65-100%, 70-100%, 75-100%, 80-100%, 85-100%, 90-100%, 95-100%, 5-95%, 5-90%, 5-85%, 5-80%, 5-75%, 5-70%, 5-65%, 5-60%, 5-55%, 5-50%, 5-45%, 5-40%, 5-35%, 5-30%, 5-25%, 5-20%, 5-15%, 5-10%, 10-90%, 20-80%, 30-70%, or 40-60%.

Also provided in certain embodiments of the present disclosure is a method for degrading IRAK4 in a subject in need thereof, the method comprising administering to the subject an effective amount of a compound of Formula (I). Degradation of IRAK4 can be assessed and demonstrated by a wide variety of ways known in the art. Kits and commercially available assays, including cell-based assays, can be utilized for determining whether and to what degree IRAK4 has been degraded.

In one aspect, provided herein is a method of degrading IRAK4 comprising contacting IRAK4 with an effective amount of a compound of Formula (I) or any embodiment or variation thereof. In some embodiments, the compound of Formula (I) partially degrades IRAK3. In some embodiments, the compound of Formula (I) fully degrades IRAK4.

In some embodiments, a compound of Formula (I) degrades IRAK4 by about 1%, 5%, 10%, 15%, 20%, 25%, 30%, 35%, 40%, 45%, 50%, 55%, 60%, 65%, 70%, 75%, 80%, 85%, 90%, 95%, or 100%. In some embodiments, a compound of Formula (I) degrades IRAK4 by about 1-100%, 5-100%, 10-100%, 15-100%, 20-100%, 25-100%, 30-100%, 35-100%, 40-100%, 45-100%, 50-100%, 55-100%, 60-100%, 65-100%, 70-100%, 75-100%, 80-100%, 85-100%, 90-100%, 95-100%, 5-95%, 5-90%, 5-85%, 5-80%, 5-75%, 5-70%, 5-65%, 5-60%, 5-55%, 5-50%, 5-45%, 5-40%, 5-35%, 5-30%, 5-25%, 5-20%, 5-15%, 5-10%, 10-90%, 20-80%, 30-70%, or 40-60%.

In another aspect, provided herein is a method for treating an inflammatory or autoimmune disease in a subject in need thereof, comprising administering to the subject an effective amount of a compound of Formula (I). In some embodiments, provided herein is a method for treating an inflammatory disease in a subject in need thereof, comprising administering to the subject an effective amount of a compound of Formula (I). In some embodiments, provided herein is a method for treating an autoimmune disease in a subject in need thereof, comprising administering to the subject an effective amount of a compound of Formula (I). In some embodiments, provided herein is a method for preventing an inflammatory or autoimmune disease in a subject in need thereof, comprising administering to the subject an effective amount of a compound of Formula (I). In some embodiments, provided herein is a method for preventing an inflammatory disease in a subject in need thereof, comprising administering to the subject an effective amount of a compound of Formula (I). In some embodiments, provided herein is a method for preventing an autoimmune disease in a subject in need thereof, comprising administering to the subject an effective amount of a compound of Formula (I). Non-limiting examples of an inflammatory or autoimmune disease include atopic dermatitis, asthma, lupus, rheumatoid arthritis, familial mediterranean fever, psoriasis, generalized pustular psoriasis, cryoprin-associated periodic syndrome, hidradenitis suppurativa, Bechet's syndrome, or familial cold autoinflammatory syndrome.

In some embodiments, administering a compound of Formula (I) to a subject that is predisposed to an inflammatory or autoimmune disease prevents the subject from developing any symptoms of the inflammatory or autoimmune disease. In some embodiments, administering a compound of Formula (I) to a subject that does not yet display symptoms of an inflammatory or autoimmune disease prevents the subject from developing any symptoms of the inflammatory or autoimmune disease. In some embodiments, administering a compound of Formula (I) to a subject in need thereof diminishes the extent of the inflammatory or autoimmune disease in the subject. In some embodiments, administering a compound of Formula (I) to a subject in need thereof stabilizes the inflammatory or autoimmune disease (prevents or delays the worsening of the inflammatory or autoimmune disease). In some embodiments, administering a compound of Formula (I) to a subject in need thereof delays the occurrence or recurrence of the inflammatory or autoimmune disease. In some embodiments, administering a compound of Formula (I) to a subject in need thereof slows the progression of the inflammatory or autoimmune disease. In some embodiments, administering a compound of Formula (I) to a subject in need thereof provides a partial remission of the inflammatory or autoimmune disease. In some embodiments, administering a compound of Formula (I) to a subject in need thereof provides a total remission of the inflammatory or autoimmune disease. In some embodiments, administering a compound of Formula (I) to a subject in need thereof decreases the dose of one or more other medications required to treat the inflammatory or autoimmune disease. In some embodiments, administering a compound of Formula (I) to a subject in need thereof enhances the effect of another medication used to treat the inflammatory or autoimmune disease. In some embodiments, administering a compound of Formula (I) to a subject in need thereof delays the progression of the inflammatory or autoimmune disease. In some embodiments, administering a compound of Formula (I) to a subject in need thereof increases the quality of life of the subject having an inflammatory or autoimmune disease. In some embodiments, administering a compound of Formula (I) to a subject in need thereof prolongs survival of a subject having an inflammatory or autoimmune disease.

In one aspect, provided herein is method of preventing a subject that is predisposed to an inflammatory or autoimmune disease from developing any symptoms of the inflammatory or autoimmune disease, the method comprising administering a compound of Formula (I) to the subject. In some embodiments, provided herein is a method of preventing a subject that does not yet display symptoms of an inflammatory or autoimmune disease from developing any symptoms of the inflammatory or autoimmune disease, the method comprising administering a compound of Formula (I) to the subject.

In some aspects, provided herein is a method of diminishing the extent of an inflammatory or autoimmune disease in a subject, the method comprising administering a compound of Formula (I) to the subject. In some embodiments, provided herein is a method of stabilizing an inflammatory or autoimmune disease in a subject, the method comprising administering a compound of Formula (I) to the subject. In some embodiments, the method prevents the worsening of the inflammatory or autoimmune disease. In some embodiments, the method delays the worsening of the inflammatory or autoimmune disease.

In another aspect, provided herein is a method of delaying the occurrence or recurrence of an inflammatory or autoimmune disease in a subject, the method comprising administering a compound of Formula (I) to the subject.

In some embodiments, provided herein is a method of slowing the progression of an inflammatory or autoimmune disease in a subject, the method comprising administering a compound of Formula (I) to the subject. In some embodiments, the method provides a partial remission of the inflammatory or autoimmune disease. In some embodiments, the method provides a total remission of the inflammatory or autoimmune disease.

In further aspects, provided herein is a method of decreasing the dose of one or more other medications required to treat an inflammatory or autoimmune disease in a subject, the method comprising administering a compound of Formula (I) to the subject. In some embodiments, provided herein is a method of enhancing the effect of another medication used to treat an inflammatory or autoimmune disease in a subject, the method comprising administering a compound of Formula (I) to the subject.

Also provided here is a method of delaying the progression of an inflammatory or autoimmune disease in a subject, the method comprising administering a compound of Formula (I) to the subject. In some embodiments, the method increases the quality of life of the subject having an inflammatory or autoimmune disease. In some embodiments, the method prolongs survival of the subject having an inflammatory or autoimmune disease.

In another aspect, provided herein is a method for treating inflammatory or autoimmune symptoms caused by a disease in a subject in need thereof, comprising administering to the subject an effective amount of a compound of Formula (I). In some embodiments, provided herein is a method for preventing inflammatory or autoimmune symptoms caused by a disease in a subject in need thereof, comprising administering to the subject an effective amount of a compound of Formula (I). In some embodiments, administering a compound of Formula (I) to a subject that is predisposed to a disease which causes inflammatory or autoimmune symptoms prevents the subject from developing any inflammatory or autoimmune symptoms. In some embodiments, administering a compound of Formula (I) to a subject that does not yet display inflammatory or autoimmune symptoms of a disease which causes inflammatory or autoimmune symptoms prevents the subject from developing any inflammatory or autoimmune symptoms. In some embodiments, administering a compound of Formula (I) to a subject in need thereof diminishes the extent of the inflammatory or autoimmune symptoms caused by the disease in the subject. In some embodiments, administering a compound of Formula (I) to a subject in need thereof stabilizes the inflammatory or autoimmune symptoms of the disease (prevents or delays the worsening of the inflammatory or autoimmune symptoms). In some embodiments, administering a compound of Formula (I) to a subject in need thereof delays the occurrence or recurrence of the inflammatory or autoimmune symptoms caused by the disease. In some embodiments, administering a compound of Formula (I) to a subject in need thereof slows the progression of the inflammatory or autoimmune symptoms caused by the disease. In some embodiments, administering a compound of Formula (I) to a subject in need thereof provides a partial remission of the disease which causes inflammatory or autoimmune symptoms. In some embodiments, administering a compound of Formula (I) to a subject in need thereof provides a total remission of the disease which causes inflammatory or autoimmune symptoms. In some embodiments, administering a compound of Formula (I) to a subject in need thereof decreases the dose of one or more other medications required to treat the disease which causes inflammatory or autoimmune symptoms. In some embodiments, administering a compound of Formula (I) to a subject in need thereof enhances the effect of another medication used to treat the inflammatory or autoimmune symptoms of the disease. In some embodiments, administering a compound of Formula (I) to a subject in need thereof delays the progression of the disease which causes inflammatory or autoimmune symptoms. In some embodiments, administering a compound of Formula (I) to a subject in need thereof increases the quality of life of the subject having a disease which causes inflammatory or autoimmune symptoms. In some embodiments, administering a compound of Formula (I) to a subject in need thereof prolongs survival of a subject having a disease which causes inflammatory or autoimmune symptoms. In some embodiments, the disease is atopic dermatitis, asthma, lupus, rheumatoid arthritis, familial mediterranean fever, psoriasis, generalized pustular psoriasis, cryoprin-associated periodic syndrome, hidradenitis suppurativa, Bechet's syndrome, or familial cold autoinflammatory syndrome.

In some embodiments, compounds of Formula (I) are useful for treating a disorder selected from atopic dermatitis, asthma, lupus, rheumatoid arthritis, familial mediterranean fever, psoriasis, generalized pustular psoriasis, cryoprin-associated periodic syndrome, hidradenitis suppurativa, Bechet's syndrome, and familial cold autoinflammatory syndrome.

### Pharmaceutical Compositions and Routes of Administration

The compounds provided herein can be administered to a subject orally, topically or parenterally in the conventional form of preparations, such as capsules, microcapsules, tablets, granules, powder, troches, pills, suppositories, injections, suspensions, syrups, patches, creams, lotions, ointments, gels, sprays, solutions and emulsions.

The compounds disclosed herein can be administered to a subject orally, topically or parenterally in the conventional form of preparations, such as capsules, microcapsules, tablets, granules, powder, troches, pills, suppositories, injections, suspensions, syrups, patches, creams, lotions, ointments, gels, sprays, solutions and emulsions. Suitable formulations can be prepared by methods commonly employed using conventional, organic or inorganic additives, such as an excipient (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylpyrrolidone, gelatin, gum arabic, polyethyleneglycol, sucrose or starch), a disintegrator (e.g., starch, carboxymethylcellulose, hydroxypropylstarch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g, sodium benzoate, sodium bisulfite, methylparaben or propylparaben), a stabilizer (e.g., citric acid, sodium citrate or acetic acid), a suspending agent (e.g., methylcellulose, polyvinyl pyrroliclone or aluminum stearate), a dispersing agent (e.g., hydroxypropylmethylcellulose), a diluent (e.g., water), and base wax (e.g., cocoa butter, white petrolatum or polyethylene glycol). The effective amount of the compounds of Formula (I) in the pharmaceutical composition may be at a level that will exercise the desired effect; for example, about 0.005 mg/kg of a subject's body weight to about 10 mg/kg of a subject's body weight in unit dosage for both oral and parenteral administration.

The dose of a compound of Formula (I) to be administered to a subject is rather widely variable and can be subject to the judgment of a health-care practitioner. In general, the compounds disclosed herein can be administered one to four times a day in a dose of about 0.001 mg/kg of a subject's body weight to about 10 mg/kg of a subject's body weight, but the above dosage may be properly varied depending on the age, body weight and medical condition of the subject and the type of administration. In any given case, the amount of the compound of Formula (I) administered may depend on such factors as the solubility of the active component, the formulation used and the route of administration.

A compound of Formula (I) can be administered orally for reasons of convenience. In one embodiment, when administered orally, a compound of Formula (I) is administered with a meal and water. In another embodiment, the compound of Formula (I) is dispersed in water or juice (e.g., apple juice or orange juice) or any other liquid and administered orally as a solution or a suspension.

The compounds disclosed herein can also be administered intradermally, intramuscularly, intraperitoneally, percutaneously, intravenously, subcutaneously, intranasally, epidurally, sublingually, intracerebrally, intravaginally, transdermally, rectally, mucosally, by inhalation, or topically to the ears, nose, eyes, or skin. The mode of administration is left to the discretion of the health-care practitioner, and can depend inpart upon the site of the medical condition.

In one embodiment, provided herein are capsules containing a compound of Formula (I) without an additional carrier, excipient or vehicle.

In another embodiment, provided herein are compositions comprising an effective amount of a compound of Formula (I) and a pharmaceutically acceptable carrier or vehicle, wherein a pharmaceutically acceptable carrier or vehicle can comprise an excipient, diluent, or a mixture thereof. In one embodiment, the composition is a pharmaceutical composition.

The compositions can be in the form of tablets, chewable tablets, capsules, solutions, parenteral solutions, troches, suppositories and suspensions and the like. Compositions can be formulated to contain a daily dose, or a convenient fraction of a daily dose, in a dosage unit, which may be a single tablet or capsule or convenient volume of a liquid. In one embodiment, the solutions are prepared from water-soluble salts, such as the hydrochloride salt. In general, all of the compositions are prepared according to known methods in pharmaceutical chemistry. Capsules can be prepared by mixing a compound of Formula (I) with a suitable carrier or diluent and filling the proper amount of the mixture in capsules. The usual carriers and diluents include, but are not limited to, inert powdered substances such as starch of many different kinds, powdered cellulose, especially crystalline and microcrystalline cellulose, sugars such as fructose, mannitol and sucrose, grain flours and similar edible powders.

Tablets can be prepared by direct compression, by wet granulation, or by dry granulation. Their formulations usually incorporate diluents, binders, lubricants and disintegrators as well as the compound. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

A lubricant might be necessary in a tablet formulation to prevent the tablet and punches from sticking in the dye. The lubricant can be chosen from such slippery solids as talc, magnesium and calcium stearate, stearic acid and hydrogenated vegetable oils. Tablet disintegrators are substances that swell when wetted to break up the tablet and release the compound. They include starches, clays, celluloses, algins and gums. More particularly, corn and potato starches, methylcellulose, agar, bentonite, wood cellulose, powdered natural sponge, cation-exchange resins, alginic acid, guar gum, citrus pulp and carboxymethyl cellulose, for example, can be used as well as sodium lauryl sulfate. Tablets can be coated with sugar as a flavor and sealant, or with film-forming protecting agents to modify the dissolution properties of the tablet. The compositions can also be formulated as chewable tablets, for example, by using substances such as mannitol in the formulation.

When it is desired to administer a compound of Formula (I) as a suppository, typical bases can be used. Cocoa butter is a traditional suppository base, which can be modified by addition of waxes to raise its melting point slightly. Water-miscible suppository bases comprising, particularly, polyethylene glycols of various molecular weights are in wide use.

The effect of the compound of Formula (I) can be delayed or prolonged by proper formulation. For example, a slowly soluble pellet of the compound of Formula (I) can be prepared and incorporated in a tablet or capsule, or as a slow-release implantable device. The technique also includes making pellets of several different dissolution rates and filling capsules with a mixture of the pellets. Tablets or capsules can be coated with a film that resists dissolution for a predictable period of time. Even the parenteral preparations can be made long-acting, by dissolving or suspending the compound of Formula (I) in oily or emulsified vehicles that allow it to disperse slowly in the serum.

### EXEMPLARY EMBODIMENTS

The present disclosure is further described by the following embodiments.

Embodiment P1. A compound of Formula (I):

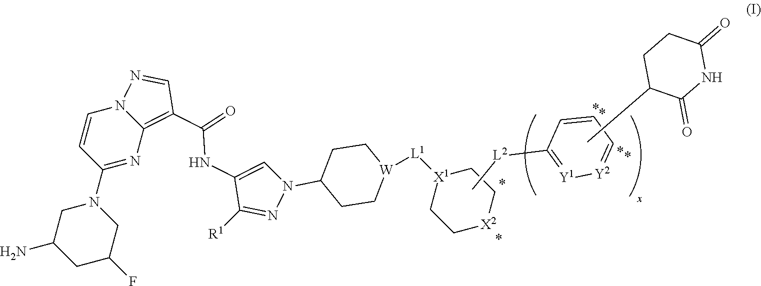

or a pharmaceutically acceptable salt thereof, wherein:
R¹ is C₁-C₆ haloalkyl;
W is CH or N;
X¹ is CH or N;
X² is CH₂, CH, C, N, or NH;
Y¹ and Y² are independently CH or N;
L¹ is —C(O)N(H)—, —C(O)—, or C₁-C₆ alkylene;
L² is a bond, —C(O)—, —NR²—, —CH₂N(R²)—, —CH₂—, or

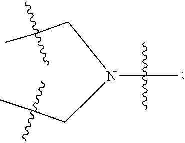 [SMILES: *CN(*)C*]

R² is H or C₁-C₆ alkyl;
x is 0 or 1;
* indicates an attachment point for L²; and
** indicates an attachment point for the piperidinyl-2,6-dione moiety.

Embodiment P2. The compound of embodiment P1, or a pharmaceutically acceptable salt thereof, wherein the compound is of Formula (IIa), (IIb), or (IIc):

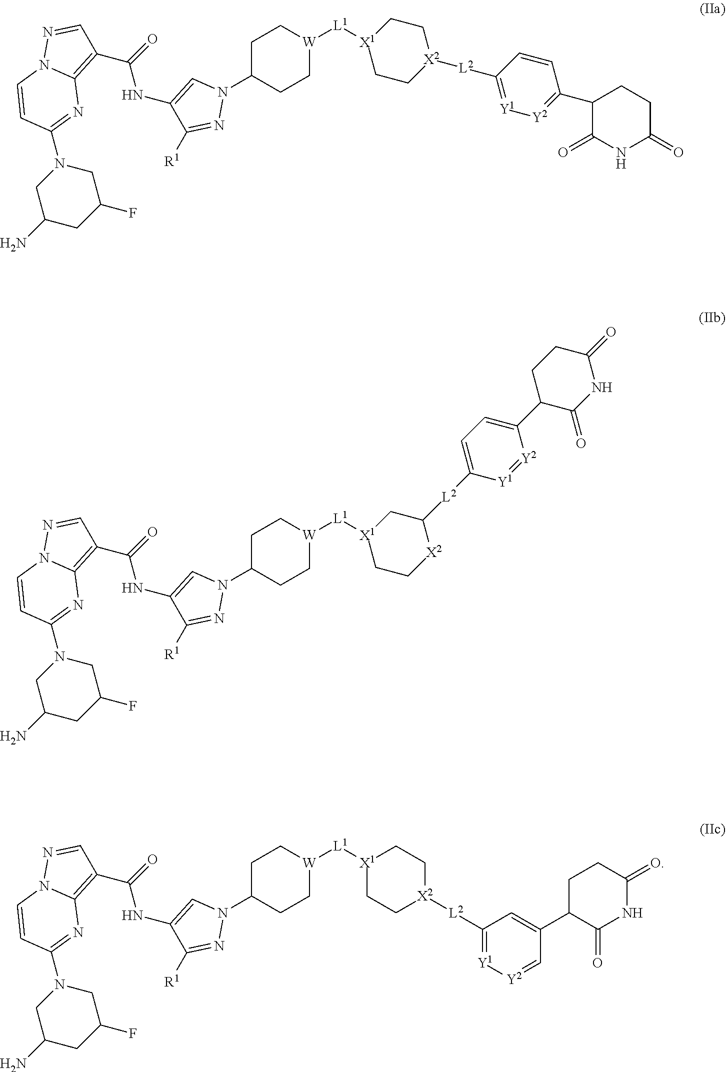

Embodiment P3. The compound of embodiment P1 or P2, or a pharmaceutically acceptable salt thereof, wherein:
R¹ is C₁-C₃ haloalkyl.

Embodiment P4. The compound of embodiment P3, or a pharmaceutically acceptable salt thereof, wherein:
R¹ is —CHF₂ or —CF₃.

Embodiment P5. The compound of any one of embodiments P1-P4, or a pharmaceutically acceptable salt thereof, wherein:
W is CH.

Embodiment P6. The compound of any one of embodiments P1-P4, or a pharmaceutically acceptable salt thereof, wherein:
W is N.

Embodiment P7. The compound of any one of embodiments P1-P6, or a pharmaceutically acceptable salt thereof, wherein:
X¹ is CH.

Embodiment P8. The compound of any one of embodiments P1-P6, or a pharmaceutically acceptable salt thereof, wherein:
X¹ is N.

Embodiment P9. The compound of any one of embodiments P1-P8, or a pharmaceutically acceptable salt thereof, wherein:
X² is CH₂, CH, or C.

Embodiment P10. The compound of any one of embodiments P1-P8, or a pharmaceutically acceptable salt thereof, wherein:
X² is N or NH.

Embodiment P11. The compound of any one of embodiments P1-P10, or a pharmaceutically acceptable salt thereof, wherein:
Y¹ is CH.

Embodiment P12. The compound of any one of embodiments P1-P10, or a pharmaceutically acceptable salt thereof, wherein:
Y¹ is N.

Embodiment P13. The compound of any one of embodiments P1-P12, or a pharmaceutically acceptable salt thereof, wherein:
Y² is CH.

Embodiment P14. The compound of any one of embodiments P1-P12, or a pharmaceutically acceptable salt thereof, wherein: Y² is N.
Embodiment P15. The compound of any one of embodiments P1-P14, or a pharmaceutically acceptable salt thereof, wherein:
L¹ is —C(O)N(H)—, —C(O)—, or C₁-C₃ alkylene.

Embodiment P16. The compound of embodiment P15, or a pharmaceutically acceptable salt thereof, wherein:
L¹ is —C(O)N(H)—, —C(O)—, —CH₂—, or —CH₂CH₂—.

Embodiment P17. The compound of any one of embodiments P1-P16, or a pharmaceutically acceptable salt thereof, wherein:
L² is a bond, —C(O)—, —NR²—, —CH₂N(R²)—, —CH₂—, or

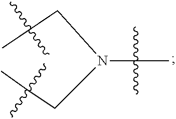 [SMILES: *CN(*)C*]

and
R² is H or C₁-C₃ alkyl.

Embodiment P18. The compound of embodiment P17, or a pharmaceutically acceptable salt thereof, wherein:
R² is H or —CH₃.

Embodiment P19. The compound of any one of embodiments P1-P18, or a pharmaceutically acceptable salt thereof, wherein:

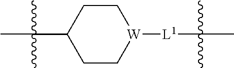

is

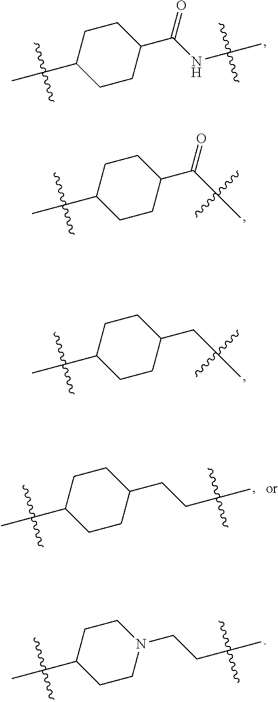 [SMILES: *NC(=O)C1CCC(*)CC1 ; *C(=O)C1CCC(*)CC1 ; *CC1CCC(*)CC1 ; *CCC1CCC(*)CC1 ; *CCN1CCC(*)CC1]

Embodiment P20. The compound of any one of embodiments P1-P19, or a pharmaceutically acceptable salt thereof, wherein:

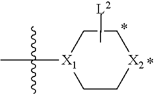

is

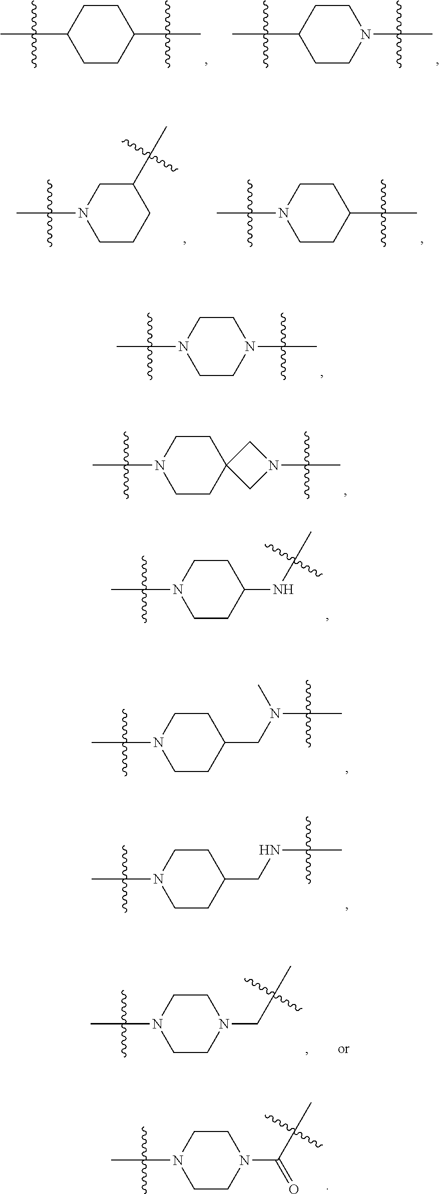

Embodiment P21. The compound of any one of embodiments P1-P20, or a pharmaceutically acceptable salt thereof, wherein:
x is 0.

Embodiment P22. The compound of any one of embodiments P1-P20, or a pharmaceutically acceptable salt thereof, wherein:
x is 1.

Embodiment P23. The compound of any one of embodiments P1-P20 and P22, or a pharmaceutically acceptable salt thereof, wherein:

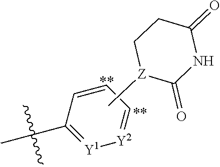

is

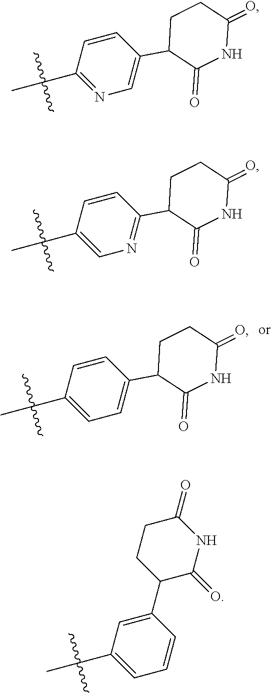 [SMILES: *c1ccc(C2CCC(=O)NC2=O)cn1 ; *c1ccc(C2CCC(=O)NC2=O)nc1 ; *c1ccc(C2CCC(=O)NC2=O)cc1 ; *c1cccc(C2CCC(=O)NC2=O)c1]

Embodiment P24. A compound selected from the compounds of Table 1 and pharmaceutically acceptable salts thereof.

Embodiment P25. A pharmaceutical composition comprising the compound of any one of embodiments P1-P24, or a pharmaceutically acceptable salt thereof, and a pharmaceutically acceptable excipient.

Embodiment P26. A method of modulating interleukin-1 (IL1) receptor-associated kinase 4 (IRAK4) comprising contacting IRAK4 with an effective amount of the compound of any one of embodiments P1-P24, or a pharmaceutically acceptable salt thereof, or the pharmaceutical composition of embodiment P25.

Embodiment P27. A method of treating an inflammatory or autoimmune disease in a subject in need thereof, comprising administering to the subject an effective amount of the compound of any one of embodiments P1-P24, or a pharmaceutically acceptable salt thereof, or the pharmaceutical composition of embodiment P25.

Embodiment P28. The method of embodiment P27, wherein the inflammatory or autoimmune disease is atopic dermatitis, asthma, lupus, rheumatoid arthritis, familial mediterranean fever, psoriasis, generalized pustular psoriasis, cryoprin-associated periodic syndrome, hidradenitis suppurativa, Bechet's syndrome, or familial cold autoinflammatory syndrome.

### EXAMPLES

The following Examples are presented by way of illustration, not limitation. Compounds are named using the automatic name generating tool provided in ChemBiodraw Ultra (Cambridgesoft), which generates systematic names for chemical structures, with support for the Cahn-Ingold-Prelog rules for stereochemistry. One skilled in the art can modify the procedures set forth in the illustrative examples to arrive at the desired products.

Salts of the compounds described herein can be prepared by standard methods, such as inclusion of an acid (for example TFA, formic acid, or HCl) in the mobile phases during chromatography purification, or stirring of the products after chromatography purification, with a solution of an acid (for example, aqueous HCl).

The following abbreviations may be relevant for the application.

### Abbreviations

AcOH: acetic acid
aq or aq.: aqueous
BCA assay: Bicinchoninic acid assay
BTEAC: benzyltriethylammonium chloride
BuOH: butanol
CBM: Cereblon Binding Moiety
CV: column volume
DCE: 1,2-dichloroethane
DCM: dichloromethane
DIPEA: N, N-diisopropylethylamine
DMF: dimethylformamide
DMS: dimethyl sulfide
DMSO: dimethylsulfoxide
eq.: equivalents
ESI: electrospray ionization
Et₃N: triethylamine
EtOAc: ethyl acetate
EtOH: ethanol
FA: formic acid
FBS: fetal bovine serum
FC: flash chromatography
h: hour(s)
HATU: 1-[bis(dimethylamino)methylene]-1H-1,2,3-triazolo[4,5-b]pyridinium 3-oxide hexafluorophosphate
HPLC: high-performance liquid chromatography
IBX: 2-iodoxybenzoic acid
LCMS: liquid chromatography mass spectrometry
MeCN: acetonitrile
MeOH: methanol
min: minute(s)
MsCl: mesyl chloride
MSD: mass selective detector
MsOH: methanesulfonic acid
MTBE: methyl tert-butyl ether
NaOtBu: sodium tert-butoxide
NMI: 1-methylimidazole
NMO: N-methylmorpholine N-oxide
PCy₃: tricyclohexylphosphine
Pd(PPh₃)₄: tetrakis(triphenylphosphine) palladium (0)
Pd₂(dba)₃: tris(dibenzylideneacetone) dipalladium (0)
PhMe: toluene
prep: preparative
quant.: quantitative
RP: reverse phase
rt or r.t.: room temperature
RT: retention time
SFC: supercritical-fluid chromatography
SM: starting material
t-BuOK: potassium tert-butoxide
TCFH: chloro-N,N,N′,N′-tetramethylformamidinium hexafluorophosphate
TEA: triethylamine
TFA: trifluoroacetic acid
THF: tetrahydrofuran
TLC: thin layer chromatography

### SYNTHETIC EXAMPLES

### LCMS Methods

### LCMS Method 1

Column: Luna C18 (2) 50×3 mm, 3 μm. Temperature: 45° C., Flow: 1.5 mL/min, run time: 2.5 min. Mobile phase conditions: Initial 95% H₂O 0.1% FA/5% MeCN 0.1% FA, linear gradient to 95% MeCN 0.1% FA over 1.3 min then hold for 1.2 minute at 95% MeCN 0.1% FA. MSD: ESI Positive

### LCMS Method 2

Column: Luna C18 (2) 50×3 mm, 3 μm. Temperature: 45° C. Flow: 1.5 mL/min, run time: 3.5 min. Mobile phase conditions: Initial 95% H₂O 0.1% FA/5% MeCN 0.1% FA, linear gradient to 95% MeCN 0.1% FA over 1.3 min then hold for 2.2 minute at 95% MeCN 0.1% FA. MSD: ESI Positive

### LCMS Method 3

Column: SunFire C18 75×4.6 mm, 3.5 μm. Temperature: 45° C., Flow: 1.5 mL/min, run time: 6 min. Mobile phase conditions: Initial 95% H₂O+0.1% FA/5% MeCN+0.1% FA then linear gradient to 95% MeCN+0.1% FA for 4 min then hold for 2 min at 95% MeCN+0.1% FA. MSD: Positive

### LCMS Method 4

Column: XBridge C18 4.6×75 mm 5 μm, Temperature: 45° C., Flow 1.5 mL/min, run time: 6 min. Mobile phase conditions: Initial Gradient at 95% NH₄HCO₃/5% MeCN linear gradient 0 to 3 min at 95% MeCN and hold for 3 minutes.

### Synthesis of Intermediates

Reference to a particular intermediate compound by number, such as 1 or 2, is specific to the example in which it is described. As such, multiple examples may refer to the same intermediate compound number, such as 1 or 2, but the chemical structure of the compound will be different across the different examples.

### Example I-1. Synthesis of Common Intermediate T-1

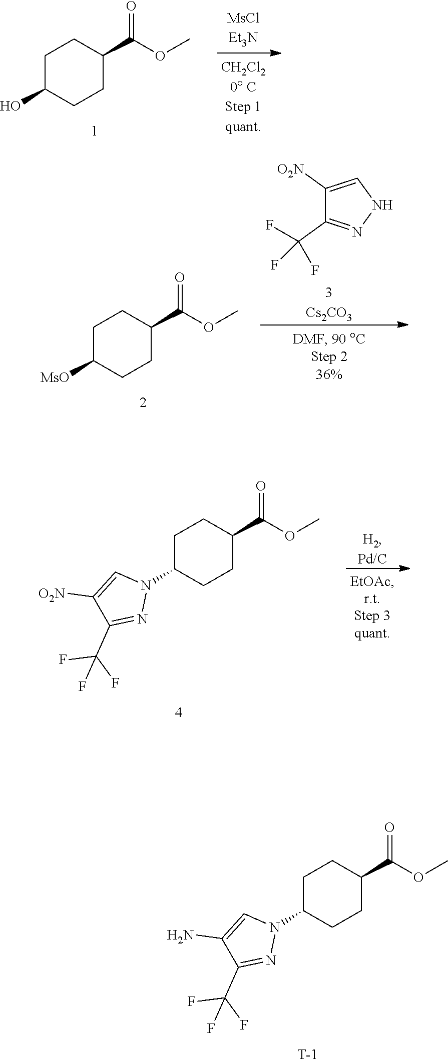

Step 1. Preparation of Methyl 4-methylsulfonyloxycyclohexanecarboxylate (2). To a solution of methyl 4-hydroxycyclohexanecarboxylate (1) (5.0 g, 31.61 mmol, 1.0 equiv.) and Et₃N (6.61 mL, 47.41 mmol, 1.5 equiv.) in CH₂Cl₂ (158 mL, 0.2 M) at 0° C. was added methanesulfonyl chloride (3.18 mL, 41.09 mmol, 1.3 equiv.) dropwise. The resulting mixture was stirred at 0° C. for 30 minutes. TLC (KMnO₄): SM: Rf=0.25, product: Rf=0.30 (60% EtO Ac/heptanes). Water was added and the aqueous phase was extracted with CH₂Cl₂ (3×). The combined organics were washed with aqueous 1 M HCl (1×), dried over Na₂SO₄, filtered, then concentrated to dryness under reduced pressure to afford pure 2 (7.47 g, quantitative yield) without any further purification.

LCMS Method 1: 99.9% purity at 215 nm; [M-MsOH+H]+=141.2 m/z, [M+Na]⁺=259.2 m/z.

¹H NMR (400 MHz, CDCl₃) δ ppm 1.67-1.82 (m, 4H), 1.87-1.97 (m, 2H), 1.97-2.07 (m, 2H), 2.36-2.45 (m, 1H), 3.01 (s, 3H), 3.68 (s, 3H), 4.88-4.94 (m, 1H).

Step 2. Preparation of Methyl (1r,4r)-4-(4-nitro-3-(trifluoromethyl)-1H-pyrazol-1-yl)cyclohexane-1-carboxylate (4). To a solution of 4-nitro-3-(trifluoromethyl)-1H-pyrazole 3 (1.0 g, 5.52 mmol, 1.0 eq.) and methyl 4-methylsulfonyloxycyclohexanecarboxylate 2 (1.3 g, 5.52 mmol, 2.0 eq.) in dry DMF (18.41 mL, 0.3 M) was added Cs₂CO₃ (3.6 g, 11.05 mmol, 2.0 eq.). After stirring at 90° C. overnight, LCMS showed complete conversion into 4. The reaction was quenched with water and the aqueous layer was extracted three times with EtOAc. The combined organic layers were washed two times with water, one time with brine, dried over Na₂SO₄, filtered and concentrated under reduced pression. The crude product was purified by flash column chromatography (80 g SiO₂ eluting with 0 to 35% MTBE in Heptane over 15 CVs, then 35% MTBE in Heptane over 3 CVs). The desired product came out 30% MTBE in Heptane (Rf-0.30 on TLC eluting 60% MTBE in Heptane, revealing UV+KMnO₄). The fractions were combined and concentrated to dryness to give 4 (671 mg, 36% yield) as light yellow solid.

LCMS method 1: retention time: 1.819 min, 99.9% purity at 215 nm, [M+H]⁺=322.2.

¹H NMR (400 MHz, DMSO-d6) δ ppm 1.52 (qd, J=13.0, 3.0 Hz, 2H), 1.83 (br dd, J=12.5, 3.1 Hz, 2H), 2.00-2.17 (m, 4H), 2.42 (tt, J=12.2, 3.5 Hz, 1H), 3.61 (s, 3H), 4.39 (tt, J=11.9, 3.8 Hz, 1H), 9.19 (s, 1H).

¹⁹F NMR (377 MHz, DMSO-d6) δ ppm −61.45 (s, 3F).

Step 3. Preparation of Methyl 4-[4-amino-3-(trifluoromethyl)pyrazol-1-yl]cyclohexanecarboxylate (T-1). To a solution of methyl 4-[4-nitro-3-(trifluoromethyl)pyrazol-1-yl]cyclohexanecarboxylate 4 (671 mg, 2.09 mmol, 1.0 eq.) in ethyl acetate (13.93 mL, 0.15 M) was added 10% Pd/C (444 mg, 0.42 mmol, 0.2 eq.). Nitrogen was bubbled through the solution for 10 min, then hydrogen was bubbled through the solution for 5 minutes, and the resulting mixture was stirred under a hydrogen atmosphere (1 atm). After stirring for 5 hours at room temperature, LCMS showed complete conversion into T-1. The reaction mixture was filtered through a pad of celite and washed with EtOAc. The filtrate was concentrated under reduced pressure to give T-1 (608 mg, quantitative yield) as a pink solid. The crude product was used in the next step without further purification.

LCMS method 1: retention time: 1.562 min, 99.9% purity at 215 nm, [M+H]₊=292.2.

¹H NMR (400 MHz, DMSO-d6) δ ppm 1.41-1.60 (m, 2H), 1.64-1.81 (m, 2H), 1.91-2.06 (m, 4H), 2.32-2.45 (m, 1H), 3.60 (s, 3H), 4.00-4.13 (m, 1H), 4.21 (s, 2H), 7.22 (s, 1H).

¹⁹F NMR (377 MHz, DMSO-d6) δ ppm −58.85 (s, 3F).

### Example I-2. Synthesis of Common Intermediate T-2

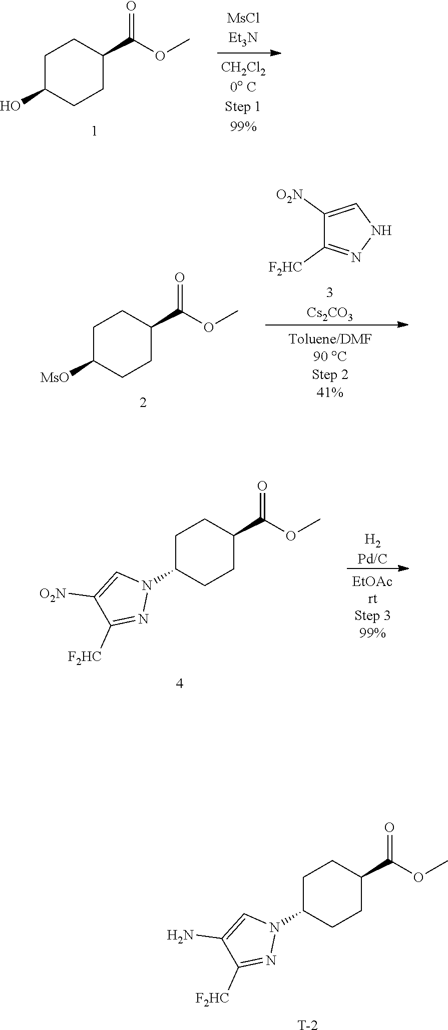

Step 1. Preparation of Methyl 4-methylsulfonyloxycyclohexanecarboxylate (2). Under nitrogen, a solution of methyl 4-hydroxycyclohexanecarboxylate 1 (5.0 g, 31.61 mmol, 1.0 eq.) in CH₂Cl₂ (105 mL, 0.3 M) was cooled to 0° C., then methanesulfonyl chloride (2.69 mL, 34.77 mmol, 1.1 eq.) and triethylamine (5.28 mL, 37.93 mmol, 1.2 eq.) were added, the latter dropwise. After stirring 2 h at 0° C., TLC (CH₂Cl₂/MeOH 5.5:0.5, KMnO₄ stain) showed full conversion. The reaction was quenched by addition of water, then phases were separated and the aqueous phase was extracted 3 times with CH₂Cl₂. The combined organic phases were washed twice with brine, dried over magnesium sulfate, filtered and concentrated to give 2 (7.45 g, 99% yield) as a yellow oil.

¹H NMR (400 MHz, CDCl₃) δ ppm 1.67-1.84 (m, 4H), 1.87-1.99 (m, 2H), 2.00-2.09 (m, 2H), 2.36-2.46 (m, 1H), 3.02 (s, 3H), 3.69 (s, 3H), 4.87-4.96 (m, 1H).

Step 2. Preparation of Methyl 4-[3-(difluoromethyl)-4-nitro-pyrazol-1-yl]cyclohexanecarboxylate (4). Under nitrogen, in a flame-dried round-bottom flask, a solution of 2 (1.16 g, 4.91 mmol, 1.0 eq.), 3 (0.80 g, 4.91 mmol, 1.0 eq.) and DMF (12.3 mL, 0.4 M) was stirred at room temperature for 5 minutes before Cs₂CO₃ (3.20 g, 9.81 mmol, 2.0 eq.) was added. The resulting mixture was stirred at 90° C. for 16 h. Incomplete conversion of 3 was observed by LCMS (method 1), then a second portion of 2 (1.16 g, 4.91 mmol, 1.0 eq.) was added and stirring at 90° C. was resumed for 48 h. A 90% conversion of 3 was observed by LCMS. The reaction was quenched by addition of water. Ethyl acetate was added and phases were separated. The aqueous phase was extracted 3 times with ethyl acetate, then the combined organic phases were washed once with water and once with brine, dried over magnesium sulfate and concentrated. The residue was taken up in MTBE and water, then phases were separated. The organic phase was washed 5 times with water, then once with brine, dried over magnesium sulfate, filtered and concentrated to give an orange oil. The residue was then purified by normal phase flash chromatography (80 g silica column, elution: 0 to 30% MTBE/heptanes over 10 CV). Fractions were combined and concentrated to give impure 4 (883 mg). The residue was then purified by reverse phase flash chromatography (100 g C18 RediSep Rf Gold column, liquid deposit (DMSO), elution: 5% MeOH/0.1% HCOOH over 4 CV, then 5% to 100% MeOH/0.1% HCOOH over 15 CV). Fractions were combined and concentrated to give 4 (618 mg, 41% yield) as a white solid.

LCMS method 1: retention time: 1.745 min, 99.9% purity at 215 nm, [M+H]⁺=304.2.

¹H NMR (400 MHz, DMSO-d6) δ ppm 1.45-1.60 (m, 2H), 1.76-1.89 (m, 2H), 2.00-2.15 (m, 4H), 2.36-2.47 (m, 1H), 3.61 (s, 3H), 4.28-4.40 (m, 1H), 7.14-7.47 (m, 1H), 9.05 (s, 1H).

¹⁹F NMR (377 MHz, DMSO-d6) δ ppm −117.40 (s, 2F).

Step 3. Preparation of Methyl 4-[4-amino-3-(difluoromethyl)pyrazol-1-yl]cyclohexanecarboxylate (T-2). Under nitrogen, a solution of 4 (625 mg, 2.06 mmol, 1.0 eq.), and ethyl acetate (20.6 mL, 0.1 M) was degassed at room temperature by sparging with nitrogen for 15 minutes. Pd/C (438 mg, 10% w/w, 0.41 mmol, 0.2 eq.) was added and sparging was resumed for 15 minutes. The mixture was sparged with H₂ for 15 minutes, then the needle was kept just over the surface of the solvent and the mixture was stirred at room temperature for 16 h. Complete conversion of 4 was observed by LCMS (method 1). The mixture was filtered over celite, then the filter cake was washed thoroughly with ethyl acetate and the resulting solution was concentrated to give T-2 (563 mg, 99% yield) as a light orange solid.

LCMS method 1: retention time: 1.297 min, 99.9% purity at 215 nm, [M+H]⁺=274.2.

¹H NMR (400 MHz, CDCl₃) δ ppm 1.53-1.80 (m, 5H), 2.12-2.42 (m, 9H), 3.93-4.03 (m, 1H), 6.54-6.84 (m, 1H), 7.07 (s, 1H).

¹⁹F NMR (377 MHz, CDCl₃) δ ppm −112.22 (s, 2F).

### Example I-3. Synthesis of Common Intermediate C-1

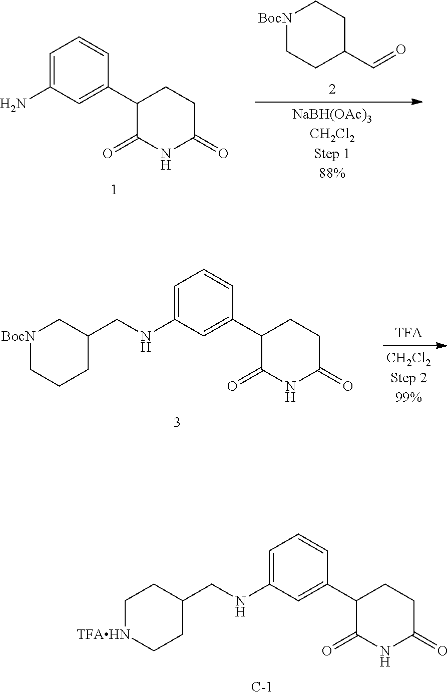

Step 1. Preparation of tert-Butyl 4-[[3-(2,6-dioxo-3-piperidyl)anilino]methyl]piperidine-1-carboxylate (2). To a solution of 3-(3-aminophenyl) piperidine-2,6-dione 1 (500 mg, 2.45 mmol) in dry CH₂Cl₂ (12.24 mL) at room temperature was added tert-butyl 4-formylpiperidine-1-carboxylate 2 (548 mg, 2.57 mmol). The resulting mixture was stirred at room temperature for 20 minutes after which NaBH(OAc)₃ (1.56 g, 7.34 mmol) was added and the resulting mixture was stirred at room temperature for 30 minutes. MeOH and silica gel were added and the mixture was concentrated to dryness and purified by silica gel flash chromatography (gradient elution 0-8% MeOH/CH₂Cl₂) afforded 882 mg (88%) of 3 as a white solid.

LCMS method 1: 97.9% (UV 215 nm); exact mass calculated=401.23; found [M-Boc+H]⁺=302.2

¹H NMR (400 MHz, DMSO-d6) δ ppm 0.95-1.14 (m, 2H), 1.39 (s, 9H), 1.62-1.80 (m, 3H), 1.98-2.17 (m, 2H), 2.39-2.48 (m, 1H), 2.55-2.65 (m, 2H), 2.87 (br s, 2H), 3.32 (s, 1H), 3.67 (dd, J=9.8, 5.4 Hz, 1H), 3.94 (br d, J=11.7 Hz, 2H), 5.65 (br s, 1H), 6.34 (d, J=7.3 Hz, 1H), 6.40 (s, 1H), 6.45 (br d, J=8.1 Hz, 1H), 7.00 (t, J=7.7 Hz, 1H), 10.78 (s, 1H).

Step 2. Preparation of 3-[3-(4-Piperidylmethylamino)phenyl]piperidine-2,6-dione 2,2,2-trifluoroacetic acid salt (C-1). To a solution of tert-butyl 4-[[3-(2,6-dioxo-3-piperidyl)anilino]methyl]piperidine-1-carboxylate 3 (882 mg, 2.20 mmol) in CH₂Cl₂ (10.98 mL) was added TFA (5.11 mL, 65.9 mmol). The resulting mixture was stirred at room temperature for 1 h. The reaction mixture was concentrated and chased with MeCN/PhMe (3×) followed by THF/heptanes (2×) and dried in vacuo affording 913 mg (99%) of C-1 as an off-white semi-solid.

LCMS method 1: 99.9% (UV 215 nm); exact mass calculated (free base)=301.18; found [M-TFA+H]⁺=302.4

### Example I-4. Synthesis of Common Intermediate C-2

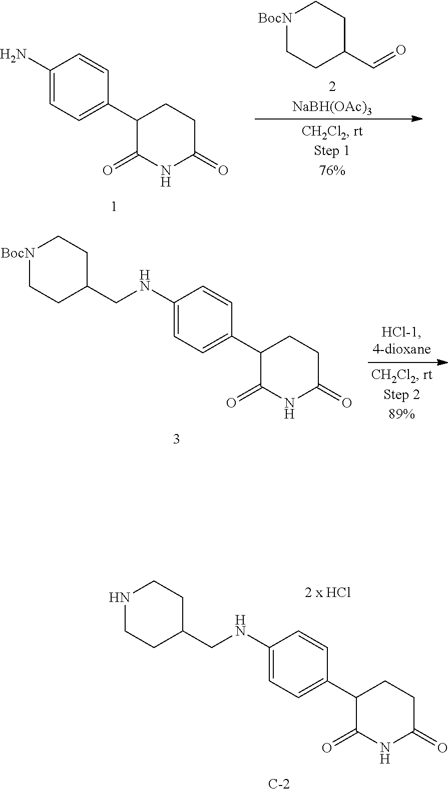

Step 1. Preparation of tert-Butyl 4-[[4-(2,6-dioxo-3-piperidyl)anilino]methyl]piperidine-1-carboxylate (3). To a solution of 3-(4-aminophenyl)piperidine-2,6-dione 1 (200.0 mg, 0.98 mmol) in dry CH₂Cl₂ (10 mL) was added tert-Butyl 4-formylpiperidine-1-carboxylate 2 (219.3 mg, 1.03 mmol) and the resulting mixture was stirred at room temperature for 20 minutes. Then, NaBH(OAc)₃ (622.65 mg, 2.94 mmol) was added and the resulting mixture was stirred at room temperature for 20 h. LCMS showed full conversion. MeOH and Silica was added, and the solvent was evaporated under reduce pressure. The crude mixture was purified by flash column chromatography normal phase eluting with 0 to 10% MeOH in CH₂Cl₂ (2 CV at 0%, then 15 CV to 10%). The desired fractions came out impure (at 3% MeOH) and contained SM. A second purification by reverse phase flash chromatography (80 g C18 gold column, liquid deposit with DMSO, eluted with MeCN/0.1% HCOOH, 3 CV at 5% MeCN, then 9 CV to 60% MeCN) afforded 3 (300 mg, 76% yield) as an off-white solid.

LCMS method 1: 99.9% purity at 215 nm, ([M-Boc+H]⁺=302.2; [M-tBu+H]⁺=346.2.

¹H NMR (400 MHz, DMSO-d6) δ ppm 0.97-1.09 (m, 2H), 1.40 (s, 9H), 1.73 (br d, J=12.2 Hz, 3H), 1.95-2.14 (m, 3H), 2.41-2.49 (m, 1H), 2.56-2.66 (m, 2H), 2.89 (br t, J=5.7 Hz, 2H), 3.63 (dd, J=10.5, 4.9 Hz, 1H), 3.95 (br d, J=11.5 Hz, 2H), 5.63 (t, J=5.9 Hz, 1H), 6.52 (d, J=8.6 Hz, 2H), 6.89 (d, J=8.6 Hz, 2H), 10.73 (s, 1H).

Step 2. Preparation of 3-[4-(4-Piperidylmethylamino)phenyl]piperidine-2,6-dione dihydrochloride (C-2). To a round bottom flask was solubilized tert-butyl 4-[[4-(2,6-dioxo-3-piperidyl)anilino]methyl]piperidine-1-carboxylate 3 (300.0 mg, 0.75 mmol) in CH₂Cl₂ (5 mL). 4 M HCl in 1,4-dioxane (1.87 mL, 7.47 mmol) was added, and the mixture was stirred at rt. After 4 h, HPLC showed complete conversion. The crude was co-evaporated using MeCN and toluene 3 times. Compound was purified by reverse phase flash chromatography (50 g C18 gold column, liquid deposit with DMSO, eluted with 5% MeCN/0.02 M HCl aq. over 10 CV). Fractions were concentrated to give C-2 (275 mg, 89%) as an orange semi-solid

LCMS method 1: 99.9% purity at 215 nm, [M−2HCl+H]⁺=302.2.

¹H NMR (400 MHz, DMSO-d6) δ ppm 1.22-1.38 (m, 2H), 1.77-1.85 (m, 1H), 1.86-1.94 (m, 2H), 1.95-2.03 (m, 1H), 2.43 (br t, J=4.5 Hz, 1H), 2.47 (br t, J=4.8 Hz, 1H), 2.57-2.69 (m, 1H), 2.85 (q, J=11.6 Hz, 2H), 2.94 (br d, J=6.4 Hz, 2H), 3.29 (br d, J=12.2 Hz, 2H), 3.65 (dd, J=10.8, 4.9 Hz, 1H), 6.57 (br d, J=8.3 Hz, 2H), 6.93 (d, J=8.6 Hz, 2H), 8.12-8.25 (m, 1H), 8.48-8.59 (m, 1H), 10.74 (s, 1H).

### Example I-5. Synthesis of Common Intermediate C-3

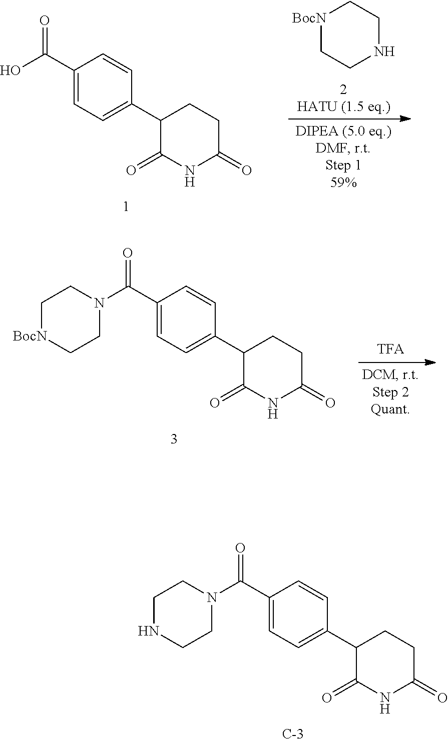

Step 1. Preparation of tert-butyl 4-[4-(2,6-dioxo-3-piperidyl)benzoyl]piperazine-1-carboxylate (3). A solution of tert-butyl piperazine-1-carboxylate 2 (135.76 mg, 0.7300 mmol), 4-(2,6-dioxo-3-piperidyl)benzoic acid 1 (200 .mg, 0.8600 mmol), HATU (489.1 mg, 1.29 mmol) and DIPEA (0.75 mL, 4.29 mmol) in DMF (2.5 mL) was stirred 16 hours at room temperature. Product material precipitated out of solution and the mixture was filtered and washed several times with nanopure water, before drying to afford compound 3 (203 mg, 59.0% yield) as a white solid.

LCMS method 1: 99% purity at 215 nm, [M+H]⁺=402.2; [M+H-tBu]⁺=346.2

Step 2. Preparation of 3-[4-(piperazine-1-carbonyl)phenyl]piperidine-2,6-dione (C-3). A solution of tert-butyl 4-[4-(2,6-dioxo-3-piperidyl)benzoyl]piperazine-1-carboxylate 3 (203.4 mg, 0.5100 mmol) and trifluoroacetic acid (1.5 mL, 19.59 mmol) in DCM (1.5 mL) was stirred overnight at room temperature. Upon completion, solvent was evaporated and the crude C-3 was used without purification in the next step.

### Example I-6. Synthesis of Common Intermediate C-4

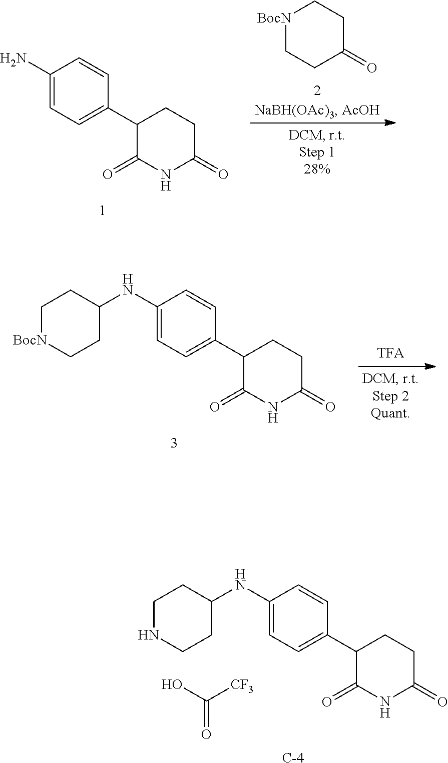

Step 1. Preparation of tert-butyl 4-[4-(2,6-dioxo-3-piperidyl)anilino]piperidine-1-carboxylate (3). To a solution of tert-butyl 4-oxopiperidine-1-carboxylate 2 (195 mg, 0.980 mmol, 1 eq.) and 3-(4-aminophenyl) piperidine-2,6-dione 1 (250 mg, 1.22 mmol, 1.25 eq.) in DCM (4.9 mL) was added NaBH(OAc)₃ (385 mg, 6.12 mmol, 6.2 eq.) followed by AcOH (96 mg, 1.59 mmol, 1.6 eq.). The reaction was stirred at room temperature. After 18 h, LCMS showed full conversion. Brine was added to the reaction and the product was then extracted 3 times with EtOAc. The combined organics were washed with brine, dried over Na₂SO₄ and concentrated to dryness. The residue was then purified by reverse phase flash chromatography (30 g C18 gold column, liquid deposit (DMSO), elution: 5% MeCN/0.1% HCOOH over 3 CV, then 5 to 60% MeCN/0.1% HCOOH over 13 CV). Fractions were combined and concentrated to give 3 (135 mg, 28% yield) as a white solid.

LCMS method 1: 99.9% purity at 215 nm, [M-tBu+H]⁺=332.2.

¹H NMR (400 MHz, DMSO-d6) δ ppm 1.17-1.27 (m, 2H), 1.40 (s, 9H), 1.86 (br d, J=11.7 Hz, 2H), 1.95-2.12 (m, 3H), 2.54-2.65 (m, 1H), 2.85-2.99 (m, 2H), 3.35-3.43 (m, 1H), 3.63 (dd, J=10.8, 5.1 Hz, 1H), 3.85 (br d, J=12.5 Hz, 2H), 5.42 (br d, J=8.6 Hz, 1H), 6.54 (d, J=8.3 Hz, 2H), 6.89 (d, J=8.3 Hz, 2H), 10.72 (s, 1H).

Step 2. Preparation of 3-[4-(4-piperidylamino)phenyl]piperidine-2,6-dione;2,2,2-trifluoroacetic acid (C-4). A solution of tert-butyl 4-[4-(2,6-dioxo-3-piperidyl)anilino]piperidine-1-carboxylate 3 (135 mg, 0.350 mmol, 1 eq.) and trifluoroacetic acid (1.5 mL, 19.6 mmol, 56 eq.) in DCM (1.5 mL) was stirred at room temperature. After 18 h, LCMS showed full conversion. Volatiles were removed under reduced pressure to give C-4 (139 mg, quantitative yield) as a white solid.

LCMS method 1: 99.9% purity at 215 nm, [M-CF₃COOH+H]⁺=288.2.

¹H NMR (400 MHz, DMSO-d6) δ ppm 1.45-1.58 (m, 2H), 1.93-2.13 (m, 4H), 2.58-2.65 (m, 1H), 2.94-3.07 (m, 2H), 3.24-3.34 (m, 3H), 3.64 (br dd, J=11.0, 4.9 Hz, 2H), 5.55-5.73 (m, 1H), 6.57 (d, J=8.3 Hz, 2H), 6.92 (d, J=8.3 Hz, 2H), 8.21-8.36 (m, 1H), 8.36-8.50 (m, 1H), 10.73 (s, 1H).

¹⁹F NMR (377 MHz, DMSO-d6) δ ppm −73.65 (s, 3F).

### Example I-7. Synthesis of Common Intermediate C-5

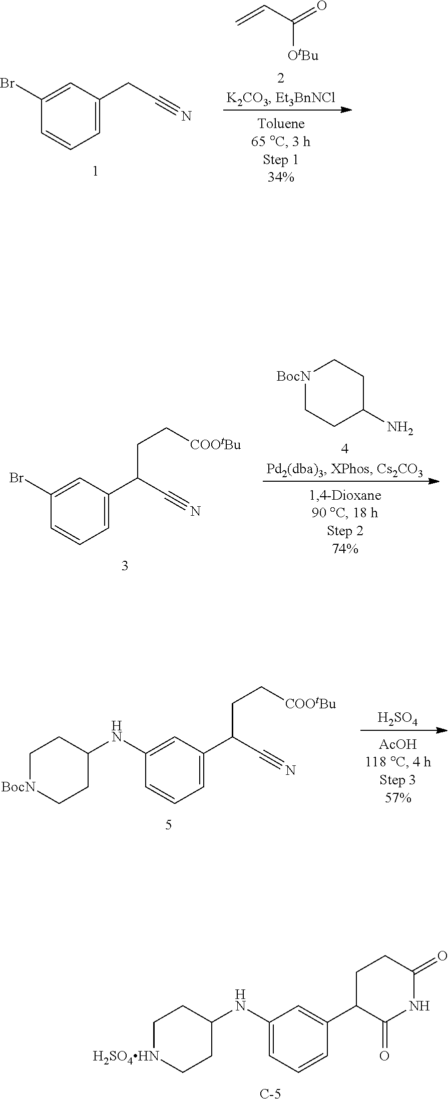

Step 1. Preparation of tert-Butyl 4-(3-bromophenyl)-4-cyano-butanoate (3). 2-(3-bromophenyl) acetonitrile 1 (4.0 g, 20.4 mmol, 1.0 equiv), tert-butyl prop-2-enoate 2 (2.62 g, 20.4 mmol, 1.0 equiv), benzyltriethylammonium chloride (465 mg, 2.04 mmol, 0.1 equiv), and potassium carbonate (2.82 g, 20.4 mmol, 1.0 equiv) were combined in toluene (20 mL), then the reaction mixture was stirred at 65° C. for 3 hours. The reaction mixture was evaporated to dryness, then the crude was dry loaded on silica and purified by reversed-phase flash chromatography (MeCN in 0.1% HCOOH(aq), 5% (3 CV)→80%, 120 g RediSep Rf Gold® C18Aq, 20 CV, λ=214-254 nm, product with 65% MeCN) to afford 3 (2.26 g, 6.97 mmol, 34% yield) as an orange oil.

LCMS method 1: 99.9% purity at 215 nm, [M+H]⁺=324.2.

¹H NMR (400 MHz, DMSO-d6) δ ppm 1.37-1.40 (m, 9H), 1.98-2.19 (m, 2H), 2.20-2.37 (m, 2H), 4.27 (t, J=7.5 Hz, 1H), 7.36-7.46 (m, 2H), 7.57 (dt, J=7.0, 1.9 Hz, 1H), 7.61 (s, 1H).

Step 2. Preparation of tert-Butyl 4-[3-(4-tert-butoxy-1-cyano-4-oxo-butyl)anilino]piperidine-1-carboxylate (5). In a sealed tube, tert-butyl 4-(3-bromophenyl)-4-cyano-butanoate 3 (200.0 mg, 0.617 mmol, 1.0 equiv), tert-butyl 4-aminopiperidine-1-carboxylate 4 (154.4 mg, 0.740 mmol, 1.2 equiv), cesium carbonate (402.0 mg, 1.23 mmol, 2.0 equiv) and XPhos (44.1 mg, 0.093 mmol, 0.15 equiv) were sparged with nitrogen, then anhydrous 1,4-dioxane (2 mL) was added and nitrogen was bubbled trough the mixture for 15 minutes under sonication. Pd₂(dba)₃ (56.5 mg, 0.062 mmol, 0.1 equiv) was then quickly added and nitrogen bubbling was continued for additional 5 minutes. The tube was sealed and the reaction mixture was stirred at 90° C. overnight. The reaction mixture was cooled to room temperature then water (5 mL) and DCM (5 mL) were added. The phases were separated and the aqueous layer was extracted with DCM (3×5 mL). The organic layers were combined, dried over MgSO₄, filtered and evaporated under reduced pressure. The crude red oil was dry loaded on silica and purified by normal phase flash chromatography (Heptanes/EtOAc, 100:0 to 30:70, 24 g RediSep Rf Gold® Normal-Phase Silica, 15 CV, λ=254-280 nm, product with 40-50% EtOAc) to afford 5 (226 mg, 0.456 mmol, 74% yield) as an orange viscous oil.

LCMS method 1: 89.4% purity at 215 nm, [M−2t-Bu+H]⁺=332.4.

Step 3. Preparation of 3-[3-(Piperidin-1-ium-4-ylamino)phenyl]piperidine-2,6-dione hydrogen sulfate (C-5). tert-butyl 4-[3-(4-tert-butoxy-1-cyano-4-oxo-butyl) anilino]piperidine-1-carboxylate 5 (226.0 mg, 0.456 mmol, 1.0 equiv) was dissolved in acetic acid (2.6 mL), then sulfuric acid (90 μL, 1.64 mmol, 3.6 equiv) was added dropwise and the mixture was stirred at 118° C. for 4 hours. Acetic acid was co-evaporated with toluene (3×10 mL) and the residue was dried under high vacuum. The residue was dissolved in the minimum amount of water and purified by reversed-phase flash chromatography (MeCN in 0.1% HCOOH(aq), 0% (5 CV)→20%, 20 g Claricep™ Spherical AQ C18 Column, 20 CV, λ=214-254 nm, product with 5-10% MeCN, broad band) to afford C-5 (100 mg, 0.259 mmol, 57% yield) as a white solid (after lyophilisation).

LCMS method 1: 99.9% purity at 215 nm, [M+H]⁺=288.4.

¹H NMR (400 MHz, DMSO-d6) δ ppm 0.89-1.66 (m, 3H), 1.72-2.25 (m, 4H), 2.71-2.97 (m, 2H), 2.99-3.50 (m, 5H), 3.52-4.37 (m, 3H), 5.32-5.86 (m, 1H), 5.97-6.74 (m, 3H), 6.76-7.35 (m, 1H), 7.62-9.08 (m, 2H), 10.40-11.23 (m, 1H).

### Synthesis of Common Intermediate C-6, C-9 and C-14

### Example I-8. Synthesis of Intermediate C-9

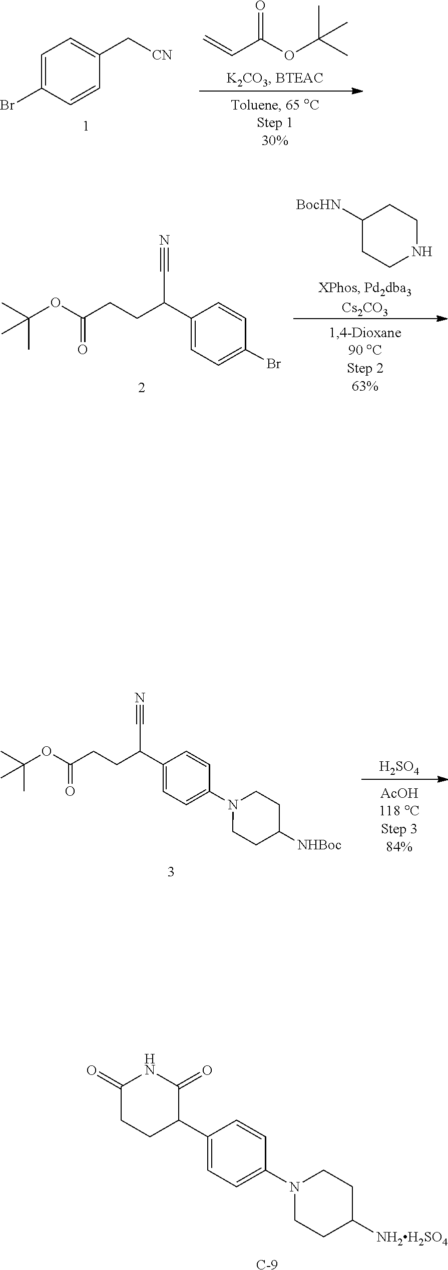

Step 1. Preparation of tert-Butyl 4-(4-bromophenyl)-4-cyano-butanoate (2). To a solution of 2-(4-bromophenyl) acetonitrile 1 (27.25 g, 139 mmol) in toluene (278 mL) was added tert-butyl prop-2-enoate (20.36 mL, 139 mmol), N-benzyl-N,N-diethylethanaminium chloride (3.17 g, 13.9 mmol), and potassium carbonate (19.21 g, 139 mmol). The mixture was stirred at 65° C. under nitrogen for 3 h then cooled to room temperature and filtered through a glass fritted funnel. The mixture was concentrated and the crude mixture was purified in 5 batches on RP-FC (415 g, C18 RediSep Rf Gold, 5% MeCN/0.1% Formic acid, 1 CV then 5 to 100% MeCN/0.1% Formic acid, 10 CV) giving 13.31 g (29.5%) of 2.

LCMS method 1: 98.1% purity at 215 nm, [M+H]⁺=324.0, [M+Na]⁺=346.0.

¹H NMR (400 MHz, CDCl₃) δ ppm 1.46 (s, 9H), 2.15 (q, J=7.3 Hz, 2H), 2.32-2.49 (m, 2H), 3.91 (t, J=7.3 Hz, 1H), 7.24 (d, J=8.3 Hz, 2H), 7.53 (d, J=8.6 Hz, 2H).

Step 2. Preparation of tert-Butyl 4-[4-[4-(tert-butoxycarbonylamino)-1-piperidyl]phenyl]-4-cyano-butanoate (3). To a vial charged with Cs₂CO₃ (1.09 g, 3.33 mmol), tert-butyl N-(4-piperidyl) carbamate (400 mg, 2.00 mmol), tert-butyl 4-(4-bromophenyl)-4-cyano-butanoate 2 (540 mg, 1.67 mmol), XPhos (119 mg, 0.250 mmol) and Pd₂(dba)₃ (152.5 mg, 0.1700 mmol) was added anhydrous 1,4-dioxane (8.33 mL) and the solution was sparged with nitrogen for 15 minutes. The reaction mixture was heated to 90° C. and stirred overnight. The reaction mixture was filtered through celite, rinsing with EtOAc and concentrated. Purification first by silica gel flash chromatography (gradient elution 0-100% EtOAc/heptane) followed by C18 reverse phase chromatography (gradient elution, 5-100% MeCN/0.1% aq. HCO₂H) gave 466 mg (63%) of 3 as an orange oil.

LCMS method 2:99.9% (UV 215 nm); exact mass calculated=443.28; found [M+H]⁺=444.4

¹H NMR (400 MHz, DMSO-d6) δ ppm 1.38 (s, 18H), 1.44 (br dd, J=11.6, 2.6 Hz, 2H), 1.78 (br d, J=10.5 Hz, 2H), 1.90-2.09 (m, 2H), 2.18-2.31 (m, 2H), 2.73 (br t, J=11.4 Hz, 2H), 3.33-3.48 (m, 1H), 3.65 (br d, J=12.7 Hz, 2H), 4.06 (t, J=7.5 Hz, 1H), 6.84 (br d, J=7.6 Hz, 1H), 6.94 (d, J=8.8 Hz, 2H), 7.16 (d, J=8.6 Hz, 2H).

Step 3. Preparation of 3-[4-(4-Amino-1-piperidyl)phenyl]piperidine-2,6-dione sulfuric acid salt (C-9). To a solution of tert-butyl 4-[4-[4-(tert-butoxycarbonylamino)-1-piperidyl]phenyl]-4-cyano-butanoate 3 (466 mg, 1.05 mmol) in acetic acid (5.25 mL) was added concentrated sulfuric acid (0.170 mL, 3.15 mmol). The mixture was stirred at 118° C. monitoring for completion by HPLC/LCMS (ca. 1 h). The reaction mixture was concentrated to remove AcOH, chased with PhMe (3×) and purified by C18 reverse phase column chromatography (gradient elution, 5-100% MeCN/0.1% aq HCO₂H) and lyophilised giving 254 mg (84%) of C-9 as a pale orange solid.

LCMS method 2:99.9% (215 nm), exact mass calculated (free base)=287.16; found [M−H₂SO₄+H]⁺=288.4.

¹H NMR (400 MHz, DMSO-d6) δ ppm 1.48-1.63 (m, 2H), 1.91 (br d, J=13.0 Hz, 2H), 1.97-2.06 (m, 1H), 2.08-2.20 (m, 1H), 2.40-2.49 (m, 1H), 2.57-2.66 (m, 1H), 2.74 (br t, J=12.1 Hz, 2H), 3.15 (s, 1H), 3.72 (br dd, J=10.9, 4.0 Hz, 3H), 6.90 (br d, J=8.6 Hz, 2H), 7.05 (br d, J=8.3 Hz, 2H), 7.56-9.15 (m, 3H), 10.77 (br s, 1H), 1H not apparent.

### Example I-9. Synthesis of Intermediate C-14

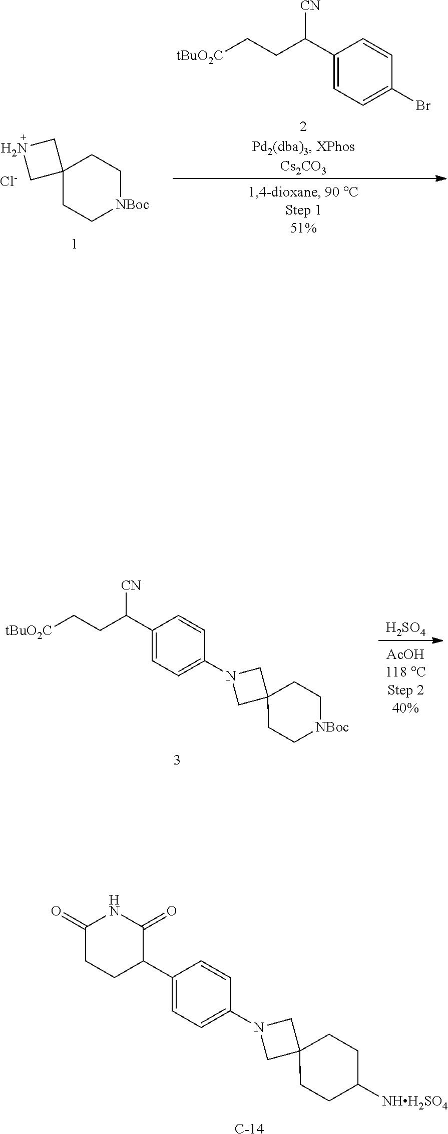

Step 1. Preparation of tert-Butyl 2-[4-(4-tert-butoxy-1-cyano-4-oxo-butyl)phenyl]-2,7-diazaspiro[3.5]nonane-7-carboxylate (3). A suspension of Cs₂CO₃ (1.04 g, 3.180 mmol, 4 eq.), tert-butyl 2,7-diazaspiro[3.5]nonane-7-carboxylate hydrochloride 1 (251 mg, 0.950 mmol, 1.2 eq.), tert-butyl 4-(4-bromophenyl)-4-cyano-butanoate 2 (258 mg, 0.800 mmol, 1 eq.), XPhos (57 mg, 0.120 mmol, 0.15 eq.) and Pd₂(dba)₃ (73 mg, 0.080 mmol, 0.1 eq.) in anhydrous 1,4-dioxane (4 mL) was degassed with nitrogen for 15 min. The mixture was stirred at 90° C. After 18 h, LCMS showed full conversion. The reaction mixture was filtered through a pad of Celite and rinsed with EtOAc. The mother liquors were concentrated to dryness and the residue was purified by normal phase flash chromatography (80 g silica column, preabsorbed, elution: 0 to 50% EtOAc/Heptane over 20 CV). Fractions were combined and concentrated to give an impure product, which was then purified by reverse phase flash chromatography (50 g C18 gold column, liquid deposit (DMSO), elution: 5% MeCN/0.1% HCOOH over 3 CV, then 5 to 100% MeCN/0.1% HCOOH over 28 CV). Fractions were combined and concentrated to give 3 (189 mg, 51% yield) as a colorless oil.

LCMS method 1: 99.9% purity at 215 nm, [M-Boc+H]⁺=370.4.

¹H NMR (400 MHz, DMSO-d6) δ ppm 1.38 (s, 9H), 1.40 (s, 9H), 1.64-1.72 (m, 4H), 1.90-2.10 (m, 2H), 2.21-2.27 (m, 2H), 3.27-3.33 (m, 4H), 3.56 (s, 4H), 4.03 (t, J=7.5 Hz, 1H), 6.43 (d, J=8.6 Hz, 2H), 7.14 (d, J=8.6 Hz, 2H).

Step 2. Preparation of 3-[4-(2,7-Diazaspiro[3.5]nonan-2-yl)phenyl]piperidine-2,6-dione sulfuric acid (C-14). To a solution of tert-butyl 2-[4-(4-tert-butoxy-1-cyano-4-oxo-butyl)phenyl]-2,7-diazaspiro[3.5]nonane-7-carboxylate 3 (188.9 mg, 0.400 mmol, 1 eq.) in acetic acid (2 mL) was added concentrated sulfuric acid (118 mg, 1.21 mmol, 3 eq.). The mixture was stirred at 118° C. After 80 min, LCMS showed full conversion. Acetic acid was co-evaporated with toluene and the residue was purified by reverse phase flash chromatography (20 g C18 Aq column, liquid deposit (H₂O), elution: 0% MeCN/0.1% HCOOH over 5 CV, then 5 to 10% MeCN/0.1% HCOOH over 40 CV). Fractions were combined, concentrated and lyophilised to give C-14 (67 mg, 40% yield) as a white solid as a sulfuric acid salt.

LCMS method 1: 99.9% purity at 215 nm, [M−H₂SO₄+H]⁺=314.4.

¹H NMR (400 MHz, DMSO-d6) δ ppm 1.87-1.93 (m, 4H), 1.94-2.09 (m, 3H), 2.55-2.66 (m, 1H), 2.99-3.07 (m, 4H), 3.56 (s, 4H), 3.67 (dd, J=10.8, 4.9 Hz, 1H), 6.39 (d, J=8.3 Hz, 2H), 6.99 (d, J=8.6 Hz, 2H).

The following compounds were synthesized via the same general route with modifications to amine 1 in step 1 (Table 2).

TABLE 2

| | Amine | Structure |
|---|---|---|
| C-6 | 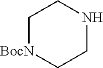 [SMILES: CC(C)(C)OC(=O)N1CCNCC1] | 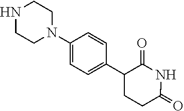 [SMILES: O=C1CCC(c2ccc(N3CCNCC3)cc2)C(=O)N1] |
| C-9 | 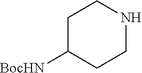 [SMILES: CC(C)(C)OC(=O)NC1CCNCC1] | 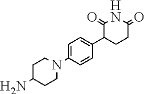 [SMILES: NC1CCN(c2ccc(C3CCC(=O)NC3=O)cc2)CC1] |
| C-14 | 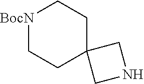 [SMILES: CC(C)(C)OC(=O)N1CCC2(CC1)CNC2] | 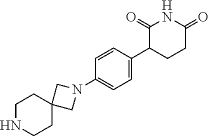 [SMILES: O=C1CCC(c2ccc(N3CC4(CCNCC4)C3)cc2)C(=O)N1] |

### Example I-10. Synthesis of Common Intermediate C-7

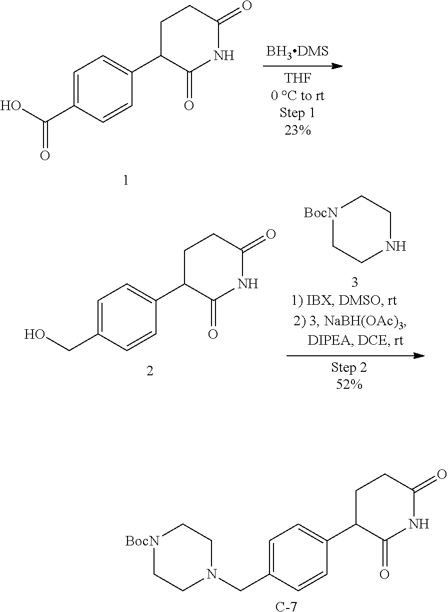

Step 1. Preparation of 3-[4-(Hydroxymethyl)phenyl]piperidine-2,6-dione (2). To a solution of 1 (500.0 mg, 2.14 mmol, 1.0 eq.) in dry THF (10.7 mL, 0.2 M) was added BH₃ DMS complex (0.81 mL, 8.58 mmol, 4.0 eq.) at 0° C. and the reaction was stirred at rt. After 16 h, the reaction was quenched with 1 M HCl until there was no more formation of gas under stirring. The mixture was brought to dryness by concentration. The crude was dissolved in a minimum of DMSO before being directly loaded on a 50 g C18 gold column and purified by reverse phase flash chromatography using 5-30% MeCN in water (+0.1% formic acid). Pure fractions were combined and concentrated to afford 2 (114 mg, 23% yield) as a white solid.

LCMS method 1: retention time: 1.137 min, 99.9% purity at 215 nm, [M+H]⁺=220.1.

¹H NMR (400 MHz, DMSO-d6) δ ppm 1.99-2.08 (m, 1H), 2.12-2.24 (m, 1H), 2.43-2.48 (m, 1H), 2.66 (ddd, J=17.1, 11.7, 5.4 Hz, 1H), 3.83 (br dd, J=11.4, 5.0 Hz, 1H), 4.47 (br d, J=5.1 Hz, 2H), 5.14 (br t, J=5.6 Hz, 1H), 7.17 (d, J=8.1 Hz, 2H), 7.27 (br d, J=8.1 Hz, 2H), 10.81 (br s, 1H).

Step 2. Preparation of tert-Butyl 4-[[4-(2,6-Dioxo-3-piperidyl)]methyl]piperazine-1-carboxylate (C-7). To a solution of 2 (114.5 mg, 0.52 mmol, 1.0 eq.) in dry DMSO (2.1 mL, 0.25 M) was added IBX (190.12 mg, 0.68 mmol, 1.3 eq.). The resulting mixture was stirred at room temperature overnight. To a second solution of 3 (82.31 mg, 0.44 mmol, 0.8 eq.) in dry DCE (1.8415 mL, 0.8 M) was added DIPEA (0.51 mL, 2.95 mmol, 8.0 eq.). The resulting mixture was stirred at room temperature for 15 minutes. NaBH(OAc)₃ (273.19 mg, 1.29 mmol, 3.5 eq.) was added followed by the previous DMSO solution from the IBX oxidation reaction. The resulting mixture was stirred at rt. After 16 h, DCE was removed under reduced pressure. The crude mixture was purified by reverse phase flash chromatography (30 g C18 gold column, elution: 35 to 55% MeCN in H₂O (0.1% formic acid) over 20 CV). The desired fractions were concentrated to dryness. Co-evaporation of the oily material with a mixture of heptanes/MTBE affords C-7 (111 mg, 52% yield) as a white solid.

LCMS method 1: retention time: 1.199 min, 96.5% purity at 215 nm, [M+H]⁺=388.2.

¹H NMR (400 MHz, DMSO-d6) δ ppm 1.39 (s, 9H), 2.00-2.07 (m, 1H), 2.12-2.24 (m, 2H), 2.27-2.35 (m, 4H), 2.44-2.48 (m, 1H), 2.60-2.72 (m, 2H), 3.28-3.31 (m, 2H), 3.46 (br s, 2H), 3.84 (br dd, J=11.5, 4.9 Hz, 1H), 7.17-7.21 (m, 2H), 7.26 (br d, J=8.1 Hz, 2H), 10.83 (s, 1H).

### Synthesis of Common Intermediates C-8, C-10, C-11 and C-13

### Example I-11. Synthesis of Intermediate C-10

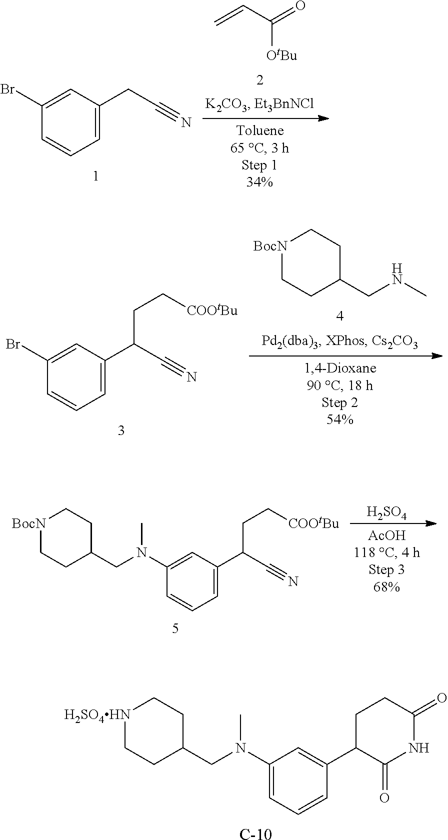

Step 1. Preparation of tert-Butyl 4-(3-bromophenyl)-4-cyano-butanoate (3). 2-(3-bromophenyl) acetonitrile 1 (4.0 g, 20.4 mmol, 1.0 equiv), tert-butyl prop-2-enoate 2 (2.62 g, 20.4 mmol, 1.0 equiv), benzyltriethylammonium chloride (465 mg, 2.04 mmol, 0.1 equiv), and potassium carbonate (2.82 g, 20.4 mmol, 1.0 equiv) were combined in in toluene (20 mL), then the reaction mixture was stirred at 65° C. for 3 hours. The reaction mixture was evaporated to dryness, then the crude was dry loaded on silica and purified by reversed-phase flash chromatography (MeCN in 0.1% HCOOH(aq), 5% (3 CV)→80%, 120 g RediSep Rf Gold® C18Aq, 20 CV, λ=214-254 nm, product with 65% MeCN) to afford 3 (2.26 g, 6.97 mmol, 34% yield) as an orange oil.

LCMS method 1: 99.9% purity at 215 nm, [M+H]⁺=324.2.

¹H NMR (400 MHz, DMSO-d6) δ ppm 1.37-1.40 (m, 9H), 1.98-2.19 (m, 2H), 2.20-2.37 (m, 2H), 4.27 (t, J=7.5 Hz, 1H), 7.36-7.46 (m, 2H), 7.57 (dt, J=7.0, 1.9 Hz, 1H), 7.61 (s, 1H).

Step 2. Preparation of tert-Butyl 4-[[3-(4-tert-butoxy-1-cyano-4-oxo-butyl)-N-methyl-anilino]methyl]piperidine-1-carboxylate (5). In a sealed tube, tert-butyl 4-(3-bromophenyl)-4-cyano-butanoate 3 (400.0 mg, 1.23 mmol, 1.0 equiv), tert-butyl 4-(methylaminomethyl) piperidine-1-carboxylate 4 (338.0 mg, 1.48 mmol, 1.2 equiv), cesium carbonate (804.0 mg, 2.46 mmol, 2.0 equiv) and XPhos (88.2 mg, 0.185 mmol, 0.15 equiv) were sparged with nitrogen, then anhydrous 1,4-dioxane (4 mL) was added and nitrogen was bubbled trough the mixture for 15 minutes under sonication. Pd₂(dba)₃ (113.0 mg, 0.123 mmol, 0.1 equiv) was then quickly added and nitrogen bubbling was continued for additional 5 minutes. The tube was sealed and the reaction mixture was stirred at 90° C. overnight. The reaction mixture was cooled to room temperature then water (20 mL) and DCM (20 mL) were added. The phases were separated and the aqueous layer was extracted with DCM (3×20 mL). The organic layers were combined, dried over MgSO₄, filtered and evaporated under reduced pressure. The crude red oil was dry loaded on silica and purified by normal phase flash chromatography (Heptanes/EtOAc, 100:0 to 50:50, 80 g RediSep Rf Gold® Normal-Phase Silica, 15 CV, λ=254-280 nm, product with 35-45% EtOAc) to afford 5 (316 mg, 0.670 mmol, 54% yield) as yellow resin.

LCMS method 1: 99.9% purity at 215 nm, [M+Na]⁺=494.4, [M−2t-Bu+H]⁺=360.4, [M-Boc-t-Bu+H]⁺=316.4.

¹H NMR (400 MHz, CDCl₃) δ ppm 1.11-1.22 (m, 2H), 1.46 (d, J=3.7 Hz, 18H), 1.68 (br d, J=12.3 Hz, 2H), 1.80-1.98 (m, 1H), 2.10-2.25 (m, 2H), 2.31-2.52 (m, 2H), 2.56-2.76 (m, 2H), 2.99 (s, 3H), 3.21 (d, J=7.1 Hz, 2H), 3.89 (t, J=7.5 Hz, 1H), 4.14 (br s, 2H), 6.58 (s, 1H), 6.62 (br dd, J=6.9, 5.1 Hz, 2H), 7.21 (t, J=7.9 Hz, 1H).

Step 3. Preparation of 3-[3-[Methyl(piperidin-1-ium-4-ylmethyl)amino]phenyl]piperidine-2,6-dione hydrogen sulfate (C-10). tert-butyl 4-[[3-(4-tert-butoxy-1-cyano-4-oxo-butyl)-N-methyl-anilino]methyl]piperidine-1-carboxylate 5 (316.0 mg, 0.670 mmol) was dissolved in acetic acid (4 mL), then sulfuric acid (133 μL, 2.43 mmol, 3.6 equiv) was added dropwise and the mixture was stirred at 118° C. for 2 hours. Acetic acid was co-evaporated with toluene (3×5 mL) and the residue was dried under high vacuum. The residue was dissolved in the minimum amount of water and purified by reversed-phase flash chromatography (MeCN in 0.1% HCOOH(aq), 0% (5 CV)→40%, 40 g Claricep™ Spherical AQ C18, 20 CV, λ=214-254 nm, product with 20-25% MeCN, multiple bands) to afford C-10 (187.5 mg, 0.453 mmol, 68% yield) as a yellow solid.

LCMS method 1: 99.9% purity at 215 nm, [M−H₂SO₄+H]⁺=316.4.

¹H NMR (400 MHz, DMSO-d6) δ ppm 1.33 (q, J=11.4 Hz, 2H), 1.74 (br d, J=12.7 Hz, 2H), 1.88-1.98 (m, 1H), 1.99-2.10 (m, 1H), 2.10-2.26 (m, 1H), 2.39-2.48 (m, 1H), 2.63 (ddd, J=17.1, 11.2, 5.5 Hz, 1H), 2.76 (br t, J=11.9 Hz, 2H), 2.90 (s, 3H), 3.12-3.25 (m, 4H), 3.74 (br dd, J=10.6, 4.9 Hz, 1H), 6.44 (br d, J=7.5 Hz, 1H), 6.51 (br s, 1H), 6.58 (br d, J=8.4 Hz, 1H), 7.11 (t, J=7.8 Hz, 1H), 8.33 (s, 1H), 10.80 (br s, 1H).

The following compounds were synthesized via the same general route with modifications to amine 4 in step 2 (Table 3).

TABLE 3

| | Amine | Structure |
|---|---|---|
| C-8 | 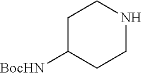 [SMILES: CC(C)(C)OC(=O)NC1CCNCC1] | 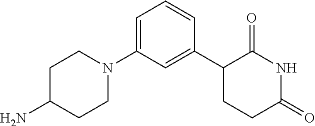 [SMILES: NC1CCN(c2cccc(C3CCC(=O)NC3=O)c2)CC1] |
| C-10 | 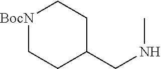 [SMILES: CNCC1CCN(C(=O)OC(C)(C)C)CC1] | 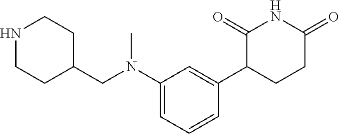 [SMILES: CN(CC1CCNCC1)c1cccc(C2CCC(=O)NC2=O)c1] |
| C-11 | 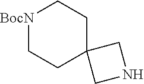 [SMILES: CC(C)(C)OC(=O)N1CCC2(CC1)CNC2] | 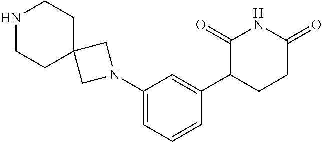 [SMILES: O=C1CCC(c2cccc(N3CC4(CCNCC4)C3)c2)C(=O)N1] |
| C-13 | 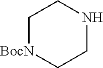 [SMILES: CC(C)(C)OC(=O)N1CCNCC1] | 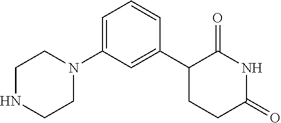 [SMILES: O=C1CCC(c2cccc(N3CCNCC3)c2)C(=O)N1] |

### Example I-12. Synthesis of Common Intermediate C-12

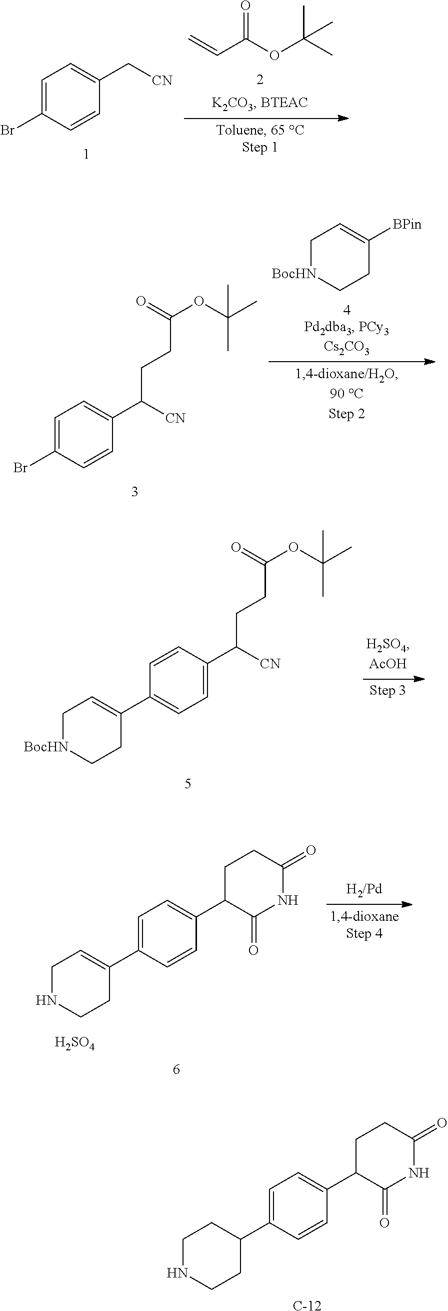

Step 1. Preparation of tert-Butyl 4-(4-bromophenyl)-4-cyano-butanoate (2). To a solution of 2-(4-bromophenyl) acetonitrile 1 (27.25 g, 139 mmol) in toluene (278 mL) was added tert-butyl prop-2-enoate 2 (20.36 mL, 139 mmol), N-benzyl-N,N-diethylethanaminium chloride (3.17 g, 13.9 mmol), and potassium carbonate (19.21 g, 139 mmol). The mixture was stirred at 65° C. under nitrogen for 3 h then cooled to room temperature and filtered through a glass fritted funnel. The mixture was concentrated and the crude mixture was purified in 5 batches on RP-FC (415 g, C18 RediSep Rf Gold, 5% MeCN/0.1% Formic acid, 1 CV then 5 to 100% MeCN/0.1% Formic acid, 10 CV) giving 13.31 g (29.5%) of 3.

LCMS method 1: 98.1% purity at 215 nm, [M+H]⁺=324.0, [M+Na]⁺=346.0.

¹H NMR (400 MHz, CDCl₃) δ ppm 1.46 (s, 9H), 2.15 (q, J=7.3 Hz, 2H), 2.32-2.49 (m, 2H), 3.91 (t, J=7.3 Hz, 1H), 7.24 (d, J=8.3 Hz, 2H), 7.53 (d, J=8.6 Hz, 2H).

Step 2. Preparation of tert-Butyl 4-[4-(4-tert-butoxy-1-cyano-4-oxo-butyl)phenyl]-3,6-dihydro-2H-pyridine-1-carboxylate (5). In a 250 mL round bottom flask, a solution of tert-butyl 4-(4-bromophenyl)-4-cyano-butanoate 3 (1.0 g, 3.08 mmol), tert-butyl 4-(4,4,5,5-tetramethyl-1,3,2-dioxaborolan-2-yl)-3,6-dihydro-2H-pyridine-1-carboxylate 4 (1.15 g, 3.70 mmol), Cs₂CO₃ (3.02 g, 9.25 mmol) and tricyclohexylphosphine (86.5 mg, 0.31 mmol) in 1,4-dioxane (24.7 mL) and water (6.2 mL) was sparged with nitrogen for 5 min then Pd₂(dba)₃ (34.62 mg, 0.15 mmol) was added to the solution. The solution was sparged again for 5 min and the solution mixture was stirred at 90° C. for 4 h. The mixture was diluted in EtOAc, filtered over Celite and the filtrate was concentrated to dryness and purified by SiO₂ normal phase chromatography (gradient elution, 0 to 20% EtOAc in Heptanes) giving 1.49 g (quant.) of 5 as a light brown oil.

LCMS method 1: 90.0% purity at 215 nm, [M-Boc+H]⁺=327.2.

¹H NMR (400 MHz, CDCl₃) δ ppm 1.25-1.29 (m, 2H), 1.44-1.47 (m, 9H), 1.50 (s, 9H), 2.17 (q, J=7.3 Hz, 2H), 2.52 (br s, 2H), 3.65 (t, J=5.6 Hz, 2H), 3.93-3.99 (m, 1H), 4.09 (br d, J=2.4 Hz, 2H), 5.99-6.14 (m, 1H), 7.29-7.34 (m, 2H), 7.37-7.42 (m, 2H).

Step 3. Preparation of 3-[4-(1,2,3,6-Tetrahydropyridin-4-yl)phenyl]piperidine-2,6-dione sulfuric acid (6). To a solution of tert-butyl 4-[4-(4-tert-butoxy-1-cyano-4-oxo-butyl)phenyl]-3,6-dihydro-2H-pyridine-1-carboxylate 5 (1.31 g, 3.07 mmol) in acetic acid (15.4 mL) was added concentrated sulfuric acid (0.49 mL, 9.21 mmol). The mixture was stirred at 118° C. for 0.75 h. The reaction mixture was concentrated and the crude mixture was purified by C18 RediSep Rf Gold reverse phase chromatography (gradient elution, 0% MeCN/0.1% aq. HCO₂H, 10 CV, then 0-100% MeCN/0.1% aq. HCO₂H, 10 CV) giving 1.03 g (91% yield) of 6 as a white solid.

LCMS method 1: 99.9% purity at 215 nm, [M−H₂SO₄+H]⁺=271.2.

¹H NMR (400 MHz, DMSO-d6) δ ppm 1.99-2.06 (m, 1H), 2.12-2.26 (m, 1H), 2.57-2.73 (m, 3H), 3.15 (s, 1H), 3.27 (br t, J=5.9 Hz, 2H), 3.71 (br s, 2H), 3.87 (dd, J=11.5, 4.9 Hz, 1H), 4.74 (br d, J=15.2 Hz, 1H), 6.18 (br s, 1H), 7.23 (d, J=8.1 Hz, 2H), 7.43 (d, J=8.3 Hz, 2H), 8.24 (br s, 1H), 9.84 (br s, 2H), 10.85 (br s, 1H).

Step 4. Preparation of 3-[4-(4-piperidyl)phenyl]piperidine-2,6-dione formic acid (C-12). To a solution of sulfuric acid, 3-[4-(1,2,3,6-tetrahydropyridin-4-yl)phenyl]piperidine-2,6-dione 6 (220 .mg, 0.60 mmol) in 1,4-Dioxane (8.10 mL) and Water (0.41 mL) at room temperature was added Pd/C (189.46 mg, 0.18 mmol), then the atmosphere of the flask was replaced with hydrogen by bubbling H₂ into the mixture. The reaction mixture was stirred at room temperature under hydrogen until completion (c.a. overnight). The mixture was filtered over Celite, rinsed with MeCN and concentrated under reduced pressure to give 135 mg (70%) of C-12 as an off-white solid.

LCMS method 1: 80.9% purity at 215 nm, [M−HCOOH+H]⁺=273.4.

¹H NMR (400 MHz, DMSO-d6) δ ppm 1.69-1.79 (m, 2H), 1.89-2.07 (m, 4H), 2.11-2.24 (m, 1H), 2.64-2.70 (m, 1H), 2.75-2.84 (m, 1H), 2.90-3.00 (m, 2H), 3.26-3.29 (m, 1H), 3.33-3.36 (m, 1H), 3.83 (dd, J=11.5, 5.1 Hz, 1H), 7.19 (s, 4H), 10.83 (s, 1H).

### Example I-13. Synthesis of Common Intermediate C-15

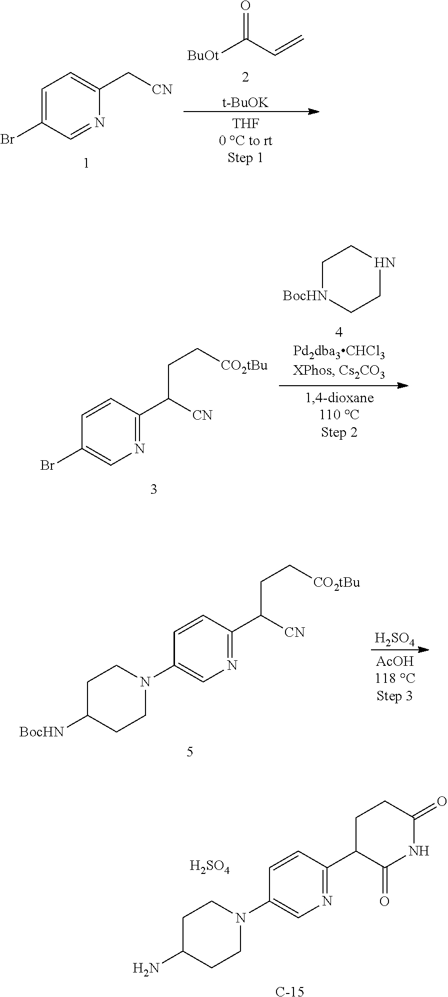

Step 1. Preparation of tert-Butyl 4-(5-bromo-2-pyridyl)-4-cyano-butanoate (3). A solution of 1 (350 .mg, 1.78 mmol, 1 eq.) and 2 (0.27 mL, 1.87 mmol, 1.05 eq.) in THF (8.88 mL, 0.2 M) was cooled down to 0° C. (ice bath). t-BuOK (19.93 mg, 0.18 mmol, 0.1 eq.) was added and the reaction was stirred at 0° C. overnight (keeping the ice bath slowly warming up to rt). The reaction mixture was concentrated to dryness. The crude was dissolved in a minimum of DMSO and injected on a C18 RediSep Rf Gold column. Purification was performed using a gradient of 5-100% MeCN in water (0.1% formic acid)-50 g gold column. Product came out at 62% MeCN. All the fractions containing the desired compound were collected and concentrated to dryness to give 3 (128 mg, 22% yield) as a light yellow solid.

LCMS method 1: retention time: 1.860 min, 99.9% purity at 215 nm, [M-tBu+H]⁺=269.0.

¹H NMR (400 MHz, DMSO-d6) δ ppm 1.39 (s, 9H), 2.15 (q, J=7.3 Hz, 2H), 2.29-2.36 (m, 2H), 4.43 (t, J=7.2 Hz, 1H), 7.46 (d, J=7.8 Hz, 1H), 8.14 (dd, J=8.3, 2.4 Hz, 1H), 8.75 (d, J=2.4 Hz, 1H).

Step 2. Preparation of tert-Butyl 4-[5-[4-(tert-butoxycarbonylamino)-1-piperidyl]-2-pyridyl]-4-cyano-butanoate (5). To a sealed tube were added 3 (128.3 mg, 0.39 mmol, 1 eq.), 4 (94.82 mg, 0.47 mmol, 1.2 eq.), Cs₂CO₃ (257.09 mg, 0.79 mmol, 2 eq.) in 1,4-dioxane (1.97 mL, 0.2 M). The reaction mixture was bubbled with nitrogen for 10 min. Then XPhos (37.62 mg, 0.08 mmol, 0.2 eq.) and Pd₂(dba)₃·CHCl₃ (40.83 mg, 0.04 mmol, 0.1 eq.) were added and nitrogen was bubbled for a further 10 min before being stirred at 110° C. overnight. The reaction mixture was filtered over a pad of Celite (rinsed with EtOAc). The filtrate was concentrated to dryness. The crude was dissolved in a minimum of DMSO. Purification was performed using 5-100% MeCN in water (+0.1% formic acid)-50 g RediSep Rf Gold column. Pure fractions were combined and the solvent was evaporated to dryness to afford 5 (71 mg, 40% yield) as a brown oil.

LCMS method 1: retention time: 1.868 min, [M+H]⁺=445.2.

¹H NMR (400 MHz, DMSO-d6) δ ppm 1.39 (s, 18H), 1.42-1.51 (m, 2H), 1.80 (br d, J=10.8 Hz, 2H), 2.09-2.13 (m, 2H), 2.26-2.31 (m, 2H), 2.77-2.86 (m, 2H), 3.38-3.48 (m, 1H), 3.69-3.76 (m, 2H), 4.21 (t, J=7.3 Hz, 1H), 6.86 (br d, J=7.8 Hz, 1H), 7.23 (d, J=8.6 Hz, 1H), 7.35 (dd, J=8.8, 2.9 Hz, 1H), 8.29 (d, J=2.9 Hz, 1H)

Step 3. Preparation of 3-[5-(4-Amino-1-piperidyl)-2-pyridyl]piperidine-2,6-dione sulfuric acid (C-15). A solution of 5 (71.2 mg, 0.16 mmol, 1 eq.) and conc. sulfuric acid (34.15 μL, 0.64 mmol, 4 eq.) in acetic acid (0.800 mL, 0.2 M) was stirred at 118° C. for 60 min. The reaction mixture was concentrated to dryness to afford C-15 (160 mg, quantitative yield) as a brown oil. The product was used without purification in the next step.

LCMS method 1: retention time: 0.207 min, 38.7% purity at 215 nm, [M−H₂SO₄+H]⁺=289.2.

¹H NMR (400 MHz, deuterium oxide) δ ppm 1.63 (qd, J=12.3, 4.0 Hz, 2H), 1.97 (s, 6H), 2.03-2.10 (m, 2H), 2.22-2.42 (m, 2H), 2.74-2.79 (m, 1H), 2.97-3.08 (m, 2H), 3.41 (tt, J=11.6, 4.1 Hz, 1H), 3.80-3.93 (m, 2H), 7.65 (d, J=9.3 Hz, 1H), 7.94 (dd, J=9.3, 2.9 Hz, 1H), 8.19 (d, J=2.9 Hz, 1H).

### Example I-14. Synthesis of Common Intermediate C-16

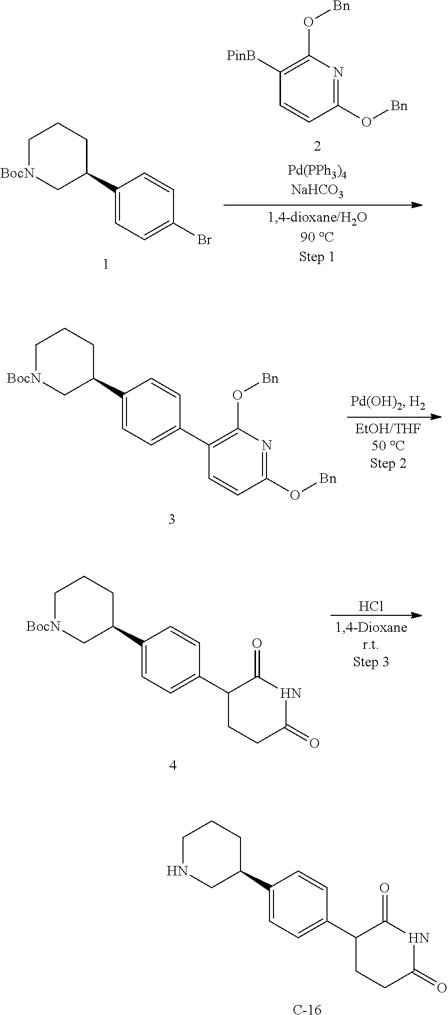

Step 1. Preparation of tert-butyl(S)-3-(4-(2,6-bis(benzyloxy)pyridin-3-yl)phenyl)piperidine-1-carboxylate (3). A sealed tube was charged with NaHCO₃ (0.1 mL, 0.7700 mmol), tert-butyl(S)-3-(4-bromophenyl) piperidine-1-carboxylate 1 (87.24 mg, 0.2600 mmol), 2,6-dibenzyloxy-3-(4,4,5,5-tetramethyl-1,3,2-dioxaborolan-2-yl)pyridine 2 (128.4 mg, 0.3100 mmol) and Pd(PPh₃)₄ (29.63 mg, 0.0300 mmol), then the tube was flushed with nitrogen for 5 minutes. A mixture of 1,4-dioxane (2.0512 mL) and water (0.5128 mL) (degassed by sparging with nitrogen for 10 minutes) was added before the mixture was sparged with nitrogen for an additional 5 minutes. The vial was sealed and the reaction was heated overnight at 90° C. To the reaction mixture, ethyl acetate and water were added and phases were separated. The aqueous phase was extracted 3 times with ethyl acetate and the combined organic phases were washed once with brine, dried over magnesium sulfate, filtered and evaporated under reduced pressure. Material was purified by normal phase chromatography using a gradient of heptanes and ethyl acetate (0% to 30% over 20 CV). The pure fractions are combined, evaporated under reduced pressure and dried under high vacuum to give 180.2 mg of 3 as a yellow oil.

LCMS Method 2: RT=2.506 min; Purity: 93.0% at 215 nm.

Step 2. Preparation of tert-butyl (3S)-3-(4-(2,6-dioxopiperidin-3-yl)phenyl)piperidine-1-carboxylate (4). A solution of tert-butyl(S)-3-(4-(2,6-bis(benzyloxy)pyridin-3-yl)phenyl) piperidine-1-carboxylate 3 (124.7 mg, 0.2300 mmol) in THF (0.8087 mL) and ethanol (0.8087 mL) was degassed for 15 minutes, before adding Pd(OH)₂ (25.55 mg, 0.0400 mmol) and sparging for an additional 5 minutes. The reactor was then fitted with an apparatus enabling a positive pressure of hydrogen (approx. 80 psi) and heated overnight at 60° C. Upon observing complete conversion to product by LCMS, the solvents were evaporated and the crude material was purified by reverse phase chromatography using a gradient of 0.1% FA water and acetonitrile (5% to 100% over 20 CV). The pure fractions were combined, evaporated and dried under high vacuum to afford 55 mg of 4.

LCMS Method 1: RT=1.765 min; Purity: 99.9% at 215 nm; [M+Na]⁺: 396.2; [M-t-Bu]⁺: 317.2; [M-Boc]⁺: 274.2.

Step 3. Preparation of 3-(4-((S)-piperidin-3-yl)phenyl)piperidine-2,6-dione (C-16). To reaction flask was added tert-butyl (3S)-3-[4-(2,6-dioxo-3-piperidyl)phenyl]piperidine-1-carboxylate 4 (84.2 mg, 0.2300 mmol) and HCL in dioxane (2.5 mL, 10 mmol), before the solution is stirred at room temperature for 2 hours. Upon completion, observed by LCMS, the volatiles were evaporated under reduced pressure and co-evaporated 3 times with acetonitrile to give 80.7 mg of C-16 as a purple solid.

LCMS Method 1: RT=0.961 min; Purity: 99.9% at 215 nm; [M+H]⁺: 273.2.

Compound C-16i was afforded by using the opposite enantiomer of tert-butyl 3-(4-bromophenyl) piperidine-1-carboxylate.

### Example I-15. Synthesis of Common Intermediate C-17

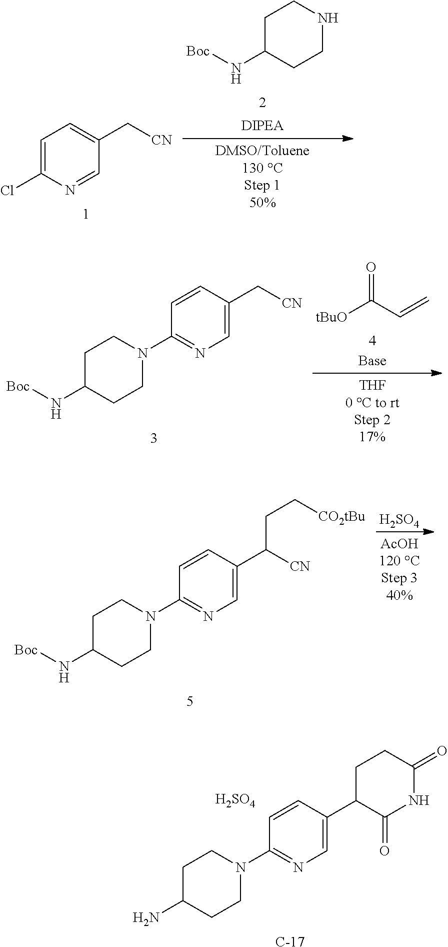

Step 1. Preparation of tert-Butyl N-[1-[5-(cyanomethyl)-2-pyridyl]-4-piperidyl]carbamate (3). To a solution of 2-(6-chloro-3-pyridyl) acetonitrile 1 (1.5 g, 9.83 mmol, 1 eq.) and tert-butyl N-(4-piperidyl) carbamate 2 (2.95 g, 14.75 mmol, 1.5 eq.) in a 1:8 Toluene/DMSO (0.9/7.9 mL) at rt, was added DIPEA (8.56 mL, 49.15 mmol, 5 eq.). The resulting mixture was stirred in an oil bath at 130° C. After 72 h, LCMS showed total conversion. Toluene was removed under reduced pressure and the residue was purified by reverse phase flash chromatography (150 g C18 RediSep Rf Gold column, liquid deposit (DMSO), elution: 5% MeOH/0.1% HCOOH over 5 CV, then 5 to 100% MeOH/0.1% HCOOH over 20 CV, then 100% MeOH/0.1% HCOOH over 3 CV). Fractions were combined and concentrated to give 3 (1.81 g, 50% yield) as an off-white solid.

LCMS method 1: 85.8% purity at 215 nm, [M+H]⁺=317.2.

¹H NMR (400 MHz, CDCl₃-d) δ ppm 1.46 (s, 9H), 1.53-1.57 (m, 2H), 2.04-2.14 (m, 2H), 3.04-3.18 (m, 2H), 3.58-3.66 (m, 2H), 4.27 (d, J=12.0 Hz, 2H), 4.47 (br s, 1H), 6.76 (d, J=8.3 Hz, 1H), 7.51-7.61 (m, 1H), 8.11 (s, 1H).

Step 2. Preparation of tert-Butyl 4-[6-[4-(tert-butoxycarbonylamino)-1-piperidyl]-3-pyridyl]-4-cyano-butanoate (5). To a solution of tert-butyl N-[1-[5-(cyanomethyl)-2-pyridyl]-4-piperidyl]carbamate 3 (0.91 g, 2.86 mmol, 1 eq.) in THF (14.30 mL) cooled down to 0° C. was added NaOtBu (286 μL, 0.2900 mmol, 0.1 eq.), directly followed by tert-butyl prop-2-enoate 4 (440 μL, 3.0 mmol, 1.05 eq.) The reaction was then slowly warmed up to rt. After 16 h at rt, LCMS showed completion. The reaction mixture was evaporated to dryness, and the residue was purified by reverse phase flash chromatography (100 g C18 RediSep Rf Gold column, liquid deposit (DMSO), elution: 5% MeCN/0.1% HCOOH over 5 CV, then 5 to 100% MeCN/0.1% HCOOH over 20 CV, then 100% MeCN/0.1% HCOOH over 3 CV, desired product was released around 70% MeCN). Fractions were combined and concentrated to give 5 (224 mg, 17% yield) as a yellow oil.

LCMS method 1: 99.9% purity at 215 nm, [M+H]⁺=445.2.

¹H NMR (400 MHz, CDCl₃-d) δ ppm 1.33-1.55 (m, 18H), 1.58-1.63 (m, 1H), 1.98-2.20 (m, 4H), 2.32-2.49 (m, 2H), 2.94-3.06 (m, 2H), 3.63-3.78 (m, 1H), 3.81-3.88 (m, 1H), 4.13-4.31 (m, 2H), 4.40-4.53 (m, 1H), 6.68 (d, J=8.8 Hz, 1H), 7.45 (dd, J=8.8, 2.7 Hz, 1H), 8.09 (d, J=2.7 Hz, 1H).

Step 3. Preparation of 3-[6-(4-Amino-1-piperidyl)-3-pyridyl]piperidine-2,6-dione hydrogen sulfate (C-17). A solution of tert-butyl 4-[6-[4-(tert-butoxycarbonylamino)-1-piperidyl]-3-pyridyl]-4-cyano-butanoate 5 (256.3 mg, 0.58 mmol, 1 eq.) and concentrated sulfuric acid (92 μL, 1.73 mmol, 3 eq.) in glacial acetic acid (99 μL, 1.73 mmol, 3 eq.) was stirred at 120° C. (pre-heated oil bath). After 1 h at this temperature, LCMS showed completion. Then, the reaction mixture was evaporated to dryness, and co-evaporated three times with toluene. The residue was purified by reverse phase flash chromatography (100 g C18 RediSep Rf Gold column, liquid deposit (Water), elution: 0% MeCN/0.1% HCOOH over 15 CV, then 0 to 100% MeCN/0.1% HCOOH over 5 CV, then 100% MeCN/0.1% HCOOH over 3 CV, desired product was released around 0% MeCN). Fractions were combined and concentrated to give C-17 (90 mg, 40% yield) as a white solid.

LCMS method 1: 99.9% purity at 254 nm, [M−H₂SO₄+H]⁺=289.2.

¹H NMR (400 MHz, DMSO-d6) δ ppm 1.36-1.47 (m, 2H), 1.84-2.00 (m, 3H), 2.10-2.23 (m, 1H), 2.61-2.74 (m, 1H), 2.80-2.94 (m, 2H), 3.17-3.24 (m, 2H), 3.73 (dd, J=12.3, 4.8 Hz, 1H), 4.29 (d, J=12.7 Hz, 2H), 6.85 (d, J=8.8 Hz, 1H), 7.40 (dd, J=8.7, 2.3 Hz, 1H), 7.96 (d, J=2.2 Hz, 1H), 8.26 (s, 2H), 10.82 (br s, 1H).

### Synthesis of Common Intermediate C-18, C-19, C-20, C-21

### Example I-16. Synthesis of Common Intermediate C-19 and C-20

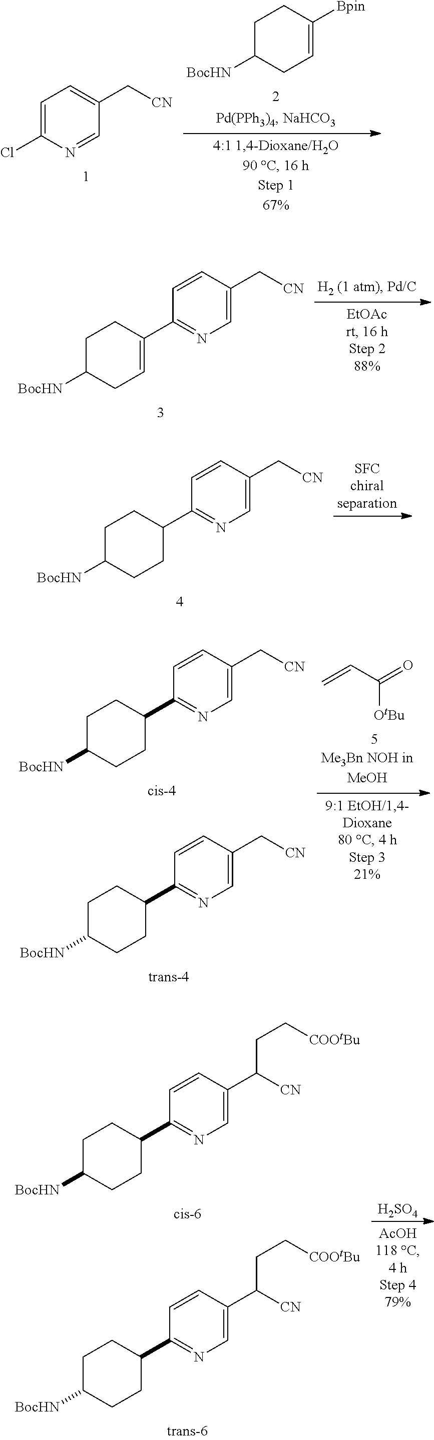

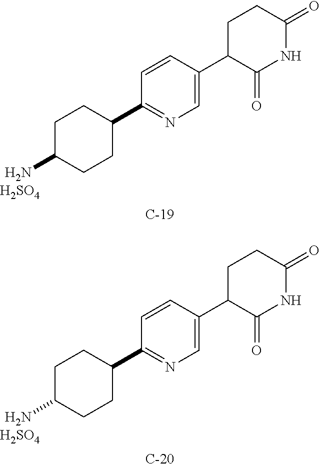

Step 1. Preparation of tert-Butyl N-[4-[5-(cyanomethyl)-2-pyridyl]cyclohex-3-en-1-yl]carbamate (3). In a sealed tube, 2-(6-chloro-3-pyridyl) acetonitrile 1 (375.0 mg, 2.46 mmol, 1.0 equiv), tert-butyl N-[4-(4,4,5,5-tetramethyl-1,3,2-dioxaborolan-2-yl)cyclohex-3-en-1-yl]carbamate 2 (1.00 g, 2.95 mmol, 1.2 equiv), and sodium bicarbonate (619.4 mg, 7.37 mmol, 3.0 equiv) were mixed with 1,4-dioxane (4 mL) and water (1 mL), then nitrogen was bubbled trough the mixture for 30 minutes under sonication. Pd(PPh₃)₄ (284.0 mg, 0.246 mmol, 0.1 equiv) was then quickly added and nitrogen bubbling was continued for additional 10 minutes. The tube was sealed and the reaction mixture was stirred at 90° C. overnight. The reaction mixture was cooled to room temperature, then water (10 mL) and DCM (10 mL) were added. The phases were separated and the aqueous layer was extracted with DCM (3×10 mL). The organic layers were combined, dried over MgSO₄, filtered and evaporated under reduced pressure. The crude was dry loaded on silica and purified by normal phase flash chromatography (heptane/EtOAc, 100:0 to 40:60, 80 g RediSep Rf Gold® Normal-Phase Silica, 20 CV, λ=254-280 nm, product with 48-56% EtOAc) to afford 3 (513 mg, 1.63 mmol, 67% yield) as a white solid.

LCMS method 1: 99.9% purity at 215 nm, [M+H]⁺=314.2, [M-t-Bu+H]⁺=258.2.

¹H NMR (400 MHz, DMSO-d6) δ ppm 1.39 (s, 9H), 1.46-1.62 (m, 1H), 1.85-1.99 (m, 1H), 2.11 (ddd, J=12.6, 9.2, 3.2 Hz, 1H), 2.36-2.48 (m, 2H), 2.66 (br d, J=17.6 Hz, 1H), 3.53 (br s, 1H), 4.06 (s, 2H), 6.63 (br s, 1H), 6.85 (br d, J=7.3 Hz, 1H), 7.55 (d, J=8.2 Hz, 1H), 7.72 (dd, J=8.3, 2.4 Hz, 1H), 8.47 (d, J=1.8 Hz, 1H).

Step 2. Preparation of trans-tert-Butyl N-[4-[5-(cyanomethyl)-2-pyridyl]cyclohexyl]carbamate (trans-4/cis-4). tert-Butyl N-[4-[5-(cyanomethyl)-2-pyridyl]cyclohex-3-en-1-yl]carbamate 3 (510.0 mg, 1.63 mmol, 1.0 equiv) was dissolved in ethyl acetate (32 mL), then the solution was degassed by bubbling nitrogen under sonication for 20 minutes. Pd/C 5% w/w (346 mg, 0.163 mmol, 0.1 equiv) was added, then the mixture was further degassed by bubbling nitrogen under sonication for 20 minutes. The nitrogen balloon was replaced with one filled with hydrogen, which was bubbled through the reaction mixture for 10 minutes, then the reaction mixture was stirred under static hydrogen atmosphere overnight. The reaction mixture was filtered through celite to remove the catalyst, then the celite was washed thoroughly with EtOAc. The solvent was evaporated under reduced pressure to yield the cis/trans mixture of the product (500 mg, 55:45 cis/trans ratio by 1H NMR) as a grey crystalline solid. The cis/trans mixture was submitted to SFC chiral separation to yield trans-4 (213 mg, 0.663 mmol, 41% yield) and cis-4 (250 mg, 0.771 mmol, 47% yield) as white solids (88% total yield).

trans-4. LCMS method 3:98.2% purity at 215 nm, [M-t-Bu+H]⁺=260.2, [M+H]⁺=316.2, [M+Na]⁺=339.2.

HPLC (chiral) method 1: 100% purity at 215 nm.

¹H NMR (400 MHz, DMSO-d6) δ ppm 1.21-1.35 (m, 2H), 1.38 (s, 9H), 1.48-1.63 (m, 2H), 1.79-1.96 (m, 4H), 2.53-2.65 (m, 1H), 3.19-3.31 (m, 1H), 4.03 (s, 2H), 6.76 (br d, J=7.7 Hz, 1H), 7.31 (d, J=8.1 Hz, 1H), 7.68 (dd, J=8.1, 2.1 Hz, 1H), 8.44 (d, J=1.6 Hz, 1H).

cis-4. LCMS method 3:97.3% purity at 215 nm, [M+H]⁺=316.2, [M+Na]⁺=339.1, [M-t-Bu+H]⁺=260.2.

HPLC (chiral) method 1: 100% purity at 215 nm.

¹H NMR (400 MHz, DMSO-d6) δ ppm 1.39 (s, 9H), 1.50-1.63 (m, 4H), 1.63-1.75 (m, 2H), 1.82-2.00 (m, 2H), 2.70 (br t, J=10.5 Hz, 1H), 3.63 (br s, 1H), 4.04 (s, 2H), 6.85 (br d, J=4.8 Hz, 1H), 7.38 (d, J=8.1 Hz, 1H), 7.71 (dd, J=8.0, 1.9 Hz, 1H), 8.44 (s, 1H)

Step 3. Preparation of trans-tert-Butyl 4-[6-[4-(tert-butoxycarbonylamino)cyclohexyl]-3-pyridyl]-4-cyano-butanoate (trans-6). trans-tert-butyl N-[4-[5-(cyanomethyl)-2-pyridyl]cyclohexyl]carbamate trans-4 (181.0 mg, 0.563 mmol, 1.0 equiv) and tert-butyl prop-2-enoate 5 (93 μL, 0.620 mmol, 1.1 equiv) were dissolved in ethanol (5.0 mL) and 1,4-dioxane (0.5 mL), then benzyltrimethylammonium hydroxide solution 40% w/w in MeOH (51 μL, 0.113 mmol, 0.2 equiv) was added and the reaction mixture was stirred at 80° C. for 4 hours. The reaction mixture was evaporated to dryness, then the crude was dry loaded on silica and purified by normal phase flash chromatography (Heptanes/EtOAc, 100:0 to 40:60, 24 g RediSep Rf Gold® Normal-Phase Silica, 25 CV, λ=254-280 nm, product with 40-48% EtOAc, starting material with 52-60% EtOAc) to afford trans-6 (64 mg, 0.116 mmol, 21% yield) as a white solid and recovered trans-4 (85 mg, 0.269 mmol, 48% yield) as a white solid.

LCMS method 1: 80.4% purity at 215 nm, [M+H]⁺=444.2.

Step 4. Preparation of trans-[4-[5-(2,6-Dioxo-3-piperidyl)-2-pyridyl]cyclohexyl]ammonium hydrogen sulfate (C-20). trans-tert-butyl 4-[6-[4-(tert-butoxycarbonylamino)cyclohexyl]-3-pyridyl]-4-cyano-butanoate trans-6 (150.0 mg, 0.288 mmol, 1.0 equiv) was dissolved in acetic acid (5.7 mL), then sulfuric acid (63.0 μL, 1.15 mmol, 4.0 equiv) was added dropwise and the mixture was stirred at 118° C. for 4 hours. Acetic acid was co-evaporated with toluene (3×10 mL) and the residue was dried under high vacuum. The residue was dissolved in the minimum amount of water and purified by reversed-phase flash chromatography (MeCN in 0.1% HCOOH(aq), 0% (5 CV)→20%, 40 g Claricep™ Spherical AQ C18 Column, 20 CV, λ=214-254 nm, product with 0% MeCN, broad band) to afford trans-7 (109 mg, 0.227 mmol, 79% yield) as a white solid as a sulfuric acid salt after lyophilisation.

LCMS method 4:80.4% purity at 215 nm, [M+H]⁺=288.1.

The following compounds were synthesized via the same general routes with modifications to starting material 1 (SM-1) in step 1 and ring geometry after separation (Cis/Trans) (Table 4)

TABLE 4

| | SM-1 | Cis/Trans | Structure |
|---|---|---|---|
| C-18 | 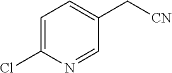 | Cis | 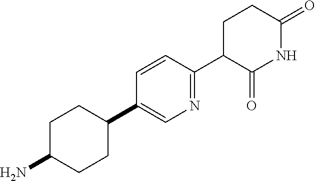 |
| C-19 | 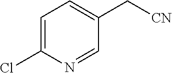 | Cis | 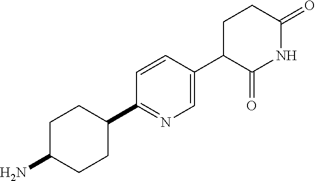 |
| C-20 | 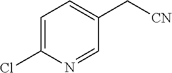 | Trans | 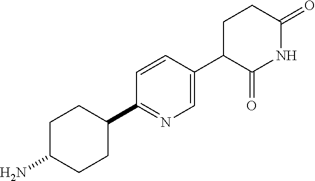 |
| C-21 | 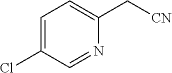 | Trans | 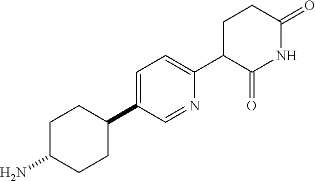 |

### Final Compound Synthesis

### Final Product General Method 1

### Example S1. Synthesis of P-1

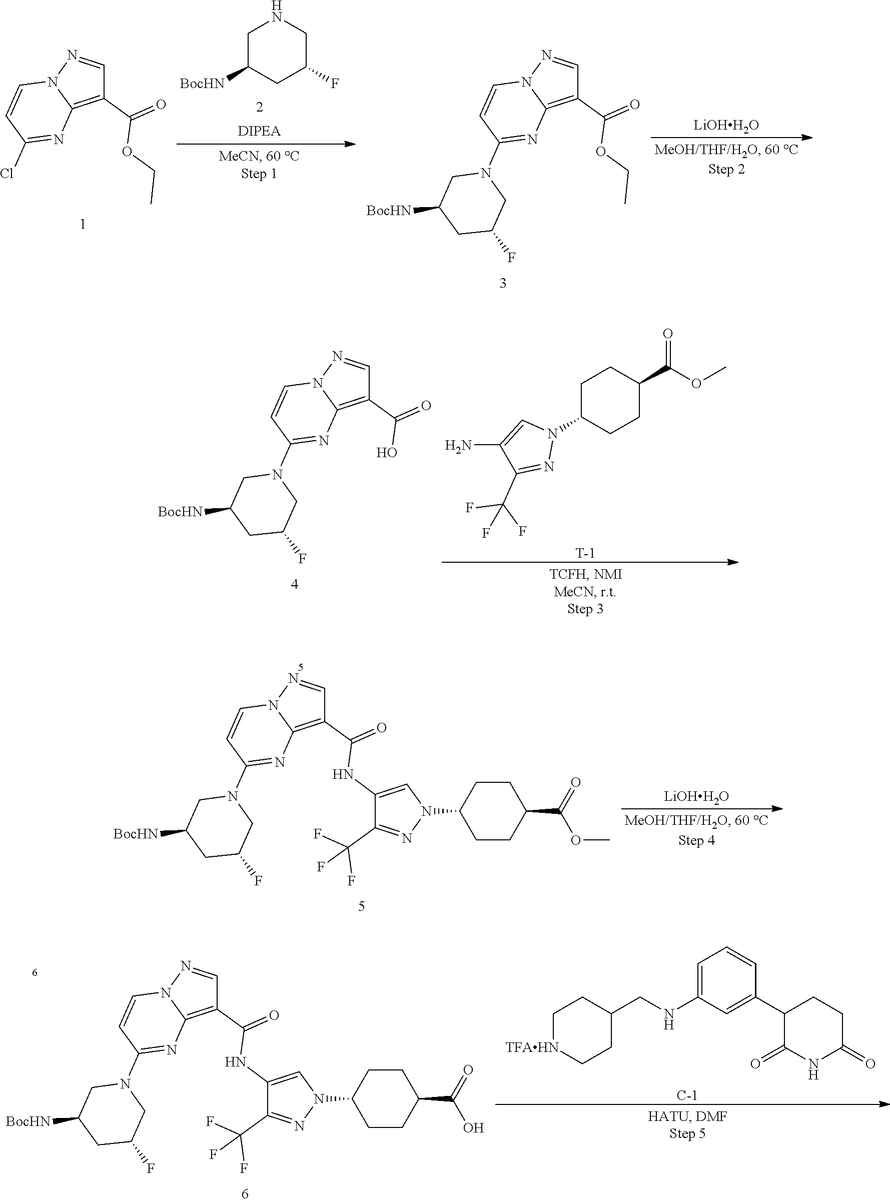

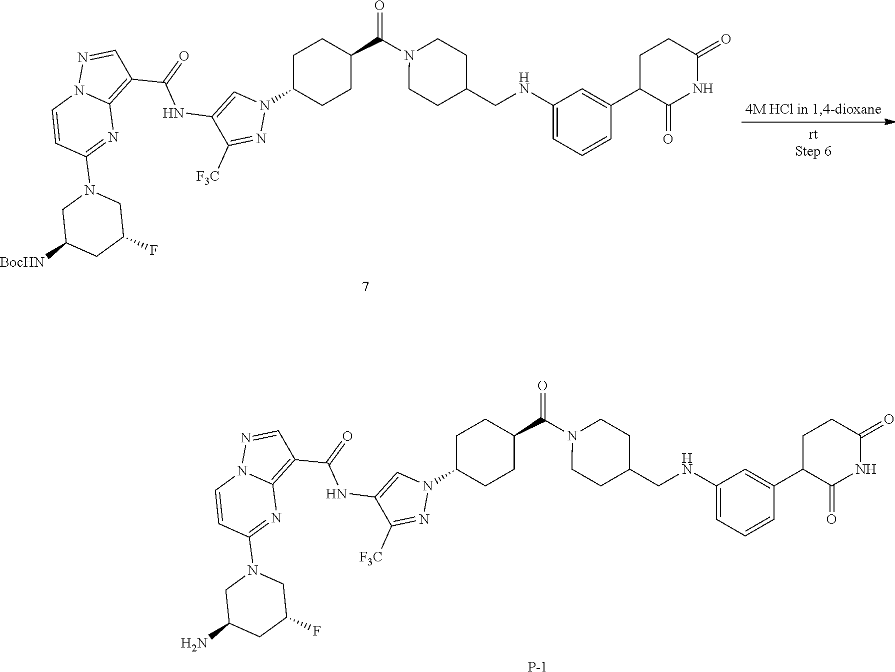

Step 1. Preparation of ethyl 5-[(3R,5R)-3-(tert-butoxycarbonylamino)-5-fluoro-1-piperidyl]pyrazolo[1,5-a]pyrimidine-3-carboxylate (3). To a solution of ethyl 5-chloropyrazolo[1,5-a]pyrimidine-3-carboxylate 1 (800 mg, 3.55 mmol, 1.0 eq.) in MeCN (17.7 mL) was added DIPEA (1.54 mL, 8.86 mmol, 2.5 eq.) and tert-butyl N-[(3R,5R)-5-fluoro-3-piperidyl]carbamate 2 (1.01 g, 4.61 mmol, 1.3 eq.). After stirring at 60° C. over the weekend, LCMS showed complete conversion into 3. The solvent was removed under reduced pressure and the residue was dried under high vacuum to give 3 (1.44 g, quantitative yield) as white solid. The crude product was used in the next step without further purification.

LCMS method 1: retention time: 1.656 min, 99.9% purity at 215 nm, [M+H]⁺=408.2.

¹H NMR (400 MHz, DMSO-d6) δ ppm 1.37-1.45 (m, 9H), 1.56-1.92 (m, 1H), 2.09-2.20 (m, 1H), 2.56-2.71 (m, 1H), 2.76-3.03 (m, 1H), 3.08-3.19 (m, 1H), 3.29 (s, 1H), 3.37 (br s, 1H), 3.56-3.73 (m, 2H), 4.11-4.25 (m, 2H), 4.41-4.73 (m, 1H), 4.99 (br s, 1H), 5.11 (br s, 1H), 6.85 (br d, J=8.1 Hz, 1H), 7.10 (br d, J=7.8 Hz, 1H), 8.22 (s, 1H), 8.73 (d, J=7.8 Hz, 1H).

¹⁹F NMR (377 MHz, DMSO-d6) δ ppm −184.32 (s, 1F).

Step 2. Preparation of 5-[(3R,5R)-3-(tert-Butoxycarbonylamino)-5-fluoro-1-piperidyl]pyrazolo[1,5-a]pyrimidine-3-carboxylic acid (4). To a solution of ethyl 5-[(3R,5R)-3-(tert-butoxycarbonylamino)-5-fluoro-1-piperidyl]pyrazolo[1,5-a]pyrimidine-3-carboxylate 3 (1.44 mg, 3.53 mmol, 1.0 eq) in THF (5.89 mL) and methanol (5.89 mL) was added a solution of LiOH·H₂O (1.48 g, 35.34 mmol, 10.0 eq.) in water (5.89 mL). After stirring at 60° C. for 18 h, LCMS showed complete conversion into desired product 4. The reaction mixture was concentrated under vacuum to remove THE/MeOH and the crude mixture was diluted with water. Under vigorous agitation, the mixture was acidified with a 6N aqueous HCl solution until pH=3 (formation of a precipitate). The suspension was filtered on a Buchner funnel and the solid was rinsed with water. The solid was dried overnight in a stove under vacuum to give 4 (1.40 g, quantitative yield) as white solid. The crude product was used in the next without further purification.

LCMS method 1: retention time: 1.487 min, 99.9% purity at 215 nm, [M+H]⁺=380.1.

¹H NMR (400 MHz, DMSO-d6) δ ppm 1.41 (s, 9H), 1.66-1.95 (m, 1H), 2.05-2.26 (m, 1H), 2.92-3.11 (m, 1H), 3.28-3.30 (m, 1H), 3.34-3.49 (m, 1H), 3.59-3.74 (m, 1H), 4.52-4.75 (m, 1H), 4.99 (br s, 1H), 5.11 (br s, 1H), 6.82 (br d, J=7.6 Hz, 1H), 7.12 (br d, J=7.8 Hz, 1H), 8.19 (s, 1H), 8.73 (d, J=7.8 Hz, 1H).

¹⁹F NMR (377 MHz, DMSO-d6) δ ppm −184.32 (s, 1F).

Step 3. Preparation of Methyl 4-[4-[[5-[(3R,5R)-3-(tert-butoxycarbonylamino)-5-fluoro-1-piperidyl]pyrazolo[1,5-a]pyrimidine-3-carbonyl]amino]-3-(trifluoromethyl)pyrazol-1-yl]cyclohexanecarboxylate (5). To a solution of 5-[(3R,5R)-3-(tert-butoxycarbonylamino)-5-fluoro-1-piperidyl]pyrazolo[1,5-a]pyrimidine-3-carboxylic acid 4 (330 mg, 0.87 mmol, 1.0 eq.) and methyl 4-[4-amino-3-(trifluoromethyl)pyrazol-1-yl]cyclohexanecarboxylate T-1 (255 mg, 0.87 mmol, 1.0 eq.) in MeCN (4.37 mL, 0.2 M) was added NMI (208 μL, 2.62 mmol, 3.0 eq.), followed by TCFH (368 mg, 1.31 mmol, 1.5 eq.). The resulting mixture was stirred at room temperature. After stirring for 1 hour, LCMS showed complete conversion into 7. Nanopure water was added to the reaction mixture. The suspension was sonicated and filtered on a Buchner funnel. The solid was rinsed with nanopure water and dried under high vacuum for 2 h to give 5 (279 mg, 46% yield) as white solid. The crude product was used in the next without further purification.

LCMS method 1: retention time: 1.812 min, 99.9% purity at 215 nm, [M+H]⁺=653.2.

¹H NMR (400 MHz, DMSO-d6) δ ppm 1.30 (br s, 9H), 1.49-1.62 (m, 3H), 1.75-1.92 (m, 4H), 2.01-2.11 (m, 5H), 3.40-3.57 (m, 1H), 3.63 (s, 3H), 4.32 (br t, J=10.9 Hz, 1H), 4.50-4.64 (m, 1H), 4.96-5.18 (m, 1H), 6.92 (br d, J=5.5 Hz, 1H), 7.09 (br s, 1H), 8.30 (s, 1H), 8.41 (br s, 1H), 8.82 (br d, J=7.8 Hz, 1H), 9.22 (br s, 1H). 2H not observed.

¹⁹F NMR (377 MHz, DMSO-d6) δ ppm −185.73-−184.64 (m, 1F), −59.01 (br s, 3F).

Step 4. Preparation of 4-[4-[[5-[(3R,5R)-3-(tert-Butoxycarbonylamino)-5-fluoro-1-piperidyl]pyrazolo[1,5-a]pyrimidine-3-carbonyl]amino]-3-(trifluoromethyl)pyrazol-1-yl]cyclohexanecarboxylic acid (6). To a solution of methyl 4-[4-[5-[(3R,5R)-3-(tert-butoxycarbonylamino)-5-fluoro-1-piperidyl]pyrazolo[1,5-a]pyrimidine-3-carbonyl]amino]-3-(trifluoromethyl)pyrazol-1-yl]cyclohexanecarboxylate 5 (279 mg, 0.43 mmol, 1.0 eq.) in THF (2.14 mL, 0.1 M) was added a solution of LiOH·H₂O (180 mg, 4.27 mmol, 10.0 eq.) in water (2.14 mL, 0.1 M) and the resulting mixture was stirred at room temperature. After an overnight stirring, LCMS showed complete conversion into 6. The reaction mixture was concentrated under reduced pressure and the residue was suspended in nanopure water. Under vigorous agitation at 0° C., a 6N aqueous solution of HCl was added until pH=3. The solid was filtered on a Buchner funnel, rinsed with nanopure water and dried under high vacuum overnight to give 6 (232 mg, 83% yield) as white solid. The product was used in the next step without further purification.

LCMS method 1: retention time: 1.667 min, 99.9% purity at 215 nm, [M+H]⁺=639.2.

¹H NMR (400 MHz, DMSO-d6) δ ppm 1.29 (br s, 9H), 1.43-1.61 (m, 3H), 1.68-1.93 (m, 4H), 1.97-2.14 (m, 5H), 2.25-2.39 (m, 1H), 3.39-3.75 (m, 2H), 4.30 (br t, J=11.7 Hz, 1H), 4.50-4.68 (m, 1H), 4.89-5.19 (m, 1H), 6.91 (br s, 1H), 7.10 (br s, 1H), 8.29 (br s, 1H), 8.41 (br s, 1H), 8.81 (br d, J=6.1 Hz, 1H), 9.21 (br s, 1H), 12.15 (br s, 1H).

¹⁹F NMR (377 MHz, DMSO-d6) δ ppm −186.50-−181.97 (m, 1F), −59.01 (br s, 3F).

Step 5. Preparation of tert-Butyl N-[(3R,5R)-1-[3-[[1-[4-[4-[[3-(2,6-dioxo-3-piperidyl)anilino]methyl]piperidine-1-carbonyl]cyclohexyl]-3-(trifluoromethyl)pyrazol-4-yl]carbamoyl]pyrazolo[1,5-a]pyrimidin-5-yl]-5-fluoro-3-piperidyl]carbamate (7). To a solution of 3-[3-(4-piperidylmethylamino)phenyl]piperidine-2,6-dione 2,2,2-trifluoroacetic acid salt C-1 (44.8 mg, 0.090 mmol) and 4-[4-[5-[(3R,5R)-3-(tert-butoxycarbonylamino)-5-fluoro-1-piperidyl]pyrazolo[1,5-a]pyrimidine-3-carbonyl]amino]-3-(trifluoromethyl)pyrazol-1-yl]cyclohexanecarboxylic acid 6 (38 mg, 0.060 mmol) in DMF (0.500 mL) was added DIPEA (103.6 μL, 0.600 mmol). The reaction mixture was stirred at room temperature for 5 minutes, then HATU (27.15 mg, 0.070 mmol) was added. The resulting mixture was stirred at room temperature overnight. The crude reaction mixture was loaded onto a C18 reverse phase column for purification (30 g C18 gold column, gradient elution, 50-65% MeCN/0.1% aq. HCO₂H) affording 40 mg (73%) of 7 as a pale yellow solid.

LCMS method 1: 99.9% (UV 215 nm); exact mass calculated=921.43; found [M+H]⁺=922.2.

¹H NMR (400 MHz, DMSO-d6) δ ppm 0.93-1.05 (m, 1H), 1.08-1.16 (m, 1H), 1.29 (br s, 9H), 1.50-1.67 (m, 2H), 1.78 (br s, 5H), 1.82-1.97 (m, 4H), 2.02-2.15 (m, 6H), 2.40-2.46 (m, 1H), 2.57-2.68 (m, 1H), 2.76 (br t, J=11.2 Hz, 1H), 2.90 (br s, 2H), 2.93-3.09 (m, 2H), 3.37-3.57 (m, 1H), 3.68 (br dd, J=9.7, 5.3 Hz, 2H), 4.04 (br d, J=13.7 Hz, 1H), 4.24-4.37 (m, 1H), 4.37-4.45 (m, 1H), 4.47-4.68 (m, 1H), 4.97-5.17 (m, 1H), 5.68 (br s, 1H), 6.35 (br d, J=7.3 Hz, 1H), 6.41 (s, 1H), 6.46 (br d, J=8.3 Hz, 1H), 6.91 (br d, J=6.8 Hz, 1H), 7.01 (t, J=7.7 Hz, 1H), 7.09 (br d, J=3.7 Hz, 1H), 8.30 (s, 1H), 8.40 (br s, 1H), 8.82 (br d, J=7.8 Hz, 1H), 9.22 (s, 1H), 10.78 (s, 1H).

¹⁹F NMR (377 MHz, DMSO-d6) δ ppm −59.00 (s, 1F).

Step 6. Preparation of N-[1-[4-[4-[[3-(2,6-dioxo-3-piperidyl)anilino]methyl]piperidine-1-carbonyl]cyclohexyl]-3-(trifluoromethyl)pyrazol-4-yl]-5-[(3R,5R)-3-amino-5-fluoro-1-piperidyl]pyrazolo[1,5-a]pyrimidine-3-carboxamide hydrochloride salt (P-1). A solution of tert-butyl N-[(3R,5R)-1-[3-[[1-[4-[4-[[3-(2,6-dioxo-3-piperidyl)anilino]methyl]piperidine-1-carbonyl]cyclohexyl]-3-(trifluoromethyl)pyrazol-4-yl]carbamoyl]pyrazolo[1,5-a]pyrimidin-5-yl]-5-fluoro-3-piperidyl]carbamate 7 (40 mg, 0.040 mmol) in 4.0 M HCl/1,4-dioxane (1.63 mL, 6.51 mmol) was stirred at room temperature until completion by HPLC analysis (ca. 1 h). The solution was concentrated and purified by C18 reverse phase column chromatography (gradient elution, 5-40% MeCN/0.02 M aq. HCl) giving after lyophilisation 26.41 mg (73%) of P-1 as a white solid as an HCl salt.

LCMS method 3:98.8% (UV 215 nm); exact mass calculated (free base)=821.37; found [M−HCl+H]⁺=822.3.

¹H NMR (400 MHz, DMSO-d6) δ ppm 0.94-1.22 (m, 2H), 1.48-1.67 (m, 2H), 1.72-2.23 (m, 13H), 2.34-2.47 (m, 1H), 2.56-2.68 (m, 2H), 2.71-2.83 (m, 1H), 2.89-3.07 (m, 3H), 3.23-3.57 (m, 4H), 3.99-4.10 (m, 1H), 4.29-4.47 (m, 2H), 4.54-4.84 (m, 2H), 5.12 (d, J=46.7 Hz, 1H), 6.34-6.79 (m, 3H), 6.91 (d, J=8.1 Hz, 1H), 7.03-7.15 (m, 1H), 8.07-8.38 (m, 4H), 8.45 (s, 1H), 8.93 (d, J=7.8 Hz, 1H), 9.24 (s, 1H), 10.81 (s, 1H), 1H not apparent.

¹⁹F NMR (377 MHz, DMSO d6) δ ppm −184.88 (s, 1F), −58.78 (s, 3F).

### Example S2. Synthesis of P-2

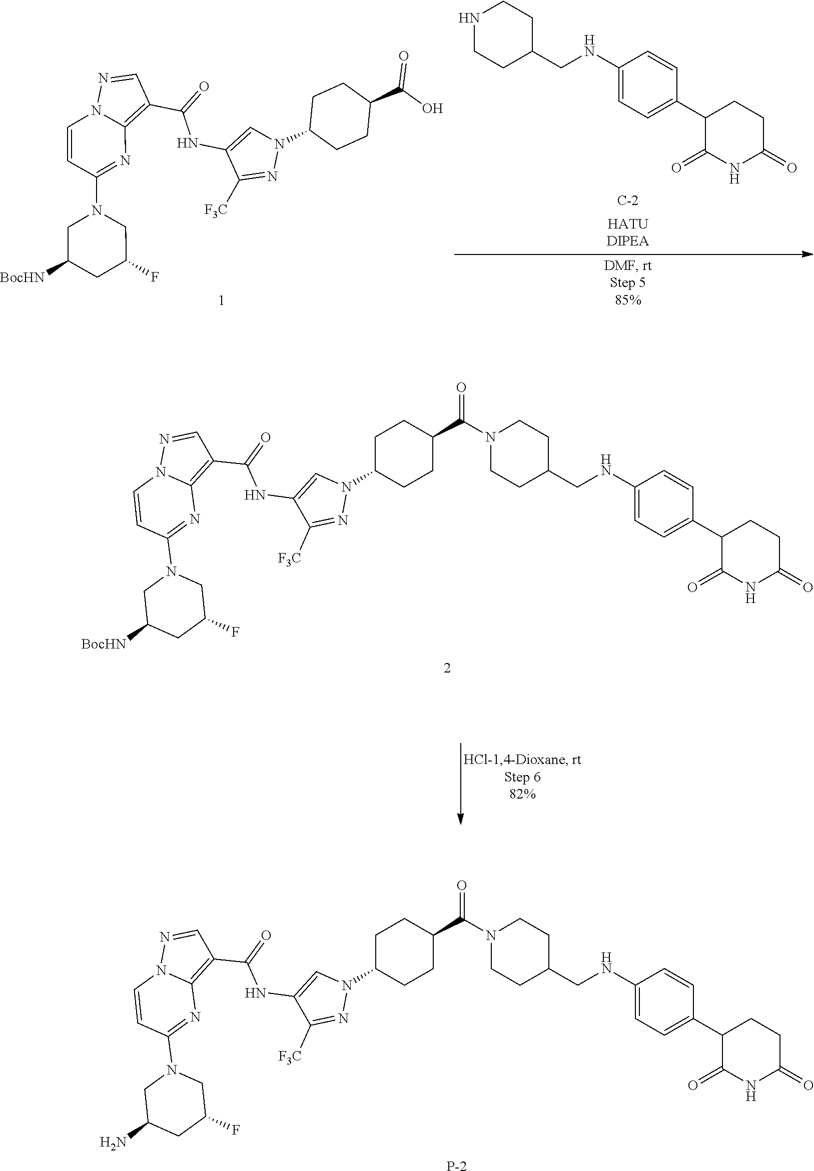

Step 5. Preparation of tert-Butyl N-[(3R,5R)-1-[3-[[1-[4-[4-[[4-(2,6-dioxo-3 piperidyl)anilino]methyl]piperidine-1-carbonyl]cyclohexyl]-3-(trifluoromethyl)pyrazol-4-yl]carbamoyl]pyrazolo[1,5-a]pyrimidin-5-yl]-5-fluoro-3-piperidyl]carbamate (2). To a solution of 3-[4-(4-piperidylmethylamino)phenyl]piperidine-2,6-dione dihydrochloride C-2 (47.13 mg, 0.09 mmol) and 4-[4-[[5-[(3R,5R)-3-(tert-butoxycarbonylamino)-5-fluoro-1piperidyl]pyrazolo[1,5-a]pyrimidine-3-carbonyl]amino]-3-(trifluoromethyl)pyrazol-1-yl]cyclohexanecarboxylic acid 1-intermediate 6 from synthesis of P-1-(40.0 mg, 0.06 mmol) in DMF (1 mL) was added DIPEA (0.11 mL, 0.63 mmol). The mixture was stirred at rt for 5 minutes, then HATU (28.58 mg, 0.08 mmol) was added. The mixture was stirred at rt for 16 h. LCMS showed complete conversion to the desired product. The mixture was injected directly into a 50 g C18 gold column and purified by reverse phase flash chromatography (liquid deposit with DMSO, eluted with MeCN/0.1% HCOOH, 4 CV at 5% MeCN; 1 CV to 55% MeCN; 15 CV to 70% MeCN). The product came out at 61% MeCN. Fractions were concentrated to give 2 (49.3 mg, 85% yield) as an orange solid.

LCMS method 1: 99.9% purity at 215 nm, [M+H]⁺=922.2, [M+2H]²⁺=461.8.

¹H NMR (400 MHz, DMSO-d6) δ ppm 0.95-1.04 (m, 1H), 1.07-1.17 (m, 1H), 1.28 (br s, 9H), 1.48-1.65 (m, 3H), 1.72-1.84 (m, 5H), 1.85-1.97 (m, 4H), 1.98-2.07 (m, 4H), 2.09-2.18 (m, 2H), 2.40-2.47 (m, 2H), 2.56-2.66 (m, 1H), 2.71-2.81 (m, 1H), 2.91 (br d, J=5.6 Hz, 2H), 2.97-3.06 (m, 1H), 3.60-3.71 (m, 2H), 4.01-4.09 (m, 1H), 4.28-4.36 (m, 1H), 4.38-4.46 (m, 1H), 4.99-5.15 (m, 1H), 5.62-5.68 (m, 1H), 6.53 (d, J=8.3 Hz, 2H), 6.87-6.95 (m, 3H), 7.11 (br s, 1H), 8.30 (br s, 1H), 8.42 (br s, 1H), 8.80 (br d, J=7.8 Hz, 1H), 9.22 (br s, 1H), 10.73 (br s, 1H).

Step 6. Preparation of N-[1-[4-[4-[[4-(2,6-Dioxo-3-piperidyl)anilino]methyl]piperidine-1-carbonyl]cyclohexyl]-3-(trifluoromethyl)pyrazol-4-yl]-5-[(3R,5R)-3-amino-5-fluoro-1-piperidyl]pyrazolo[1,5-a]pyrimidine-3-carboxamide hydrochloride (P-2). tert-Butyl N-[(3R,5R)-1-[3-[[1-[4-[4-[[4-(2,6-dioxo-3-piperidyl)anilino]methyl]piperidine-1-carbonyl]cyclohexyl]-3-(trifluoromethyl)pyrazol-4-yl]carbamoyl]pyrazolo[1,5-a]pyrimidin-5-yl]-5-fluoro-3-piperidyl]carbamate 2 (49.3 mg, 0.05 mmol) was solubilized in 4 M HCl in 1,4-dioxane (2.01 mL, 8.02 mmol). The solution was stirred at rt for 30 minutes. LCMS showed complete conversion. The solvent was evaporated under reduce pressure and the crude was purified by reverse phase flash chromatography (50 g C18 gold column, liquid deposit with DMSO, eluted with MeCN/0.02 M HCl, 3.5 CV at 5% MeCN; 10 CV to 35% MeCN). Fractions were combined, concentrated and lyophilised to give P-2 (36.4 mg, 82% yield) as a white solid as a hydrochloride salt.

LCMS method 3:98.7% purity at 215 nm, [M−HCl+H]⁺=822.4; [M−HCl+2H]²⁺=411.7.

¹H NMR (400 MHz, DMSO-d6) δ ppm 0.96-1.22 (m, 2H), 1.48-1.68 (m, 2H), 1.74-2.20 (m, 12H), 2.35-2.47 (m, 1H), 2.55-2.68 (m, 1H), 2.71-2.81 (m, 1H), 2.96-3.08 (m, 3H), 3.24-3.61 (m, 3H), 3.69-3.78 (m, 2H), 4.00-4.09 (m, 2H), 4.28-4.48 (m, 2H), 4.54-4.84 (m, 2H), 5.06 (d, J=46.5 Hz, 1H), 6.76-7.16 (m, 5H), 8.21-8.38 (m, 4H), 8.45 (s, 1H), 8.93 (d, J=7.8 Hz, 1H), 9.24 (s, 1H), 10.78 (s, 1H).

¹⁹F NMR (377 MHz, DMSO-d6) δ ppm −185.00 (s, 1F), −58.75 (s, 3F).

### Example S3. Synthesis of P-15

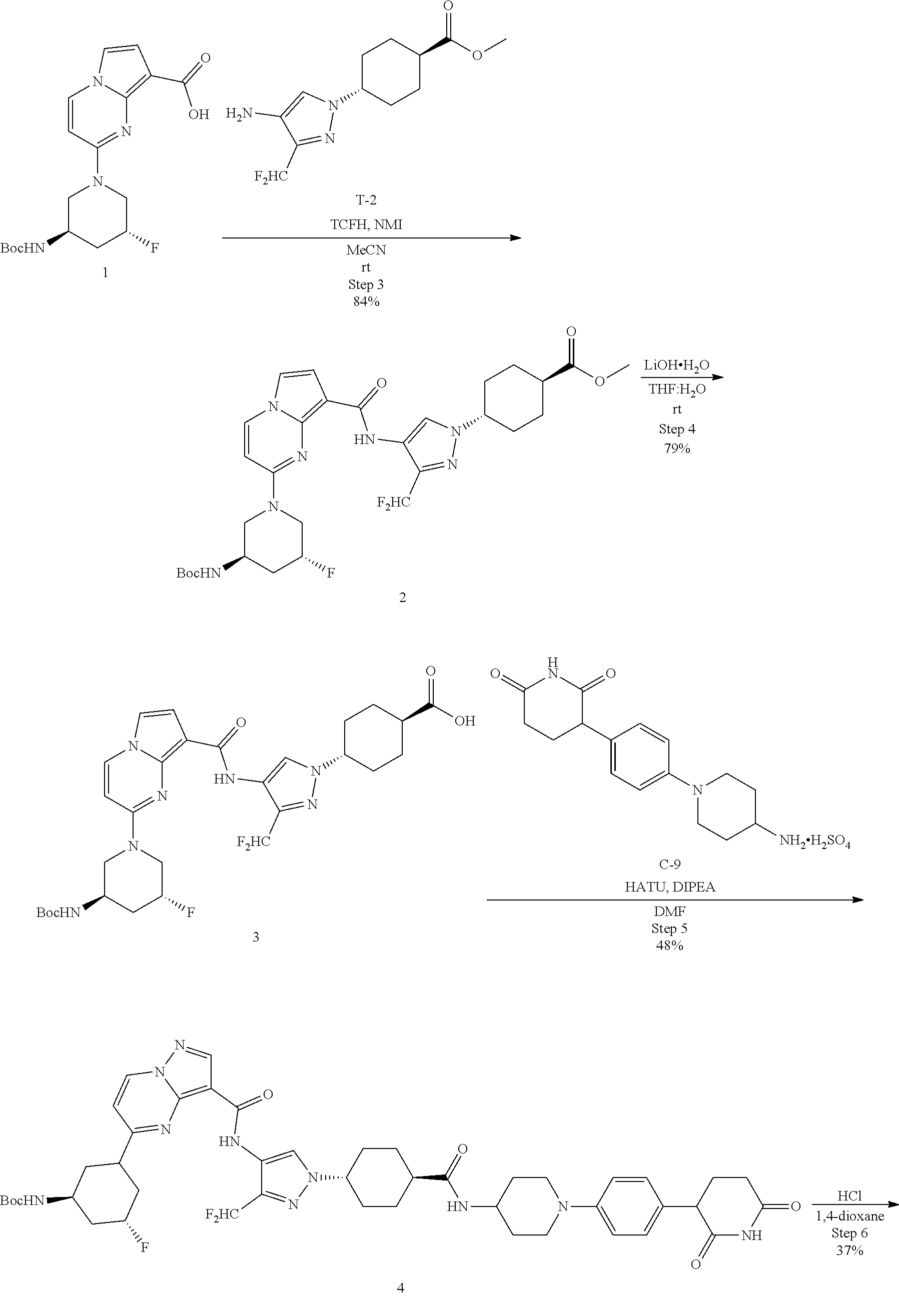

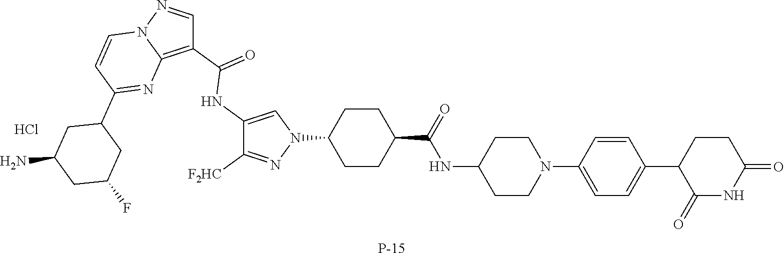

Step 3. Preparation of 5-Methyl 4-[4-[5-[(3R,5R)-3-(tert-butoxycarbonylamino)-5-fluoro-1-piperidyl]pyrazolo[1,5-a]pyrimidine-3-carbonyl]amino]-3-(difluoromethyl)pyrazol-1-yl]cyclohexanecarboxylate (2). To a solution of methyl 4-[4-amino-3-(difluoromethyl)pyrazol-1-yl]cyclohexanecarboxylate T-2 (239 mg, 0.870 mmol, 1.0 equiv.) and 5-[(3R,5R)-3-(tert-butoxycarbonylamino)-5-fluoro-1-piperidyl]pyrazolo[1,5-a]pyrimidine-3-carboxylic acid 1-intermediate 4 from synthesis of P-1-(332 mg, 0.870 mmol, 1.0 equiv.) in MeCN (4.37 mL, 0.2 M) was added NMI (208 μL, 2.62 mmol, 3.0 equiv.), followed by TCFH (368 mg, 1.31 mmol, 1.5 equiv.). The resulting mixture was stirred at rt for 1 h. Nanopure water was added to the reaction mixture. The suspension was sonicated then filtered on a Buchner funnel. The solid was washed with nanopure water and dried under high vacuum to afford the pure 2 (466 mg, 84% yield) without further purification.

LCMS method 1: 99.9% purity at 215 nm, [M+H]⁺=635.2 m/z.

¹H NMR (400 MHz, DMSO-d6) δ ppm 1.34 (s, 9H), 1.40-1.44 (m, 1H), 1.48-1.59 (m, 2H), 1.76-1.94 (m, 3H), 1.98-2.17 (m, 5H), 2.36-2.45 (m, 1H), 2.98-3.12 (m, 1H), 3.37-3.52 (m, 1H), 3.62 (s, 3H), 3.64-3.71 (m, 1H), 4.20-4.29 (m, 1H), 4.58-4.80 (m, 1H), 4.94-5.12 (m, 1H), 6.85-6.95 (m, 1H), 7.02-7.23 (m, 2H), 8.23-8.39 (m, 2H), 8.81 (d, J=7.8 Hz, 1H), 9.31 (s, 1H).

¹⁹F NMR (377 MHz, DMSO-d6) δ ppm −184.13 (s, 1F), −111.44 (s, 2F).

Step 4. Preparation of 4-[3-(Difluoromethyl)-4-[[5-[(3R,5R)-3-(tert-butoxycarbonylamino)-5-fluoro-1-piperidyl]pyrazolo[1,5-a]pyrimidine-3-carbonyl]amino]pyrazol-1-yl]cyclohexanecarboxylic acid (3). To a solution of methyl 4-[3-(difluoromethyl)-4-[5-[(3R,5R)-3-(tert-butoxycarbonylamino)-5-fluoro-1-piperidyl]pyrazolo[1,5-a]pyrimidine-3-carbonyl]amino]pyrazol-1-yl]cyclohexanecarboxylate 2 (466 mg, 0.730 mmol, 1.0 equiv.) in THF (2.45 mL, 0.15 M) was added a solution of LiOH·H₂O (308 mg, 7.34 mmol, 10 equiv.) in water (2.45 mL, 0.15 M). The resulting mixture was stirred at rt overnight. The solvents were removed under reduced pressure and the residue was suspended in nanopure water and sonicated. Under vigorous agitation at 0° C., aqueous 6 M HCl was added until pH=3. The solid was filtered on a Buchner funnel, rinsing with nanopure water to afford pure 3 (362 mg, 79% yield).

LCMS method 1: 99.9% purity at 215 nm, [M+H]⁺=621.3 m/z.

¹H NMR (400 MHz, DMSO-d6) δ ppm 1.34 (s, 9H), 1.39-1.43 (m, 1H), 1.47-1.57 (m, 2H), 1.72-1.86 (m, 3H), 2.00-2.15 (m, 5H), 2.26-2.35 (m, 1H), 2.99-3.13 (m, 1H), 3.42-3.52 (m, 2H), 3.63-3.72 (m, 1H), 4.17-4.30 (m, 1H), 4.95-5.12 (m, 1H), 6.85-6.95 (m, 1H), 7.03-7.23 (m, 2H), 8.28 (s, 1H), 8.34 (s, 1H), 8.81 (d, J=8.1 Hz, 1H), 9.31 (s, 1H), 12.09-12.23 (m, 1H).

¹⁹F NMR (377 MHz, DMSO-d6) δ ppm −184.09 (s, 1F), −111.31 −(s, 2F).

Step 5. Preparation of tert-Butyl N-[(3R,5R)-1-[3-[[3-(difluoromethyl)-1-[4-[[1-[4-(2,6-dioxo-3-piperidyl)phenyl]-4-piperidyl]carbamoyl]cyclohexyl]pyrazol-4-yl]carbamoyl]pyrazolo[1,5-a]pyrimidin-5-yl]-5-fluoro-3-piperidyl]carbamate (4). To a solution of 4-[4-[5-[(3R,5R)-3-(tert-butoxycarbonylamino)-5-fluoro-1-piperidyl]pyrazolo[1,5-a]pyrimidine-3-carbonyl]amino]-3-(difluoromethyl)pyrazol-1-yl]cyclohexanecarboxylic acid 3 (60 mg, 0.10 mmol), 3-[4-(4-amino-1-piperidyl)phenyl]piperidine-2,6-dione sulfuric acid salt C-9 (55.9 mg, 0.150 mmol) and DIPEA (0.17 mL, 0.97 mmol) in DMF (1.0 mL) was added HATU (55.14 mg, 0.1500 mmol). The solution was stirred at rt overnight to reach 68% completion by HPLC analysis. Additional 3-[4-(4-amino-1-piperidyl)phenyl]piperidine-2,6-dione sulfuric acid salt 3 (18.6 mg, 0.75 mmol) and HATU (18.4 mg, 0.75 mmol) were added and the reaction was stirred an additional 3 h at rt to reach completion. The reaction was diluted with H₂O and the mixture was stirred in an ice bath for 1 h which led to a formation of a fine precipitate. The aqueous suspension was extracted with DCM (3×) and the aqueous suspended precipitate was filtered and the cake was rinsed with H₂O. The white solid was suspended in H₂O, frozen and lyophilised overnight to afford 46 mg (48%) of 4 which was used without further purification in the subsequent step.

HPLC: 90.4% (215 nm).

LCMS method 2: exact mass calculated=889.42, found [M+2H]²⁺=446.0.

Step 6. Preparation of 5-[(3R,5R)-3-amino-5-fluoro-1-piperidyl]-N-[3-(difluoromethyl)-1-[4-[[1-[4-(2,6-dioxo-3-piperidyl)phenyl]-4-piperidyl]carbamoyl]cyclohexyl]pyrazol-4-yl]pyrazolo[1,5-a]pyrimidine-3-carboxamide hydrochloride (P-15). A solution of tert-butyl N-[(3R,5R)-1-[3-[[3-(difluoromethyl)-1-[4-[[1-[4-(2,6-dioxo-3-piperidyl)phenyl]-4-piperidyl]carbamoyl]cyclohexyl]pyrazol-4-yl]carbamoyl]pyrazolo[1,5-a]pyrimidin-5-yl]-5-fluoro-3-piperidyl]carbamate 4 (46 mg, 0.050 mmol) in 4.0 M HCl in 1,4-dioxane (3.5 mL, 0.050 mmol) was stirred at rt until completion by HPLC analysis (ca. 1.5 h). The reaction mixture was concentrated to dryness and purified by C18 reverse phase chromatography (gradient elution, 5-100% MeCN/20 mM aq. HCl) and lyophilised to afford 13.58 mg (37%) of P-15 as a white solid.

LCMS method 3:99.9% (215 nm); exact mass calculated (free base)=789.37; found [M−HCl+H]⁺=790.3.

¹H NMR (400 MHz, DMSO-d6) δ ppm 1.55-1.67 (m, 3H), 1.70-1.82 (m, 3H), 1.83-2.26 (m, 12H), 2.35-2.41 (m, 1H), 2.63-2.72 (m, 1H), 3.25-3.52 (m, 4H), 3.57-3.66 (m, 2H), 3.76-3.83 (m, 2H), 4.21-4.31 (m, 1H), 4.47-4.71 (m, 1H), 4.73-4.97 (m, 1H), 5.11 (d, J=48.0 Hz, 1H), 6.89 (d, J=8.0 Hz, 1H), 6.97-7.35 (m, 5H), 7.79-8.00 (m, 1H), 8.15-8.28 (m, 3H), 8.33 (s, 1H), 8.39 (s, 1H), 8.93 (d, J=7.6 Hz, 1H), 9.33 (s, 1H), 10.81 (s, 1H).

¹⁹F NMR (377 MHz, DMSO-d6) δ ppm −185.26 (s, 1F), −111.21 (s, 2F).

### Example S4. Synthesis of P-16

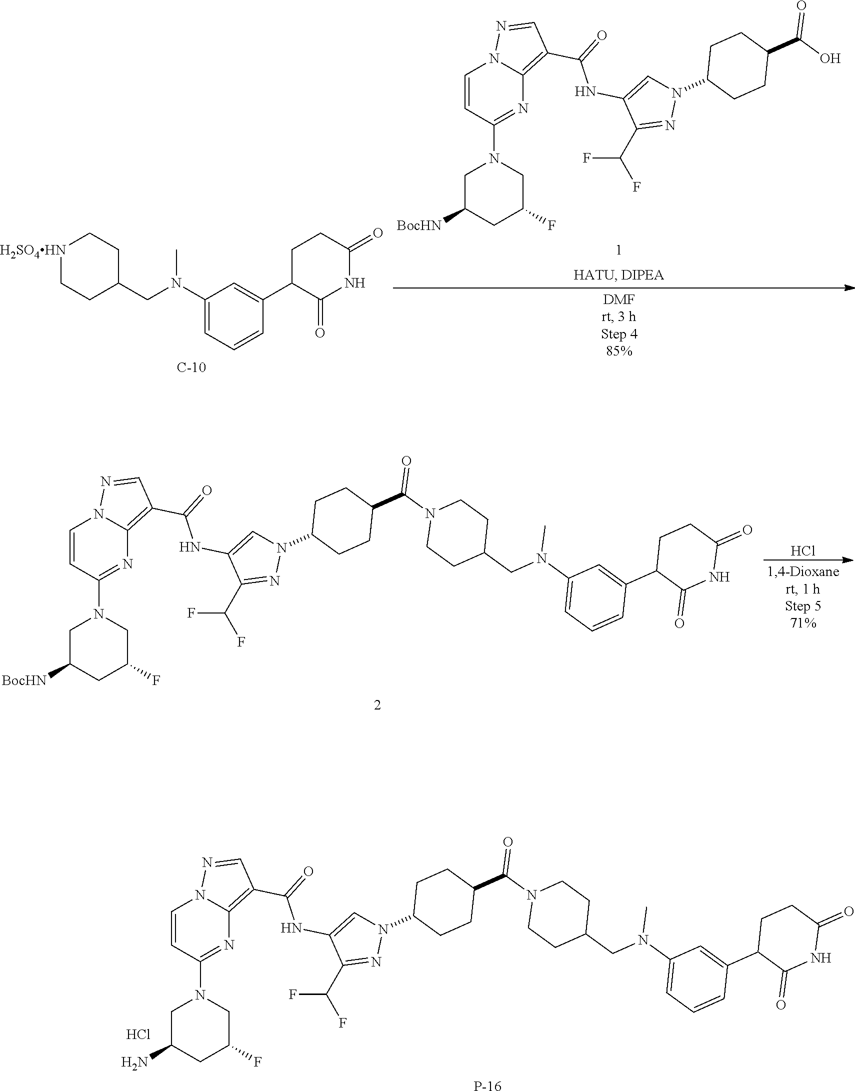

Step 5. Preparation of tert-Butyl N-[(3R,5R)-1-[3-[[3-(difluoromethyl)-1-[4-[4-[[3-(2,6-dioxo-3-piperidyl)-N-methyl-anilino]methyl]piperidine-1-carbonyl]cyclohexyl]pyrazol-4-yl]carbamoyl]pyrazolo[1,5-a]pyrimidin-5-yl]-5-fluoro-3-piperidyl]carbamate (2). 4-[3-(difluoromethyl)-4-[5-[(3R,5R)-3-(tert-butoxycarbonylamino)-5-fluoro-1-piperidyl]pyrazolo[1,5-a]pyrimidine-3-carbonyl]amino]pyrazol-1-yl]cyclohexanecarboxylic acid 1-intermediate 3 from synthesis of P-15-(70.0 mg, 0.113 mmol, 1.0 equiv) and 3-[3-[methyl(piperidin-1-ium-4-ylmethyl)amino]phenyl]piperidine-2,6-dione hydrogen sulfate C-10 (60.6 mg, 0.147 mmol, 1.3 equiv) were dissolved in DMF (0.4 mL), then DIPEA (196 μL, 1.13 mmol, 10 equiv) and HATU (51.5 mg, 0.135 mmol, 1.2 equiv) were added in sequence. The reaction mixture was stirred overnight at room temperature. More amine (0.3 equiv.) and HATU (0.6 equiv.) were added to the reaction mixture, which was stirred for 1 additional hour at room temperature. The reaction mixture was injected directly in a column for reversed-phase flash chromatography purification (MeCN in 0.1% HCOOH(aq), 5% (3 CV)→70%, 50 g RediSep Rf Gold® C18Aq, 20 CV, λ=214-254 nm, product with 60% MeCN). Evaporation of the fractions yielded 8 (88.4 mg, 0.096 mmol, 85% yield) as a white solid.

LCMS method 1: 99.9% purity at 215 nm, [M+H]⁺=919.4, [M+2H]²⁺=459.8.

¹H NMR (400 MHz, DMSO-d6) δ ppm 0.85-1.19 (m, 4H), 1.34 (br s, 9H), 1.51-1.66 (m, 4H), 1.66-1.99 (m, 8H), 1.99-2.09 (m, 4H), 2.10-2.25 (m, 2H), 2.26-2.44 (m, 2H), 2.54-2.79 (m, 2H), 2.99-3.13 (m, 1H), 3.25 (br s, 2H), 3.34-3.55 (m, 1H), 3.56-3.72 (m, 1H), 3.73-3.87 (m, 1H), 3.89-4.09 (m, 1H), 4.25 (br d, J=9.0 Hz, 1H), 4.32-4.51 (m, 1H), 4.52-4.92 (m, 1H), 4.93-5.20 (m, 1H), 6.27-6.80 (m, 2H), 6.89 (br d, J=4.9 Hz, 1 H), 6.97-7.43 (m, 3H), 8.14 (s, 1H), 8.22-8.56 (m, 2H), 8.82 (br d, J=7.0 Hz, 1H), 9.32 (br s, 1H), 10.82 (br s, 1H).

¹⁹F NMR (377 MHz, DMSO-d6) δ ppm −186.81-−182.66 (m, 1F), −114.66-−107.40 (m, 2F).

Step 6. Preparation of 5-((3R,5R)-3-amino-5-fluoropiperidin-1-yl)-N-(3-(difluoromethyl)-1-((1r,4r)-4-(4-(((3-(2,6-dioxopiperidin-3-yl)phenyl)(methyl)amino)methyl) piperidine-1-carbonyl)cyclohexyl)-1H-pyrazol-4-yl)pyrazolo[1,5-a]pyrimidine-3-carboxamide hydrochloride (P-16). HCl 4.0 M in 1,4-dioxane (3.40 mL, 13.6 mmol, 150 equiv) was added to tert-butyl N-[(3R,5R)-1-[3-[[3-(difluoromethyl)-1-[4-[4-[[3-(2,6-dioxo-3-piperidyl)-N-methyl-anilino]methyl]piperidine-1-carbonyl]cyclohexyl]pyrazol-4-yl]carbamoyl]pyrazolo[1,5-a]pyrimidin-5-yl]-5-fluoro-3-piperidyl]carbamate 8 (83.4 mg, 0.091 mmol, 1.0 equiv) and the mixture was sonicated for 30 minutes, then stirred for 1 hour. The solvent was evaporated to dryness and the residue was purified by reversed-phase flash chromatography (MeCN in 0.02 M HCl(aq), 5% (3 CV)→50%, 50 g RediSep Rf Gold® C18Aq, 15 CV, λ=214-254 nm, product with 32-35% MeCN). The fractions containing the product were evaporated to dryness, then co-evaporated with water (3×10 mL) to completely remove any trace of residual HCl, and the residue was freeze-dried overnight to afford P-16 (54.29 mg, 0.0645 mmol, 71% yield) as a white solid.

LCMS method 3:97.1% purity at 215 nm, [M−HCl+H]⁺=818.4, [M−HCl+2H]²⁺=409.8.

¹H NMR (400 MHz, DMSO-d6) δ ppm 0.86-1.30 (m, 2H), 1.38-1.95 (m, 10H), 1.95-2.13 (m, 4H), 2.14-2.29 (m, 1H), 2.41 (br d, J=10.3 Hz, 2H), 2.60-2.78 (m, 2H), 2.84-3.12 (m, 4H), 3.18-3.56 (m, 5H), 3.74-3.91 (m, 1H), 3.98 (br d, J=12.7 Hz, 1H), 4.25 (br t, J=11.7 Hz, 1H), 4.36 (br d, J=9.9 Hz, 1H), 4.52-4.75 (m, 1H), 4.76-4.98 (m, 1H), 5.10 (d, J=46.6 Hz, 1H), 6.56-7.73 (m, 6H), 8.32 (s, 1H), 8.38 (s, 1H), 8.50 (br s, 3H), 8.92 (d, J=7.8 Hz, 1H), 9.33 (s, 1H), 10.85 (br s, 1H).

¹⁹F NMR (377 MHz, DMSO-d6) δ ppm −184.65 (s, 1F), −111.30 (s, 2F).

### Example S5. Synthesis of P-32

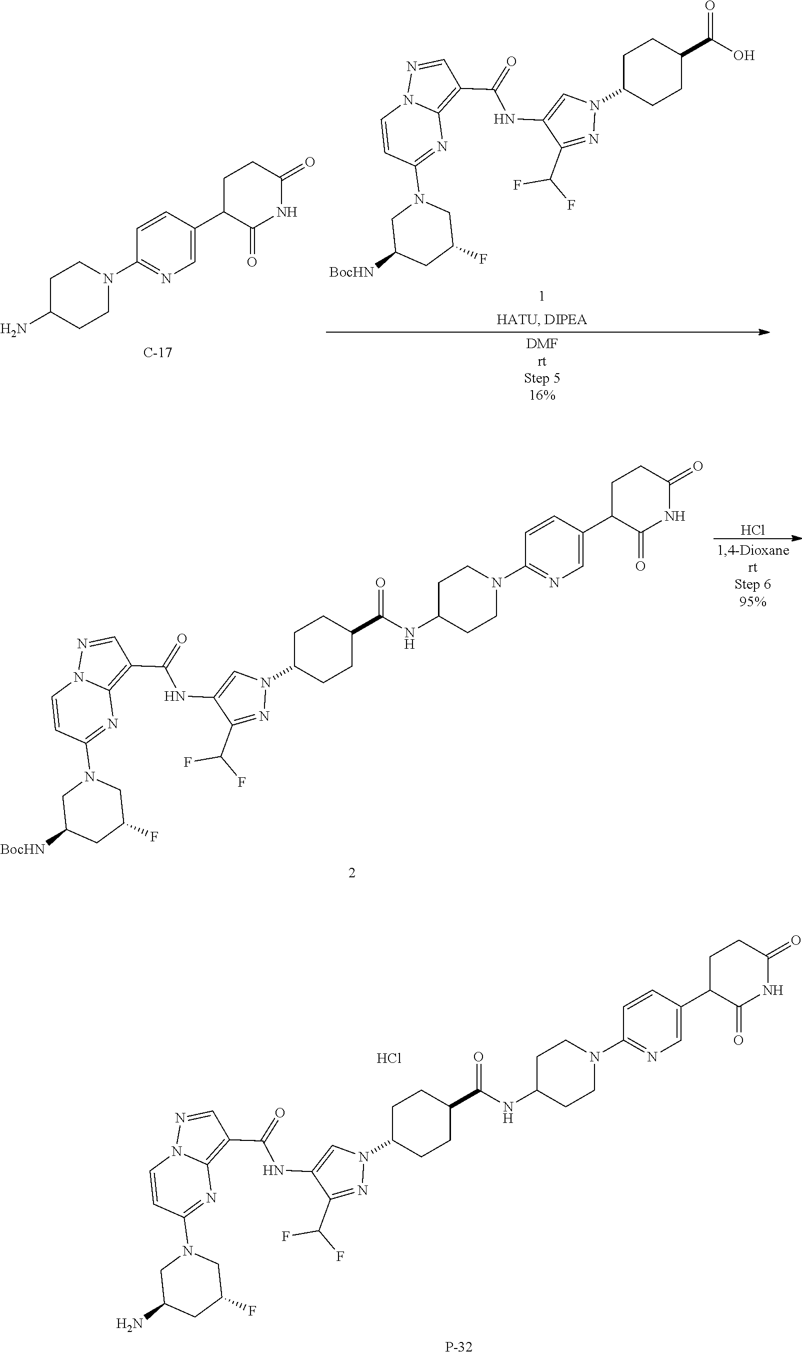

Step 5. Preparation of tert-Butyl N-[(3R,5R)-1-[3-[[3-(difluoromethyl)-1-[4-[[1-[5-(2,6-dioxo-3-piperidyl)-2-pyridyl]-4-piperidyl]carbamoyl]cyclohexyl]pyrazol-4-yl]carbamoyl]pyrazolo[1,5-a]pyrimidin-5-yl]-5-fluoro-3-piperidyl]carbamate (2). To a solution of 4-[3-(Difluoromethyl)-4-[5-[(3R,5R)-3-(tert-butoxycarbonylamino)-5-fluoro-1-piperidyl]pyrazolo[1,5-a]pyrimidine-3-carbonyl]amino]pyrazol-1-yl]cyclohexanecarboxylic acid 1-intermediate 3 from synthesis of P-15-(144.7 mg, 0.23 mmol, 1 eq.) in DMF (4.65 mL), was added DIPEA (0.41 mL, 2.33 mmol, 10 eq.) and HATU (115.13 mg, 0.30 mmol, 1.3 eq.). After 10 min at rt, 3-[6-(4-amino-1-piperidyl)-3-pyridyl]piperidine-2,6-dione hydrogen sulfate C-17 (90 mg, 0.23 mmol, 1 eq.) was added, and the reaction was stirred at rt. After 16 h, LCMS showed completion. The reaction mixture was purified by reverse phase flash chromatography (50 g C18 RediSep Rf Gold column, liquid deposit (DMF/DMSO), elution: 0% MeCN/0.1% HCOOH over 3 CV, then 0 to 30% MeCN/0.1% HCOOH over 15 CV, then 0 to 100% MeCN/0.1% HCOOH over 5 CV then 100% MeCN/0.1% HCOOH over 3 CV, desired product was released around 30% MeCN). Fractions were combined and concentrated to give 2 (57 mg, 16% yield) as a light yellow solid.

LCMS method 1:58.4% purity at 215 nm, [M+H]⁺=891.2.

¹H NMR (400 MHz, DMSO-d6) δ ppm 1.16-1.27 (m, 2H), 1.34 (br s, 9H), 1.52-1.63 (m, 2H), 1.64-1.91 (m, 8H), 1.93-2.06 (m, 3H), 2.10-2.21 (m, 3H), 2.60-2.74 (m, 1H), 2.88-2.99 (m, 2H), 3.00-3.17 (m, 1H), 3.42 (br s, 1H), 3.44-3.61 (m, 2H), 3.62-3.86 (m, 2H), 4.11-4.28 (m, 3H), 4.50-4.86 (m, 1H), 4.93-5.13 (m, 1H), 6.70-6.90 (m, 2H), 6.91-7.26 (m, 2H), 7.34-7.49 (m, 1H), 7.74 (d, J=7.6 Hz, 1H), 7.95 (d, J=2.4 Hz, 1H), 8.25-8.31 (m, 1H), 8.34 (br s, 1H), 8.81 (d, J=7.8 Hz, 1H), 9.31 (br s, 1H), 10.80 (s, 1H).

¹⁹F NMR (377 MHz, DMSO-d6) δ ppm −183.86 (br s, 1F), −111.28 (br s, 2F).

Step 6. Preparation of N-[3-(Difluoromethyl)-1-[4-[[1-[5-(2,6-dioxo-3-piperidyl)-2-pyridyl]-4-piperidyl]carbamoyl]cyclohexyl]pyrazol-4-yl]-5-[(3R,5R)-3-amino-5-fluoro-1-piperidyl]pyrazolo[1,5-a]pyrimidine-3-carboxamide hydrochloride (P-32). To tert-butyl N-[(3R,5R)-1-[3-[[3-(difluoromethyl)-1-[4-[1-[5-(2,6-dioxo-3-piperidyl)-2-pyridyl]-4-piperidyl]carbamoyl]cyclohexyl]pyrazol-4-yl]carbamoyl]pyrazolo[1,5-a]pyrimidin-5-yl]-5-fluoro-3-piperidyl]carbamate 2 (57 mg, 0.05 mmol, 1 eq.) was added a solution of 4 M HCl in 1,4-dioxane (0.32 mL, 1.3 mmol, 27 eq.). The mixture was stirred at rt. After 1h, LCMS showed full conversion. Volatiles were evaporated under vacuum. The resultant residue was purified by reverse phase flash chromatography (30 g C18 RediSep Rf Gold column, liquid deposit (Water), elution: 5% MeCN/0.2 M HCl over 5 CV, then 5 to 20% MeCN/0.2 M HCl over 15 CV, then 20% MeCN/0.2 M HCl over 5 CV, then 20 to 100% MeCN/0.2 M HCl over 2 CV then 100% MeCN/0.2% HCOOH over 7 CV). Fractions were combined and concentrated to give P-32 (37.3 mg, 95% yield) as a light yellow solid as a hydrochloride.

LCMS method 3:97.4% purity at 215 nm, [M−HCl+2H]²⁺=396.2; [M−HCl+H]⁺=791.4.

¹H NMR (400 MHz, DMSO-d6) δ ppm 1.40-1.66 (m, 3H), 1.67-1.82 (m, 2H), 1.83-1.93 (m, 4H), 1.94-2.02 (m, 1H), 2.02-2.14 (m, 3H), 2.15-2.45 (m, 3H), 2.55-2.75 (m, 3H), 3.27-3.56 (m, 5H), 3.87-3.96 (m, 1H), 3.99 (dd, J=13.1, 4.8 Hz, 1H), 4.14-4.32 (m, 3H), 4.56-4.70 (m, 1H), 4.80-4.92 (m, 1H), 5.05-5.14 (m, 1H), 6.91 (d, J=8.1 Hz, 1H), 7.14 (t, J=53.8 Hz, 1H), 7.46 (d, J=9.5 Hz, 1H), 7.82-8.03 (m, 3H), 8.32 (s, 1H), 8.35-8.52 (m, 4H), 8.93 (d, J=8.1 Hz, 1H), 9.33 (s, 1H), 10.95 (s, 1H).

¹⁹F NMR (377 MHz, DMSO-d6) δ ppm −184.64 (br s, 1F), −111.27 (s, 2F).

The following compounds were synthesized via the same general routes with modifications to Intermediate (T-X) in step 3 and CBM (C-X) in step 5 (Table 5).

TABLE 5

| Com- | | | |
|---|---|---|---|
| pound | | | |
| No. | Int | CBM | Structure |
| P-1 | T-1 | C-1 | 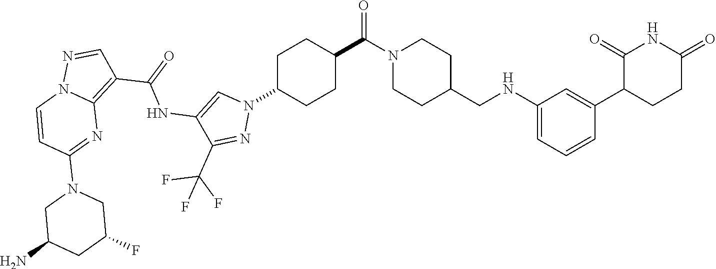 [SMILES: N[C@@H]1C[C@@H](F)CN(c2ccn3ncc(C(=O)Nc4cn([C@H]5CC[C@H](C(=O)N6CCC(CNc7cccc(C8CCC(=O)NC8=O)c7)CC6)CC5)nc4C(F)(F)F)c3n2)C1] |
| P-2 | T-1 | C-2 | 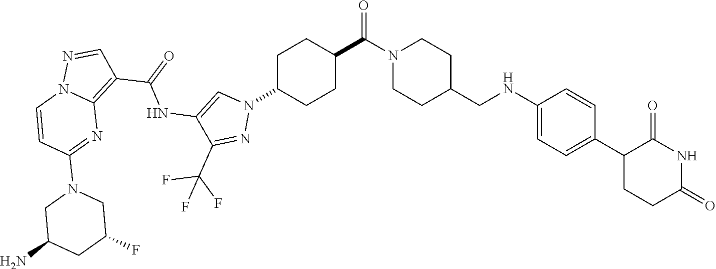 [SMILES: N[C@@H]1C[C@@H](F)CN(c2ccn3ncc(C(=O)Nc4cn([C@H]5CC[C@H](C(=O)N6CCC(CNc7ccc(C8CCC(=O)NC8=O)cc7)CC6)CC5)nc4C(F)(F)F)c3n2)C1] |
| P-7 | T-1 | C-4 | 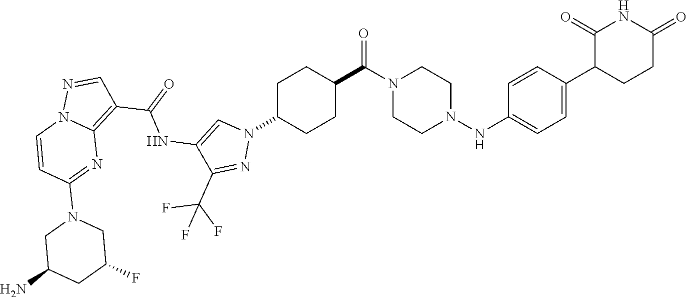 [SMILES: N[C@@H]1C[C@@H](F)CN(c2ccn3ncc(C(=O)Nc4cn([C@H]5CC[C@H](C(=O)N6CCN(Nc7ccc(C8CCC(=O)NC8=O)cc7)CC6)CC5)nc4C(F)(F)F)c3n2)C1] |
| P-9 | T-1 | C-5 | 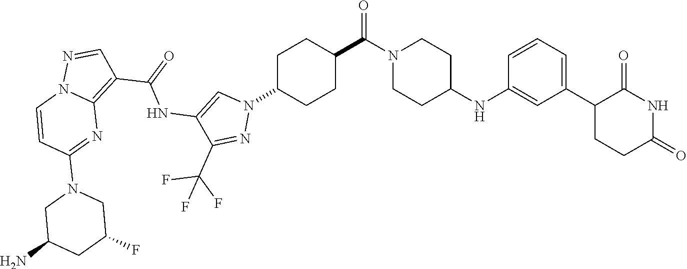 [SMILES: N[C@@H]1C[C@@H](F)CN(c2ccn3ncc(C(=O)Nc4cn([C@H]5CC[C@H](C(=O)N6CCC(Nc7cccc(C8CCC(=O)NC8=O)c7)CC6)CC5)nc4C(F)(F)F)c3n2)C1] |
| P-12 | T-1 | C-7 | 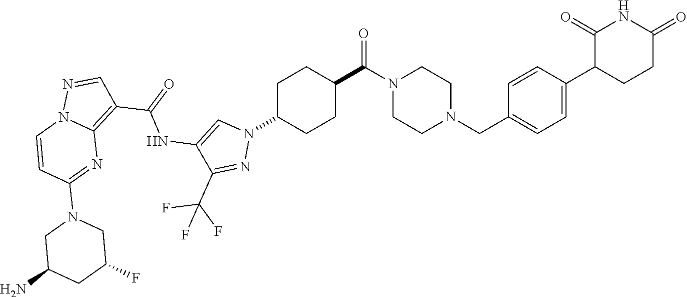 [SMILES: N[C@@H]1C[C@@H](F)CN(c2ccn3ncc(C(=O)Nc4cn([C@H]5CC[C@H](C(=O)N6CCN(Cc7ccc(C8CCC(=O)NC8=O)cc7)CC6)CC5)nc4C(F)(F)F)c3n2)C1] |
| P-14 | T-2 | C-8 | 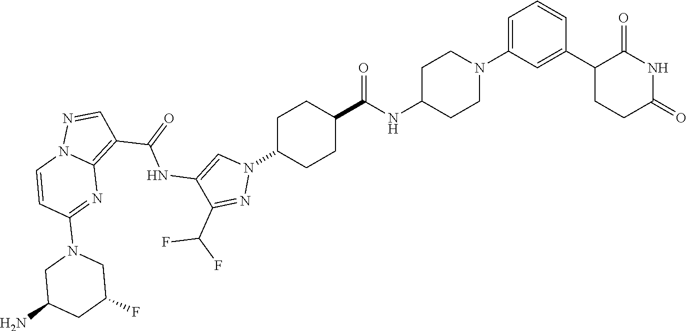 [SMILES: N[C@@H]1C[C@@H](F)CN(c2ccn3ncc(C(=O)Nc4cn([C@H]5CC[C@H](C(=O)NC6CCN(c7cccc(C8CCC(=O)NC8=O)c7)CC6)CC5)nc4C(F)F)c3n2)C1] |
| P-15 | T-2 | C-9 | 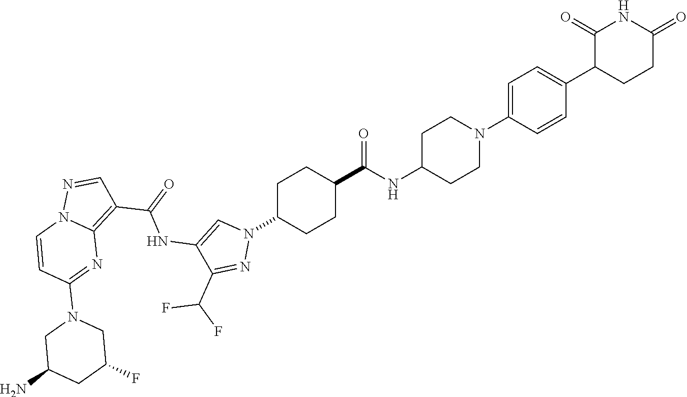 [SMILES: N[C@@H]1C[C@@H](F)CN(c2ccn3ncc(C(=O)Nc4cn([C@H]5CC[C@H](C(=O)NC6CCN(c7ccc(C8CCC(=O)NC8=O)cc7)CC6)CC5)nc4C(F)F)c3n2)C1] |
| P-16 | T-2 | C-10 | 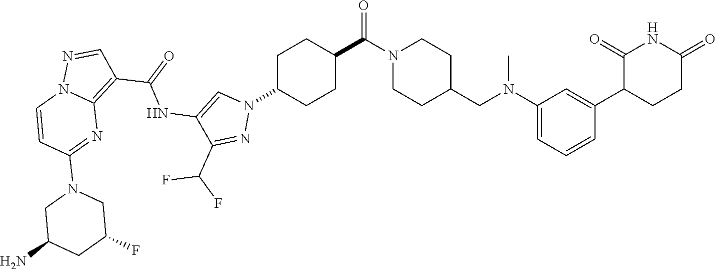 [SMILES: CN(CC1CCN(C(=O)[C@H]2CC[C@H](n3cc(NC(=O)c4cnn5ccc(N6C[C@H](N)C[C@@H](F)C6)nc45)c(C(F)F)n3)CC2)CC1)c1cccc(C2CCC(=O)NC2=O)c1] |
| P-17 | T-2 | C-11 | 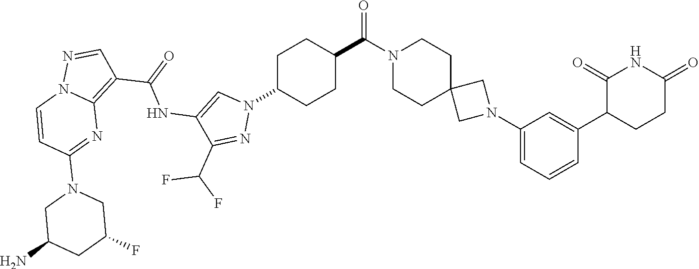 [SMILES: N[C@@H]1C[C@@H](F)CN(c2ccn3ncc(C(=O)Nc4cn([C@H]5CC[C@H](C(=O)N6CCC7(CC6)CN(c6cccc(C8CCC(=O)NC8=O)c6)C7)CC5)nc4C(F)F)c3n2)C1] |
| P-25 | T-2 | C-15 | 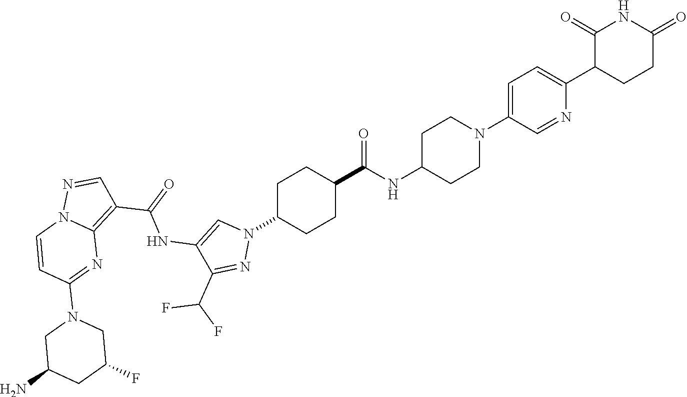 [SMILES: N[C@@H]1C[C@@H](F)CN(c2ccn3ncc(C(=O)Nc4cn([C@H]5CC[C@H](C(=O)NC6CCN(c7ccc(C8CCC(=O)NC8=O)nc7)CC6)CC5)nc4C(F)F)c3n2)C1] |
| P-30 | T-2 | C-18 | 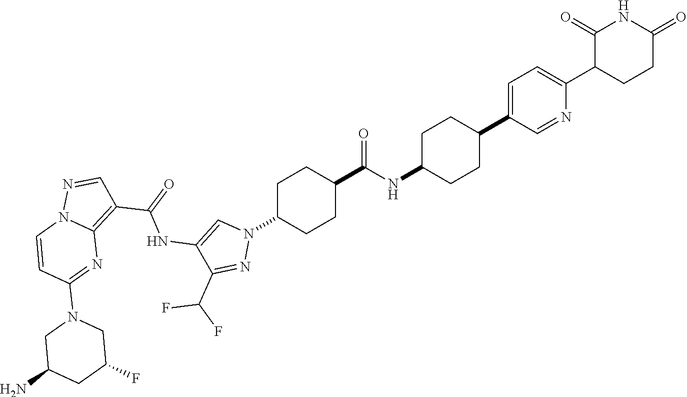 [SMILES: N[C@@H]1C[C@@H](F)CN(c2ccn3ncc(C(=O)Nc4cn([C@H]5CC[C@H](C(=O)N[C@H]6CC[C@@H](c7ccc(C8CCC(=O)NC8=O)nc7)CC6)CC5)nc4C(F)F)c3n2)C1] |
| P-31 | T-2 | C-19 | 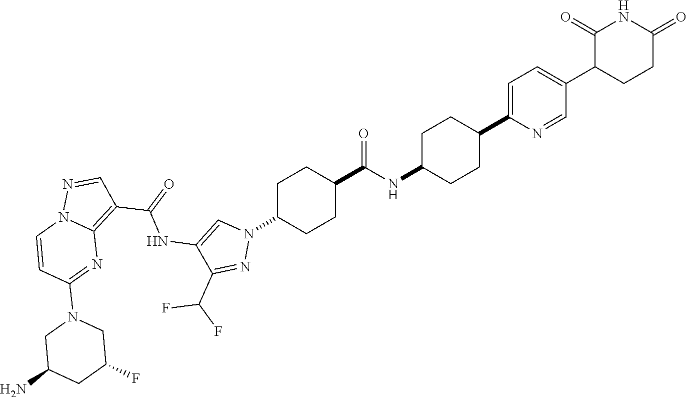 [SMILES: N[C@@H]1C[C@@H](F)CN(c2ccn3ncc(C(=O)Nc4cn([C@H]5CC[C@H](C(=O)N[C@H]6CC[C@@H](c7ccc(C8CCC(=O)NC8=O)cn7)CC6)CC5)nc4C(F)F)c3n2)C1] |
| P-32 | T-2 | C-17 | 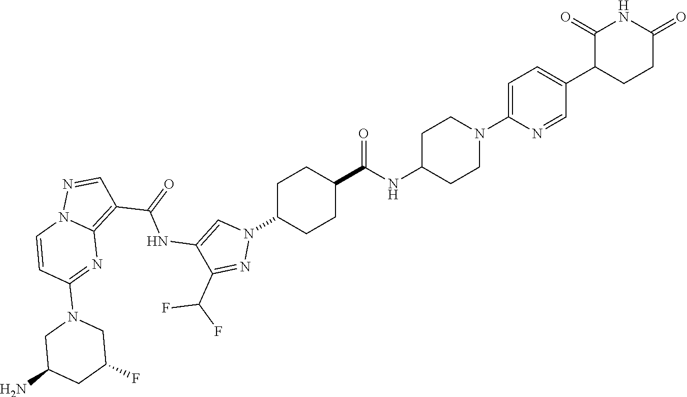 [SMILES: N[C@@H]1C[C@@H](F)CN(c2ccn3ncc(C(=O)Nc4cn([C@H]5CC[C@H](C(=O)NC6CCN(c7ccc(C8CCC(=O)NC8=O)cn7)CC6)CC5)nc4C(F)F)c3n2)C1] |
| P-33 | T-2 | C-20 | 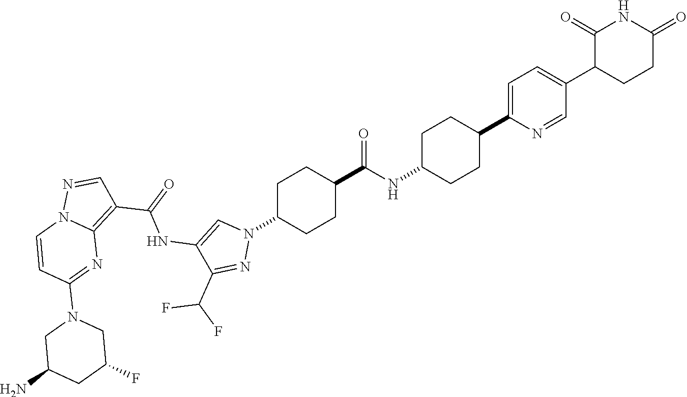 [SMILES: N[C@@H]1C[C@@H](F)CN(c2ccn3ncc(C(=O)Nc4cn([C@H]5CC[C@H](C(=O)N[C@H]6CC[C@H](c7ccc(C8CCC(=O)NC8=O)cn7)CC6)CC5)nc4C(F)F)c3n2)C1] |
| P-34 | T-2 | C-21 | 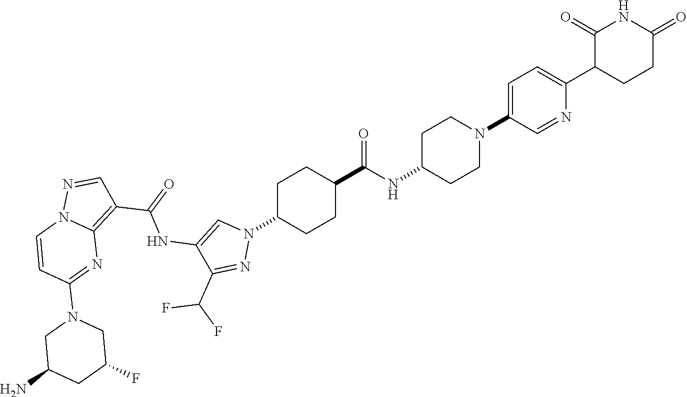 |

| Compound | |
|---|---|
| No. | Characterization Data |
| P-1 | 73% yield as a white solid. |
| | LCMS method 3: retention time: 2.266 min, 99% purity at |
| | 215 nm, [M − HCl + H]⁺ = 822.3. |
| | ¹H NMR (400 MHz, DMSO-d6) δ ppm 0.94-1.22 (m, 2 |
| | H), 1.48-1.67 (m, 2 H), 1.72-2.23 (m, 13 H), 2.34-2.47 |
| | (m, 1 H), 2.56-2.68 (m, 2 H), 2.71-2.83 (m, 1 H), 2.89- |
| | 3.07 (m, 3 H), 3.23-3.57 (m, 4 H), 3.99-4.10 (m, 1 H), |
| | 4.29-4.47 (m, 2 H), 4.54-4.84 (m, 2 H), 5.12 (d, J = 46.7 |
| | Hz, 1 H), 6.34-6.79 (m, 3 H), 6.91 (d, J = 8.1 Hz, 1 H), |
| | 7.03-7.15 (m, 1 H), 8.07-8.38 (m, 4 H), 8.45 (s, 1 H), |
| | 8.93 (d, J = 7.8 Hz, 1 H), 9.24 (s, 1 H), 10.81 (s, 1 H).- |
| | One proton obscured by water signal in ¹H NMR. |
| | ¹⁹F NMR (377 MHz, DMSO d6) δ ppm −184.88 |
| | (s, 1 F), −58.78 (s, 3 F). |
| P-2 | 82% yield as a white solid. |
| | LCMS method 3: retention time: 2.218 min, 99% purity at |
| | 215 nm, [M − HCl + H]⁺ = 822.4; [M − HCl + 2H]²⁺ = 411.7. |
| | ¹H NMR (400 MHz, DMSO-d6) δ ppm 0.96-1.22 (m, 2 |
| | H), 1.48-1.68 (m, 2 H), 1.74-2.20 (m, 12 H), 2.35-2.47 |
| | (m, 1 H), 2.55-2.68 (m, 1 H), 2.71-2.81 (m, 1 H), 2.96- |
| | 3.08 (m, 3 H), 3.24-3.61 (m, 3 H), 3.69-3.78 (m, 2 H), |
| | 4.00-4.09 (m, 2 H), 4.28-4.48 (m, 2 H), 4.54-4.84 (m, 2 |
| | H), 5.06 (d, J = 46.5 Hz, 1 H), 6.76-7.16 (m, 5 H), 8.21- |
| | 8.38 (m, 4 H), 8.45 (s, 1 H), 8.93 (d, J = 7.8 Hz, 1 H), 9.24 |
| | (s, 1 H), 10.78 (s, 1 H).-One proton is obscured by the |
| | water or DMSO peak in the ¹H NMR. |
| | ¹⁹F NMR (377 MHz, DMSO-d6) δ ppm −185.00 |
| | (s, 1 F), −58.75 (s, 3 F). |
| P-7 | 39% yield as a white solid. |
| | LCMS method 3: retention time: 2.125 min, 99% purity at |
| | 215 nm, [M − HCl + 2H]²⁺ = 404.8 ; [M − HCl + H]⁺ = 808.4. |
| | ¹H NMR (400 MHz, DMSO-d6) δ ppm 1.52-1.67 (m, 2 |
| | H), 1.77-2.20 (m, 13 H), 2.34-2.47 (m, 2 H), 2.61-2.81 |
| | (m, 4 H), 3.09-3.21 (m, 1 H), 3.25-3.62 (m, 5 H), 3.72- |
| | 3.82 (m, 1 H), 4.31-4.41 (m, 2 H), 4.55-4.80 (m, 2 H), |
| | 5.12 (d, J = 46.0 Hz, 1 H), 6.91 (d, J = 7.0 Hz, 2 H), 7.02- |
| | 7.27 (m, 3 H), 8.21-8.35 (m, 4 H), 8.45 (s, 1 H), 8.93 (d, |
| | J = 7.8 Hz, 1 H), 9.24 (s, 1 H), 10.80 (s, 1 H). |
| | ¹⁹F NMR (377 MHz, DMSO-d6) δ ppm-184.89 (s, 1 |
| | F), −58.71 (s, 3 F). |
| P-9 | 50% yield as a white solid. |
| | LCMS method 3: retention time: 2.190 min, 99% purity at |
| | 215 nm, [M − HCl + 2H]²⁺ = 404.8 ; [M − HCl + H]⁺ = 808.4. |
| | ¹H NMR (400 MHz, DMSO-d6) δ ppm 1.26-1.70 (m, 4 |
| | H), 1.71-2.30 (m, 11 H), 2.34-2.46 (m, 1 H), 2.51-2.56 |
| | (m, 1 H), 2.59-2.84 (m, 3 H), 3.04-3.19 (m, 1 H), 3.26- |
| | 3.66 (m, 4 H), 3.84-3.91 (m, 1 H), 4.02-4.14 (m, 1 H), |
| | 4.29-4.45 (m, 2 H), 4.52-4.91 (m, 2 H), 5.12 (d, J = 46.2 |
| | Hz, 1 H), 6.46-7.64 (m, 5 H), 8.17-8.59 (m, 5 H), 8.93 |
| | (d, J = 7.8 Hz, 1 H), 9.24 (s, 1 H), 10.86 (s, 1 H).-One |
| | exchangeable proton missing in ¹H NMR. Two protons |
| | partially obscured by water signal in ¹H NMR. |
| | ¹⁹F NMR (377 MHz, DMSO-d6) δ ppm −184.94 |
| | (s, 1 F), −58.80 (s, 3 F). |
| P-12 | 68% yield as a white solid. |
| | LCMS method 3: retention time: 1.811 min, 99% purity at |
| | 215 nm, [M − 2HCl + H]⁺ = 808.4, [M − 2HCl + 2H]²⁺ = 404.7. |
| | ¹H NMR (400 MHz, DMSO-d6) δ ppm 1.52-1.63 (m, 2 |
| | H), 1.81-2.08 (m, 8 H), 2.17-2.30 (m, 1 H), 2.35-2.44 |
| | (m, 1 H), 2.64-2.80 (m, 2 H), 2.91-3.11 (m, 3 H), 3.27- |
| | 3.38 (m, 4 H), 3.48-3.52 (m, 3 H), 3.93 (dd, J = 11.5, 4.6 |
| | Hz, 1 H), 4.21-4.48 (m, 5 H), 4.57-4.81 (m, 2 H), 5.13 |
| | (d, J = 48.0 Hz, 1 H), 6.91 (d, J = 7.8 Hz, 1 H), 7.32-7.36 |
| | (m, 2 H), 7.52-7.56 (m, 2 H), 8.23-8.36 (m, 4 H), 8.44 |
| | (s, 1 H), 8.94 (d, J = 7.8 Hz, 1 H), 9.24 (s, 1 H), 10.81- |
| | 10.93 (m, 2 H).-Three protons obscured by water peak by |
| | ¹H NMR. |
| | ¹⁹F NMR (377 MHz, DMSO-d6) δ ppm −184.73 |
| | (s, 1 F), −58.81 (s, 3 F). |
| P-14 | 91% yield as a white solid. |
| | LCMS method 3: retention time: 1.999 min, 99% purity at |
| | 215 nm, [M − HCl + 2H]2⁺ = 395.7; [M − HCl + H]⁺ = 790.4. |
| | ¹H NMR (400 MHz, DMSO-d6) δ ppm 1.52-1.66 (m, 2 |
| | H), 1.70-1.81 (m, 2 H), 1.84-2.12 (m, 10 H), 2.17-2.29 |
| | (m, 2 H), 2.35-2.46 (m, 1 H), 2.53-2.58 (m, 1 H), 2.64- |
| | 2.73 (m, 1 H), 3.24-3.53 (m, 5 H), 3.58-3.68 (m, 2 H), |
| | 3.86-3.98 (m, 2 H), 4.22-4.31 (m, 1 H), 4.78-4.95 (m, 2 |
| | H), 5.10 (d, J = 46.2 Hz, 1 H), 6.90 (d, J = 8.1 Hz, 1 H), |
| | 6.99-7.71 (m, 5 H), 8.06 (br s, 1 H), 8.29-8.43 (m, 5 H), |
| | 8.93 (d, J = 8.1 Hz, 1 H), 9.33 (s, 1 H), 10.88 (s, 1 H). |
| | ¹⁹F NMR (377 MHz, DMSO-d6) δ ppm −184.62 |
| | (s, 1 F), −111.26 (s, 2 F). |
| P-15 | 37% yield as a white solid. |
| | LCMS method 3: retention time: 1.979 min, 99% purity at |
| | 215 nm, [M − HCl + H]⁺ = 790.3. |
| | ¹H NMR (400 MHz, DMSO-d6) δ ppm 1.55-1.67 (m, 3 |
| | H), 1.70-1.82 (m, 3 H), 1.83-2.26 (m, 12 H), 2.35-2.41 |
| | (m, 1 H), 2.63-2.72 (m, 1 H), 3.25-3.52 (m, 4 H), 3.57- |
| | 3.66 (m, 2 H), 3.76-3.83 (m, 2 H), 4.21-4.31 (m, 1 H), |
| | 4.47-4.71 (m, 1 H), 4.73-4.97 (m, 1 H), 5.11 (d, J = 48.0 |
| | Hz, 1 H), 6.89 (d, J = 8.0 Hz, 1 H), 6.97-7.35 (m, 5 H), |
| | 7.79-8.00 (m, 1 H), 8.15-8.28 (m, 3 H), 8.33 (s, 1 H), |
| | 8.39 (s, 1 H), 8.93 (d, J = 7.6 Hz, 1 H), 9.33 (s, 1 H), 10.81 |
| | (s, 1 H).-Two protons obscured by water peak by 1H |
| | NMR. One proton partially under the peak of DMSO by 1H |
| | NMR. |
| | ¹⁹F NMR (377 MHz, DMSO- d6) δ ppm −185.26 |
| | (s, 1 F), −111.21 (s, 2 F). |
| P-16 | 71% yield as a white solid. |
| | LCMS method 3: retention time: 2.327 min, 97% purity at |
| | 215 nm, [M − HCl + H]⁺ = 818.4, [M − HCl + 2H]²⁺ = 409.8. |
| | ¹H NMR (400 MHz, DMSO-d6) δ ppm 0.86-1.30 (m, 2 |
| | H), 1.38-1.95 (m, 10 H), 1.95-2.13 (m, 4 H), 2.14-2.29 |
| | (m, 1 H), 2.41 (br d, J = 10.3 Hz, 2 H), 2.60-2.78 (m, 2 |
| | H), 2.84-3.12 (m, 4 H), 3.18-3.56 (m, 5 H), 3.74-3.91 |
| | (m, 1 H), 3.98 (br d, J = 12.7 Hz, 1 H), 4.25 (br t, J = 11.7 |
| | Hz, 1 H), 4.36 (br d, J = 9.9 Hz, 1 H), 4.52-4.75 (m, 1 H), |
| | 4.76-4.98 (m, 1 H), 5.10 (d, J = 46.6 Hz, 1 H), 6.56-7.73 |
| | (m, 6 H), 8.32 (s, 1 H), 8.38 (s, 1 H), 8.50 (br s, 3 H), 8.92 |
| | (d, J = 7.8 Hz, 1 H), 9.33 (s, 1 H), 10.85 (br s, 1 H). |
| | ¹⁹F NMR (377 MHz, DMSO-d6) δ ppm −184.65 |
| | (s, 1 F), −111.30 (s, 2 F). |
| P-17 | 17% yield as a white solid. |
| | LCMS method 3: retention time: 2.319 min, 99% purity at |
| | 215 nm, [M + H]⁺ = 816.4, [M + 2H]²⁺ = 408.8. |
| | ¹H NMR (400 MHz, DMSO-d6) δ ppm 0.98-1.17 (m, 1 |
| | H), 1.47-1.72 (m, 5 H), 1.72-1.96 (m, 7 H), 1.96-2.09 |
| | (m, 3 H), 2.10-2.29 (m, 2 H), 2.39-2.46 (m, 1 H), 2.57- |
| | 2.69 (m, 1 H), 2.76 (br t, J = 11.7 Hz, 1 H), 2.90-3.03 (m, |
| | 1 H), 3.04-3.17 (m, 1 H), 3.41-3.54 (m, 5 H), 3.54-3.68 |
| | (m, 4 H), 3.74 (dd, J = 10.7, 5.0 Hz, 1 H), 4.26 (br t, J = |
| | 11.8 Hz, 1 H), 4.32-4.62 (m, 1 H), 4.64-4.91 (m, 1 H), |
| | 5.02 (d, J = 45.0 Hz, 1 H), 6.26 (s, 1 H), 6.33 (d, J = 8.2 |
| | Hz, 1 H), 6.51 (d, J = 7.6 Hz, 1 H), 6.92 (d, J = 7.9 Hz, 1 |
| | H), 6.98-7.36 (m, 2 H), 8.29 (s, 1 H), 8.39 (s, 1 H), 8.82 |
| | (d, J = 7.9 Hz, 1 H), 9.34 (s, 1 H), 10.79 (s, 1 H). |
| | ¹⁹F NMR (377 MHz, DMSO-d6) δ ppm −183.21 |
| | (s, 1 F), −111.09 (s, 2 F). |
| P-25 | 34% yield as a light-yellow solid. |
| | LCMS method 3: retention time: 1.902 min, 95% purity at |
| | 215 nm, [M + H]⁺ = 791.4, [M + 2H]²⁺ = 396.2. |
| | ¹H NMR (400 MHz, DMSO-d6) δ ppm 1.42-1.90 (m, 12 |
| | H), 2.02-2.26 (m, 6 H), 2.52-2.63 (m, 2 H), 2.79-2.94 |
| | (m, 3 H), 2.99-3.09 (m, 1 H), 3.36-3.44 (m, 2 H), 3.65- |
| | 3.80 (m, 3 H), 3.88 (dd, J = 8.7, 5.3 Hz, 1 H), 4.20-4.28 |
| | (m, 1 H), 4.31-4.56 (m, 1 H), 4.62-4.86 (m, 1 H), 5.01 |
| | (d, J = 47.0 Hz, 1 H), 6.92 (d, J = 7.8 Hz, 1 H), 7.01-7.30 |
| | (m, 2 H), 7.33 (dd, J = 8.5, 3.1 Hz, 1 H), 7.77 (d, J = 7.8 |
| | Hz, 1 H), 8.21 (d, J = 2.7 Hz, 1 H), 8.27 (s, 1 H), 8.39 (s, 1 |
| | H), 8.80 (d, J = 7.8 Hz, 1 H), 9.33 (s, 1 H), 10.77 (s, 1 H).- |
| | Two protons partially obscured by water peak and two |
| | protons partially under the peak of DMSO by ¹H NMR. |
| | ¹⁹F NMR (377 MHz, DMSO-d6) δ ppm −182.80 |
| | (s, 1F), −111.07 (s, 2 F). |
| P-30 | 54% yield as a light-yellow solid. |
| | LCMS method 3: retention time: 2.083 min, 96% purity at |
| | 215 nm, [M − HCOOH + H]⁺ = 790.2, [M − HCOOH + 2H]²⁺ = |
| | 395.8. |
| | ¹H NMR (400 MHz, DMSO-d6) δ ppm 1.53-1.66 (m, 8 |
| | H), 1.68-1.78 (m, 4 H), 1.79-1.92 (m, 4 H), 2.06-2.18 |
| | (m, 4 H), 2.18-2.31 (m, 2 H), 2.34-2.38 (m, 1 H), 2.55- |
| | 2.65 (m, 2 H), 2.81-2.95 (m, 1 H), 2.96-3.08 (m, 1 H), |
| | 3.90-4.09 (m, 2 H), 4.25 (tt, J = 11.6, 3.2 Hz, 1 H), 4.31- |
| | 4.58 (m, 1 H), 4.60-4.92 (m, 1 H), 5.01 (d, J = 46.2 Hz, 1 |
| | H), 6.93 (d, J = 8.1 Hz, 1 H), 7.16 (t, J = 53.8 Hz, 1 H), |
| | 7.32 (d, J = 8.1 Hz, 1 H), 7.71 (dd, J = 8.1, 2.2 Hz, 1 H), |
| | 7.76 (br d, J = 7.8 Hz, 1 H), 8.21 (br s, 1 H), 8.28 (s, 1 H), |
| | 8.39 (s, 1 H), 8.45 (d, J = 2.0 Hz, 1 H), 8.80 (d, J = 7.8 Hz, |
| | 1 H), 9.34 (s, 1 H), 10.83 (s, 1 H).-Three protons missing |
| | in ¹H NMR. |
| | ¹⁹F NMR (377 MHz, DMSO-d6) δ ppm −182.92 |
| | (s, 1 F), −111.12 (s, 2 F). |
| P-31 | 38% yield as a white solid. |
| | LCMS method 3: retention time: 1.989 min, 99% purity at |
| | 215 nm, [M − HCl + H]⁺ = 790.4, [M − HCl + 2H]²⁺ = 395.7. |
| | ¹H NMR (400 MHz, DMSO-d6) δ ppm 1.51-1.65 (m, 4 |
| | H), 1.65-1.87 (m, 8 H), 1.87-2.17 (m, 8 H), 2.30-2.41 |
| | (m, 3 H), 2.55-2.64 (m, 1 H), 2.68-2.80 (m, 1 H), 2.85- |
| | 3.01 (m, 1 H), 3.25-3.31 (m, 1 H), 3.95-4.04 (m, 1 H), |
| | 4.05-4.20 (m, 1 H), 4.21-4.33 (m, 1 H), 4.48-4.71 (m, 1 |
| | H), 4.74-4.98 (m, 1 H), 5.10 (d, J = 46.5 Hz, 1 H), 6.88 |
| | (br d, J = 7.8 Hz, 1 H), 6.97-7.33 (m, 1 H), 7.60-7.77 (m, |
| | 1 H), 7.82 (br d, J = 7.5 Hz, 1 H), 7.93-8.30 (m, 4 H), |
| | 8.33 (s, 1 H), 8.38 (s, 1 H), 8.58 (br s, 1 H), 8.94 (d, J = 7.8 |
| | Hz, 1 H), 9.33 (s, 1 H), 10.99 (s, 1 H).-Mixture of |
| | rotamers as confirmed by high-temperature ¹H NMR. |
| | ¹⁹F NMR (377 MHz, DMSO-d6) δ ppm −184.66 |
| | (s, 1 F), −113.10-−108.69 (m, 2 F). |
| P-32 | 96% yield as a light-yellow solid. |
| | LCMS method 3: retention time: 1.781 min, 97% purity at |
| | 215 nm, [M − HCl + 2H]²⁺ = 396.2; [M − HCl + H]⁺ = 791.4. |
| | ¹H NMR (400 MHz, DMSO-d6) δ ppm 1.40-1.66 (m, 3 |
| | H), 1.67-1.82 (m, 2 H), 1.83-1.93 (m, 4 H), 1.94-2.02 |
| | (m, 1 H), 2.02-2.14 (m, 3 H), 2.15-2.45 (m, 3 H), 2.55- |
| | 2.75 (m, 3 H), 3.27-3.56 (m, 5 H), 3.87-3.96 (m, 1 H), |
| | 3.99 (dd, J = 13.1, 4.8 Hz, 1 H), 4.14-4.32 (m, 3 H), 4.56- |
| | 4.70 (m, 1 H), 4.80-4.92 (m, 1 H), 5.05-5.14 (m, 1 H), |
| | 6.91 (d, J = 8.1 Hz, 1 H), 7.14 (t, J = 53.8 Hz, 1 H), 7.46 |
| | (d, J = 9.5 Hz, 1 H), 7.82-8.03 (m, 3 H), 8.32 (s, 1 H), |
| | 8.35-8.52 (m, 4 H), 8.93 (d, J = 8.1 Hz, 1 H), 9.33 (s, 1 |
| | H), 10.95 (s, 1 H).-One proton partially overlaps with the |
| | water signal by ¹H NMR. |
| | ¹⁹F NMR (377 MHz, DMSO-d6) δ ppm −184.64 |
| | (br s, 1 F), −111.27 (s, 2 F). |
| P-33 | 48% yield as a white solid. |
| | LCMS method 3: retention time: 1.872 min, 97% purity at |
| | 215 nm, [M − HCl + H]⁺ = 790.4, [M − HCl + 2H]²⁺ = 395.8. |
| | ¹H NMR (400 MHz, DMSO-d6) δ ppm 1.26-1.43 (m, 2 |
| | H), 1.51-1.66 (m, 2 H), 1.66-1.81 (m, 4 H), 1.82-1.92 |
| | (m, 4 H), 1.96 (br d, J = 14.1 Hz, 4 H), 2.03-2.13 (m, 4 |
| | H), 2.13-2.23 (m, 1 H), 2.29-2.46 (m, 2 H), 2.55-2.65 |
| | (m, 1 H), 2.65-2.80 (m, 1 H), 2.94-3.09 (m, 1 H), 3.60- |
| | 3.66 (m, 1 H), 4.17 (dd, J = 12.5, 4.0 Hz, 1 H), 4.25 (tt, J = |
| | 11.6, 3.6 Hz, 1 H), 4.48-4.74 (m, 1 H), 4.75-4.99 (m, 1 |
| | H), 5.10 (d, J = 46.0 Hz, 1 H), 6.91 (d, J = 7.9 Hz, 1 H), |
| | 7.15 (t, J = 54.2 Hz, 1 H), 7.80 (d, J = 7.9 Hz, 1 H), 7.84- |
| | 7.97 (m, 1 H), 8.21-8.31 (m, 1 H), 8.32 (s, 1 H), 8.39 (s, 1 |
| | H), 8.40-8.56 (m, 3 H), 8.67 (br s, 1 H), 8.93 (d, J = 7.8 |
| | Hz, 1 H), 9.33 (s, 1 H), 11.03 (s, 1 H). -1 H overlapped |
| | with the water peak in ¹H NMR. |
| | ¹⁹F NMR (377 MHz, DMSO-d6) δ ppm −184.58 |
| | (s, 1 F), −111.27 (s, 2 F). |
| P-34 | 52% yield as a light-yellow solid. |
| | LCMS method 3: retention time: 2.031 min, 99% purity at |
| | 215 nm, [M − HCl + H]⁺ = 790.4 ; [M − HCl + 2H]²⁺ = 395.8. |
| | ¹H NMR (400 MHz, DMSO-d6) δ ppm 1.26-1.40 (m, 2 |
| | H), 1.52-1.65 (m, 4 H), 1.66-1.94 (m, 8 H), 2.01-2.31 |
| | (m, 5 H), 2.34-2.44 (m, 1 H), 2.54-2.64 (m, 3 H), 3.22- |
| | 3.45 (m, 5 H), 4.06-4.13 (m, 1 H), 4.21-4.31 (m, 1 H), |
| | 4.61 (br s, 1 H), 4.83 (br s, 1 H), 5.11 (d, J = 46.0 Hz, 1 H), |
| | 6.88 (br d, J = 8.1 Hz, 1 H), 7.14 (t, J = 54.0 Hz, 1 H), 7.40- |
| | 7.51 (m, 1 H), 7.75 (d, J = 8.1 Hz, 1 H), 7.90 (s, 1 H), |
| | 8.12-8.26 (m, 3 H), 8.33 (s, 1 H), 8.39 (s, 1 H), 8.53 (s, 1 |
| | H), 8.94 (d, J = 7.8 Hz, 1 H), 9.33 (s, 1 H), 10.92 (s, 1 H). |
| | ¹⁹F NMR (377 MHz, DMSO-d6) δ ppm −184.52 |
| | (s, 1 F), −111.24 (br dd, J = 53.8, 15.7 Hz, 2 F). |

### Final Product General Method 2

### Example S6. Synthesis of P-5

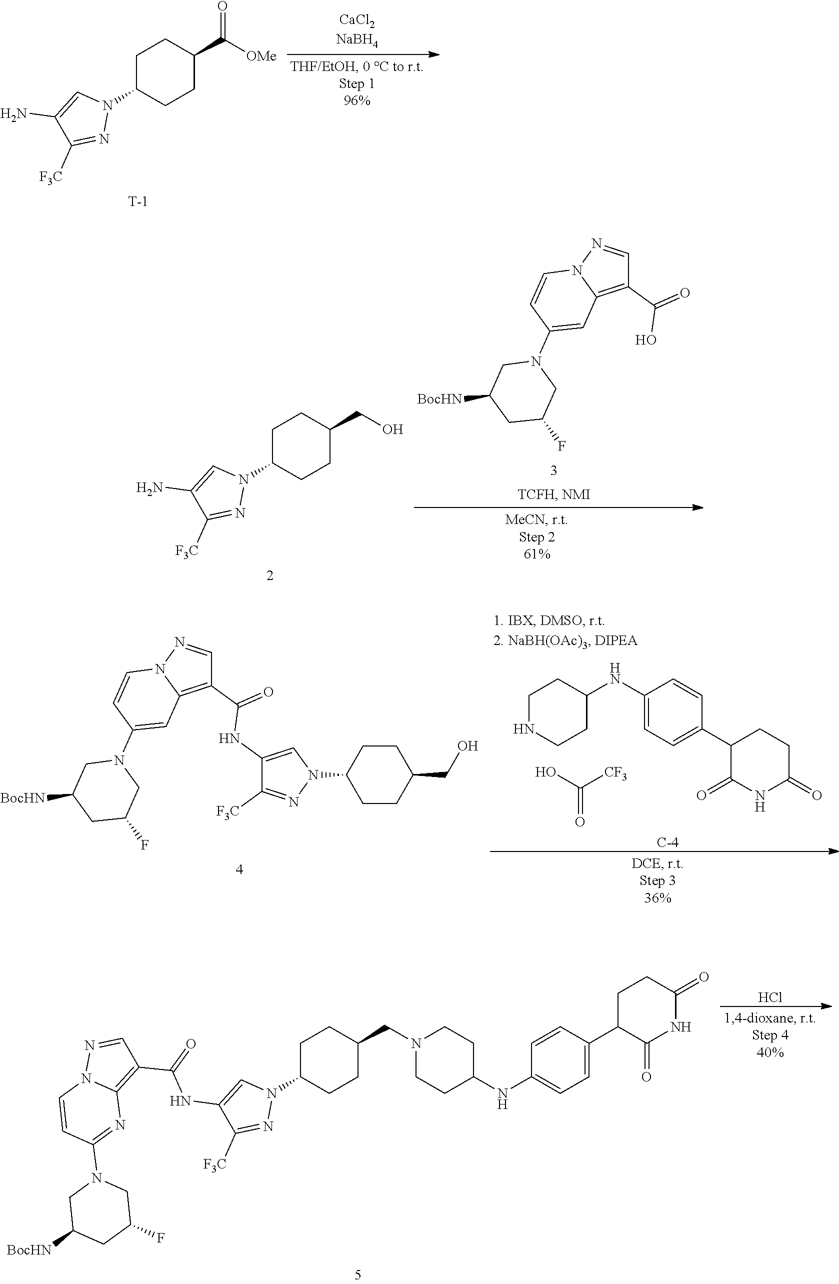

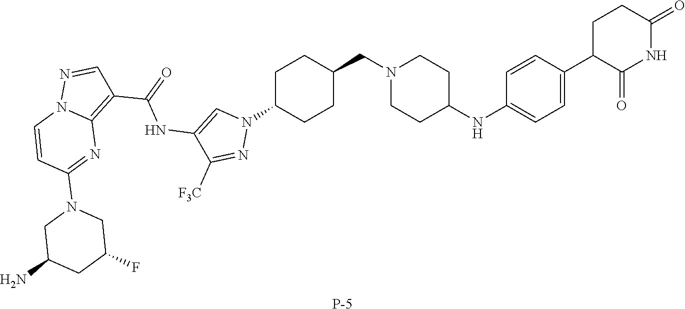

Step 1. Preparation of [4-[4-Amino-3-(trifluoromethyl)pyrazol-1-yl]cyclohexyl]methanol (2). To a solution of methyl 4-[4-amino-3-(trifluoromethyl)pyrazol-1-yl]cyclohexanecarboxylate T-1 (325 mg, 1.12 mmol, 1 eq.) in THF (3.7 mL) and ethanol (7.4 mL) at 0° C. were added CaCl₂) (248 mg, 2.23 mmol, 2 eq.) followed by NaBH₄ (169 mg, 4.46 mmol, 4 eq.). After the addition, the reaction was allowed to stir at room temperature. After 18 h, LCMS showed full conversion. Water was added and the reaction was stirred at room temperature. After 1 h, the aqueous layer was extracted 3 times with EtOAc. The organics were washed with brine, dried over Na₂SO₄ and concentrated to give 2 (283 mg, 96% yield) as a pink oil.

LCMS method 1: 99.9% purity at 215 nm, [M+H]⁺=264.2.

¹H NMR (400 MHz, DMSO-d6) δ ppm 1.04 (qd, J=12.8, 2.9 Hz, 2H), 1.34-1.47 (m, 1H), 1.63 (qd, J=12.6, 3.4 Hz, 2H), 1.83 (br d, J=11.5 Hz, 2H), 1.94-1.99 (m, 2H), 3.24 (t, J=5.7 Hz, 2H), 3.95-4.07 (m, 1H), 4.18 (s, 2H), 4.45 (t, J=5.3 Hz, 1H), 7.23 (s, 1H)

Step 2. Preparation of tert-Butyl N-[(3R,5R)-5-fluoro-1-[3-[1-[4-(hydroxymethyl)cyclohexyl]-3-(trifluoromethyl)pyrazol-4-yl]carbamoyl]pyrazolo[1,5-a]pyrimidin-5-yl]-3-piperidyl]carbamate (4). To a solution of [4-[4-amino-3-(trifluoromethyl)pyrazol-1-yl]cyclohexyl]methanol 2 (283 mg, 1.07 mmol, 1 eq.), 5-[(3R,5R)-3-(tert-butoxycarbonylamino)-5-fluoro-1-piperidyl]pyrazolo[1,5-a]pyrimidine-3-carboxylic acid 3-intermediate 4 from synthesis of P-1-(408 mg, 1.07 mmol, 1 eq.) and NMI (298 μL, 3.76 mmol, 3.5 eq.) in MeCN (5.4 mL) was added TCFH (362 mg, 1.29 mmol, 1.2 eq.). The reaction was stirred at room temperature. After 18 h, LCMS showed full conversion. Water was added and the reaction was stirred at 0° C. After 30 min, the solid was filtered on a Buchner funnel, rinsed with a water/MeCN mixture and then dried under high vacuum to give 4 (554 mg, 61% yield) as a tan solid.

LCMS method 1:73.7% purity at 215 nm, [M+H]⁺=625.2.

¹H NMR (400 MHz, DMSO-d6) δ ppm 1.02-1.15 (m, 2H), 1.29 (br s, 9H), 1.69-1.93 (m, 6H), 1.99-2.16 (m, 5H), 3.26 (br t, J=5.0 Hz, 2H), 3.39-3.56 (m, 1H), 3.58-3.72 (m, 1H), 4.23 (br t, J=11.4 Hz, 1H), 4.47 (br t, J=4.9 Hz, 1H), 4.51-4.64 (m, 1H), 4.97-5.15 (m, 1H), 6.91 (br d, J=5.6 Hz, 1H), 7.10 (br s, 1H), 8.29 (s, 1H), 8.40 (br s, 1H), 8.81 (br d, J=7.6 Hz, 1H), 9.21 (br s, 1H).

¹⁹F NMR (377 MHz, DMSO-d6) δ ppm −185.41-−184.01 (m, 1F), −58.98 (br s, 3F).

Step 3. Preparation of tert-Butyl N-[(3R,5R)-1-[3-[1-[4-[[4-[4-(2,6-dioxo-3-piperidyl)anilino]-1-piperidyl]methyl]cyclohexyl]-3-(trifluoromethyl)pyrazol-4-yl]carbamoyl]pyrazolo[1,5-a]pyrimidin-5-yl]-5-fluoro-3-piperidyl]carbamate (5).

Preparation of Solution A: To a solution of tert-butyl N-[(3R,5R)-5-fluoro-1-[3-[1-[4-(hydroxymethyl)cyclohexyl]-3-(trifluoromethyl)pyrazol-4-yl]carbamoyl]pyrazolo[1,5-a]pyrimidin-5-yl]-3-piperidyl]carbamate 4 (100 mg, 0.160 mmol, 1 eq.) in anhydrous DMSO (1.5 mL) was added IBX (58 mg, 0.210 mmol, 1.3 eq.). The resulting mixture was stirred at room temperature. After 18 h, LCMS showed full conversion toward tert-butyl N-[(3R,5R)-5-fluoro-1-[3-[1-(4-formylcyclohexyl)-3-(trifluoromethyl)pyrazol-4-yl]carbamoyl]pyrazolo[1,5-a]pyrimidin-5-yl]-3-piperidyl]carbamate.

To a solution of 3-[4-(4-piperidylamino)phenyl]piperidine-2,6-dione 2,2,2-trifluoroacetic acid C-4 (77 mg, 0.190 mmol, 1.2 eq.) in anhydrous DCE (1.6 mL) was added DIPEA (0.22 mL, 1.28 mmol, 8 eq.). The resulting mixture was stirred at room temperature. After 15 min, NaBH(OAc)₃ (51 mg, 0.240 mmol, 1.5 eq.) was added followed by Solution A and the reaction was stirred at room temperature. After 1 h, LCMS showed full conversion. Volatiles were removed under reduce pressure and the residue was purified by reverse phase flash chromatography (30 g C18 gold column, liquid deposit (DMSO), elution: 5% MeCN/0.1% HCOOH over 3 CV, then 5 to 35% MeCN/0.1% HCOOH over 5 CV, then 35 to 40% MeCN/0.1% HCOOH over 5 CV, then 40 to 100% MeCN/0.1% HCOOH over 5 CV). Fractions were combined and concentrated to give 5 (59.2 mg, 36% yield) as a white solid, which was used as-is for the next step.

LCMS method 1: 87.2% purity at 215 nm, [M+H]⁺=894.4.

Step 4. Preparation of N-[1-[4-[[4-[4-(2,6-dioxo-3-piperidyl)anilino]-1-piperidyl]methyl]cyclohexyl]-3-(trifluoromethyl)pyrazol-4-yl]-5-[(3R,5R)-3-amino-5-fluoro-1-piperidyl]pyrazolo[1,5-a]pyrimidine-3-carboxamide hydrochloride (P-5). A solution of tert-butyl N-[(3R,5R)-1-[3-[[1-[4-[4-[4-(2,6-dioxo-3-piperidyl)anilino]-1-piperidyl]methyl]cyclohexyl]-3-(trifluoromethyl)pyrazol-4-yl]carbamoyl]pyrazolo[1,5-a]pyrimidin-5-yl]-5-fluoro-3-piperidyl]carbamate 5 (59.2 mg, 0.066 mmol, 1 eq.) in a 4 M HCl solution in 1,4-dioxane (3.5 mL, 14.0 mmol, 210 eq.) was stirred at room temperature. After 1 h, LCMS showed full conversion. Solvents were removed under reduced pressure and the residue was purified by reverse phase flash chromatography (30 g C18 gold column, liquid deposit (DMSO), elution: 5% MeCN/0.02 M HCl over 3 CV, then 5 to 30% MeCN/0.02 M HCl over 17 CV). Fractions were combined and concentrated. The residue was purified by preparative HPLC (MeCN/0.2 M HCl). Fractions were combined, concentrated and lyophilised to give P-5 (21.29 mg, 40% yield) as a white solid as a hydrochloride.

LCMS method 3:99.9% purity at 215 nm, [M−HCl+H]⁺=794.4.

¹H NMR (400 MHz, DMSO-d6) δ ppm 1.16-1.28 (m, 3H), 1.79-2.03 (m, 8H), 2.06-2.15 (m, 5H), 2.35-2.46 (m, 2H), 2.57-2.69 (m, 2H), 2.94-3.08 (m, 3H), 3.21-3.62 (m, 7H), 3.65-3.71 (m, 1H), 4.28-4.38 (m, 1H), 4.51-4.87 (m, 2H), 5.12 (d, J=48.0 Hz, 1H), 6.60-6.73 (m, 2H), 6.92 (d, J=7.8 Hz, 1H), 6.97 (d, J=7.5 Hz, 2H), 8.28-8.37 (m, 3H), 8.46 (s, 1H), 8.94 (d, J=8.0 Hz, 1H), 9.24 (s, 1H), 9.53-9.73 (m, 1H), 10.76 (s, 1H).

¹⁹F NMR (377 MHz, DMSO-d6) δ ppm −185.01 (s, 1F), −58.97 (s, 3F).

### Example S7. Synthesis of P-6

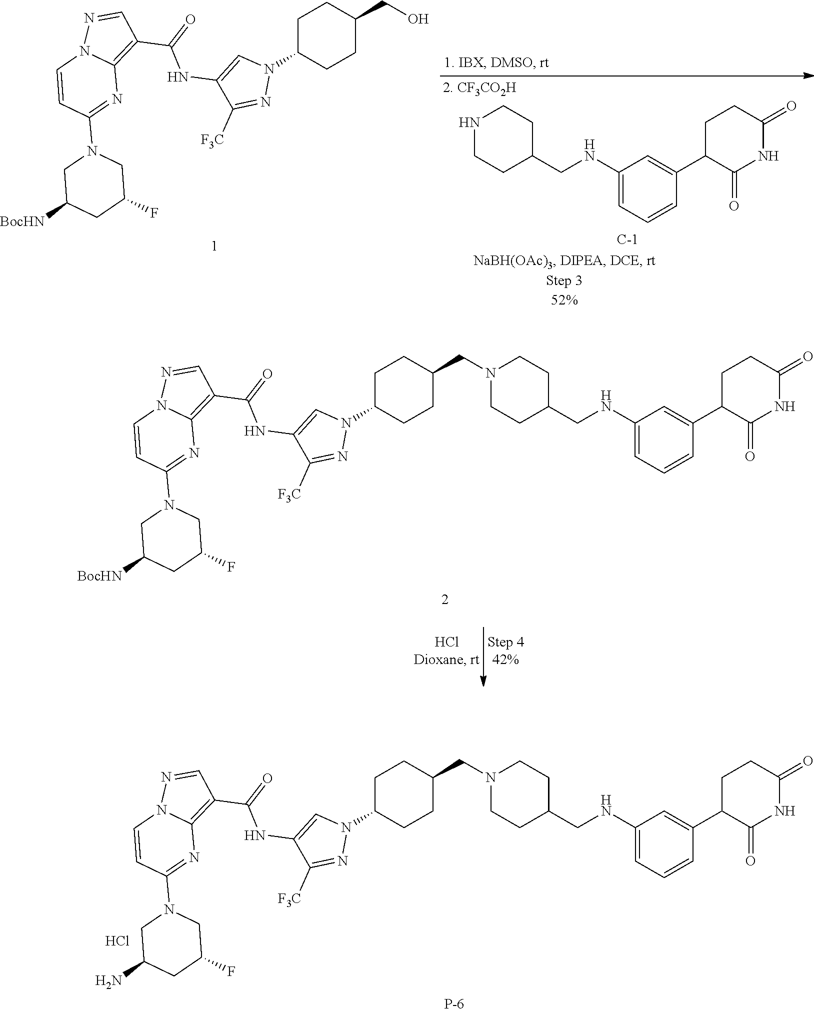

Step 3. Preparation of tert-butyl N-[(3R,5R)-1-[3-[[1-[4-[4-[[3-(2,6-dioxo-3-piperidyl)anilino]methyl]-1-piperidyl]methyl]cyclohexyl]-3-(trifluoromethyl)pyrazol-4-yl]carbamoyl]pyrazolo[1,5-a]pyrimidin-5-yl]-5-fluoro-3-piperidyl]carbamate (2). To a solution of tert-butyl N-[(3R,5R)-5-fluoro-1-[3-[1-[4-(hydroxymethyl)cyclohexyl]-3-(trifluoromethyl)pyrazol-4-yl]carbamoyl]pyrazolo[1,5-a]pyrimidin-5-yl]-3-piperidyl]carbamate 1-intermediate 4 for synthesis of P-5-(100 mg, 0.16 mmol) in dry DMSO (1.6 mL) was added IBX (58.3 mg, 0.21 mmol). The resulting mixture was stirred at room temperature overnight. In a second flask, crude 3-[3-(4-piperidylmethylamino)phenyl]piperidine-2,6-dione trifluoroacetic acid salt C-1 (80.1 mg, 0.19 mmol) was suspended in dry DCE (1.6 mL), where DIPEA (223 μL, 1.28 mmol) was added, and the resulting mixture was stirred at room temperature for 0.25 h. Additionally, NaBH(OAc)₃ (44.3 mg, 0.21 mmol) was added. Then, the the solution containing the aldehyde intermediate of 1 was added dropwise via cannula. The resulting mixture was stirred at room temperature for 1 hour. The DCE was then evaporated under reduced pressure. The resulting DMSO solution was directly loaded on a 30 g gold C18 Isco chromatography column. Elution was performed with MeCN/0.1% aqueous formic acid (40 to 60%). The product eluted out at 45% MeCN/0.1% aqueous formic acid. The desired fractions were combined and concentrated to give 2 (75 mg, 52% yield) as a light-yellow solid.

LCMS method 3:98.9% purity at 215 nm, [M+H]⁺=908.5, [M+2H]²⁺=454.8.

¹H NMR (400 MHz, DMSO-d6) δ ppm 0.97-1.25 (m, 6H), 1.47-1.69 (m, 3H), 1.70-1.83 (m, 5H), 1.83-2.00 (m, 6H), 2.00-2.16 (m, 8H), 2.20 (br d, J=6.8 Hz, 2H), 2.55-2.69 (m, 2H), 2.82-3.00 (m, 5H), 3.38-3.57 (m, 2H), 3.58-3.72 (m, 3H), 4.24 (br t, J-11.4 Hz, 1H), 4.46-4.67 (m, 1H), 4.97-5.15 (m, 1H), 5.58-5.70 (m, 1H), 6.34 (d, J=7.3 Hz, 1H), 6.40 (s, 1H), 6.45 (br d, J=8.3 Hz, 1H), 6.91 (br d, J=7.3 Hz, 1H), 7.00 (t, J=7.7 Hz, 1H), 7.05-7.17 (m, 1H), 8.29 (s, 1H), 8.39 (br s, 1H), 8.82 (br d, J=8.1 Hz, 1H), 9.21 (s, 1H), 10.78 (s, 1H).

Step 4. Preparation of 5-((3R,5R)-3-amino-5-fluoropiperidin-1-yl)-N-(1-((1r,4r)-4-((4-(((3-(2,6-dioxopiperidin-3-yl)phenyl)amino)methyl) piperidin-1-yl)methyl)cyclohexyl)-3-(trifluoromethyl)-1H-pyrazol-4-yl) pyrazolo[1,5-a]pyrimidine-3-carboxamide hydrochloride (P-6). In a round-bottom flask, tert-butyl N-[(3R,5R)-1-[3-[1-[4-[[4-[[3-(2,6-dioxo-3-piperidyl)anilino]methyl]-1-piperidyl]methyl]cyclohexyl]-3-(trifluoromethyl)pyrazol-4-yl]carbamoyl]pyrazolo[1,5-a]pyrimidin-5-yl]-5-fluoro-3-piperidyl]carbamate 2 (75 mg, 0.08 mmol) was solubilized in a 4 M HCl solution in dioxane (3.0 mL, 12.00 mmol). The solution was stirred at room temperature for 0.5 hour. The solvent was then evaporated under reduced pressure. The residue was dissolved in water, and the solution was loaded on a 30 g gold C18 Isco chromatography column. Elution was performed with MeCN/0.02 M aqueous HCl (5 to 40%). The product eluted out at 25% MeCN/0.02 M aqueous HCl. The desired fractions were combined and concentrated to give the product, but in only 92.4% purity (LCMS method 3). The impure product was further purified by preparative HPLC. The desired fractions were combined and concentrated. The residue was solubilized in water, frozen, and lyophilized to give P-6 (29 mg, 42% yield) as a white solid as a hydrochloride salt.

LCMS method 3:99.9% purity at 215 nm, [M−HCl+H]⁺=808.4, [M−HCl+2H]²⁺=404.8, [M−HCl+3H]3+=270.2.

¹H NMR (400 MHz, DMSO-d6) δ ppm 1.09-1.31 (m, 2H), 1.51-2.18 (m, 15H), 2.35-2.47 (m, 2H), 2.54-2.65 (m, 2H), 2.82-3.14 (m, 5H), 3.16-3.60 (m, 5H), 3.72 (dd, J=10.0, 5.1 Hz, 1H), 4.26-4.39 (m, 2H), 4.53-4.90 (m, 2H), 5.12 (d, J=45.7 Hz, 1H), 6.28-6.78 (m, 3H), 6.93 (d, J=7.6 Hz, 1H), 7.01-7.16 (m, 1H), 8.29-8.56 (m, 4H), 8.94 (d, J=7.8 Hz, 1H), 9.24 (s, 1H), 9.47-9.86 (m, 1H), 10.81 (s, 1H).

¹⁹F NMR (377 MHz, DMSO-d6) δ ppm −184.84 (s, 1F), −58.65 (s, 3F).

### Example S8. Synthesis of P-19

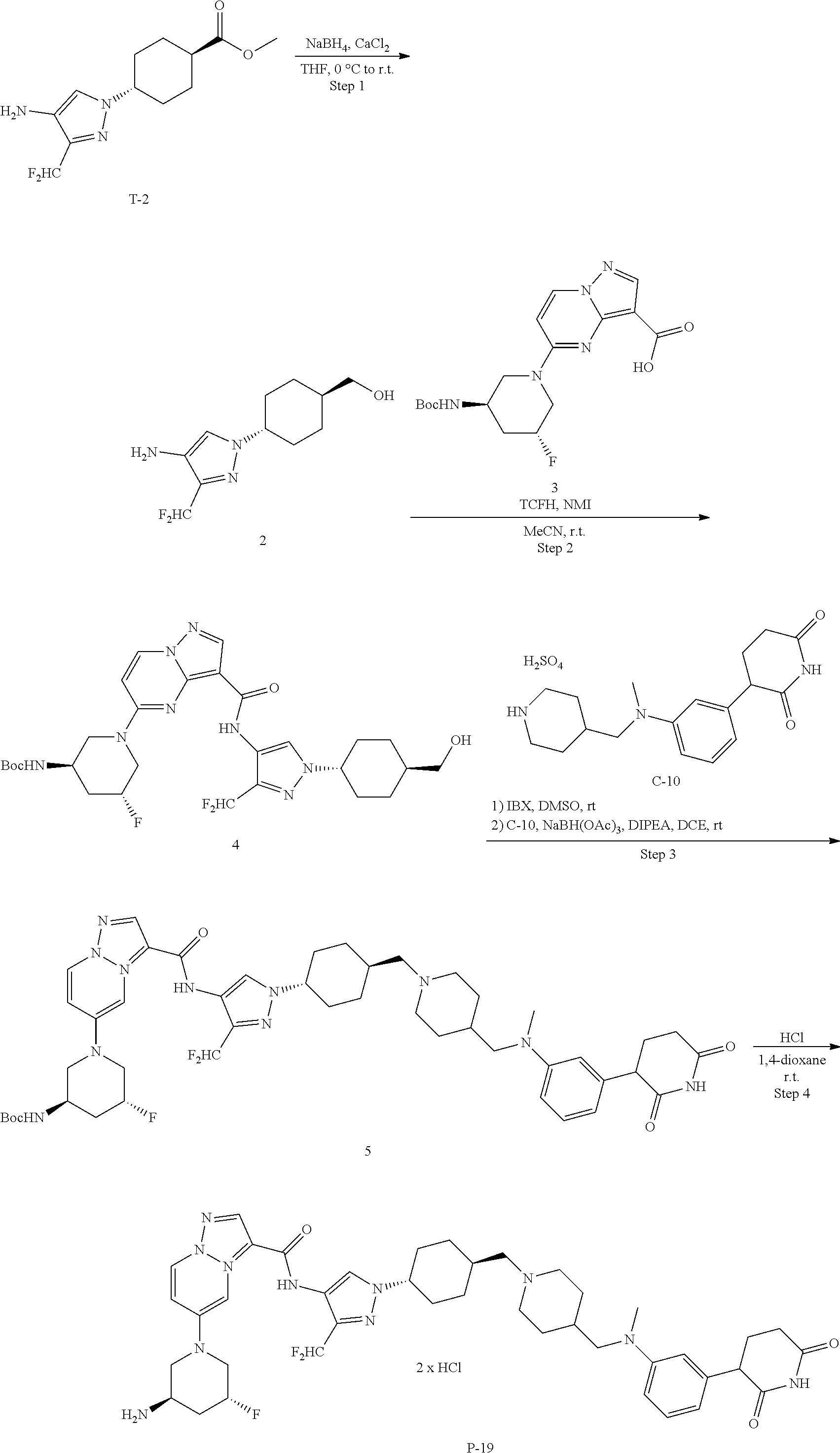

Step 1. Preparation of [4-[4-Amino-3-(difluoromethyl)pyrazol-1-yl]cyclohexyl]methanol (2). To a solution of methyl 4-[4-amino-3-(difluoromethyl)pyrazol-1-yl]cyclohexanecarboxylate T-2 (1.23 g, 4.5 mmol, 1.0 eq.) in THF (15.0 mL, 0.1 M) and ethanol (30.0 mL) at 0° C. was added CaCl₂) (1.0 g, 9.0 mmol, 2.0 eq,), followed by NaBH₄ (0.68 g, 18.0 mmol, 4.0 eq.). The resulting mixture was stirred overnight, allowing it to warm up to room temperature. After overnight stirring, water was added, and the reaction mixture was stirred at room temperature for 1 hour. The aqueous phase was extracted with EtOAc (3 x). The organic layers were washed with brine, dried over Na₂SO₄ and concentrated to dryness, which afforded [4-[4-amino-3-(difluoromethyl)pyrazol-1-yl]cyclohexyl]methanol 2 (1.1 g, 99% yield) as an orange oil.

LCMS method 1: retention time: 0.989 min, 99.0% purity at 215 nm, [M+H]⁺=246.2.

¹H NMR (400 MHz, DMSO-d6) δ ppm 1.04 (qd, J=12.9, 3.2 Hz, 2H), 1.32-1.43 (m, 1H), 1.62 (qd, J=12.6, 3.3 Hz, 2H), 1.82 (br d, J=11.5 Hz, 2H), 1.93-1.98 (m, 2H), 3.24 (t, J=5.7 Hz, 2H), 3.94 (tt, J=11.9, 3.8 Hz, 1H), 4.00-4.06 (m, 2H), 4.44 (t, J=5.3 Hz, 1H), 6.72-7.02 (m, 1H), 7.14 (s, 1H).

¹⁹F NMR (377 MHz, DMSO-d6) δ ppm −111.21 (s, 2F).

Step 2. Preparation of tert-Butyl N-[(3r,5r)-1-[3-[[3-(difluoromethyl)-1-[4-(hydroxymethyl)cyclohexyl]pyrazol-4-yl]carbamoyl]pyrazolo[1,5-a]pyrimidin-5-yl]-5-fluoro-3-piperidyl]carbamate (4). To a solution of [4-[4-amino-3-(difluoromethyl)pyrazol-1-yl]cyclohexyl]methanol 2 (511.8 mg, 1.88 mmol, 1.5 eq.), 5-[(3r,5r)-3-(tert-butoxycarbonylamino)-5-fluoro-1-piperidyl]pyrazolo[1,5-a]pyrimidine-3-carboxylic acid 3-intermediate 4 from synthesis of P-1-(475.0 mg, 1.25 mmol, 1.0 eq.) and NMI (362.72 μL, 4.58 mmol, 3.6 eq.) in MeCN (12.5 mL, 0.1 M) was added TCFH (440.43 mg, 1.57 mmol, 1.2 eq.). The resulting mixture was stirred at room temperature overnight. Water was added and the reaction mixture was stirred at room temperature for 1 hour. The solid was filtered on a Buchner funnel and rinsed with a water/MeCN mixture. Purification by reverse phase chromatography (50 g C18 RediSep Rf Gold column, liquid deposit (DMSO), elution: 5% MeOH/0.1% HCOOH over 4 CV, then 5% to 100% MeOH/0.1% HCOOH over 15 CV). The pure fractions were combined, concentrated under reduced pressure to afford tert-butyl N-[(3r,5r)-1-[3-[[3-(difluoromethyl)-1-[4-(hydroxymethyl)cyclohexyl]pyrazol-4-yl]carbamoyl]pyrazolo[1,5-a]pyrimidin-5-yl]-5-fluoro-3-piperidyl]carbamate 4 (421 mg, 55% yield) as a tan solid.

LCMS method 1: retention time: 1.629 min, 99.9% purity at 215 nm, [M+H]⁺=607.2.

¹H NMR (400 MHz, DMSO-d6) δ ppm 1.03-1.15 (m, 2H), 1.34 (br s, 9H), 1.64-1.80 (m, 3H), 1.82-1.93 (m, 3H), 1.99-2.07 (m, 2H), 2.08-2.18 (m, 1H), 2.95-3.14 (m, 1H), 3.26 (t, J=5.7 Hz, 2H), 3.37-3.52 (m, 1H), 3.62-3.71 (m, 1H), 4.12-4.21 (m, 1H), 4.47 (t, J=5.3 Hz, 1H), 4.69 (br s, 1H), 4.95-5.12 (m, 1H), 6.86-6.94 (m, 1H), 7.01-7.22 (m, 2H), 8.28 (s, 1H), 8.34 (br s, 1H), 8.82 (d, J=7.8 Hz, 1H), 9.31 (br s, 1H).

¹⁹F NMR (377 MHz, DMSO-d6) δ ppm −186.02-−179.79 (m, 1F), −113.10-−108.69 (m, 2F).

Step 3. Preparation of tert-butyl N-[(3R,5R)-1-[3-[[3-(difluoromethyl)-1-[4-[4-[[3-(2,6-dioxo-3-piperidyl)-N-methyl-anilino]methyl]-1-piperidyl]methyl]cyclohexyl]pyrazol-4-yl]carbamoyl]pyrazolo[1,5-a]pyrimidin-5-yl]-5-fluoro-3-piperidyl]carbamate (5). To a solution of 4 (150.0 mg, 0.127 mmol, 1.0 eq.) in dry DMSO (1.4 mL, 0.1 M) was added IBX (64.0 mg, 0.152 mmol, 1.2 eq.). The resulting mixture was stirred at room temperature overnight. Separately, a solution of C-10 (1.0 mL, 0.1400 mmol, 1.1 eq.) in DCE (1 mL) and DIPEA (215.66 μL, 1.24 mmol, 10 eq.) was stirred at rt for 10 minutes, to which the DMSO solution containing the oxidated 4 was added. After 10 minutes of stirring, NaBH(OAc)₃ (34.18 mg, 0.1600 mmol, 1.3 eq.) was added. The resulting mixture was stirred at room temperature for 1 hour. DCE was removed under reduced pressure. The crude mixture was purified by column chromatography C18 (5 CV at 5%; 15 CV to 35%; 15 CV to 35% MeCN in H₂O (0.1% formic acid)). The desired fractions were concentrated to dryness to afford 5 (122 mg, 99% yield) as a yellow solid.

LCMS method 1: retention time: 1.508 min, 35.3% purity at 215 nm, [M+2H]²⁺=452.6.

¹H NMR (400 MHz, DMSO-d6) δ ppm 1.01-1.09 (m, 2H), 1.11-1.12 (m, 2H), 1.22-1.42 (m, 9H), 1.58-1.83 (m, 6H), 1.85-1.96 (m, 3H), 2.00-2.08 (m, 2H), 2.09-2.25 (m, 4H), 2.42-2.49 (m, 2H), 2.57-2.66 (m, 1H), 2.88-2.95 (m, 3H), 3.16-3.22 (m, 2H), 3.38-3.49 (m, 2H), 3.63-3.71 (m, 1H), 3.75 (br dd, J=10.5, 4.9 Hz, 1H), 4.12-4.23 (m, 2H), 4.98-5.12 (m, 2H), 6.42 (br d, J=7.3 Hz, 1H), 6.51 (s, 1H), 6.54-6.59 (m, 1H), 6.86-6.94 (m, 1H), 7.04-7.15 (m, 2H), 7.18-7.26 (m, 1H), 7.43-7.50 (m, 1H), 7.65-7.70 (m, 1H), 7.96 (d, J=7.8 Hz, 1H), 8.15 (s, 1H), 8.27-8.31 (m, 1H), 8.32-8.37 (m, 1H), 8.82 (br d, J=8.1 Hz, 1H), 9.32 (br d, J=3.2 Hz, 1H), 10.80 (s, 1H).

¹⁹F NMR (377 MHz, DMSO-d6) δ ppm −184.86 (s, 1F), −111.10 (s, 2F).

Step 4. Preparation of N-[3-(Difluoromethyl)-1-[4-[4-[[3-(2,6-dioxo-3-piperidyl)-N-methyl-anilino]methyl]-1-piperidyl]methyl]cyclohexyl]pyrazol-4-yl]-5-[(3R,5R)-3-amino-5-fluoro-1-piperidyl]pyrazolo[1,5-a]pyrimidine-3-carboxamide dihydrochloride (P-19). In a round bottom flask, 5 (60 mg, 0.07 mmol) was solubilized in a 4 M HCl solution in 1,4-dioxane (2.49 mL, 9.96 mmol, 150 eq.). The mixture was stirred at rt for 1.5h. The solvent was removed under reduced pressure. Water was added to the crude and the resulting solution was directly loaded on a 30 g C18 gold column and purified using MeCN/HCl 0.02M (3 CV at 5%, 15 CV to 35%). The product eluted out at 28% MeCN. Fractions were combined, concentrated and lyophilized to afford P-19 (19.09 mg, 35% yield) as a white solid as a dihydrochloride salt.

LCMS method 3: retention time: 2.478 min, 98.8% purity at 215 nm, [M−2HCl+H]⁺=804.3, [M−2HCl+H]²⁺=402.8.

¹H NMR (400 MHz, DMSO-d6) δ ppm 1.12-1.28 (m, 2H), 1.52-2.11 (m, 14H), 2.12-2.25 (m, 1H), 2.35-2.48 (m, 3H), 2.57-2.65 (m, 1H), 2.78-2.98 (m, 6H), 3.04-3.58 (m, 7H), 3.72-3.78 (m, 1H), 4.18-4.29 (m, 1H), 4.50-4.97 (m, 2H), 5.10 (d, J=47.0 Hz, 1H), 6.39-6.72 (m, 3H), 6.84-6.95 (m, 1H), 6.96-7.30 (m, 2H), 8.19-8.35 (m, 4H), 8.39 (s, 1H), 8.94 (d, J=7.8 Hz, 1H), 9.21-9.60 (m, 2H), 10.80 (s, 1H).

¹⁹F NMR (377 MHz, DMSO-d6) δ ppm −184.67 (s, 1F), −111.29 (s, 2F).

The following compounds were synthesized via the same general routes with modifications to Intermediate (T-X) in step 1 and CBM (C-X) in step 3 (Table 6).

TABLE 6

| Com- | | | |
|---|---|---|---|
| pound | | | |
| No. | Int | CBM | Structure |
| P-3 | T-1 | C-2 | 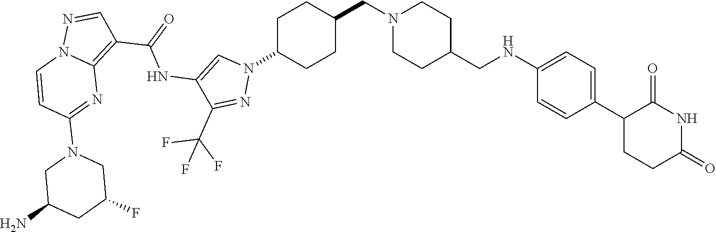 [SMILES: N[C@@H]1C[C@@H](F)CN(c2ccn3ncc(C(=O)Nc4cn([C@H]5CC[C@H](CN6CCC(CNc7ccc(C8CCC(=O)NC8=O)cc7)CC6)CC5)nc4C(F)(F)F)c3n2)C1] |
| P-4 | T-1 | C-3 | 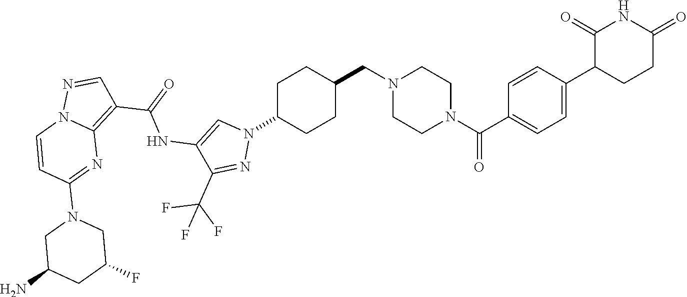 [SMILES: N[C@@H]1C[C@@H](F)CN(c2ccn3ncc(C(=O)Nc4cn([C@H]5CC[C@H](CN6CCN(C(=O)c7ccc(C8CCC(=O)NC8=O)cc7)CC6)CC5)nc4C(F)(F)F)c3n2)C1] |
| P-5 | T-1 | C-4 | 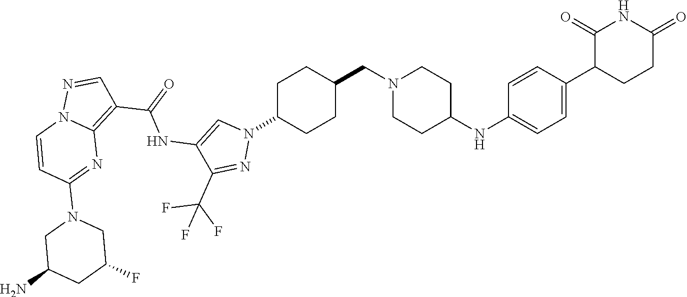 [SMILES: N[C@@H]1C[C@@H](F)CN(c2ccn3ncc(C(=O)Nc4cn([C@H]5CC[C@H](CN6CCC(Nc7ccc(C8CCC(=O)NC8=O)cc7)CC6)CC5)nc4C(F)(F)F)c3n2)C1] |
| P-6 | T-1 | C-1 | 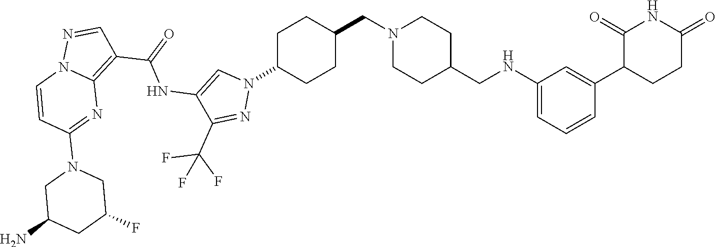 [SMILES: N[C@@H]1C[C@@H](F)CN(c2ccn3ncc(C(=O)Nc4cn([C@H]5CC[C@H](CN6CCC(CNc7cccc(C8CCC(=O)NC8=O)c7)CC6)CC5)nc4C(F)(F)F)c3n2)C1] |
| P-8 | T-1 | C-5 | 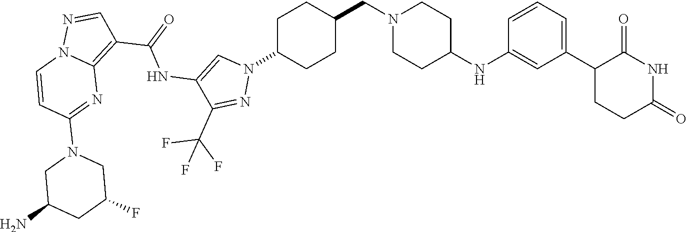 [SMILES: N[C@@H]1C[C@@H](F)CN(c2ccn3ncc(C(=O)Nc4cn([C@H]5CC[C@H](CN6CCC(Nc7cccc(C8CCC(=O)NC8=O)c7)CC6)CC5)nc4C(F)(F)F)c3n2)C1] |
| P-10 | T-1 | C-6 | 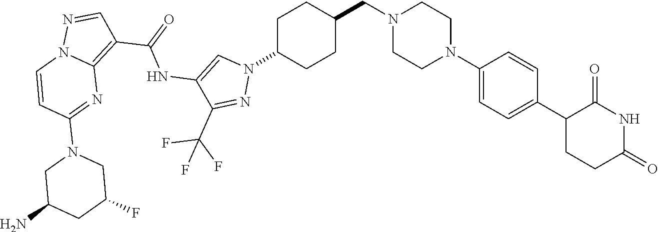 [SMILES: N[C@@H]1C[C@@H](F)CN(c2ccn3ncc(C(=O)Nc4cn([C@H]5CC[C@H](CN6CCN(c7ccc(C8CCC(=O)NC8=O)cc7)CC6)CC5)nc4C(F)(F)F)c3n2)C1] |
| P-13 | T-1 | C-7 | 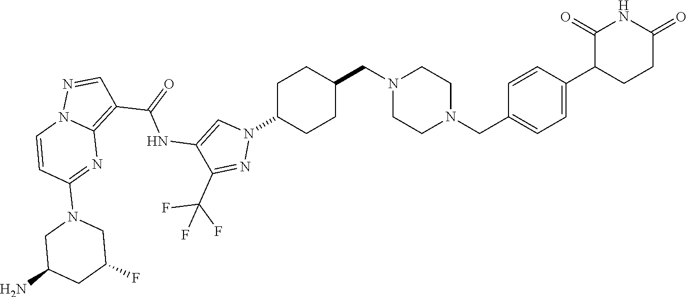 [SMILES: N[C@@H]1C[C@@H](F)CN(c2ccn3ncc(C(=O)Nc4cn([C@H]5CC[C@H](CN6CCN(Cc7ccc(C8CCC(=O)NC8=O)cc7)CC6)CC5)nc4C(F)(F)F)c3n2)C1] |
| P-18 | T-2 | C-11 | 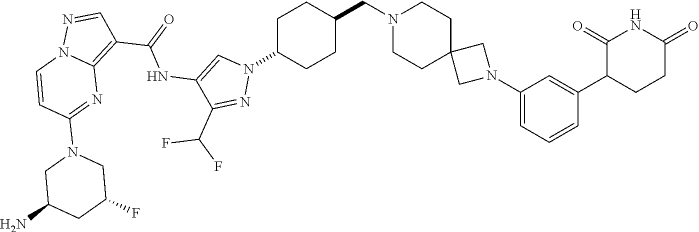 [SMILES: N[C@@H]1C[C@@H](F)CN(c2ccn3ncc(C(=O)Nc4cn([C@H]5CC[C@H](CN6CCC7(CC6)CN(c6cccc(C8CCC(=O)NC8=O)c6)C7)CC5)nc4C(F)F)c3n2)C1] |
| P-19 | T-2 | C-10 | 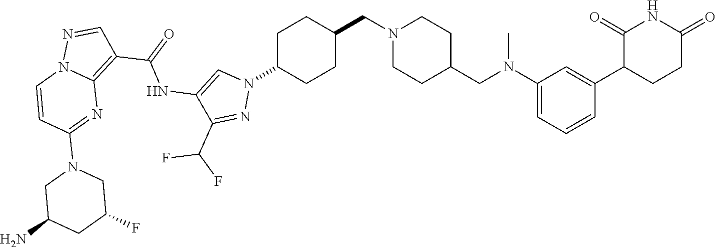 [SMILES: CN(CC1CCN(C[C@H]2CC[C@H](n3cc(NC(=O)c4cnn5ccc(N6C[C@H](N)C[C@@H](F)C6)nc45)c(C(F)F)n3)CC2)CC1)c1cccc(C2CCC(=O)NC2=O)c1] |

| Compound | |
|---|---|
| No. | Characterization Data |
| P-3 | 80% yield as a white solid. |
| | LCMS method 3: retention time: 1.843 min, 99% purity |
| | at 215 nm, [M − HCl + H]⁺ = 808.4. |
| | ¹H NMR (400 MHz, DMSO-d6) δ ppm 1.13-1.27 (m, 2 |
| | H), 1.52-1.66 (m, 2 H), 1.76-2.14 (m, 13 H), 2.35- |
| | 2.47 (m, 2 H), 2.57-2.66 (m, 1 H), 2.83-3.02 (m, 5 H), |
| | 3.03-3.59 (m, 6 H), 3.64-3.72 (m, 2 H), 4.27-4.37 (m, |
| | 1 H), 4.53-4.83 (m, 2 H), 5.12 (d, J = 46.7 Hz, 1 H), |
| | 6.53-6.82 (m, 2 H), 6.87-7.10 (m, 3 H), 8.32-8.43 (m, |
| | 3 H), 8.45 (s, 1 H), 8.94 (d, J = 8.1 Hz, 1 H), 9.24 (s, 1 |
| | H), 9.40-9.77 (m, 1 H), 10.75 (s, 1 H). |
| | ¹⁹F NMR (377 MHz, DMSO-d6) δ ppm −184.95 |
| | (s, 1 F), −58.86 (s, 3 F). |
| P-4 | 39% yield as a white solid. |
| | LCMS method 3: retention time: 1.768 min, 99% purity |
| | at 215 nm, [M − HCl + 2H]²⁺ = 404.8 ; [M − HCl + H]⁺ = |
| | 808.4. |
| | ¹H NMR (400 MHz, DMSO-d6) δ ppm 1.15-1.30 (m, 2 |
| | H), 1.75-2.15 (m, 10 H), 2.17-2.45 (m, 3 H), 2.53- |
| | 2.56 (m, 1 H), 2.64-2.75 (m, 2 H), 3.00-3.19 (m, 5 H), |
| | 3.25-3.48 (m, 5 H), 3.96 (dd, J = 11.5, 5.0 Hz, 1 H), |
| | 4.27-4.39 (m, 1 H), 4.52-4.82 (m, 2 H), 5.12 (d, J = |
| | 42.0 Hz, 1 H), 6.91 (d, J = 7.8 Hz, 1 H), 7.33-7.38 (m, 2 |
| | H), 7.43-7.48 (m, 2 H), 8.23-8.36 (m, 3 H), 8.46 (s, 1 |
| | H), 8.94 (d, J = 7.8 Hz, 1 H), 9.24 (s, 1 H), 10.16-10.31 |
| | (m, 1 H), 10.89 (s, 1 H).-One proton partially under the |
| | peak of DMSO by ¹H NMR. |
| | ¹⁹F NMR (377 MHz, DMSO-d6) δ ppm −185.10 |
| | (s, 1 F), −58.76 (s, 3 F). |
| P-5 | 40% yield as a white solid. |
| | LCMS method 3: retention time: 1.823 min, 99% purity |
| | at 215 nm, [M − HCl+H]⁺ = 794.4. |
| | ¹H NMR (400 MHz, DMSO-d6) δ ppm 1.16-1.28 (m, 3 |
| | H), 1.79-2.03 (m, 8 H), 2.06-2.15 (m, 5 H), 2.35-2.46 |
| | (m, 2 H), 2.57-2.69 (m, 2 H), 2.94-3.08 (m, 3 H), 3.21- |
| | 3.62 (m, 7 H), 3.65-3.71 (m, 1 H), 4.28-4.38 (m, 1 |
| | H), 4.51-4.87 (m, 2 H), 5.12 (d, J = 48.0 Hz, 1 H), 6.60- |
| | 6.73 (m, 2 H), 6.92 (d, J = 7.8 Hz, 1 H), 6.97 (d, J = 7.5 |
| | Hz, 2 H), 8.28-8.37 (m, 3 H), 8.46 (s, 1 H), 8.94 (d, J = |
| | 8.0 Hz, 1 H), 9.24 (s, 1 H), 9.53-9.73 (m, 1 H), 10.76 (s, |
| | 1 H).-Two protons partially under the peak of DMSO |
| | by ¹H NMR. |
| | ¹⁹F NMR (377 MHz, DMSO-d6) δ ppm −185.01 |
| | (s, 1 F), −58.97 (s, 3 F). |
| P-6 | 44% yield as a white solid. |
| | LCMS method 3: retention time: 1.877 min, 99% purity |
| | at 215 nm, [M − HCl + H]⁺ = 808.4, [M − HCl + 2H]²⁺ = |
| | 404.8, [M − HCl+3H]3 + = 270.2. |
| | ¹H NMR (400 MHz, DMSO-d6) δ ppm 1.09-1.31 (m, 2 |
| | H), 1.51-2.18 (m, 15 H), 2.35-2.47 (m, 2 H), 2.54- |
| | 2.65 (m, 2 H), 2.82-3.14 (m, 5 H), 3.16-3.60 (m, 5 H), |
| | 3.72 (dd, J = 10.0, 5.1 Hz, 1 H), 4.26-4.39 (m, 2 H), |
| | 4.53-4.90 (m, 2 H), 5.12 (d, J = 45.7 Hz, 1 H), 6.28- |
| | 6.78 (m, 3 H), 6.93 (d, J = 7.6 Hz, 1 H), 7.01-7.16 (m, 1 |
| | H), 8.29-8.56 (m, 4 H), 8.94 (d, J = 7.8 Hz, 1 H), 9.24 |
| | (s, 1 H), 9.47-9.86 (m, 1 H), 10.81 (s, 1 H). |
| | ¹⁹F NMR (377 MHz, DMSO-d6) δ ppm −184.84 |
| | (s, 1 F), −58.65 (s, 3 F). |
| P-8 | 62% yield as a light pink solid. |
| | LCMS method 3: retention time: 1.851 min, 99% purity |
| | at 215 nm, [M − HCl + 2H]²⁺ = 397.8 ; [M − HCl + H]⁺ = |
| | 794.4 |
| | ¹H NMR (400 MHz, DMSO-d6) δ ppm 1.13-1.29 (m, 2 |
| | H), 1.75-2.20 (m, 15 H), 2.36-2.46 (m, 1 H), 2.60- |
| | 2.69 (m, 1 H), 2.88-3.10 (m, 4 H), 3.21-3.62 (m, 7 H), |
| | 3.69-3.79 (m, 1 H), 4.27-4.37 (m, 2 H), 5.12 (d, J = |
| | 46.0 Hz, 1 H), 6.51-6.79 (m, 3 H), 6.93 (d, J = 8.1 Hz, 1 |
| | H), 7.09-7.23 (m, 1 H), 8.33 (s, 1 H), 8.36-8.51 (m, 4 |
| | H), 8.94 (d, J = 7.8 Hz, 1 H), 9.24 (s, 1 H), 9.84-10.00 |
| | (m, 1 H), 10.82 (s, 1 H). |
| | ¹⁹F NMR (377 MHz, DMSO-d6) δ ppm −184.94 |
| | (s, 1 F), −58.85 (s, 3 F). |
| P-10 | 88% yield as a off-white solid. |
| | LCMS method 3: retention time: 1.858 min, 99% purity |
| | at 215 nm, [M − HCl + 2H]2⁺ = 390.8 ; [M − HCl + H]⁺ = |
| | 780.4. |
| | ¹H NMR (400 MHz, DMSO-d6) δ ppm 1.16-1.30 (m, 2 |
| | H), 1.78-1.91 (m, 2 H), 1.92-2.22 (m, 8 H), 2.35-2.47 |
| | (m, 2 H), 2.60-2.71 (m, 1 H), 3.03-3.64 (m, 11 H), |
| | 3.72-3.84 (m, 3 H), 4.28-4.39 (m, 1 H), 4.56-4.82 (m, |
| | 2 H), 5.12 (d, J = 46.2 Hz, 1 H), 6.93 (d, J = 8.3 Hz, 1 H), |
| | 6.98 (d, J = 8.8 Hz, 2 H), 7.12 (d, J = 8.6 Hz, 2 H), 8.33 |
| | (s, 1 H), 8.39-8.50 (m, 3 H), 8.94 (d, J = 8.1 Hz, 1 H), |
| | 9.24 (s, 1 H), 10.27 (br s, 1 H), 10.79 (s, 1 H). |
| | ¹⁹F NMR (377 MHz, DMSO-d6) δ ppm-184.95 |
| | (s, 1 F), −58.85 (s, 3 F). |
| P-13 | 40% yield as a white solid. |
| | LCMS method 3: retention time: 1.810 min, 99% purity |
| | at 215 nm, [M − 2HCl+H]⁺ = 794.4, [M − 2HCl + 2H]²⁺ = |
| | 397.7. |
| | ¹H NMR (400 MHz, DMSO-d6) δ ppm 1.11-1.27 (m, 2 |
| | H), 1.74-2.14 (m, 10 H), 2.17-2.31 (m, 1 H), 2.35- |
| | 2.45 (m, 1 H), 2.63-2.76 (m, 2 H), 2.97-3.15 (m, 2 H), |
| | 3.27-3.64 (m, 10 H), 3.66-3.84 (m, 2 H), 3.93 (dd, J = |
| | 11.5, 4.6 Hz, 1 H), 4.31-4.33 (m, 1 H), 4.63-4.84 (m, 2 |
| | H), 5.12 (d, J = 48.0 Hz, 1 H), 6.92 (d, J = 7.8 Hz, 1 H), |
| | 7.34 (d, J = 7.8 Hz, 2 H), 7.59 (d, J = 7.1 Hz, 2 H), 8.29- |
| | 8.51 (m, 5 H), 8.94 (d, J = 7.8 Hz, 1 H), 9.23 (s, 1 H), |
| | 10.68-11.39 (m, 2 H).-One proton obscured by water |
| | peak by ¹H NMR. |
| | ¹⁹F NMR (377 MHz, DMSO-d6) δ ppm-184.91 |
| | (s, 1 F), −58.84 (s, 3 F). |
| P-18 | 33% yield as a white solid. |
| | LCMS method 3: retention time: 1.876 min, 99% purity |
| | at 215 nm, [M + H]⁺ = 802.5, [M + 2H]²⁺ = 401.8. |
| | ¹H NMR (400 MHz, DMSO-d6) δ ppm 0.95-1.15 (m, 2 |
| | H), 1.50-1.66 (m, 1 H), 1.68-1.80 (m, 7 H), 1.90 (br d, |
| | J = 11.6 Hz, 2 H), 2.00-2.08 (m, 3 H), 2.10-2.15 (m, 2 |
| | H), 2.16-2.27 (m, 2 H), 2.29-2.41 (m, 3 H), 2.43 (br t, |
| | J = 4.2 Hz, 1 H), 2.57-2.72 (m, 1 H), 2.96-3.07 (m, 1 |
| | H), 3.12-3.18 (m, 1 H), 3.33-3.41 (m, 4 H), 3.51 (s, 4 |
| | H), 3.73 (dd, J = 10.8, 5.0 Hz, 1 H), 4.11-4.27 (m, 1 H), |
| | 4.30-4.62 (m, 1 H), 4.64-4.92 (m, 1 H), 5.03 (d, J = |
| | 45.0 Hz, 1 H), 6.25 (s, 1 H), 6.31 (d, J = 8.3 Hz, 1 H), |
| | 6.49 (d, J = 7.5 Hz, 1 H), 6.91 (d, J = 7.9 Hz, 1 H), 6.97- |
| | 7.32 (m, 2 H), 8.29 (s, 1 H), 8.37 (s, 1 H), 8.84 (d, J = 7.9 |
| | Hz, 1 H), 9.33 (s, 1 H), 10.79 (br s, 1 H). |
| | ¹⁹F NMR (377 MHz, DMSO-d6) δ ppm −183.43 |
| | (s, 1 F), −111.06 (s, 2 F). |
| P-19 | 35% yield as a white solid. |
| | LCMS method 3: retention time: 2.478 min, 99% purity |
| | at 215 nm, [M − 2HCl+H]⁺ = 804.3, [M − 2HCl + H]²⁺ = |
| | 402.8. |
| | ¹H NMR (400 MHz, DMSO-d6) δ ppm 1.12-1.28 (m, 2 |
| | H), 1.52-2.11 (m, 14 H), 2.12-2.25 (m, 1 H), 2.35- |
| | 2.48 (m, 3 H), 2.57-2.65 (m, 1 H), 2.78-2.98 (m, 6 H), |
| | 3.04-3.58 (m, 7 H), 3.72-3.78 (m, 1 H), 4.18-4.29 (m, |
| | 1 H), 4.50-4.97 (m, 2 H), 5.10 (d, J = 47.0 Hz, 1 H), |
| | 6.39-6.72 (m, 3 H), 6.84-6.95 (m, 1 H), 6.96-7.30 (m, |
| | 2 H), 8.19-8.35 (m, 4 H), 8.39 (s, 1 H), 8.94 (d, J = 7.8 |
| | Hz, 1 H), 9.21-9.60 (m, 2 H), 10.80 (s, 1 H). |
| | ¹⁹F NMR (377 MHz, DMSO-d6) δ ppm −184.67 |
| | (s, 1 F), −111.29 (s, 2 F). |

### Final Product General Method 3

### Example S9. Synthesis of P-11

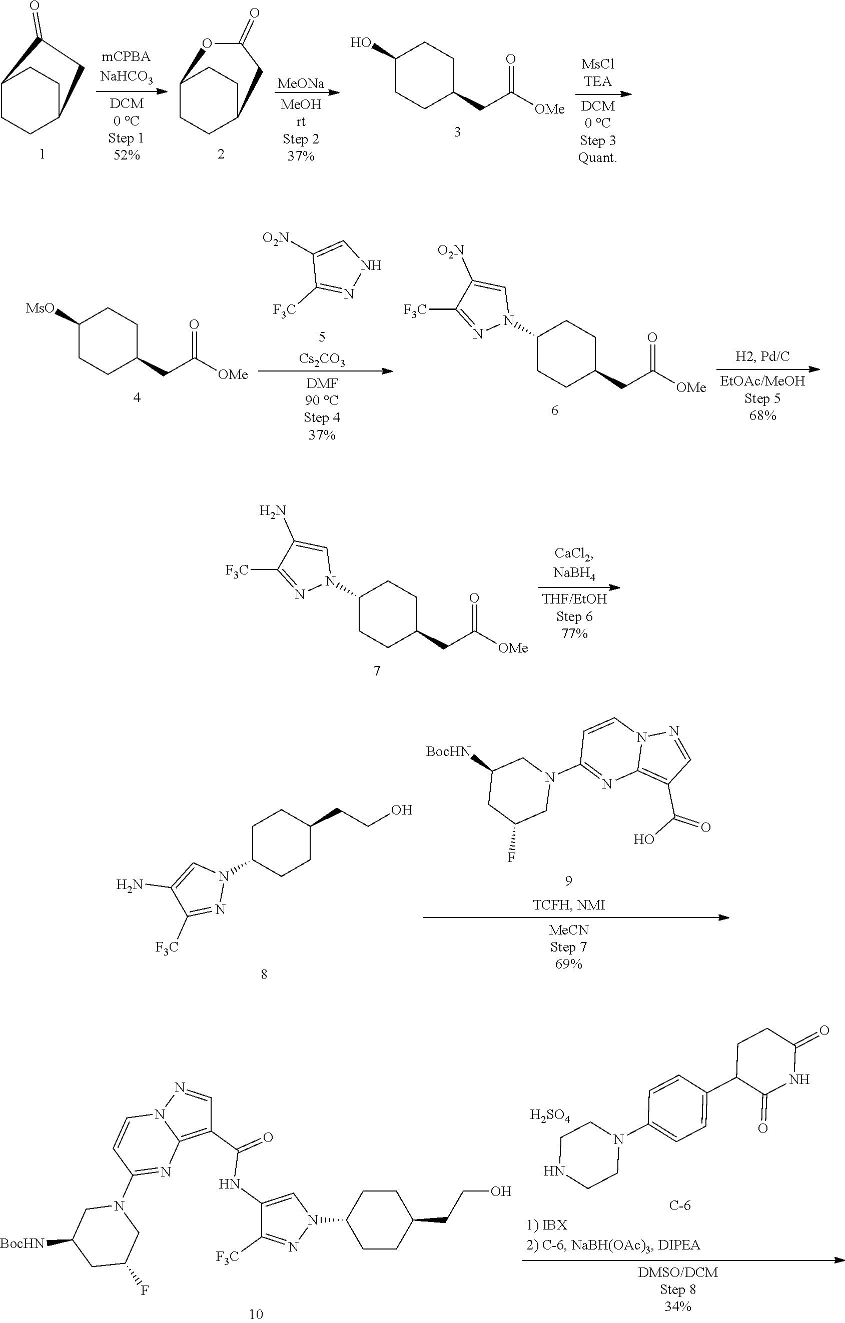

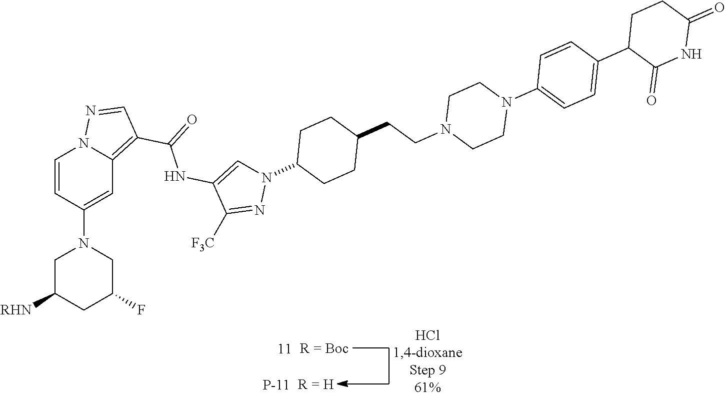

Step 1. Preparation of 2-Oxabicyclo[3.2.2]nonan-3-one (2). To a solution of 1 (800. mg, 6.44 mmol, 1.0 eq.) in dry DCM (42.9 mL, 0.15 M) at 0° C. was added NaHCO₃ (541.15 mg, 6.44 mmol, 1.0 eq.) and mCPBA (1.5 g, 6.44 mmol, 1.0 eq.). The resulting mixture was stirred for 4 hours, allowing it to warm up to room temperature. The reaction mixture was diluted with DCM. The organics were washed once with 1 N aqueous NaOH and once with brine, dried over Na₂SO₄, and concentrated to dryness. The crude mixture was purified by flash chromatography eluting with a gradient from 40 to 75% EtOAc in Heptanes. The desired fractions (which eluted out at 50% EtOAc in Heptanes, Rf=0.2 on TLC eluting 50% EtOAc in Heptanes, vanillin stain (grey-purple spot)) was concentrated to dryness to afford 2 (478 mg, 52% yield) as a white solid.

LCMS method 1: retention time: 1.292 min, 99.9% purity at 200 nm, [M+H]⁺=141.1.

¹H NMR (400 MHz, CDCl₃) δ ppm 1.72-1.96 (m, 6H), 2.04-2.10 (m, 1H), 2.17-2.27 (m, 2H), 2.87 (d, J=4.2 Hz, 2H), 4.50-4.55 (m, 1H).

Step 2. Preparation of Methyl 2-(4-hydroxycyclohexyl)acetate (3). To a solution of 2 (565 mg, 4.03 mmol, 1.0 eq.) in methanol (20.15 mL, 0.2 M) was added MeONa (1.09 g, 20.15 mmol, 5.0 eq.). The resulting mixture was stirred at room temperature for 1 hour. TLC showed completion of reaction (eluting 70% MTBE in Heptanes, revealing Vanillin, Rf SM=0.3 (grey/purple spot) Rf EP=0.4 (purple spot)). MeOH was evaporated under reduced pressure. The residue was dissolved in MTBE and saturated aqueous NH₄Cl was added. The aqueous phase was extracted 3× with MTBE. Organic layers were dried over Na₂SO₄ and concentrated to dryness to afford 3 (258 mg, 37% yield).

¹H NMR (400 MHz, CDCl₃) δ ppm 1.38-1.65 (m, 8H), 1.68-1.78 (m, 2H), 1.82-1.94 (m, 1H), 2.26 (d, J=7.3 Hz, 2H), 3.68 (s, 3H), 3.96-4.03 (m, 1H).

Step 3. Preparation of Methyl 2-(4-methylsulfonyloxycyclohexyl)acetate (4). To a solution of 3 (258 mg, 1.5 mmol, 1.0 eq.) and TEA (313.2 μL, 2.25 mmol, 1.5 eq.) in DCM (7.50 mL, 0.2 M) at 0° C. was added methanesulfonyl chloride (150.73 μL, 1.95 mmol, 1.3 eq.) dropwise. The resulting mixture was stirred at 0° C. for 30 minutes. TLC (eluting 60% EtOAc in Heptanes, revealing KMnO₄, Rf SM=0.35, Rf=0.50) showed completion of the reaction. Water was added and the aqueous phase was extracted 3× with DCM. Organic layers were washed once with 1 N aqueous HCl, dried over Na₂SO₄ and concentrated to dryness to afford 4 (375 mg, 99% yield) as a light-yellow liquid.

¹H NMR (400 MHz, CDCl₃) δ ppm 1.36-1.50 (m, 2H), 1.61-1.72 (m, 4H), 1.83-1.95 (m, 1H), 2.03-2.11 (m, 2H), 2.26 (d, J=7.1 Hz, 2H), 3.01 (s, 3H), 3.67-3.68 (m, 3H), 4.97 (br s, 1H).

Step 4. Preparation of Methyl 2-[4-[4-nitro-3-(trifluoromethyl)pyrazol-1-yl]cyclohexyl acetate (6). To a solution of 4 (207.36 mg, 0.83 mmol, 1.0 eq.) and 5 (150. mg, 0.83 mmol, 1.0 eq.) in dry DMF (2.76 mL) was added Cs₂CO₃ (539.82 mg, 1.66 mmol, 2.0 eq.). The resulting mixture was stirred at 90° C. for 24 hours. Additional 4 (207.36 mg, 0.83 mmol, 1.0 eq.) was added and the resulting mixture was stirred at 90° C. over 72 h. Water was added to the reaction mixture. The aqueous phase was extracted 3× with EtOAc. Organic layers were washed 3× with water and once with brine, dried over Na₂SO₄ and concentrated to dryness. The crude mixture was purified by flash column chromatography eluting with 0 to 35% MTBE in Heptanes (20 CVs), then 35% MTBE in Heptanes (6 CVs). The desired fractions (which eluted out at 35% MTBE in Heptanes, Rf=0.30 on TLC eluting 60% MTBE in Heptanes, revealing UV+KMnO₄) were concentrated to dryness to afford 6 (103 mg, 37% yield) as a light-yellow solid.

LCMS method 1: retention time: 1.853 min, 99.9% purity at 215 nm, [M+H]⁺=336.2.

¹H NMR (400 MHz, DMSO-d6) δ ppm 1.14-1.27 (m, 3H), 1.73-1.86 (m, 5H), 2.07-2.11 (m, 1H), 2.26 (d, J=6.8 Hz, 2H), 3.60 (s, 3H), 4.32 (tt, J=11.7, 3.5 Hz, 1H), 9.20 (s, 1H).

¹⁹F NMR (377 MHz, DMSO-d6) δ ppm −61.43 (s, 3F).

Step 5. Preparation of Methyl 2-[4-[4-amino-3-(trifluoromethyl)pyrazol-1-yl]cyclohexyl acetate (7). Nitrogen was bubbled through a solution of 6 (103 mg, 0.31 mmol, 1.0 eq.) in ethyl acetate (3 mL, 0.08 M) and methanol (1 mL, 0.08 M) for 5 minutes. Then, 10% Pd/C (65.39 mg, 0.06 mmol, 20 mol %) was added and nitrogen was bubbled through the solution for another 5 minutes. Hydrogen was then bubbled through the solution for 5 minutes, and the resulting mixture was then stirred under a hydrogen atmosphere (1 atm) for 3 hours. The reaction mixture was filtered on Celite, rinsing with EtOAc. The filtrate was concentrated under reduced pressure. The crude mixture was purified by reverse phase flash chromatography (30 g C18 gold column, elution: 45 to 70% MeOH in H₂O (0.1% formic acid) over 20 CV). The desired fractions (came out 60% MeOH in H₂O (0.1% formic acid)) were concentrated to dryness to afford 7 (64 mg, 68% yield) as a tan oil.

LCMS method 1: retention time: 1.618 min, 99.9% purity at 215 nm, [M+H]⁺=306.1.

¹H NMR (400 MHz, DMSO-d6) δ ppm 1.06-1.22 (m, 2H), 1.60-1.84 (m, 5H), 1.91-2.03 (m, 2H), 2.24 (d, J=6.6 Hz, 2H), 3.59 (s, 3H), 4.00 (ddd, J=11.9, 8.2, 3.9 Hz, 1H), 4.20 (br s, 2H), 7.23 (s, 1H).

¹⁹F NMR (377 MHz, DMSO-d6) δ ppm −58.82 (s, 3F).

Step 6. Preparation of 2-[4-[4-Amino-3-(trifluoromethyl)pyrazol-1-yl]cyclohexyl]ethanol (8). To a solution of methyl 7 (64.0 mg, 0.210 mmol, 1 eq.) in THF (0.70 mL, 0.1 M) and ethanol (1.40 mL, 0.1 M) at 0° C. was added CaCl₂) (46.53 mg, 0.420 mmol, 2.0 eq.), followed by NaBH₄ (31.72 mg, 0.840 mmol, 4.0 eq.). The resulting mixture was stirred overnight, allowing it to warm up to room temperature. After overnight stirring, NaBH₄ (10 mg, 0.210 mmol, 1.0 eq.) was added at 0° C. The resulting mixture was stirred at 0° C. for 1 hour, then at room temperature for 20 hours. Then water was added and the reaction mixture was stirred at room temperature for 1 hour. The aqueous phase was extracted 3× with EtOAc. The organics were washed with brine, dried over Na₂SO₄ and concentrated to dryness to afford 8 (49 mg, 77% yield) as a light-orange oil.

LCMS method 1: retention time: 1.412 min, 91.9% purity at 215 nm, [M+H]⁺=278.1.

¹H NMR (400 MHz, DMSO-d6) δ ppm 1.01-1.10 (m, 2H), 1.31-1.47 (m, 3H), 1.56-1.70 (m, 2H), 1.81 (br d, J=12.2 Hz, 2H), 1.95 (br s, 2H), 3.40-3.51 (m, 2H), 3.93-4.01 (m, 1H), 4.18 (s, 2H), 4.33 (t, J=5.1 Hz, 1H), 7.22 (s, 1H).

¹⁹F NMR (377 MHz, DMSO-d6) δ ppm −58.80 (s, 3F).

Step 7. Preparation of tert-Butyl N-[(3R,5R)-5-fluoro-1-[3-[[1-[4-(2-hydroxyethyl)cyclohexyl]-3-(trifluoromethyl)pyrazol-4-yl]carbamoyl]pyrazolo[1,5-a]pyrimidin-5-yl]-3-piperidyl]carbamate (10). To a solution of 8 (49.65 mg, 0.160 mmol, 1.25 eq.) and 9-intermediate 4 from synthesis of P-1-(50. mg, 0.130 mmol, 1.0 eq.) in MeCN (1 mL, 1.3 M) was added NMI (31.32 μL, 0.400 mmol, 3.5 eq.), followed by TCFH (55.47 mg, 0.200 mmol, 1.5 eq.). The resulting mixture was stirred at room temperature overnight. Nanopure water was added and the resulting suspension was stirred at 0° C. for 30 minutes. The solid was filtered on a Buchner funnel, rinsing with water, co-evaporated with MeCN and dried under high vacuum to afford 10 (81 mg, 69% yield) as a tan solid.

LCMS method 1: retention time: 1.720 min, 72.2% purity at 215 nm, [M+H]⁺=639.2.

¹H NMR (400 MHz, DMSO-d6) δ ppm 1.06 (br s, 16H), 1.60-1.91 (m, 6H), 1.99-2.20 (m, 4H), 3.41-3.49 (m, 2H), 3.60-3.71 (m, 1H), 4.19-4.29 (m, 1H), 4.35 (br t, J=5.0 Hz, 1H), 4.50-4.61 (m, 1H), 4.98-5.14 (m, 1H), 6.82-6.97 (m, 1H), 7.06-7.19 (m, 1H), 8.39 (br s, 1H), 8.82 (br d, J=8.1 Hz, 1H), 9.21 (br s, 1H).

¹⁹F NMR (377 MHz, DMSO-d6) δ ppm −185.35-−183.28 (m, 1F), −58.99 (s, 3F).

Step 8. Preparation of tert-Butyl N-[(3R,5R)-1-[3-[1-[4-[2-[4-[4-(2,6-dioxo-3-piperidyl)phenyl]piperazin-1-ylethyl]cyclohexyl]-3-(trifluoromethyl)pyrazol-4-yl]carbamoyl]pyrazolo[1,5-a]pyrimidin-5-yl]-5-fluoro-3-piperidyl]carbamate (11). To a solution of 10 (81 mg, 0.13 mmol, 1.0 eq.) in DMSO (1 mL, 1.3 M) was added IBX (46.17 mg, 0.16 mmol, 1.3 eq.). The resulting mixture was stirred at room temperature overnight. To a second solution of C-6 (56.01 mg, 0.15 mmol, 1.2 eq.) in DCM (1 mL) was added DIPEA (87.39 μL, 0.50 mmol, 4.0 eq.). The resulting mixture was stirred at room temperature for 5 minutes. NaBH(OAc)₃ (34.62 mg, 0.16 mmol, 1.3 eq.) was then added, followed by the addition of the previous DMSO solution from the IBX oxidation. The reaction mixture was stirred at room temperature for 1 hour. DCE was evaporated under reduced pressure. The crude mixture was purified by column chromatography C18 (30 g C18 gold column, elution: 5 to 50% MeCN in H₂O (0.1% formic acid) over 15 CV). The desired fractions (came out 40% MeCN in H₂O (0.1% formic acid)) were concentrated to dryness to afford 11 (39 mg, 34% yield) as a yellow semi-solid.

LCMS method 1: retention time: 1.457 min, 98.5% purity at 215 nm, [M+H]⁺=894.2.

¹H NMR (400 MHz, DMSO-d6) δ ppm 1.07-1.45 (m, 15H), 1.68-1.92 (m, 6H), 1.96-2.16 (m, 6H), 2.36-2.42 (m, 2H), 2.56-2.64 (m, 2H), 3.12 (br s, 5H), 3.45-3.55 (m, 1H), 3.58-3.68 (m, 1H), 3.72 (br dd, J=10.8, 4.4 Hz, 1H), 4.19-4.28 (m, 1H), 4.52-4.63 (m, 1H), 4.97-5.15 (m, 2H), 6.83-6.95 (m, 3H), 7.00-7.14 (m, 3H), 8.14 (s, 1H), 8.29 (s, 1H), 8.39 (br s, 1H), 8.82 (br d, J=7.3 Hz, 1H), 9.21 (br d, J=1.0 Hz, 1H), 10.77 (s, 1H).

¹⁹F NMR (377 MHz, DMSO-d6) δ ppm −184.67 (s, 1F), −58.99 (s, 3F).

Step 9. Preparation of 5-((3R,5R)-3-Amino-5-fluoropiperidin-1-yl)-N-(1-((1r,4r)-4-(2-(4-(4-(2,6-dioxopiperidin-3-yl)phenyl) piperazin-1-yl)ethyl)cyclohexyl)-3-(trifluoromethyl)-1H-pyrazol-4-yl) pyrazolo[1,5-a]pyrimidine-3-carboxamide hydrochloride (P-11). A solution of tert-butyl 11 (39. mg, 0.04 mmol, 1.0 eq.) in a 4 M HCl solution in dioxane (1.64 mL, 6.54 mmol, 150 eq.) was stirred at room temperature for 30 minutes. The crude mixture was purified by column chromatography C18 (30 g C18 gold column, elution: 5 to 40% MeCN in H₂O (0.02M in HCl) over 15 CV). The desired fractions (came out 32% MeCN in H₂O (0.02M in HCl)) were concentrated to dryness and freeze-dried to afford P-11 (21.3 mg, 61% yield) as a white solid

LCMS method 3: retention time: 1.917 min, 99.9% purity at 215 nm, [M−HCl+H]⁺=794.4, [M−HCl+2H]²⁺=397.8.

¹H NMR (400 MHz, DMSO-d6) δ ppm 1.09-1.28 (m, 2H), 1.37-1.50 (m, 1H), 1.64-2.23 (m, 11H), 2.34-2.48 (m, 2H), 2.59-2.72 (m, 1H), 3.03-3.25 (m, 6H), 3.26-3.34 (m, 1H), 3.41-3.67 (m, 4H), 3.71-3.90 (m, 3H), 4.25-4.38 (m, 1H), 4.47-4.97 (m, 2H), 5.12 (d, J=46.0 Hz, 1H), 6.92 (d, J=7.8 Hz, 1H), 6.97 (d, J=8.8 Hz, 2H), 7.12 (d, J=8.6 Hz, 2H), 8.28-8.53 (m, 4H), 8.94 (d, J=7.8 Hz, 1H), 9.23 (s, 1H), 10.40 (br s, 1H), 10.79 (s, 1H).

¹⁹F NMR (377 MHz, DMSO-d6) δ ppm −184.93 (s, 1F), 58.81 (s, 3F).

### Example S10. Synthesis of P-21

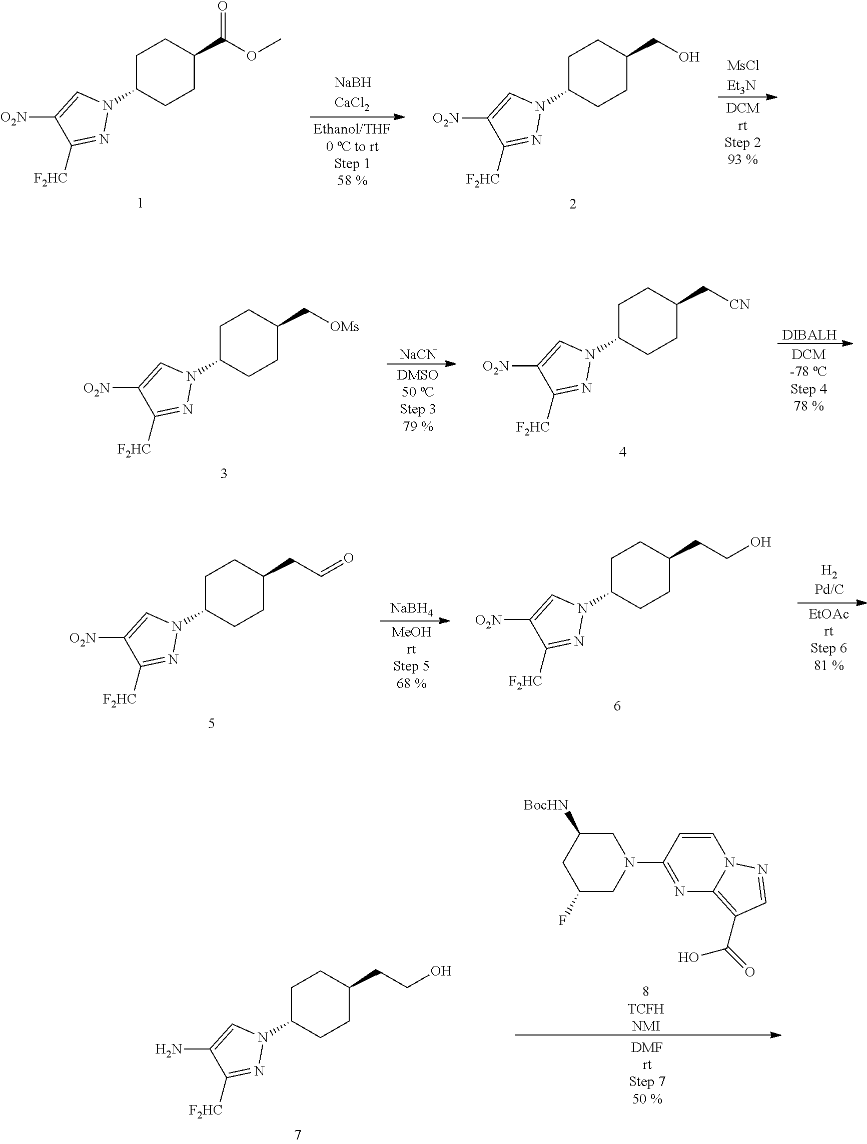

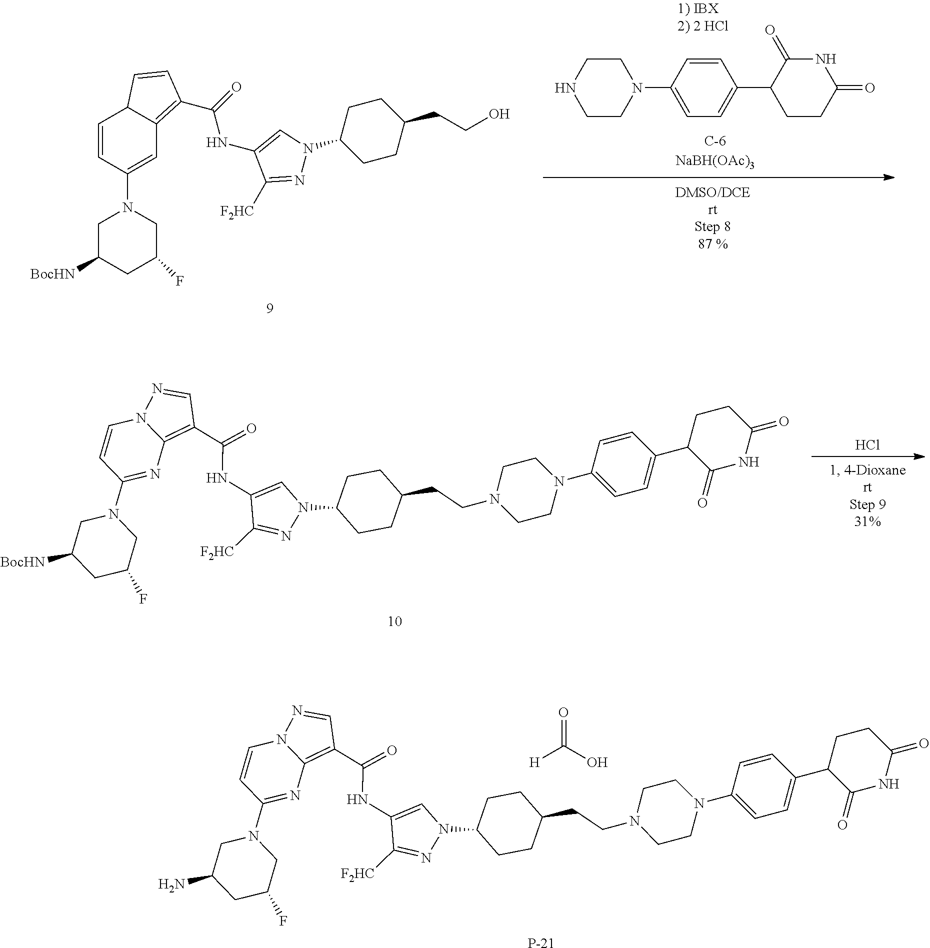

Step 1. Preparation of [4-[3-(Difluoromethyl)-4-nitro-pyrazol-1-yl]cyclohexyl]methanol (2). To a solution of methyl 4-[3-(difluoromethyl)-4-nitro-pyrazol-1-yl]cyclohexanecarboxylate 1-intermediate 4 from synthesis of T-2-(730.0 mg, 2.26 mmol, 1 eq.) in 1:2 THF (3.8 mL)/ethanol (7.5 mL) at 0° C. was added CaCl₂) (501.2 mg, 4.52 mmol, 2 eq.) followed by the addition of NaBH₄ (341.6 mg, 9.03 mmol, 4 eq.). The resulting mixture was stirred at rt. After 16 h at rt, LCMS showed total conversion. The reaction was quenched by adding water dropwise and stirring at rt for 1 h. Then, product was extracted three times with EtOAc, and the organic layers were combined and washed once with water and once with brine, and finally dried over MgSO₄. EtOAc was removed under reduced pressure and the residue was purified by normal phase flash chromatography (80 g silica column, liquid deposit (DCM), elution: 0% EtOAc/Heptane over 3 CV, then 0 to 100% EtOAc/Heptane over 10 CV, then 100% EtOAc/Heptane over 3 CV (desired product was released around 50% EtOAc). Fractions were combined and concentrated to give 2 (371.9 mg, 58% yield) as a colorless oil.

LCMS method 1: 97.5% purity at 215 nm, [M+H]⁺=276.2.

¹H NMR (400 MHz, CDCl₃-d) δ ppm 1.14-1.30 (m, 3H), 1.70-1.87 (m, 2H), 1.97-2.09 (m, 2H), 2.25-2.34 (m, 2H), 3.54 (d, J=6.1 Hz, 2H), 4.18 (tt, J=12.1, 3.9 Hz, 1H), 7.10 (t, J=53.3 Hz, 1H), 8.22 (s, 1H).

¹⁹F NMR (377 MHz, CDCl₃-d) δ ppm −117.64 (d, J=53.13, 2F).

Step 2. Preparation of [4-[3-(Difluoromethyl)-4-nitro-pyrazol-1-yl]cyclohexyl]methyl methanesulfonate (3). To a solution of [4-[3-(difluoromethyl)-4-nitro-pyrazol-1-yl]cyclohexyl]methanol 2 (428.5 mg, 1.52 mmol) and Et₃N (0.28 mL, 1.98 mmol) in DCM (7.62 mL) at 0° C. was added dropwise MsCl (0.13 mL, 1.68 mmol). Then, the reaction slowly warm up to rt and stirred at this temperature. After 3h, TLC (3:7 Heptane/EtOAc) showed completion of the reaction. The reaction was then partitioned between water and EtOAc. The phases were separated and the organic phase was washed three times with water, once with 1 N HCl and once with brine, before being dried over MgSO₄. EtOAc was removed under reduced pressure to give 3 (529 mg, 93% yield) as a light-yellow oil. Product was used without purification in the next step.

LCMS method 1: 94.8% purity at 215 nm, [M+H]⁺=354.0.

¹H NMR (400 MHz, CDCl₃-d) δ ppm 1.22-1.37 (m, 3H), 1.80-1.90 (m, 2H), 2.05-2.13 (m, 2H), 2.27-2.36 (m, 2H), 3.04 (s, 3H), 4.08-4.14 (m, 2H), 4.15-4.24 (m, 1H), 7.11 (t, J=53.4 Hz, 1H), 8.22 (s, 1H).

¹⁹F NMR (377 MHz, CDCl₃-d) δ ppm −117.64 (d, J=53.13, 2F).

Step 3. Preparation of 2-[4-[3-(Difluoromethyl)-4-nitro-pyrazol-1-yl]cyclohexyl]acetaldehyde (4). A solution of [4-[3-(difluoromethyl)-4-nitro-pyrazol-1-yl]cyclohexyl]methyl methanesulfonate 3 (529.0 mg, 1.5 mmol, 1 eq.) and NaCN (183.4 mg, 3.74 mmol, 2.5 eq.) in DMSO (7.49 mL) was heated to 50° C. and stirred at this temperature. After 16 h at 50° C., LCMS showed total completion of the reaction. The reaction mixture was cooled down to rt and was quenched with water under vigorous agitation. Then, EtOAc was added, and the product was extracted two times with EtOAc. The combined organic layers were then washed once with a concentrated NaHCO₃ aqueous solution and once with brine. Finally, the product solution was dried over MgSO₄ and EtOAc was removed under reduced pressure to give 4 (342 mg, 79% yield) as a yellow oil.

LCMS method 1: 98.8% purity at 215 nm, [M+H]⁺=285.2.

¹H NMR (400 MHz, CDCl₃-d) δ ppm 1.30-1.46 (m, 2H), 1.76-1.96 (m, 3H), 2.06-2.16 (m, 2H), 2.29-2.37 (m, 2H), 2.37-2.40 (m, 2H), 4.20 (tt, J=12.0, 3.9 Hz, 1H), 7.12 (t, J=54.2 Hz, 1H), 8.22 (s, 1H).

¹⁹F NMR (377 MHz, CDCl₃-d) δ ppm −117.68 (s, 2F).

Step 4. Preparation of 2-[4-[3-(Difluoromethyl)-4-nitro-pyrazol-1-yl]cyclohexyl]acetaldehyde (5). To a solution of 2-[4-[3-(difluoromethyl)-4-nitro-pyrazol-1-yl]cyclohexyl]acetonitrile 4 (342 mg, 1.18 mmol, 1 eq.) in DCM (5.88 mL) was added a 1 M solution of DIBAL-H in DCM (3.53 mL, 3.53 mmol, 3 eq.) at −78° C. The reaction was then stirred at that temperature. After 2 h, LCMS showed total completion of the reaction. The reaction was slowly quenched with a solution of Rochelle salt at −78° C., and this was stirred for 1 h at rt. Then, the product was extracted three times with EtOAc, and the combined organic layers were washed two times with 1 M HCl and once with brine. Finally, the product solution was dried over MgSO₄ and EtOAc was removed under reduced pressure to give 5 (264 mg, 78% yield) as a yellow oil.

LCMS method 1: 99.9% purity at 215 nm, [M+H]⁺=288.2.

¹H NMR (400 MHz, DMSO-d6) δ ppm 1.36 (q, J=6.5 Hz, 2H), 1.75 (qd, J=12.5, 3.2 Hz, 2H), 1.85 (d, J=12.5 Hz, 2H), 2.02-2.15 (m, 2H), 3.40-3.50 (m, 2H), 4.19-4.32 (m, 1H), 4.32-4.40 (m, 1H), 7.30 (t, J=52.3 Hz, 1H), 9.06 (s, 1H).

¹⁹F NMR (377 MHz, DMSO-d6) δ ppm −117.36 (s, 2F).

Step 5. Preparation of 2-[4-[3-(Difluoromethyl)-4-nitro-pyrazol-1-yl]cyclohexyl]ethanol (6). To a solution of 2-[4-[3-(difluoromethyl)-4-nitro-pyrazol-1-yl]cyclohexyl]acetaldehyde 5 (264 mg, 0.92 mmol, 1 eq.) in methanol (4.59 mL) at 0° C. was added NaBH₄ (69.52 mg, 1.84 mmol). The resulting mixture was stirred at rt After 16 h. LCMS showed total conversion. Water was added and the mixture was stirred at rt for 1 h. Product was extracted three times with EtOAc and the combined organic layers were washed twice with brine. Finally, the product solution was dried over MgSO₄ and EtOAc was removed under reduced pressure to give 6 (216 mg, 68% yield) as a yellow oil.

LCMS method 1: 83.3% purity at 215 nm, [M+H]⁺=290.2

¹H NMR (400 MHz, DMSO-d6) δ ppm 1.03-1.15 (m, 2H), 1.36 (q, J=6.5 Hz, 2H), 1.41-1.52 (m, 1H), 1.69-1.81 (m, 2H), 1.85 (br d, J=12.5 Hz, 2H), 2.02-2.12 (m, 2H), 3.37-3.52 (m, 2H), 4.22-4.32 (m, 1H), 4.33-4.39 (m, 1H), 7.30 (t, J=53.2 Hz, 1H), 9.06 (s, 1H).

¹⁹F NMR (377 MHz, DMSO-d6) δ ppm −117.36 (s, 2F).

Step 6. Preparation of 2-[4-[4-Amino-3-(difluoromethyl)pyrazol-1-yl]cyclohexyl]ethanol (7). N₂ was bubbled through a solution of 2-[4-[3-(difluoromethyl)-4-nitro-pyrazol-1-yl]cyclohexyl]ethanol 6 (216.1 mg, 0.75 mmol, 1 eq.) in EtOAc (3.74 mL) for 5 min. Then 10% Pd/C (238.5 mg, 0.22 mmol, 0.3 eq.) was added and N₂ was bubbled through the solution for another 5 min. Then H₂ was bubbled through the solution for 5 min and the resulting mixture was stirred at rt under 1 atm H₂. After 3 h, LCMS showed total conversion. The solution was filtered through a celite pad, and washed thoroughly with EtOAc. Finally, the filtrate was concentrated under reduced pressure to give 7 (169 mg, 81% yield) as a yellow oil. The product was used without purification in the next step.

LCMS method 1: 92.9% purity at 215 nm, [M+H]⁺=260.2

Step 7. Preparation of tert-Butyl N-[(3R,5R)-1-[3-[[3-(difluoromethyl)-1-[4-(2-hydroxyethyl)cyclohexyl]pyrazol-4-yl]carbamoyl]pyrazolo[1,5-a]pyrimidin-5-yl]-5-fluoro-3-piperidyl]carbamate (9). To a solution of 2-[4-[4-amino-3-(difluoromethyl)pyrazol-1-yl]cyclohexyl]ethanol 7 (168.8 mg, 0.65 mmol, 1.3 eq.), 5-[(3R,5R)-3-(tert-butoxycarbonylamino)-5-fluoro-1-piperidyl]pyrazolo[1,5-a]pyrimidine-3-carboxylic acid 8-intermediate 4 from synthesis of P-1-(190 mg, 0.50 mmol, 1 eq.) and NMI (0.14 mL, 1.75 mmol, 3.5 eq.) in MeCN (2.50 mL) was added TCFH (168.6 mg, 0.60 mmol, 1.2 eq.) at 0° C. The reaction was then stirred at rt. After 16 h, LCMS showed total completion of the reaction. MeCN was removed under reduced pressure and the residue was purified by reverse phase flash chromatography (30 g C18 column, liquid deposit (DMSO), elution: 5% MeOH/0.1% HCOOH over 3 CV, then 5 to 100% MeOH/0.1% HCOOH over 15 CV, then 100% MeOH/0.1% HCOOH over 3 CV (desired product was released around 80% MeCN)). Fractions were combined and concentrated to give 9 (155.6 mg, 50% yield) as an off-white solid.

LCMS method 1: 99.9% purity at 215 nm, [M+H]⁺=621.2

¹H NMR (400 MHz, DMSO-d6) δ ppm 1.04-1.17 (m, 3H), 1.29-1.44 (m, 12H), 1.65-1.77 (m, 3H), 1.80-1.93 (m, 3H), 1.97-2.07 (m, 2H), 2.10-2.18 (m, 1H), 2.94-3.14 (m, 1H), 3.36-3.53 (m, 3H), 3.61-3.72 (m, 1H), 4.11-4.23 (m, 1H), 4.31-4.40 (m, 1H), 4.94-5.13 (m, 1H), 6.83-6.90 (m, 1H), 6.91-7.23 (m, 2H), 8.28 (s, 1H), 8.33 (br s, 1H), 8.82 (d, J=8.1 Hz, 1H), 9.31 (br s, 1H).

¹⁹F NMR (377 MHz, DMSO-d6) δ ppm −184.27-−183.87 (m, 1F), −112.01-−110.10 (m, 2F).

Step 8. Preparation of tert-Butyl N-[(3R,5R)-1-[3-[3-(difluoromethyl)-1-[4-[2-[4-[4-(2,6-dioxo-3-piperidyl)phenyl]piperazin-1-ylethyl]cyclohexyl]pyrazol-4-yl]carbamoyl]pyrazolo[1,5-a]pyrimidin-5-yl]-5-fluoro-3-piperidyl]carbamate (10). To a solution of tert-butyl N-[(3R,5R)-1-[3-[[3-(difluoromethyl)-1-[4-(2-hydroxyethyl)cyclohexyl]pyrazol-4-yl]carbamoyl]pyrazolo[1,5-a]pyrimidin-5-yl]-5-fluoro-3-piperidyl]carbamate 9 (140 mg, 0.23 mmol, 1 eq.) in dry DMSO (2.2 mL) was added IBX (75.8 mg, 0.27 mmol, 1.2 eq.). The resulting mixture was stirred at rt. After 16 h, LCMS showed total conversion to the aldehyde. To a solution of 3-(4-Piperazin-1-ylphenyl) piperidine-2,6-dione;dihydrochloride C-6 (43.0 mg, 0.12 mmol, 1.1 eq.) in DCE (1.0 mL) was added DIPEA (196 μL, 1.13 mmol, 10 eq.). The mixture was stirred at rt for 10 minutes, and the previous aldehyde solution was added. After 10 minutes of stirring, NaBH(OAc)₃ (31.1 mg, 0.15 mmol, 1.3 eq.) was added. The resulting mixture was then stirred at room temperature. After 1 h, LCMS showed total completion of the reaction. DCE was removed under reduced pressure and the residue was purified by reverse phase flash chromatography (50 g C18 column, liquid deposit (DMSO), elution: 5% MeCN/0.1% HCOOH over 3 CV, then 5 to 35% MeCN/0.1% HCOOH over 15 CV, then 35 to 100% MeCN/0.1% HCOOH over 1 CV, then 100% MeCN/0.1% HCOOH over 3 CV (desired product was released around 30% MeCN)). Fractions were combined and concentrated to give 10 (86 mg, 87% yield) as a white solid. Product was used without purification in the next step.

LCMS method 1: 99.9% purity at 215 nm, [M+H]⁺=876.4

Step 9. Preparation of N-[3-(Difluoromethyl)-1-[4-[2-[4-[4-(2,6-dioxo-3-piperidyl)phenyl]piperazin-1-ylethyl]cyclohexyl]pyrazol-4-yl]-5-[(3R,5R)-3-amino-5-fluoro-1-piperidyl]pyrazolo[1,5-a]pyrimidine-3-carboxamide formic acid salt (P-21). tert-butyl N-[(3R,5R)-1-[3-[3-(difluoromethyl)-1-[4-[2-[4-[4-(2,6-dioxo-3-piperidyl)phenyl]piperazin-1-yl]ethyl]cyclohexyl]pyrazol-4-yl]carbamoyl]pyrazolo[1,5-a]pyrimidin-5-yl]-5-fluoro-3-piperidyl]carbamate 10 (86 mg, 0.10 mmol, 1 eq.) was solubilized in 4 M HCl in 1,4-dioxane (3.68 mL, 14.73 mmol, 150 eq.). The mixture was stirred at rt. After 5h, LCMS showed total completion of the reaction. Volatiles were evaporated under vacuum. The resulting residue was purified by reverse phase flash chromatography (30 g C18 RediSep Rf Gold column, liquid deposit (Water), elution: 5% MeCN/0.02 M HCl over 5 CV, then 5 to 20% MeCN/0.02 M HCl over 15 CV, then 20% MeCN/0.02 M HCl over 5 CV, then 20 to 100% MeCN/0.02 M over 2 CV then 100% MeCN/0.02 M over 7 CV). Fractions were combined, concentrated and lyophilised to give P-21 (24.1 mg, 31% yield) as a white solid as a formic acid salt.

LCMS method 3:98.7% purity at 215 nm, [M−HCOOH+2H]²⁺=388.7; [M−HCOOH+H]⁺=776.5.

¹H NMR (400 MHz, DMSO-d6) δ ppm 1.02-1.24 (m, 2H), 1.32-1.49 (m, 3H), 1.54-1.81 (m, 3H), 1.83-1.94 (m, 2H), 1.97-2.25 (m, 5H), 2.30-2.40 (m, 2H), 2.41-2.49 (m, 2H), 2.58-2.70 (m, 1H), 2.86-2.97 (m, 1H), 2.99-3.07 (m, 1H), 3.08-3.16 (m, 4H), 3.21-3.54 (m, 4H), 3.72 (dd, J=10.9, 5.0 Hz, 1H), 4.13-4.24 (m, 1H), 4.26-4.88 (m, 2H), 5.01 (d, J=47.0 Hz, 1H), 6.80-6.96 (m, 3H), 6.98-7.31 (m, 3H), 8.20 (s, 1H), 8.27 (s, 1H), 8.38 (s, 1H), 8.81 (d, J=7.8 Hz, 1H), 9.33 (s, 1H), 10.77 (s, 1H).

¹⁹F NMR (377 MHz, DMSO-d6) δ ppm −182.92 (s, 1F), −111.03 (s, 2F).

### Example S11. Synthesis of P-23

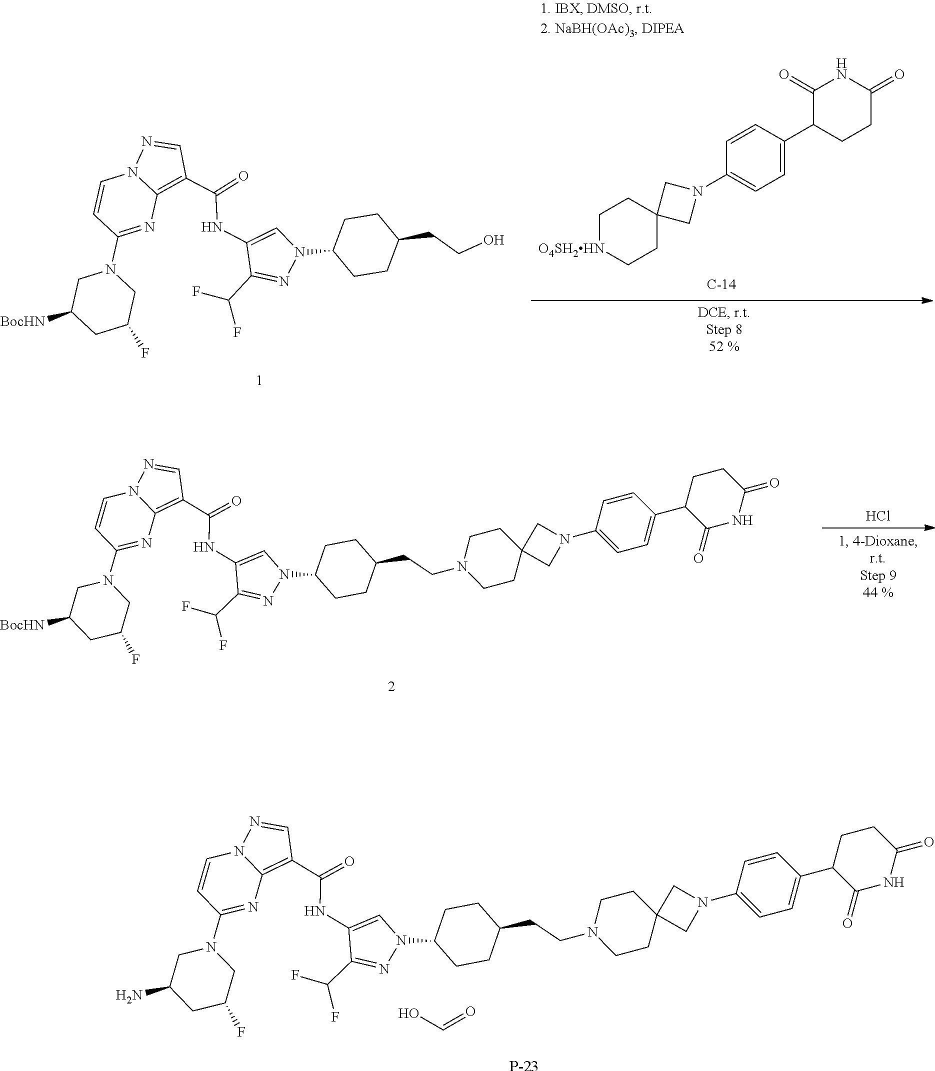

### Step 8. Preparation of tert-Butyl N-[(3R,5R)-1-[3-[[3-(difluoromethyl)-1-[4-[2-[2-[4-(2,6-dioxo-3-piperidyl)phenyl]-2,7-diazaspiro[3.5]nonan-7-yl]ethyl]cyclohexyl]pyrazol-4-yl]carbamoyl]pyrazolo[1,5-a]pyrimidin-5-yl]-5-fluoro-3-piperidyl]carbamate (2)

Preparation of Solution A: To a solution of tert-butyl N-[(3R,5R)-1-[3-[[3-(difluoromethyl)-1-[4-(2-hydroxyethyl)cyclohexyl]pyrazol-4-yl]carbamoyl]pyrazolo[1,5-a]pyrimidin-5-yl]-5-fluoro-3-piperidyl]carbamate 1-intermediate 9 from synthesis of P-21-(60 mg, 0.097 mmol, 1 eq.) in anhydrous DMSO (0.6 mL) was added IBX (33 mg, 0.116 mmol, 1.2 eq.). The resulting mixture was stirred at room temperature. After 18 h, LCMS showed full conversion toward tert-butyl N-[(3R,5R)-1-[3-[[3-(difluoromethyl)-1-[4-(2-oxoethyl)cyclohexyl]pyrazol-4-yl]carbamoyl]pyrazolo[1,5-a]pyrimidin-5-yl]-5-fluoro-3-piperidyl]carbamate.

To a solution of 3-[4-(2,7-diazaspiro[3.5]nonan-2-yl)phenyl]piperidine-2,6-dione sulfuric acid C-14 (65 mg, 0.160 mmol, 1.6 eq.) in DCE (2 mL) was added DIPEA (0.17 mL, 0.970 mmol, 10 eq.). The reaction was stirred at room temperature for 10 min followed by the addition of Solution A. After 10 min., NaBH(OAc)₃ (26.7 mg, 0.130 mmol, 1.3 eq.) was added and the reaction was stirred at room temperature. After 72 h, LCMS showed full conversion. Volatiles were removed under reduce pressure and the residue was purified by reverse phase flash chromatography (30 g C18 gold column, liquid deposit (DMSO), elution: 5% MeCN/0.1% HCOOH over 3 CV, then 5 to 35% MeCN/0.1% HCOOH over 8 CV, then 35% MeCN/0.1% HCOOH over 3 CV, then 35 to 100% MeCN/0.1% HCOOH over 6 CV). Fractions were combined and concentrated to give 2 (46.3 mg, 52% yield) as a white solid.

LCMS method 1: 99.0% purity at 215 nm, [M+H]⁺=917.4.

¹H NMR (400 MHz, DMSO-d6) δ ppm 1.07-1.17 (m, 2H), 1.28-1.43 (m, 13H), 1.69-1.79 (m, 7H), 1.80-1.91 (m, 3H), 1.95-2.17 (m, 7H), 2.28-2.40 (m, 6H), 2.57-2.65 (m, 1H), 3.51 (s, 4H), 3.69 (br dd, J=11.0, 4.9 Hz, 2H), 4.13-4.22 (m, 1H), 4.95-5.12 (m, 1H), 6.38 (d, J=8.6 Hz, 2H), 6.89 (br d, J=7.8 Hz, 1H), 6.99 (d, J=8.6 Hz, 2H), 7.03-7.14 (m, 1H), 8.16 (s, 1H), 8.28 (s, 1H), 8.33 (br s, 1H), 8.82 (d, J=8.1 Hz, 1H), 9.31 (br s, 1H), 10.74 (s, 1H).

Step 9. Preparation of N-[3-(Difluoromethyl)-1-[4-[2-[2-[4-(2,6-dioxo-3-piperidyl)phenyl]-2,7-diazaspiro[3.5]nonan-7-yl]ethyl]cyclohexyl]pyrazol-4-yl]-5-[(3R,5R)-3-amino-5-fluoro-1-piperidyl]pyrazolo[1,5-a]pyrimidine-3-carboxamide formic acid salt (P-23). A solution of tert-butyl N-[(3R,5R)-1-[3-[[3-(difluoromethyl)-1-[4-[2-[2-[4-(2,6-dioxo-3-piperidyl)phenyl]-2,7-diazaspiro[3.5]nonan-7-yl]ethyl]cyclohexyl]pyrazol-4-yl]carbamoyl]pyrazolo[1,5-a]pyrimidin-5-yl]-5-fluoro-3-piperidyl]carbamate 6 (46.3 mg, 0.051 mmol, 1 eq.) in a 4 M HCl solution in 1,4-dioxane (4 mL, 16.000 mmol, 310 eq.) was stirred at room temperature. After 18 h, HPLC showed full conversion. Solvents were removed under reduced pressure and the residue was purified by reverse phase flash chromatography (30 g C18 gold column, liquid deposit (H₂O), elution: 5% MeCN/0.02 M HCl over 3 CV, then 5 to 20% MeCN/0.02 M HCl over 15 CV). Fractions were combined and concentrated. The residue was purified by preparative HPLC (0.1% formic acid in MeCN/H₂O). Fractions were combined, concentrated and lyophilised to give P-23 (18.5 mg, 44% yield) as a white solid as a formic acid salt.

LCMS method 3:98.7% purity at 215 nm, [M−HCOOH+H]⁺=816.5.

¹H NMR (400 MHz, DMSO-d6) δ ppm 1.04-1.20 (m, 2H), 1.29-1.43 (m, 3H), 1.56-1.78 (m, 6H), 1.82-1.93 (m, 2H), 1.94-2.24 (m, 5H), 2.28-2.41 (m, 5H), 2.41-2.48 (m, 1H), 2.55-2.70 (m, 1H), 2.85-2.98 (m, 1H), 3.03-3.10 (m, 1H), 3.30-3.42 (m, 5H), 3.48-3.52 (m, 4H), 3.69 (dd, J=10.9, 5.0 Hz, 1H), 4.13-4.24 (m, 1H), 4.29-4.55 (m, 1H), 4.60-4.85 (m, 1H), 5.01 (d, J=48.0 Hz, 1H), 6.38 (d, J=8.6 Hz, 2H), 6.92 (d, J=8.1 Hz, 1H), 6.95-7.31 (m, 3H), 8.19 (s, 1H), 8.28 (s, 1H), 8.37 (s, 1H), 8.81 (d, J=8.1 Hz, 1H), 9.33 (s, 1H), 10.74 (s, 1H).

¹⁹F NMR (377 MHz, DMSO-d6) δ ppm −183.46 (s, 1F), −110.98 (s, 2F).

### Example S12. Synthesis of P-24

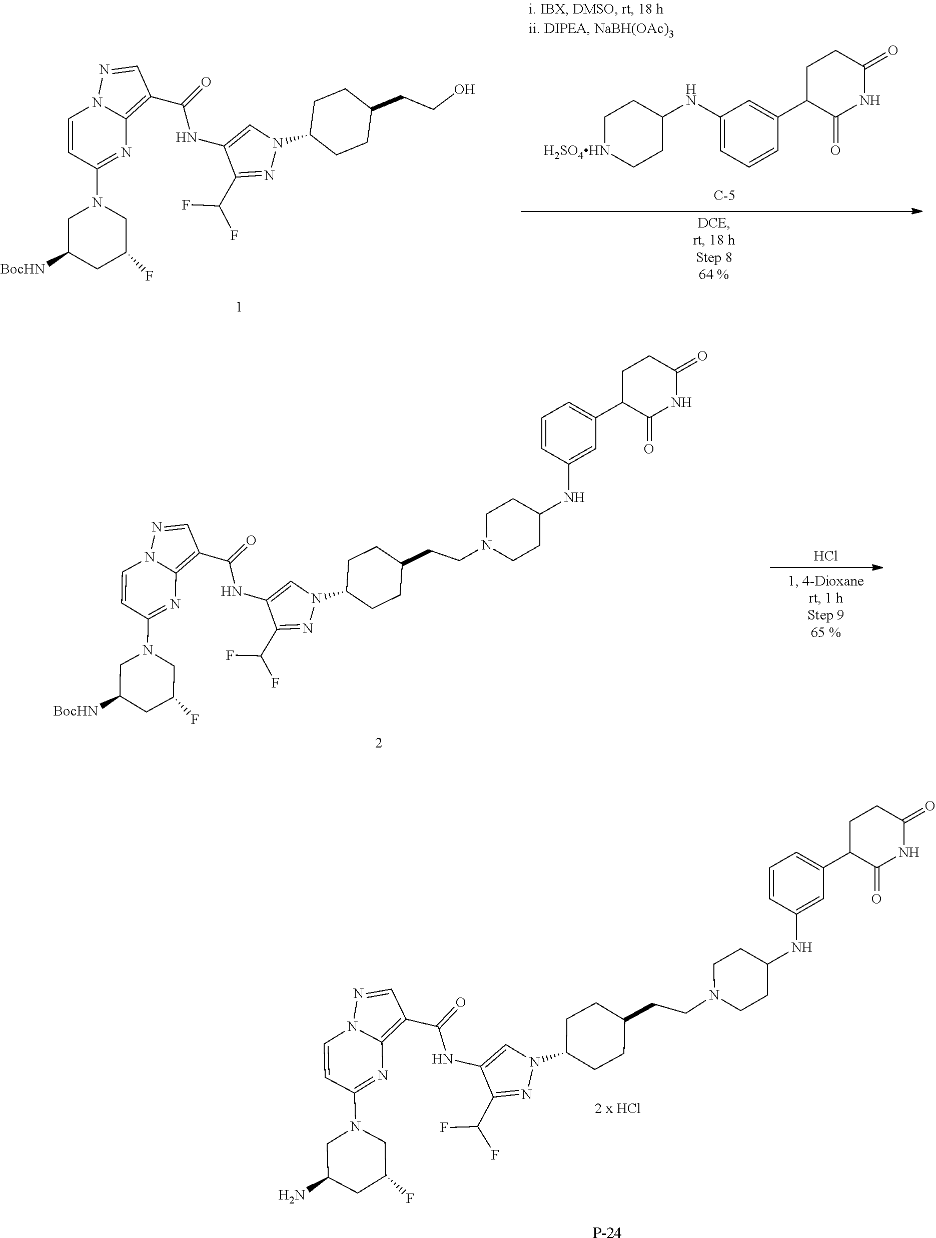

Step 8. Preparation of tert-Butyl N-[(3R,5R)-1-[3-[[3-(difluoromethyl)-1-[4-[2-[4-[3-(2,6-dioxo-3-piperidyl)anilino]-1-piperidyl]ethyl]cyclohexyl]pyrazol-4-yl]carbamoyl]pyrazolo[1,5-a]pyrimidin-5-yl]-5-fluoro-3-piperidyl]carbamate (2). IBX (37.9 mg, 0.135 mmol, 1.2 equiv) was added to a solution of tert-butyl N-[(3R,5R)-1-[3-[[3-(difluoromethyl)-1-[4-(2-hydroxyethyl)cyclohexyl]pyrazol-4-yl]carbamoyl]pyrazolo[1,5-a]pyrimidin-5-yl]-5-fluoro-3-piperidyl]carbamate 1-intermediate 9 from synthesis of P-21-(70.0 mg, 0.113 mmol, 1.0 equiv) in DMSO (1 mL). The reaction mixture was then stirred at room temperature overnight under nitrogen atmosphere. DIPEA (196 μL, 1.13 mmol, 10.0 equiv) was added to a suspension of 3-[3-(piperidin-1-ium-4-ylamino)phenyl]piperidine-2,6-dione hydrogen sulfate C-5 (47.8 mg, 0.124 mmol, 1.1 equiv) in DCE (1 mL), which was stirred for 10 minutes under nitrogen atmosphere. Subsequently, the DMSO solution containing the aldehyde (oxidized 7) (69.7 mg, 0.113 mmol, 1.0 equiv) prepared beforehand was added to the reaction mixture and stirring was continued for an additional 30 minutes, after which sodium triacetoxyborohydride (31.0 mg, 0.146 mmol, 1.3 equiv) was added in one portion. The reaction mixture was stirred at room temperature overnight under nitrogen atmosphere. The volatiles were evaporated under reduced pressure, then the remaining DMSO solution was injected directly in a column for reversed-phase flash chromatography purification (MeCN in 0.1% HCOOH(aq), 5% (3 CV)→50%, 30 g RediSep Rf Gold® C18Aq, 20 CV, λ=214-254 nm, product with 36-41% MeCN). Co-evaporation of the fractions with MeCN yielded 2 (86 mg, 0.072 mmol, 64% yield) as a white solid.

LCMS method 1:75.0% purity at 215 nm, [M+H]⁺=890.6, [M+2H]²⁺=446.0.

¹H NMR (400 MHz, DMSO-d6) δ ppm 0.96-1.20 (m, 4H), 1.34 (br s, 9H), 1.39-1.44 (m, 2H), 1.72-1.87 (m, 8H), 1.95-2.07 (m, 4H), 2.09-2.22 (m, 2H), 2.42-2.47 (m, 3 H), 2.58-2.72 (m, 1H), 2.98-3.09 (m, 1H), 3.40 (br d, J=13.7 Hz, 2H), 3.52 (s, 4H), 3.66 (br d, J=3.7 Hz, 1H), 3.73 (br dd, J=10.8, 4.9 Hz, 1H), 4.18 (br t, J=11.7 Hz, 1H), 4.24-4.51 (m, 1H), 4.59-4.90 (m, 1H), 4.93-5.15 (m, 1H), 6.25 (s, 1H), 6.31 (br d, J=7.8 Hz, 1H), 6.49 (d, J=7.5 Hz, 1H), 6.83-6.96 (m, 1H), 7.02-7.25 (m, 3H), 8.28 (s, 1H), 8.33 (br s, 1H), 8.82 (d, J=7.8 Hz, 1H), 9.31 (br s, 1H), 10.79 (s, 1H).

¹⁹F NMR (377 MHz, DMSO-d6) δ ppm −186.03-−182.66 (m, 1F), −114.14-−108.43 (m, 2F).

Step 9. Preparation of 5-((3R,5R)-3-Amino-5-fluoropiperidin-1-yl)-N-(3-(difluoromethyl)-1-((1r,4r)-4-(2-(4-((3-(2,6-dioxopiperidin-3-yl)phenyl)amino) piperidin-1-yl)ethyl)cyclohexyl)-1H-pyrazol-4-yl) pyrazolo[1,5-a]pyrimidine-3-carboxamide dihydrochloride (P-24). HCl 4.0 M in 1,4-dioxane (2.62 mL, 10.5 mmol, 150 equiv) was added to tert-butyl N-[(3R,5R)-1-[3-[3-(difluoromethyl)-1-[4-[2-[4-[3-(2,6-dioxo-3-piperidyl)anilino]-1-piperidyl]ethyl]cyclohexyl]pyrazol-4-yl]carbamoyl]pyrazolo[1,5-a]pyrimidin-5-yl]-5-fluoro-3-piperidyl]carbamate 2 (83.0 mg, 0.070 mmol, 1.0 equiv), and the mixture was sonicated for 30 minutes, then stirred for 1 hour. The solvent was evaporated to dryness and the residue was purified by reversed-phase flash chromatography (MeCN in 0.02 M HCl(aq), 5% (3CV)→50%, 50 g RediSep Rf Gold® C18Aq, 15 CV, λ=214-254 nm, product with 24-30% MeCN, broad band) to yield the impure product (69 mg). The pure product was obtained after prep HPLC purification (MeCN in HCl(aq)): the fractions containing the product were evaporated to dryness, then co-evaporated with water (3×10 mL) to completely remove any trace of residual HCl, and the residue was freeze-dried overnight to afford P-24 (36.38 mg, 0.0458 mmol, 65% yield) as a white solid as a dihydrochloride.

LCMS method 3:99.4% purity at 215 nm, [M−2HCl+H]⁺=790.4, [M−2HCl+2H]²⁺=395.7.

¹H NMR (400 MHz, DMSO-d6) δ ppm 1.05-1.27 (m, 2H), 1.31-1.52 (m, 1H), 1.56-1.70 (m, 2H), 1.70-1.82 (m, 2H), 1.82-1.99 (m, 5H), 2.00-2.25 (m, 7H), 2.34-2.47 (m, 1H), 2.59-2.76 (m, 1H), 2.85-3.19 (m, 4H), 3.20-3.43 (m, 4H), 3.44-3.66 (m, 3H), 3.66-3.93 (m, 1H), 4.15-4.32 (m, 1H), 4.55-4.74 (m, 1H), 4.77-4.97 (m, 1H), 5.10 (d, J=45.8 Hz, 1H), 6.52-6.97 (m, 4H), 6.99-7.31 (m, 2H), 8.27-8.40 (m, 2H), 8.41-8.64 (m, 3H), 8.93 (d, J=7.8 Hz, 1H), 9.33 (s, 1H), 10.36-10.70 (m, 1H), 10.76-10.93 (m, 1H).

¹⁹F NMR (377 MHz, DMSO-d6) δ ppm −184.60 (s, 1F), −111.27 (s, 2F).

The following compounds were synthesized via the same general routes with modifications to CBM (C-X) in step 8 (Table 7).

TABLE 7

| Com- | | |
|---|---|---|
| pound | | |
| No. | CBM | Structure |
| P-11 | C-6 | 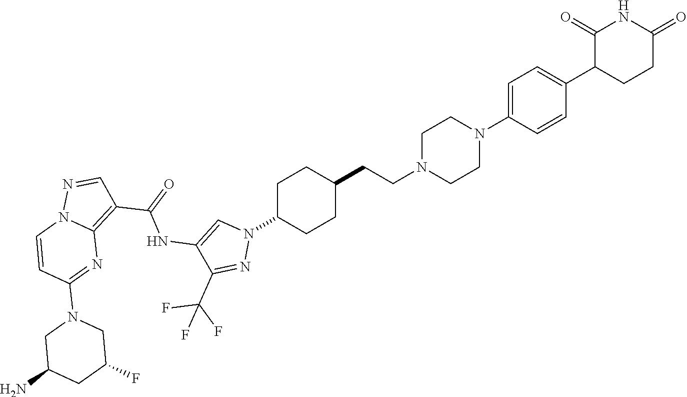 [SMILES: N[C@@H]1C[C@@H](F)CN(c2ccn3ncc(C(=O)Nc4cn([C@H]5CC[C@H](CCN6CCN(c7ccc(C8CCC(=O)NC8=O)cc7)CC6)CC5)nc4C(F)(F)F)c3n2)C1] |
| P-20 | C-12 | 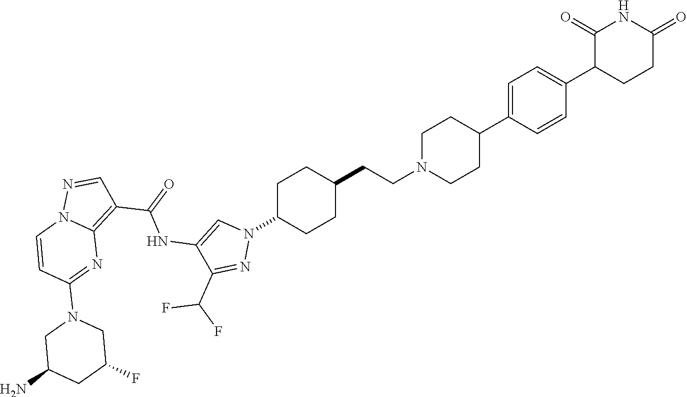 [SMILES: N[C@@H]1C[C@@H](F)CN(c2ccn3ncc(C(=O)Nc4cn([C@H]5CC[C@H](CCN6CCC(c7ccc(C8CCC(=O)NC8=O)cc7)CC6)CC5)nc4C(F)F)c3n2)C1] |
| P-21 | C-6 | 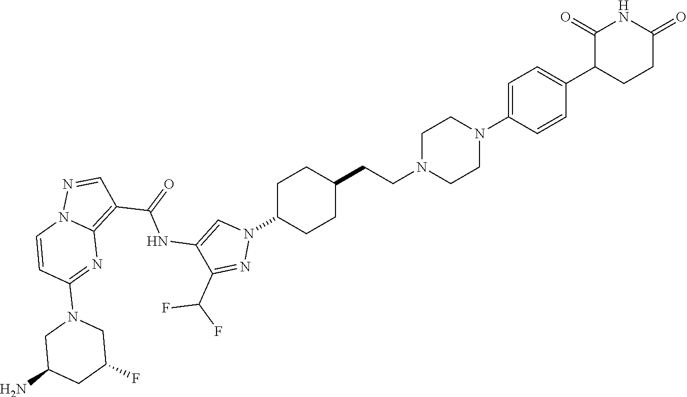 [SMILES: N[C@@H]1C[C@@H](F)CN(c2ccn3ncc(C(=O)Nc4cn([C@H]5CC[C@H](CCN6CCN(c7ccc(C8CCC(=O)NC8=O)cc7)CC6)CC5)nc4C(F)F)c3n2)C1] |
| P-22 | C-13 | 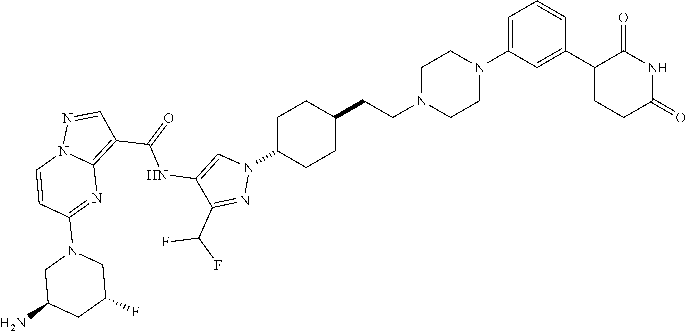 [SMILES: N[C@@H]1C[C@@H](F)CN(c2ccn3ncc(C(=O)Nc4cn([C@H]5CC[C@H](CCN6CCN(c7cccc(C8CCC(=O)NC8=O)c7)CC6)CC5)nc4C(F)F)c3n2)C1] |
| P-23 | C-14 | 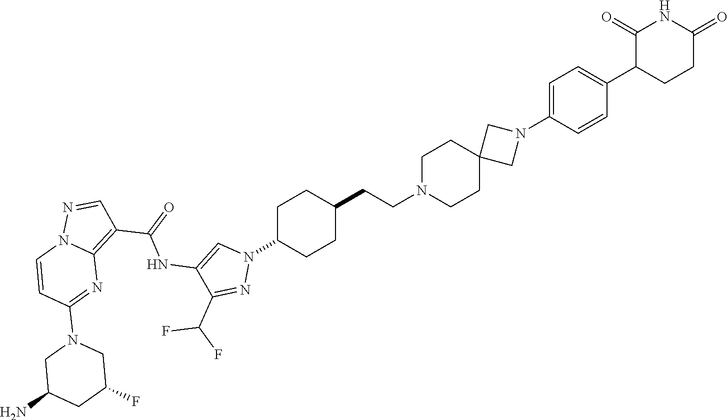 [SMILES: N[C@@H]1C[C@@H](F)CN(c2ccn3ncc(C(=O)Nc4cn([C@H]5CC[C@H](CCN6CCC7(CC6)CN(c6ccc(C8CCC(=O)NC8=O)cc6)C7)CC5)nc4C(F)F)c3n2)C1] |
| P-24 | C-5 | 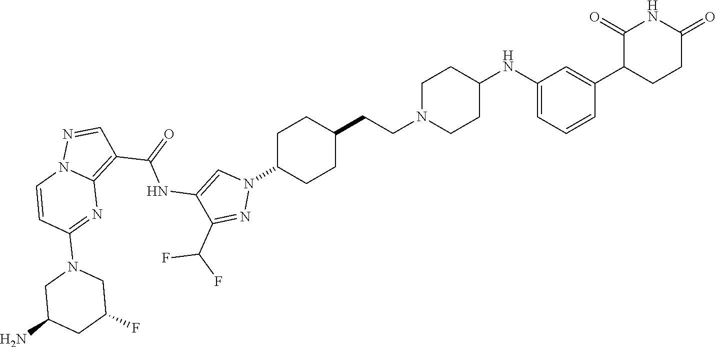 [SMILES: N[C@@H]1C[C@@H](F)CN(c2ccn3ncc(C(=O)Nc4cn([C@H]5CC[C@H](CCN6CCC(Nc7cccc(C8CCC(=O)NC8=O)c7)CC6)CC5)nc4C(F)F)c3n2)C1] |
| P-26 | C-11 | 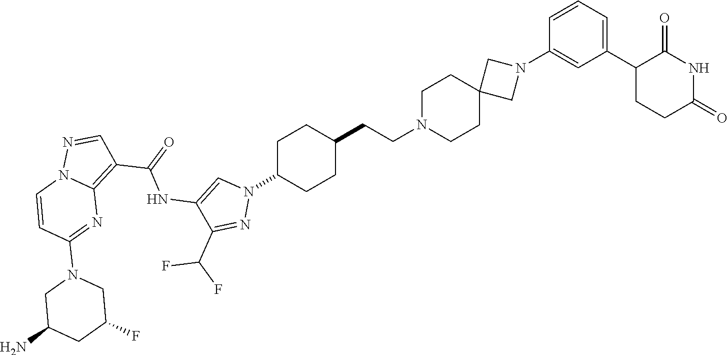 [SMILES: N[C@@H]1C[C@@H](F)CN(c2ccn3ncc(C(=O)Nc4cn([C@H]5CC[C@H](CCN6CCC7(CC6)CN(c6cccc(C8CCC(=O)NC8=O)c6)C7)CC5)nc4C(F)F)c3n2)C1] |
| P-28 | C-16i | 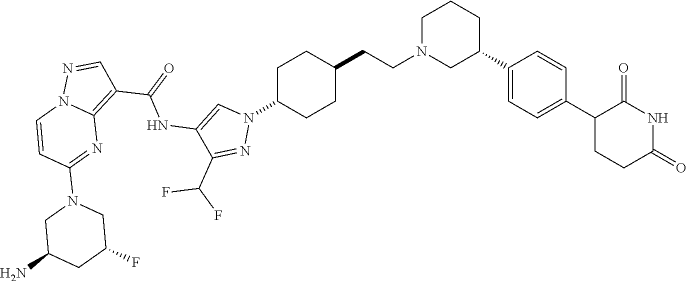 [SMILES: N[C@@H]1C[C@@H](F)CN(c2ccn3ncc(C(=O)Nc4cn([C@H]5CC[C@H](CCN6CCC[C@H](c7ccc(C8CCC(=O)NC8=O)cc7)C6)CC5)nc4C(F)F)c3n2)C1] |
| P-29 | C-16 | 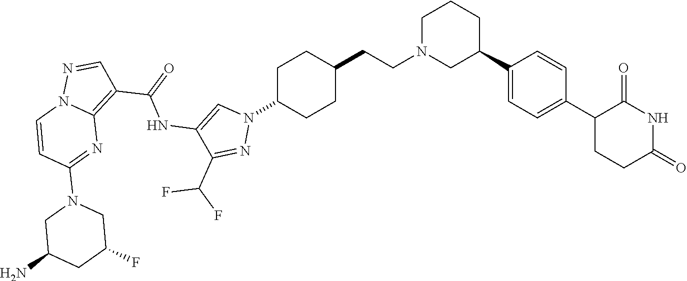 [SMILES: N[C@@H]1C[C@@H](F)CN(c2ccn3ncc(C(=O)Nc4cn([C@H]5CC[C@H](CCN6CCC[C@@H](c7ccc(C8CCC(=O)NC8=O)cc7)C6)CC5)nc4C(F)F)c3n2)C1] |

| Compound | |
|---|---|
| No. | Characterization Data |
| P-11 | 61% yield as a white solid. |
| | LCMS method 3: retention time: 1.917 min, 99% purity at 215 |
| | nm, [M − HCl + H]⁺ = 794.4, [M − HCl + 2H]²⁺ = 397.8. |
| | ¹H NMR (400 MHz, DMSO-d6) δ ppm 1.09-1.28 (m, 2 H), |
| | 1.37-1.50 (m, 1 H), 1.64-2.23 (m, 11 H), 2.34-2.48 (m, 2 H), |
| | 2.59-2.72 (m, 1 H), 3.03-3.25 (m, 6 H), 3.26-3.34 (m, 1 H), |
| | 3.41-3.67 (m, 4 H), 3.71-3.90 (m, 3 H), 4.25-4.38 (m, 1 H), |
| | 4.47-4.97 (m, 2 H), 5.12 (d, J = 46.0 Hz, 1 H), 6.92 (d, J = 7.8 |
| | Hz, 1 H), 6.97 (d, J = 8.8 Hz, 2 H), 7.12 (d, J = 8.6 Hz, 2 H), |
| | 8.28-8.53 (m, 4 H), 8.94 (d, J = 7.8 Hz, 1 H), 9.23 (s, 1 H), |
| | 10.40 (br s, 1 H), 10.79 (s, 1 H) |
| | ¹⁹F NMR (377 MHz, DMSO-d6) δ ppm −184.93 (s, 1 F), 58.81 |
| | (s, 3 F). |
| P-20 | 81% yield as a white solid. |
| | LCMS method 3: retention time: 1.909 min, 99% purity at 215 |
| | nm, [M − 2HCl + 2H]²⁺ = 388.3; [M − 2HCl + H]⁺ = 775.4. |
| | ¹H NMR (400 MHz, DMSO-d6) 8 1.10-1.26 (m, 2 H), 1.35- |
| | 1.49 (m, 1 H), 1.58-1.91 (m, 6 H), 1.96-2.09 (m, 7 H), 2.12- |
| | 2.24 (m, 1 H), 2.34-2.48 (m, 2 H), 2.60-2.74 (m, 1 H), 2.77- |
| | 2.88 (m, 1 H), 2.94-3.17 (m, 4 H), 3.26-3.62 (m, 6 H), 3.84 |
| | (dd, J = 11.5, 4.9 Hz, 1 H), 4.18-4.29 (m, 1 H), 4.48-4.96 (m, |
| | 2 H), 5.10 (d, J = 46.2 Hz, 1 H), 6.90 (d, J = 8.1 Hz, 1 H), 6.97- |
| | 7.30 (m, 5 H), 8.26-8.41 (m, 5 H), 8.94 (d, J = 7.8 Hz, 1 H), |
| | 9.33 (s, 1 H), 9.99-10.16 (m, 1 H), 10.83 (s, 1 H). |
| | ¹⁹F NMR (377 MHz, DMSO-d6) δ ppm −184.63 (s, 1 F), −111.23 |
| | (s, 2 F). |
| P-21 | 31% yield as a white solid. |
| | LCMS method 3: retention time: 1.403 min, 99% purity at 215 |
| | nm, [M − HCOOH + 2H]²⁺ = 388.7; [M − HCOOH + H]⁺ = 776.5. |
| | ¹H NMR (400 MHz, DMSO-d6) δ ppm 1.02-1.24 (m, 2 H), |
| | 1.32-1.49 (m, 3 H), 1.54-1.81 (m, 3 H), 1.83-1.94 (m, 2 H), |
| | 1.97-2.25 (m, 5 H), 2.30-2.40 (m, 2 H), 2.41-2.49 (m, 2 H), |
| | 2.58-2.70 (m, 1 H), 2.86-2.97 (m, 1 H), 2.99-3.07 (m, 1 H), |
| | 3.08-3.16 (m, 4 H), 3.21-3.54 (m, 4 H), 3.72 (dd, J = 10.9, 5.0 |
| | Hz, 1 H), 4.13-4.24 (m, 1 H), 4.26-4.88 (m, 2 H), 5.01 (d, J = |
| | 47.0 Hz, 1 H), 6.80-6.96 (m, 3 H), 6.98-7.31 (m, 3 H), 8.20 (s, |
| | 1 H), 8.27 (s, 1 H), 8.38 (s, 1 H), 8.81 (d, J = 7.8 Hz, 1 H), 9.33 |
| | (s, 1 H), 10.77 (s, 1 H). |
| | ¹⁹F NMR (377 MHz, DMSO-d6) δ ppm −182.92 (s, 1 F), −111.03 |
| | (s, 2 F). |
| P-22 | 35% yield as a light yellow solid. |
| | LCMS method 3: retention time: 1.869 min, 99% purity at 215 |
| | nm, [M − 2HCl + H]⁺ = 776.4, [M − 2HCl + 2H]²⁺ = 388.7. |
| | ¹H NMR (400 MHz, DMSO-d6) δ ppm 1.11-1.26 (m, 2 H), |
| | 1.36-1.50 (m, 1 H), 1.65-1.84 (m, 4 H), 1.85-1.94 (m, 2 H), |
| | 1.96-2.14 (m, 4 H), 2.15-2.30 (m, 1 H), 2.31-2.48 (m, 2 H), |
| | 2.59-2.72 (m, 1 H), 3.04-3.23 (m, 6 H), 3.27-3.63 (m, 5 H), |
| | 3.75-3.86 (m, 3 H), 4.18-4.29 (m, 1 H), 4.58-4.70 (m, 1 H), |
| | 4.80-4.88 (m, 1 H), 5.01-5.18 (m, 1 H), 6.72 (d, J = 7.6 Hz, 1 |
| | H), 6.86-6.94 (m, 3 H), 6.99-7.30 (m, 2 H), 8.33 (s, 1 H), 8.36- |
| | 8.52 (m, 4 H), 8.93 (d, J = 8.1 Hz, 1 H), 9.33 (s, 1 H), 10.70- |
| | 10.86 (m, 2 H).-One proton partially obscured by the the water |
| | peak in ¹H NMR. |
| | ¹⁹F NMR (377 MHz, DMSO-d6) δ ppm −184.85 |
| | (s, 1 F), −111.00 (s, 2 F). |
| P-23 | 44% yield as a white solid. |
| | LCMS method 3: retention time: 1.926 min, 99% purity at 215 |
| | nm, [M − HCOOH + H]⁺ = 816.5. |
| | ¹H NMR (400 MHz, DMSO-d6) δ ppm 1.04-1.20 (m, 2 H), |
| | 1.29-1.43 (m, 3 H), 1.56-1.78 (m, 6 H), 1.82-1.93 (m, 2 H), |
| | 1.94-2.24 (m, 5 H), 2.28-2.41 (m, 5 H), 2.41-2.48 (m, 1 H), |
| | 2.55-2.70 (m, 1 H), 2.85-2.98 (m, 1 H), 3.03-3.10 (m, 1 H), |
| | 3.30-3.42 (m, 5 H), 3.48-3.52 (m, 4 H), 3.69 (dd, J = 10.9, 5.0 |
| | Hz, 1 H), 4.13-4.24 (m, 1 H), 4.29-4.55 (m, 1 H), 4.60-4.85 |
| | (m, 1 H), 5.01 (d, J = 48.0 Hz, 1 H), 6.38 (d, J = 8.6 Hz, 2 H), |
| | 6.92 (d, J = 8.1 Hz, 1 H), 6.95-7.31 (m, 3 H), 8.19 (s, 1 H), 8.28 |
| | (s, 1 H), 8.37 (s, 1 H), 8.81 (d, J = 8.1 Hz, 1 H), 9.33 (s, 1 H), |
| | 10.74 (s, 1 H).-One proton non apparent in ¹H NMR. |
| | ¹⁹F NMR (377 MHz, DMSO-d6) δ ppm −183.46 (s, 1 F), −110.98 |
| | (s, 2 F). |
| P-24 | 65% yield as a white solid. |
| | LCMS method 3: retention time: 1.902 min, 99% purity at 215 |
| | nm, [M − 2HCl + H]⁺ = 790.4, [M − 2HCl + 2H]²⁺ = 395.7. |
| | ¹H NMR (400 MHz, DMSO-d6) δ ppm 1.05-1.27 (m, 2 H), |
| | 1.31-1.52 (m, 1 H), 1.56-1.70 (m, 2 H), 1.70-1.82 (m, 2 H), |
| | 1.82-1.99 (m, 5 H), 2.00-2.25 (m, 7 H), 2.34-2.47 (m, 1 H), |
| | 2.59-2.76 (m, 1 H), 2.85-3.19 (m, 4 H), 3.20-3.43 (m, 4 H), |
| | 3.44-3.66 (m, 3 H), 3.66-3.93 (m, 1 H), 4.15-4.32 (m, 1 H), |
| | 4.55-4.74 (m, 1 H), 4.77-4.97 (m, 1 H), 5.10 (d, J = 45.8 Hz, 1 |
| | H), 6.52-6.97 (m, 4 H), 6.99-7.31 (m, 2 H), 8.27-8.40 (m, 2 |
| | H), 8.41-8.64 (m, 3 H), 8.93 (d, J = 7.8 Hz, 1 H), 9.33 (s, 1 H), |
| | 10.36-10.70 (m, 1 H), 10.76-10.93 (m, 1 H). |
| | ¹⁹F NMR (377 MHz, DMSO-d6) δ ppm −184.60 (s, 1 F), −111.27 |
| | (s, 2 F). |
| P-26 | 56% yield as a white solid. |
| | LCMS method 3: retention time: 1.940 min, 98% purity at 215 |
| | nm, [M − HCOOH + H]⁺ = 816.5, [M − HCOOH + 2H]²⁺ = 408.8. |
| | ¹H NMR (400 MHz, DMSO-d6) δ ppm 1.03-1.22 (m, 2 H), |
| | 1.28-1.48 (m, 3 H), 1.63-1.80 (m, 7 H), 1.82-1.93 (m, 2 H), |
| | 1.96-2.08 (m, 3 H), 2.10-2.27 (m, 2 H), 2.34-2.44 (m, 5 H), |
| | 2.46-2.48 (m, 1 H), 2.56-2.72 (m, 1 H), 2.90-3.06 (m, 1 H), |
| | 3.07-3.20 (m, 1 H), 3.26-3.47 (m, 2 H), 3.47-3.58 (m, 4 H), |
| | 3.73 (dd, J = 10.8, 5.0 Hz, 1 H), 4.12-4.26 (m, 1 H), 4.32-4.59 |
| | (m, 1 H), 4.64-4.91 (m, 1 H), 5.03 (d, J = 46.6 Hz, 1 H), 6.25 |
| | (s, 1 H), 6.31 (d, J = 8.1 Hz, 1 H), 6.49 (d, J = 7.6 Hz, 1 H), 6.92 |
| | (d, J = 7.9 Hz, 1 H), 6.97-7.44 (m, 2 H), 8.19 (s, 1 H), 8.29 (s, 1 |
| | H), 8.37 (s, 1 H), 8.83 (d, J = 7.9 Hz, 1 H), 9.33 (s, 1 H), 10.79 |
| | (br s, 1 H).-Three protons not apparent in ¹H NMR spectrum. |
| | ¹⁹F NMR (377 MHz, DMSO-d6) δ ppm −183.33 |
| | (s, 1 F), −111.05 (s, 2 F). |
| P-28 | 38% yield as a light yellow solid. |
| | LCMS method 3: retention time: 1.884 min, 99% purity at 215 |
| | nm, [M − 2HCl + H]⁺ = 775.4, [M − 2HCl + 2H]²⁺ = 388.2. |
| | ¹H NMR (400 MHz, DMSO-d6) δ ppm 1.08-1.29 (m, 3 H), |
| | 1.31-1.45 (m, 1 H), 1.57-1.81 (m, 6 H), 1.81-1.98 (m, 6 H), |
| | 2.00-2.08 (m, 3 H), 2.14-2.26 (m, 1 H), 2.35-2.40 (m, 1 H), |
| | 2.61-2.72 (m, 1 H), 2.85-2.99 (m, 1 H), 3.01-3.18 (m, 4 H), |
| | 3.24-3.34 (m, 2 H), 3.37-3.42 (m, 2 H), 3.85 (dd, J = 11.5, 4.9 |
| | Hz, 1 H), 4.15-4.30 (m, 1 H), 4.42-4.97 (m, 2 H), 5.10 (d, J = |
| | 46.3 Hz, 1 H), 6.88 (d, J = 7.8 Hz, 1 H), 6.97-7.28 (m, 5 H), |
| | 8.13-8.28 (m, 3 H), 8.32 (s, 1 H), 8.38 (s, 1 H), 8.94 (d, J = 8.1 |
| | Hz, 1 H), 9.33 (s, 1 H), 9.74-10.09 (m, 1 H), 10.84 (s, 1 H). |
| | ¹⁹F NMR (377 MHz, DMSO-d6) δ ppm −186.81 −182.77 (m, 1 |
| | F), −111.53-−110.92 (m, 2 F). |
| P-29 | 55% yield as an off-white solid. |
| | LCMS method 4: retention time: 1.885 min, 99% purity at 215 |
| | nm, [M − 2HCl + H]⁺ = 775.4, [M − 2HCl + 2H]²⁺ = 388.3. |
| | ¹H NMR (400 MHz, DMSO-d6) δ ppm 1.09-1.29 (m, 2 H), |
| | 1.32-1.46 (m, 1 H), 1.59-2.12 (m, 14 H), 2.13-2.27 (m, 1 H), |
| | 2.35-2.48 (m, 1 H), 2.61-2.73 (m, 1 H), 2.85-2.98 (m, 1 H), |
| | 3.00-3.23 (m, 4 H), 3.24-3.43 (m, 3 H), 3.44-3.58 (m, 3 H), |
| | 3.85 (dd, J = 11.5, 4.9 Hz, 1 H), 4.16-4.26 (m, 1 H), 4.58-4.71 |
| | (m, 1 H), 4.77-4.98 (m, 1 H), 5.10 (d, J = 48.0 Hz, 1 H), 6.91 |
| | (d, J = 8.1 Hz, 1 H), 6.98-7.29 (m, 5 H), 8.32 (s, 1 H), 8.35- |
| | 8.49 (m, 4 H), 8.93 (d, J = 7.8 Hz, 1 H), 9.32 (s, 1 H), 10.31- |
| | 10.58 (m, 1 H), 10.84 (s, 1 H). |
| | ¹⁹F NMR (377 MHz, DMSO-d6) δ ppm −184.65 (s, 1 F), −111.26 |
| | (s, 2 F). |

### Final Product Method 4

### Example S13. Synthesis of P-27

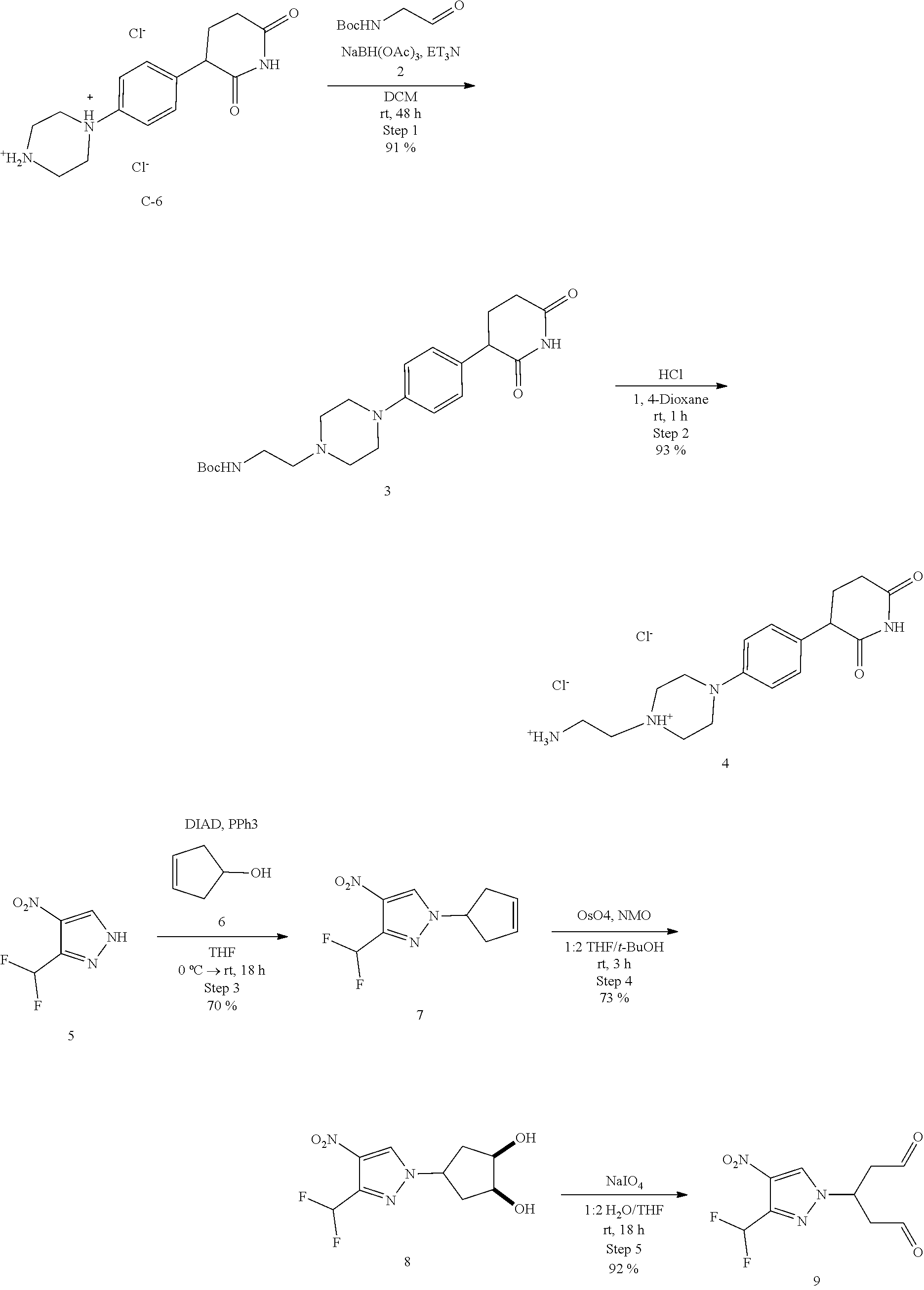

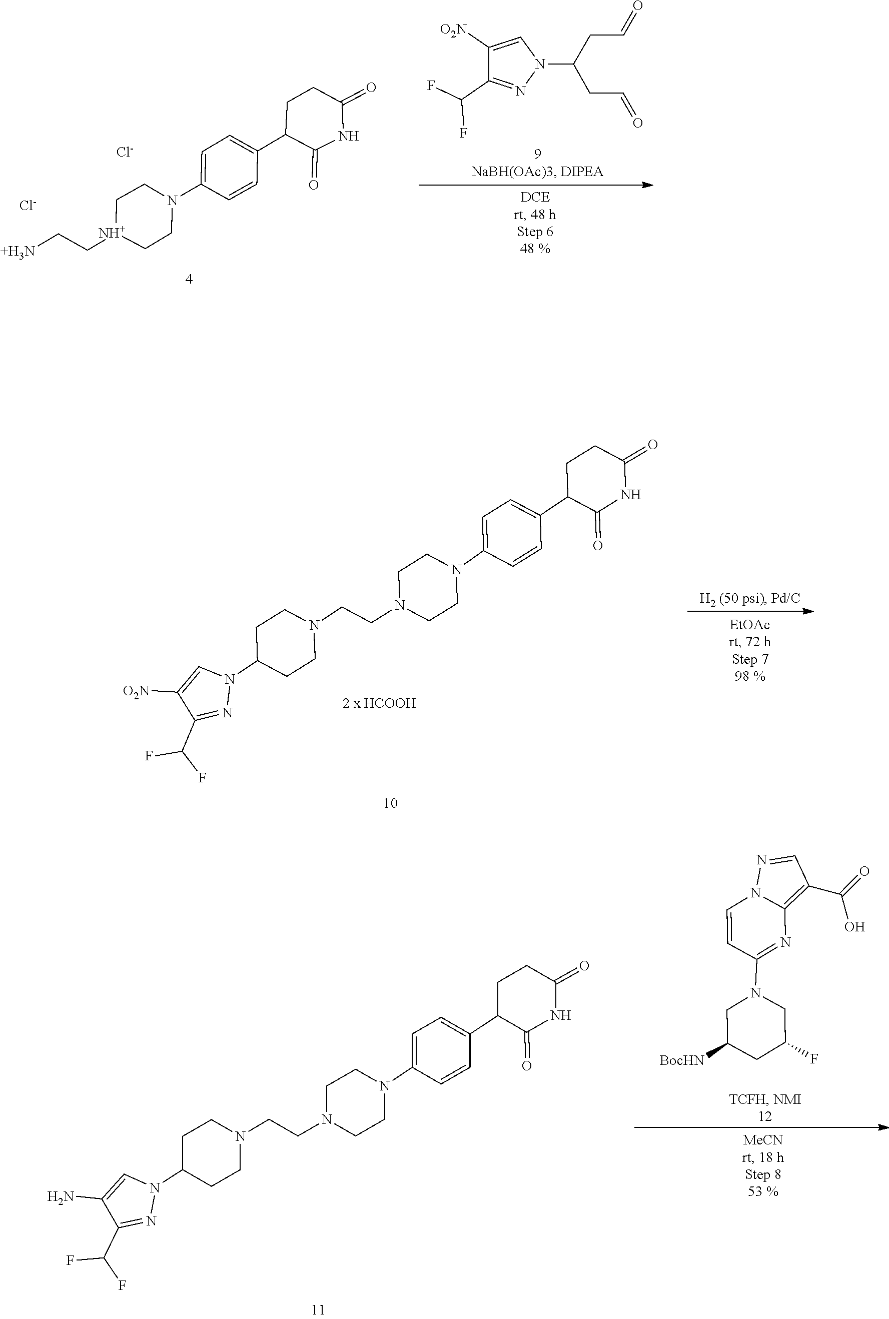

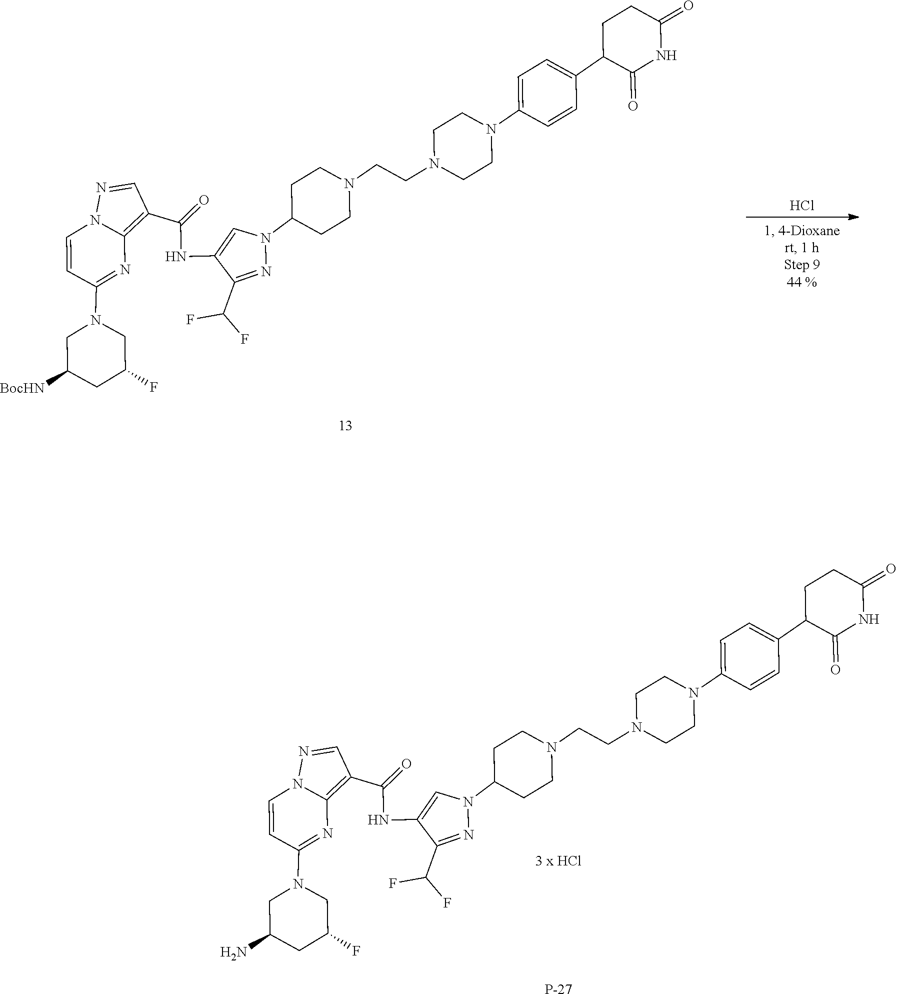

Step 1. Preparation of tert-Butyl N-[2-[4-[4-(2,6-dioxo-3-piperidyl)phenyl]piperazin-1-yl]ethyl]carbamate (3). 3-(4-piperazine-1,4-diium-1-ylphenyl) piperidine-2,6-dione dichloride C-6 (1.0 g, 2.89 mmol, 1.0 equiv) was added to DCM (25 mL) followed by triethylamine (2.42 mL, 17.3 mmol, 6.0 equiv), then the mixture was stirred until a clear solution was obtained. tert-butyl N-(2-oxoethyl) carbamate 2 (532.3 mg, 3.18 mmol, 1.1 equiv) was added, then the reaction mixture was stirred at room temperature for 30 minutes, after which sodium triacetoxyborohydride (757.2 mg, 3.47 mmol, 1.2 equiv) was added in one portion, and the stirring was continued for 24 hours under nitrogen atmosphere. A second portion of tert-butyl N-(2-oxoethyl) carbamate 2 (532.3 mg, 3.18 mmol, 1.1 equiv) and sodium triacetoxyborohydride (757.2 mg, 3.47 mmol, 1.2 equiv) were added to the reaction mixture and stirring was continued for an additional 24 hours under nitrogen atmosphere. The reaction mixture was quenched with water (25 mL), the phases were separated, and the aqueous phase was extracted with DCM (3×25 mL). The organic layers were washed with brine (25 mL), dried over MgSO₄ and evaporated to dryness. The crude was dry loaded on silica and purified by normal phase flash chromatography (DCM/MeOH, 100:0 to 90:10, 120 g cartridge, 15 CV, λ=254-280 nm, product with 5-8% MeOH) to afford 3 (product also contained the hemiaminal adduct between product and aldehyde starting material, which is suitable as starting material for the next step; 1.37 g, 2.63 mmol, 91% yield) as a white solid.

LCMS method 1: 80.1% purity at 215 nm, [M+H]⁺=417.4 (hemiaminal by-product: [M+H]⁺=576.4).

¹H NMR (400 MHz, DMSO-d6) δ ppm 1.04 (d, J=6.0 Hz, 1H), 1.37 (s, 9H), 1.93-2.06 (m, 1H), 2.06-2.20 (m, 1H), 2.36 (br t, J=6.6 Hz, 2H), 2.42-2.48 (m, 1H), 2.52-2.55 (m, 2H), 2.55-2.72 (m, 2H), 2.95-3.21 (m, 6H), 3.72 (br dd, J=10.9, 4.8 Hz, 1H), 6.55-6.75 (m, 1H), 6.88 (br d, J=8.7 Hz, 2H), 7.04 (br d, J=8.6 Hz, 2H), 10.77 (s, 1H).

Step 2. Preparation of 2-[4-[4-(2,6-Dioxo-3-piperidyl)phenyl]piperazin-1-ium-1-yl]ethylammonium dichloride (4). HCl 4.0 M in 1,4-dioxane (24.1 mL, 96.3 mmol, 50.0 equiv) was added to tert-butyl N-[2-[4-[4-(2,6-dioxo-3-piperidyl)phenyl]piperazin-1-yl]ethyl]carbamate 3 (1.37 g, 2.63 mmol, 1.0 equiv) and the mixture was stirred for 1 hour. The volatiles were evaporated under reduced pressure to yield the crude product as an off-white solid. The crude was dissolved in the minimum amount of water and purified by reversed-phase flash chromatography (MeCN in 0.02 M HCl(aq), 0% (5 CV)→30%, 120 g Claricep™ Spherical AQ C18 Column, 15 CV, λ=214-254 nm, first broad band with 5-9% MeCN and second sharper band with 9-11% MeCN, both containing only desired product). After evaporation of the fractions to dryness, co-evaporation of the residue with water (3×25 mL) to remove all the residual HCl, and lyophilisation, 4 (1.13 g, 2.45 mmol, 93% yield) was isolated as a tan solid. LCMS method 4:84.3% purity at 215 nm, [M−2HCl+H]⁺=317.2.

¹H NMR (400 MHz, DMSO-d6) δ ppm 1.94-2.04 (m, 1H), 2.07-2.22 (m, 1H), 2.40-2.49 (m, 1H), 2.57-2.72 (m, 1H), 3.20 (br s, 4H), 3.31-3.39 (m, 3H), 3.41-3.51 (m, 3H), 3.67 (br s, 3H), 6.98 (d, J=8.8 Hz, 2H), 7.11 (d, J=8.7 Hz, 2H), 8.49 (br s, 3H), 10.78 (s, 1H), 11.37 (br s, 1H).

Step 3. Preparation of 1-cyclopent-3-en-1-yl-3-(difluoromethyl)-4-nitro-pyrazole (7). 3-(difluoromethyl)-4-nitro-1H-pyrazole 5 (750.0 mg, 4.60 mmol, 1.0 equiv), cyclopent-3-en-1-ol 6 (464.2 mg, 5.52 mmol, 1.2 equiv), and triphenylphosphine (1.81 g, 6.90 mmol, 1.5 equiv) were dissolved in THF (9 mL). The mixture was cooled to 0° C., then diisopropyl azodicarboxylate (1.35 mL, 6.90 mmol, 1.5 equiv) was added dropwise. The reaction mixture was stirred at 0° C. for one hour then at room temperature overnight under nitrogen atmosphere. The volatiles were evaporated under reduced pressure and the residue was purified by normal phase flash chromatography (Heptanes/EtOAc, 100:0 to 75:25, 80 g RediSep Rf Gold® Normal-Phase Silica, 15 CV, λ=254-280 nm, undesired minor regioisomer with 8-9% EtOAc, desired major regioisomer with 17-21% EtOAc) to afford 7 (755 mg, 3.23 mmol, 70% yield) as an orange oil (the minor regioisomer was not isolated).

LCMS method 1: 98.0% purity at 215 nm, [M+H]⁺=230.1.

¹H NMR (400 MHz, DMSO-d6) δ ppm 2.71 (dd, J=15.6, 4.5 Hz, 2H), 2.91 (dd, J=15.7, 8.4 Hz, 2H), 5.19 (tt, J=8.4, 4.5 Hz, 1H), 5.78 (m, 2H), 7.30 (t, J=52.9 Hz, 1H), 9.03 (s, 1H).

¹⁹F NMR (377 MHz, DMSO-d6) δ ppm −117.35 (d, J=52.9 Hz, 2F).

Step 4. Preparation of (1s,2r)-4-[3-(Difluoromethyl)-4-nitro-pyrazol-1-yl]cyclopentane-1,2-diol (8). Osmium tetroxide 4% w/w in H₂O (205 μL, 0.032 mmol, 0.01 equiv) was added to a solution of 1-cyclopent-3-en-1-yl-3-(difluoromethyl)-4-nitro-pyrazole 7 (755.0 mg, 3.23 mmol, 1.0 equiv) and NMO (467.9 mg, 3.87 mmol, 1.2 equiv) in THF (5 mL) and tert-butanol (10 mL), then the reaction mixture was stirred at room temperature for 3 hours. The mixture was evaporated to dryness under reduced pressure, then the residue was dissolved in DCM, dry loaded on silica and purified by normal phase flash chromatography (DCM/MeOH, 100:0 to 90:10, 80 g RediSep Rf Gold® Normal-Phase Silica, 15 CV, λ=254-280 nm, product with 6-7% MeOH) to afford 8 (620 mg, 2.35 mmol, 73% yield) as an orange oil.

LCMS method 1: 99.9% purity at 215 nm, [M+H]⁺=264.2.

¹H NMR (400 MHz, DMSO-d6) δ ppm 1.97-2.09 (m, 2H), 2.10-2.20 (m, 2H), 4.09-4.18 (m, 2H), 4.70 (d, J=4.2 Hz, 2H), 5.06 (tt, J=8.8, 6.2 Hz, 1H), 7.30 (t, J=53.0 Hz, 1H), 9.08 (s, 1H).

¹⁹F NMR (377 MHz, DMSO-d6) δ ppm −117.34 (d, J=53.0 Hz, 2F).

Step 5. Preparation of 3-[3-(Difluoromethyl)-4-nitro-pyrazol-1-yl]pentanedial (9). Sodium periodate (604.0 mg, 2.82 mmol, 1.2 equiv) was added to a solution of (1s,2r)-4-[3-(difluoromethyl)-4-nitro-pyrazol-1-yl]cyclopentane-1,2-diol 8 (620.0 mg, 2.35 mmol, 1.0 equiv) in THF (8 mL) and water (4 mL), then the reaction mixture was stirred overnight. The solvents were evaporated to dryness, then the residue was taken up in DCM (15 mL) and dried over MgSO₄ overnight to dehydrate the product. The reaction mixture was filtered and the volatiles evaporated to dryness under reduced pressure to yield 9 (20:80 dialdehyde/cyclic monohydrate; 598 mg, 2.17 mmol, 92% yield) as a yellow sticky solid.

LCMS method 1: 99.9% purity at 215 nm, [M+H]⁺=262.1.

¹H NMR (400 MHz, DMSO-d6) δ ppm 1.81-2.46 (m, 2H), 3.03-3.26 (m, 2H), 4.59-5.15 (m, 1H), 5.19-5.69 (m, 1H), 7.08-7.56 (m, 1H), 8.97-9.30 (m, 1H), 9.59 (s, 1H)

¹⁹F NMR (377 MHz, DMSO-d6) δ ppm −117.84-−117.23 (m, 2F).

Step 6. Preparation of 3-[4-[4-[2-[4-[3-(Difluoromethyl)-4-nitro-pyrazol-1-yl]piperidin-1-ium-1-yl]ethyl]piperazin-4-ium-1-yl]phenyl]piperidine-2,6-dione diformate (10). 2-[4-[4-(2,6-dioxo-3-piperidyl)phenyl]piperazin-1-ium-1-yl]ethylammonium dichloride 4 (729.9 mg, 1.63 mmol, 1.0 equiv) was suspended in DCE (30 mL) under nitrogen atmosphere, then DIPEA (2.27 mL, 13.0 mmol, 8.0 equiv) was added and the mixture was sonicated for 20 minutes to break down the suspension and help the partial dissolution of the starting material. 3-[3-(difluoromethyl)-4-nitro-pyrazol-1-yl]pentanedial 9 (80% monohydrate, 540 mg, 1.96 mmol, 1.2 equiv) was added and the reaction mixture was stirred for 10 minutes. Sodium triacetoxyborohydride (1.04 g, 4.89 mmol, 3.0 equiv) was added in one portion and the reaction mixture was stirred overnight under nitrogen atmosphere. Another portion of sodium triacetoxyborohydride (1.04 g, 4.89 mmol, 3.0 equiv) was added to the reaction mixture, which was stirred overnight under nitrogen atmosphere. The reaction mixture was quenched with water (30 mL) and extracted with DCM (3×30 mL). The combined organic layers were dried over MgSO₄ and concentrated under reduced pressure. The crude was purified by reversed-phase flash chromatography (MeCN in 0.1% HCOOH(aq), 5% (3 CV)→30%, 150 g RediSep Rf Gold® C18Aq, 20 CV, λ=214-254 nm, product with 18-21% MeCN) to afford, after evaporation of the fractions and lyophilisation, 10 (540 mg, 0.787 mmol, 48% yield) as a light orange solid as a bis formic acid salt.

LCMS method 1: 92.6% purity at 215 nm, [M−2HCOOH+H]⁺=546.2, [M-2HCOOH+2H]²⁺=273.6.

¹H NMR (400 MHz, DMSO-d6) δ ppm 1.74-2.28 (m, 10H), 2.52-2.82 (m, 8H), 3.04 (br s, 3H), 3.46-4.55 (m, 2H), 4.58-6.11 (m, 1H), 6.60-7.12 (m, 4H), 7.14-7.62 (m, 1H), 8.16 (br s, 2H), 9.08 (br s, 1H), 10.77 (br s, 1H).

¹⁹F NMR (377 MHz, DMSO-d6) δ ppm −117.44 (br d, J=53.1 Hz, 2F).

Step 7. Preparation of 3-[4-[4-[2-[4-[4-Amino-3-(difluoromethyl)pyrazol-1-yl]-1-piperidyl]ethyl]piperazin-1-yl]phenyl]piperidine-2,6-dione (11). 3-[4-[4-[2-[4-[3-(difluoromethyl)-4-nitro-pyrazol-1-yl]piperidin-1-ium-1-yl]ethyl]piperazin-4-ium-1-yl]phenyl]piperidine-2,6-dione diformate 10 (493.0 mg, 0.772 mmol, 1.0 equiv) was dissolved in ethyl acetate (40 mL), then the solution was placed in a hydrogenation reactor and degassed by bubbling nitrogen for 20 minutes. Pd/C 5% w/w (164.4 mg, 0.077 mmol, 0.1 equiv) was added, then the mixture was further degassed by bubbling nitrogen for 20 minutes. The reactor was pressurized with 50 psi of hydrogen, then the reaction mixture was stirred for 72 hours at room temperature. The reaction mixture was filtered through celite to remove the catalyst, then the celite was washed thoroughly with 1:1 MeOH/DCM (5×20 mL). The volatiles were evaporated to yield 11 (391 mg, 0.758 mmol, 98% yield) as a light orange solid.

LCMS method 1: 99.9% purity at 215 nm, [M+H]⁺=516.2, [M+2H]²⁺=258.8.

¹H NMR (400 MHz, DMSO-d6) δ ppm 1.76-1.96 (m, 4H), 1.97-2.18 (m, 4H), 2.40-2.47 (m, 2H), 2.47 (s, 2H), 2.53-2.58 (m, 4H), 2.58-2.71 (m, 1H), 2.96 (br d, J=11.6 Hz, 2H), 3.04-3.15 (m, 4H), 3.17 (d, J=4.8 Hz, 1H), 3.72 (dd, J=11.0, 4.9 Hz, 1H), 3.89-4.18 (m, 3H), 6.72-7.02 (m, 3H), 7.04 (br d, J=8.7 Hz, 2H), 7.17 (s, 1H), 10.77 (s, 1H). 19F NMR (377 MHz, DMSO-d6) δ ppm −111.35 (d, J=54.5 Hz, 2F).

Step 8. Preparation of tert-Butyl N-[(3R,5R)-1-[3-[[3-(difluoromethyl)-1-[1-[2-[4-[4-(2,6-dioxo-3-piperidyl)phenyl]piperazin-1-yl]ethyl]-4-piperidyl]pyrazol-4-yl]carbamoyl]pyrazolo[1,5-a]pyrimidin-5-yl]-5-fluoro-3-piperidyl]carbamate (13). TCFH (32.6 mg, 0.116 mmol, 1.2 equiv) was added in one portion to a solution of 5-[(3R,5R)-3-(tert-butoxycarbonylamino)-5-fluoro-1-piperidyl]pyrazolo[1,5-a]pyrimidine-3-carboxylic acid 12-intermediate 4 from synthesis of P-1-(36.8 mg, 0.097 mmol, 1.0 equiv), 3-[4-[4-[2-[4-[4-amino-3-(difluoromethyl)pyrazol-1-yl]-1-piperidyl]ethyl]piperazin-1-yl]phenyl]piperidine-2,6-dione 11 (50.0 mg, 0.097 mmol, 1.0 equiv) and NMI (27 μL, 0.339 mmol, 3.5 equiv) in MeCN (1.9 mL). The reaction mixture was then stirred at room temperature overnight. The volatiles were evaporated under reduced pressure, then the crude was dissolved in DMSO and injected directly into a column for reversed-phase flash chromatography purification (MeCN in 0.1% HCOOH(aq), 5% (3 CV)→50%, 50 g RediSep Rf Gold® C18Aq, 20 CV, λ=214-254 nm, product with 30-35% MeCN). Co-evaporation of the fractions with MeCN yielded 13 (45 mg, 0.051 mmol, 53% yield) as a white solid.

LCMS method 1: 99.9% purity at 215 nm, [M-t-Bu+H]⁺=821.0, [M+2H]²⁺=439.2, [M-t-Bu+2H]²⁺=411.2.

¹H NMR (400 MHz, DMSO-d6) δ ppm 1.34 (br s, 9H), 1.70-2.05 (m, 7H), 2.05-2.26 (m, 5H), 2.55-2.70 (m, 6H), 3.01 (br d, J=10.4 Hz, 4H), 3.11 (br s, 4H), 3.58-3.78 (m, 4H), 4.06-4.44 (m, 2H), 4.47-4.91 (m, 1H), 5.03 (d, J=46.2 Hz, 1H), 6.89 (br d, J=8.1 Hz, 3H), 6.98-7.33 (m, 4H), 8.29 (s, 1H), 8.35 (br s, 1H), 8.81 (br d, J=7.7 Hz, 1H), 9.31 (br s, 1H), 10.76 (s, 1H).

¹⁹F NMR (377 MHz, DMSO-d6) δ ppm −186.27-−181.60 (m, 1F), −114.92-−107.65 (m, 2F).

Step 9. Preparation of 5-((3R,5R)-3-Amino-5-fluoropiperidin-1-yl)-N-(3-(difluoromethyl)-1-(1-(2-(4-(4-(2,6-dioxopiperidin-3-yl)phenyl) piperazin-1-yl)ethyl) piperidin-4-yl)-1H-pyrazol-4-yl) pyrazolo[1,5-a]pyrimidine-3-carboxamide trihydrochloride (P-27). HCl 4.0 M in 1,4-dioxane (1.84 mL, 7.35 mmol, 150 equiv) was added to tert-butyl N-[(3R,5R)-1-[3-[[3-(difluoromethyl)-1-[1-[2-[4-[4-(2,6-dioxo-3-piperidyl)phenyl]piperazin-1-yl]ethyl]-4-piperidyl]pyrazol-4-yl]carbamoyl]pyrazolo[1,5-a]pyrimidin-5-yl]-5-fluoro-3-piperidyl]carbamate 13 (43.0 mg, 0.049 mmol, 1.0 equiv) and the mixture was sonicated for 30 minutes, then stirred for 1 hour. The solvent was evaporated to dryness and the residue was purified by reversed-phase flash chromatography (MeCN in 0.02 M HCl(aq), 5% (3 CV)→30%, 50 g RediSep Rf Gold® C18Aq, 20 CV, λ=214-254 nm, product with 17-20% MeCN, broad band). The fractions containing the product were evaporated to dryness, then co-evaporated with water (3×10 mL) to completely remove any trace of residual HCl, and the residue was freeze-dried overnight to yield P-27 (17.29 mg, 0.021 mmol, 44% yield) as a white solid as a trihydrochloride salt.

LCMS method 3:95.9% purity at 215 nm, [M-3HCl+H]⁺=777.5, [M-3HCl+2H]²⁺=389.3.

¹H NMR (400 MHz, DMSO-d6) δ ppm 1.88-2.04 (m, 2H), 2.05-2.23 (m, 2H), 2.27-2.39 (m, 4H), 2.39-2.43 (m, 1H), 2.44-2.47 (m, 1H), 2.59-2.72 (m, 1H), 3.06-3.27 (m, 6H), 3.27-3.44 (m, 4H), 3.44-3.51 (m, 2H), 3.67-3.72 (m, 2H), 3.74-3.80 (m, 2H), 3.84-3.96 (m, 3H), 4.47-4.74 (m, 2H), 4.75-5.00 (m, 1H), 5.10 (d, J=46.5 Hz, 1H), 6.90 (d, J=7.9 Hz, 1H), 7.00 (d, J=8.6 Hz, 2H), 7.05-7.41 (m, 3H), 8.16-8.42 (m, 4H), 8.47 (s, 1H), 8.94 (d, J=7.8 Hz, 1H), 9.37 (s, 1H), 10.54-11.05 (m, 3H).

¹⁹F NMR (377 MHz, DMSO-d6) δ ppm −184.62 (s, 1F), −113.10-−109.99 (m, 2F).

TABLE 8

| Compound | | |
|---|---|---|
| No. | CBM | Structure |
| P-27 | C-6 | 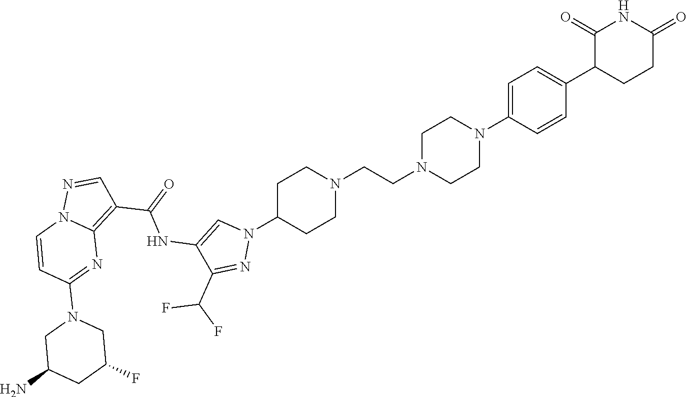 [SMILES: N[C@@H]1C[C@@H](F)CN(c2ccn3ncc(C(=O)Nc4cn(C5CCN(CCN6CCN(c7ccc(C8CCC(=O)NC8=O)cc7)CC6)CC5)nc4C(F)F)c3n2)C1] |
| | Compound | |
| | No. | Characterization Data |
| | P-27 | 44% yield as a white solid. |
| | | LCMS method 3: retention time: 1.647 min, 96% purity at |
| | | 215 nm, [M − 3HCl + H]⁺ = 777.5, [M − 3HCl + 2H]²⁺ = 389.3. |
| | | ¹H NMR (400 MHz, DMSO-d6) δ ppm 1.88-2.04 (m, 2 H), |
| | | 2.05-2.23 (m, 2 H), 2.27-2.39 (m, 4 H), 2.39-2.43 (m, 1 |
| | | H), 2.44-2.47 (m, 1 H), 2.59-2.72 (m, 1 H), 3.06-3.27 (m, |
| | | 6 H), 3.27-3.44 (m, 4 H), 3.44-3.51 (m, 2 H), 3.67-3.72 |
| | | (m, 2 H), 3.74-3.80 (m, 2 H), 3.84-3.96 (m, 3 H), 4.47- |
| | | 4.74 (m, 2 H), 4.75-5.00 (m, 1 H), 5.10 (d, J = 46.5 Hz, 1 H), |
| | | 6.90 (d, J = 7.9 Hz, 1 H), 7.00 (d, J = 8.6 Hz, 2 H), 7.05-7.41 |
| | | (m, 3 H), 8.16-8.42 (m, 4 H), 8.47 (s, 1 H), 8.94 (d, J = 7.8 |
| | | Hz, 1 H), 9.37 (s, 1 H), 10.54-11.05 (m, 3 H). |
| | | ¹⁹F NMR (377 MHz, DMSO-d6) δ ppm −184.62 |
| | | (s, 1 F), −113.10-−109.99 (m, 2 F). |

### BIOLOGICAL EXAMPLES

### Example B1. IRAK4 PhosphoSens Biochemical Assay (BIOCHEM) Procedure

The PhosphoSens® biochemical assay was performed as described by the vendor (AssayQuant Technologies Inc. Marlborough, MA). A 1.25× stock solution of IRAK4 (PV4002, ThermoFisher Scientific, Waltham, MA) and a 5× stock solution of ATP and Sox conjugated peptide substrate, AQT0326 (CSKS-AQT0326B, AssayQuant Technologies), were prepared in 1× kinase reaction buffer consisting of 50 mM HEPES, pH 7.5, 0.01% Brij-35, 0.5 mM EGTA, 20 mM MgCl₂ and 1 mM DTT. 10 μL of the ATP and substrate solution mix, for a final concentration of 200 μM ATP and 10 μM peptide substrate, was added to a Corning 3574 384-well, white, non-binding surface microtiter plate containing 0.5 μL of serially diluted test compounds prepared in DMSO. The reactions were started with the addition of 40 μL of the enzyme solution, for a final IRAK4 concentration of 1 nM, and monitored every 71 seconds for 240 minutes at λ_{EX} 360/λ_{EM} 485 in a BioTek Synergy H4 plate reader (Agilent Technologies, Santa Clara, CA) at room temperature. The initial linear portions of the progress curves were fit according to a linear equation to yield the slopes and converted to % inhibition based on a value of 100% activity for the no inhibitor treated control. IC₅₀ values of each compound were obtained by fitting the % inhibition-compound concentration curves using Dotmatics software (Dotmatics, Bishops Stortford, Hertfordshire, England).

### Example B2. Reagent Preparations

Cell culture media was prepared in a tissue culture hood in a sterile environment by adding 10% FBS and 1% Penicillin Streptomycin to 500 mL no phenol red RPMI 1640 media. The media was filtered through a Nalgene Bottle Top Filter and stored at 4° C.

The Cell titer Glo (CTG) buffer and substrate (CellTiter-Glo Luminescent Cell Viability Assay, Promega Ref. #G7573) were stored at −20° C. The CTG buffer (100 mL) was warmed in a bead bath and added to the CTG substrate bottle in a tissue culture hood. The solution was mixed with a pipette until it became homogenous. CTG reagent was aliquoted into 15 mL falcon tubes and stored at −20° C.

For Homogenous Time Resolved Fluorescence (HTRF) assays, a Cisbio HTRF kit was used, which included: Lysis Buffer #1 4×, Blocking Reagent #3 100×, 20× Antibody 1 (Anti-IRAK4 d2), 20× Antibody 2 (Anti-IRAK4 k), and Detection Buffer.

4× Lysis Buffer was stored at 4° C. For use as 1× Lysis buffer, the 4× solution was diluted with de-ionized water (distilled water, Gibco Cat. #15230279) and 100× Blocking Reagent in a 1:3:0.04 volume ratio.

20× Antibody Solution aliquots were stored at −80° C. and the Detection Buffer was stored at 4° C. For use as a 1× Antibody Solution, the 20× Antibody Solution aliquot was diluted with Detection Buffer in a 1:19 volume ratio.

### Example B3. Advanced Lipoxidation End Product THP1 Homogeneous Time Resolved Fluorescence (ALE THP1 HTRF) Procedure

Cells were lysed at room temperature for 45 min with shaking. A BCA protein assay was performed and normalization was conducted to the desired total protein concentration with 1× lysis buffer. Next, 1× Antibody Solutions were prepared by adding 380 μL Detection Buffer to 20 μL 20× Antibody Solution aliquots and mixing well. 1× Antibody Solutions were combined 1:1 and briefly vortexed. For control wells, 20 μL 1× anti IRAK4-k Antibody Solution was saved. The 384-well plate (ProxiPlate-384 Plus, Perkin Elmer Cat. #6008289) was loaded by adding 4 μL of the mixed Antibody Solution to empty wells using a single channel repeater. Using a multi-channel repeater, 16 μL lysate was added per well, and any bubbles that were formed were popped with 20 μL pipette tips and Kimwipe edges. In column 10, triplicates of each control were prepared. In wells A10, B10, and C10, buffer control was prepared by adding 16 μL Lysis Buffer and 4 μL Detection Buffer. In wells D10, E10, and F10, cryptate control was prepared by adding 16 μL Lysis Buffer, 2 μL Detection Buffer, and 2 μL 1× anti-IRAK4-k Antibody Solution. In wells G10, H10, and I10, a negative control was prepared by adding 16 μL Lysis Buffer and 4 μL mixed Antibody Solution. The plate was sealed with a clear seal and covered with an aluminum lid. The plate was spun down at 800 g for 5 min and incubated in the dark at room temperature overnight. The next day, the plate was spun down at 800 g for 5 min. The samples were analyzed by a plate reader (Envision, PerkinElmer) using the Desnor 384 HTRF program.

A summary of the ALE THP1 HTRF data for the tested compounds is provided in Table 9 below.

TABLE 9
BIOCHEM and ALE THP1 HTRF Results of the Compounds.

| Com- | | ALE | ALE |
|---|---|---|---|
| pound | BIOCHEM | THP1 HTRF | THP1 HTRF |
| No. | IC₅₀ (nM) | EC₅₀ (μM) | Y_{MIN} (%) |
| P-1 | 0.4 | 0.007 | 10 |
| P-2 | 0.3 | 0.096 | 68 |
| P-3 | 0.3 | 0.177 | 81 |
| P-4 | 0.2 | >10 | 32 |
| P-5 | >0.2 | 0.057 | 45 |
| P-6 | 0.2 | 0.017 | 15 |
| P-7 | 0.3 | >10 | 52 |
| P-8 | >0.2 | >10 | >95 |
| P-9 | 0.3 | >10 | 86 |
| P-10 | 0.6 | 0.964 | 20 |
| P-11 | 0.4 | 0.015 | 19 |
| P-12 | 0.2 | 6.864 | 57 |
| P-13 | >0.2 | 0.405 | 18 |
| P-14 | 0.2 | >10 | 64 |
| P-15 | >0.2 | 0.004 | 11 |
| P-16 | 0.2 | 0.028 | 18 |
| P-17 | >0.2 | >10 | 73 |
| P-18 | >0.2 | 9.182 | 13 |
| P-19 | 0.2 | 0.010 | 11 |
| P-20 | >0.2 | 0.011 | 12 |
| P-21 | >0.2 | 0.010 | 15 |
| P-22 | >0.2 | 3.012 | 43 |
| P-23 | >0.2 | 0.011 | 15 |
| P-24 | >0.2 | 0.012 | 12 |
| P-25 | >0.2 | 0.006 | 15 |
| P-26 | >0.2 | 0.015 | 7 |
| P-27 | >0.2 | 0.010 | 16 |
| P-28 | >0.2 | >10 | >95 |
| P-29 | >0.2 | >10 | >95 |
| P-30 | >0.2 | >10 | >95 |
| P-31 | >0.2 | >10 | >95 |
| P-32 | >0.2 | 0.005 | 21 |
| P-33 | >0.2 | 0.003 | 22 |
| P-34 | >0.2 | 0.005 | 13 |

Although the present invention has been described in some detail by way of illustration and example for purposes of clarity of understanding, the descriptions and examples should not be construed as limiting the scope of the invention. The disclosures of all patent and scientific literature cited herein are expressly incorporated herein in their entirety by reference.

1. A compound of Formula (I): 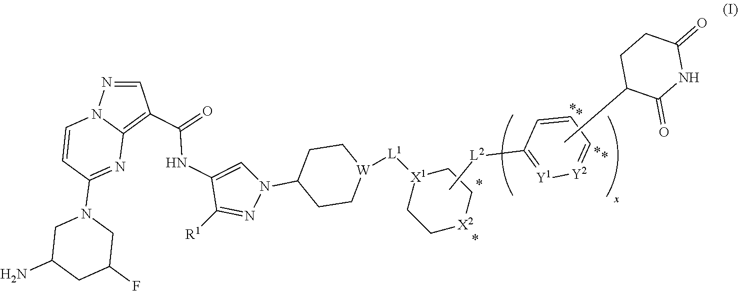
or a pharmaceutically acceptable salt thereof, wherein:
R¹ is C₁-C₆ haloalkyl;
W is CH or N;
X¹ is CH or N;
X² is CH₂, CH, C, N, or NH;
Y¹ and Y² are independently CH or N;
L¹ is —C(O)N(H)—, —C(O)—, or C₁-C₆ alkylene;
L² is a bond, —C(O)—, —NR²—, —CH₂N(R²)—, —CH₂—, or 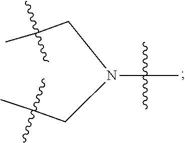 [SMILES: *CN(*)C*]
R² is H or C₁-C₆ alkyl;
x is 0 or 1;
* indicates an attachment point for L²; and
** indicates an attachment point for the piperidinyl-2,6-dione moiety.

2. The compound of claim 1, or a pharmaceutically acceptable salt thereof, wherein the compound is of Formula (IIa), (IIb), or (IIc):

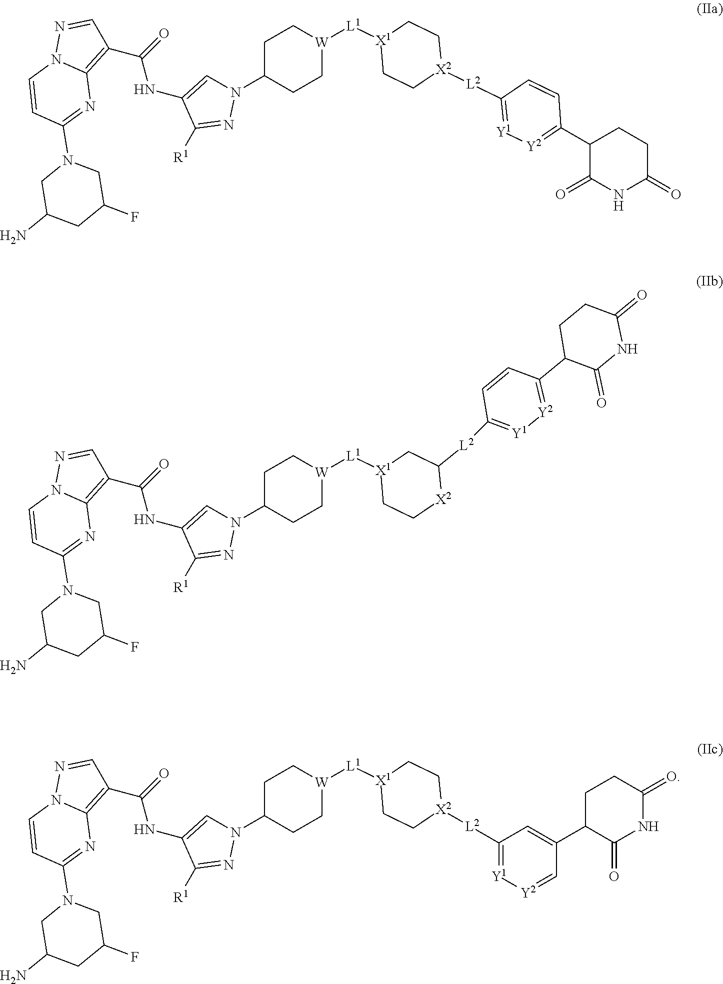

3. The compound of claim 1 or 2, or a pharmaceutically acceptable salt thereof, wherein:
R¹ is C₁-C₃ haloalkyl.

4. The compound of claim 3, or a pharmaceutically acceptable salt thereof, wherein:
R¹ is —CHF₂ or —CF₃.

5. The compound of any one of claims 1-4, or a pharmaceutically acceptable salt thereof, wherein:
W is CH.

6. The compound of any one of claims 1-5, or a pharmaceutically acceptable salt thereof, wherein:
X¹ is CH.

7. The compound of any one of claims 1-5, or a pharmaceutically acceptable salt thereof, wherein:
X¹ is N.

8. The compound of any one of claims 1-7, or a pharmaceutically acceptable salt thereof, wherein:
Y¹ is CH.

9. The compound of any one of claims 1-8, or a pharmaceutically acceptable salt thereof, wherein:
Y² is CH.

10. The compound of any one of claims 1-9, or a pharmaceutically acceptable salt thereof, wherein:
L¹ is —C(O)N(H)—, —C(O)—, or C₁-C₃ alkylene.

11. The compound of claim 10, or a pharmaceutically acceptable salt thereof, wherein:
L¹ is —C(O)N(H)—, —C(O)—, —CH₂—, or —CH₂CH₂—.

12. The compound of any one of claims 1-11, or a pharmaceutically acceptable salt thereof, wherein:
L² is a bond, —C(O)—, —NR²—, —CH₂N(R²)—, —CH₂—, or 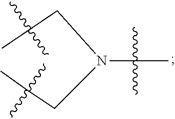 [SMILES: *CN(*)C*]
and
R² is H or C₁-C₃ alkyl.

13. The compound of claim 12, or a pharmaceutically acceptable salt thereof, wherein:
R² is H or —CH₃.

14. The compound of any one of claims 1-13, or a pharmaceutically acceptable salt thereof, wherein:

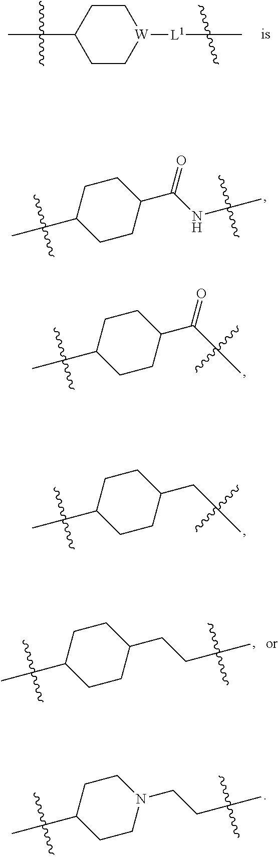

15. The compound of any one of claims 1-14, or a pharmaceutically acceptable salt thereof, wherein: 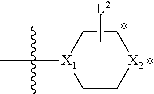
is

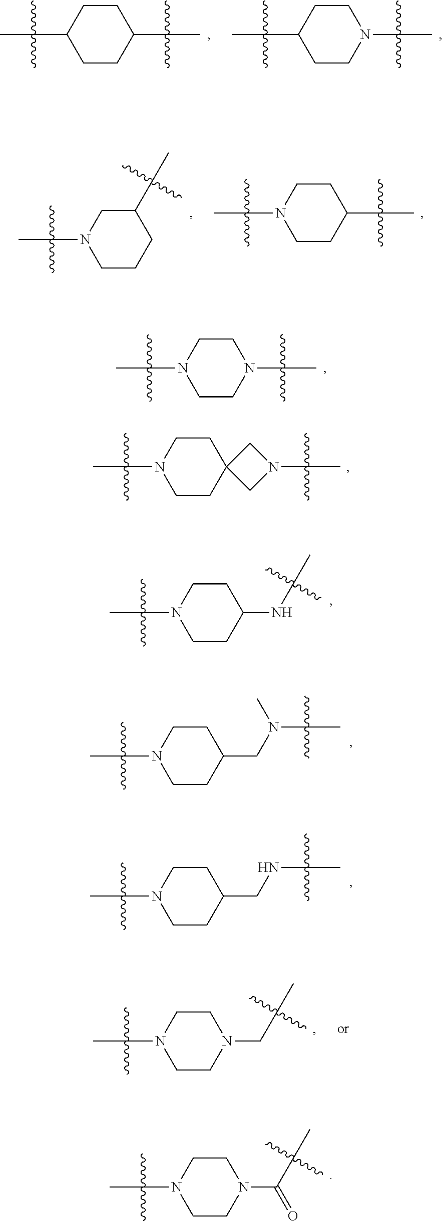

16. The compound of any one of claims 1-15, or a pharmaceutically acceptable salt thereof, wherein: 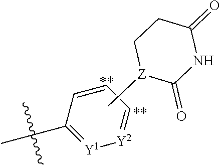
is

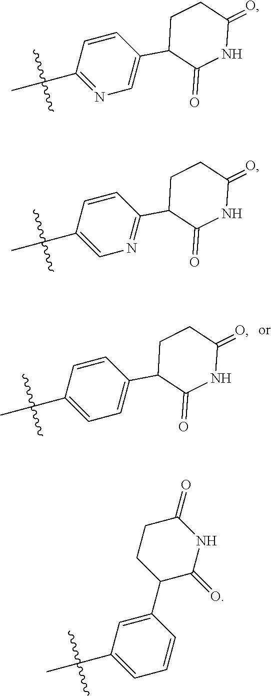 [SMILES: *c1ccc(C2CCC(=O)NC2=O)cn1 ; *c1ccc(C2CCC(=O)NC2=O)nc1 ; *c1ccc(C2CCC(=O)NC2=O)cc1 ; *c1cccc(C2CCC(=O)NC2=O)c1]

17. A compound selected from the compounds of Table 1 and pharmaceutically acceptable salts thereof.

18. A pharmaceutical composition comprising the compound of any one of claims 1-17, or a pharmaceutically acceptable salt thereof, and a pharmaceutically acceptable excipient.

19. A method of modulating interleukin-1 (IL1) receptor-associated kinase 4 (IRAK4) comprising contacting IRAK4 with an effective amount of the compound of any one of claims 1-17, or a pharmaceutically acceptable salt thereof, or the pharmaceutical composition of claim 18.

20. A method of treating an inflammatory or autoimmune disease in a subject in need thereof, comprising administering to the subject an effective amount of the compound of any one of claims 1-17, or a pharmaceutically acceptable salt thereof, or the pharmaceutical composition of claim 18, optionally wherein the inflammatory or autoimmune disease is atopic dermatitis, asthma, lupus, rheumatoid arthritis, familial mediterranean fever, psoriasis, generalized pustular psoriasis, cryoprin-associated periodic syndrome, hidradenitis suppurativa, Bechet's syndrome, or familial cold autoinflammatory syndrome.
